SEQUENCE LISTING (14 sequences)

SEQ ID NO: 1 - protein, 385 aa, Homo sapiens
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 2 - DNA, 1717 bp, Homo sapiens
  source 1..1717: mol_type = other DNA
aacgccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatggtaaaaaaacaaaacctaacactaaggatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaaaaaaaaaaaaaaa

SEQ ID NO: 3 - protein, 376 aa, Homo sapiens
MMMMALSKTFGQKPVKFQLEDDGEFYMIGSEVGNYLRMFRGSLYKRYPSLWRRLATVEERKKIVASSHDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 4 - DNA, 1690 bp, Homo sapiens
  source 1..1690: mol_type = other DNA
aacgccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatgatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaaaaaaaaaaaaaaa

SEQ ID NO: 5 - protein, 2492 aa, Homo sapiens
MTAEPMSESKLNTLVQKLHDFLAHSSEESEETSSPPRLAMNQNTDKISGSGSNSDMMENSKEEGTSSSEKSKSSGSSRSKRKPSIVTKYVESDDEKPLDDETVNEDASNENSENDITMQSLPKGTVIVQPEPVLNEDKDDFKGPEFRSRSKMKTENLKKRGEDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKKIKVDSEKSNKVYEHTSRFSPKKTSSNCNGEEKKLDDSCSGSVTYSYSALIVPKEMIKKAKKLIETTANMNSSYVKFLKQATDNSEISSATKLRQLKAFKSVLADIKKAHLALEEDLNSEFRAMDAVNKEKNTKEHKVIDAKFETKARKGEKPCALEKKDISKSEAKLSRKQVDSEHMHQNVPTEEQRTNKSTGGEHKKSDRKEEPQYEPANTSEDLDMDIVSVPSSVPEDIFENLETAMEVQSSVDHQGDGSSGTEQEVESSSVKLNISSKDNRGGIKSKTTAKVTKELYVKLTPVSLSNSPIKGADCQEVPQDKDGYKSCGLNPKLEKCGLGQENSDNEHLVENEVSLLLEESDLRRSPRVKTTPLRRPTETNPVTSNSDEECNETVKEKQKLSVPVRKKDKRNSSDSAIDNPKPNKLPKSKQSETVDQNSDSDEMLAILKEVSRMSHSSSSDTDINEIHTNHKTLYDLKTQAGKDDKGKRKRKSSTSGSDFDTKKGKSAKSSIISKKKRQTQSESSNYDSELEKEIKSMSKIGAARTTKKRIPNTKDFDSSEDEKHSKKGMDNQGHKNLKTSQEGSSDDAERKQERETFSSAEGTVDKDTTIMELRDRLPKKQQASASTDGVDKLSGKEQSFTSLEVRKVAETKEKSKHLKTKTCKKVQDGLSDIAEKFLKKDQSDETSEDDKKQSKKGTEEKKKPSDFKKKVIKMEQQYESSSDGTEKLPEREEICHFPKGIKQIKNGTTDGEKKSKKIRDKTSKKKDELSDYAEKSTGKGDSCDSSEDKKSKNGAYGREKKRCKLLGKSSRKRQDCSSSDTEKYSMKEDGCNSSDKRLKRIELRERRNLSSKRNTKEIQSGSSSSDAEESSEDNKKKKQRTSSKKKAVIVKEKKRNSLRTSTKRKQADITSSSSSDIEDDDQNSIGEGSSDEQKIKPVTENLVLSSHTGFCQSSGDEALSKSVPVTVDDDDDDNDPENRIAKKMLLEEIKANLSSDEDGSSDDEPEEGKKRTGKQNEENPGDEEAKNQVNSESDSDSEESKKPRYRHRLLRHKLTVSDGESGEEKKTKPKEHKEVKGRNRRKVSSEDSEDSDFQESGVSEEVSESEDEQRPRTRSAKKAELEENQRSYKQKKKRRRIKVQEDSSSENKSNSEEEEEEKEEEEEEEEEEEEEEEDENDDSKSPGKGRKKIRKILKDDKLRTETQNALKEEEERRKRIAEREREREKLREVIEIEDASPTKCPITTKLVLDEDEETKEPLVQVHRNMVIKLKPHQVDGVQFMWDCCCESVKKTKKSPGSGCILAHCMGLGKTLQVVSFLHTVLLCDKLDFSTALVVCPLNTALNWMNEFEKWQEGLKDDEKLEVSELATVKRPQERSYMLQRWQEDGGVMIIGYEMYRNLAQGRNVKSRKLKEIFNKALVDPGPDFVVCDEGHILKNEASAVSKAMNSIRSRRRIILTGTPLQNNLIEYHCMVNFIKENLLGSIKEFRNRFINPIQNGQCADSTMVDVRVMKKRAHILYEMLAGCVQRKDYTALTKFLPPKHEYVLAVRMTSIQCKLYQYYLDHLTGVGNNSEGGRGKAGAKLFQDFQMLSRIWTHPWCLQLDYISKENKGYFDEDSMDEFIASDSDETSMSLSSDDYTKKKKKGKKGKKDSSSSGSGSDNDVEVIKVWNSRSRGGGEGNVDETGNNPSVSLKLEESKATSSSNPSSPAPDWYKDFVTDADAEVLEHSGKMVLLFEILRMAEEIGDKVLVFSQSLISLDLIEDFLELASREKTEDKDKPLIYKGEGKWLRNIDYYRLDGSTTAQSRKKWAEEFNDETNVRGRLFIISTKAGSLGINLVAANRVIIFDASWNPSYDIQSIFRVYRFGQTKPVYVYRFLAQGTMEDKIYDRQVTKQSLSFRVVDQQQVERHFTMNELTELYTFEPDLLDDPNSEKKKKRDTPMLPKDTILAELLQIHKEHIVGYHEHDSLLDHKEEEELTEEERKAAWAEYEAEKKGLTMRFNIPTGTNLPPVSFNSQTPYIPFNLGALSAMSNQQLEDLINQGREKVVEATNSVTAVRIQPLEDIISAVWKENMNLSEAQVQALALSRQASQELDVKRREAIYNDVLTKQQMLISCVQRILMNRRLQQQYNQQQQQQMTYQQATLGHLMMPKPPNLIMNPSNYQQIDMRGMYQPVAGGMQPPPLQRAPPPMRSKNPGPSQGKSM

SEQ ID NO: 6 - DNA, 11202 bp, Homo sapiens
  source 1..11202: mol_type = other DNA
aattctcctgcctgagcctcggcccaacaaaatggcggcggcagcggtgtcgctttgtttccgcggctcctgcggcggtggcagtggtagcggcctttgagctgtggggaggttccagcagcagctacagtgacgactaagactccagtgcatttctatcgtaaccgggcgcgggggagcgcagatcggcgcccagcaatcacagaagccgacaaggcgttcaagcgaaaacatgaccgctgagcccatgagtgaaagcaagttgaatacattggtgcagaagcttcatgacttccttgcacactcatcagaagaatctgaagaaacaagttctcctccacgacttgcaatgaatcaaaacacagataaaatcagtggttctggaagtaactctgatatgatggaaaacagcaaggaagagggaactagctcttcagaaaaatccaagtcttcaggatcgtcacgatcaaagaggaaaccttcaattgtaacaaagtatgtagaatcagatgatgaaaaacctttggatgatgaaactgtaaatgaagatgcgtctaatgaaaattcagaaaatgatattactatgcagagcttgccaaaaggtacagtgattgtacagccagagccagtgctgaatgaagacaaagatgattttaaagggcctgaatttagaagcagaagtaaaatgaaaactgaaaatctcaaaaaacgcggagaagatgggcttcatgggattgtgagctgcactgcttgtggacaacaggtcaatcattttcaaaaagattccatttatagacacccttcattgcaagttcttatttgtaagaattgctttaagtattacatgagtgatgatattagccgtgactcagatggaatggatgaacaatgtaggtggtgtgcggaaggtggaaacttgatttgttgtgacttttgccataatgctttctgcaagaaatgcattctacgcaaccttggtcgaaaggagttgtccacaataatggatgaaaacaaccaatggtattgctacatttgtcacccagagcctttgttggacttggtcactgcatgtaacagcgtatttgagaatttagaacagttgttgcagcaaaataagaagaagataaaagttgacagtgaaaagagtaataaagtatatgaacatacatccagattttctccaaagaagactagttcaaattgtaatggagaagaaaagaaattagatgattcctgttctggctctgtaacctactcttattccgcactaattgtgcccaaagagatgattaagaaggcaaaaaaactgattgagaccacagccaacatgaactccagttatgttaaatttttaaagcaggcaacagataattcagaaatcagttctgctacaaaattacgtcagcttaaggcttttaagtctgtgttggctgatattaagaaggctcatcttgcattggaagaagacttaaattccgagtttcgagcgatggatgctgtaaacaaagagaaaaataccaaagagcataaagtcatagatgctaagtttgaaacaaaagcacgaaaaggagaaaaaccttgtgctttggaaaagaaggatatttcaaagtcagaagctaaactttcaagaaaacaggtagatagtgagcacatgcatcagaatgttccaacagaggaacaaagaacaaataaaagtaccggtggtgaacataagaaatctgatagaaaagaagaacctcaatatgaacctgccaacacttctgaagatttagacatggatattgtgtctgttccttcctcagttccagaagacatttttgagaatcttgagactgctatggaagttcagagttcagttgatcatcaaggggatggcagcagtggaactgaacaagaagtggagagttcatctgtaaaattaaatatttcttcaaaagacaacagaggaggtattaaatcaaaaactacagctaaagtaacaaaagaattatatgttaaactcactcctgtttccctttctaattccccaattaaaggtgctgattgtcaggaagttccacaagataaagatggctataaaagttgtggtctgaaccccaagttagagaaatgtggacttggacaggaaaacagtgataatgagcatttggttgaaaatgaagtttcattacttttagaggaatctgatcttcgaagatccccacgtgtaaagactacacccttgaggcgaccgacagaaactaaccctgtaacatctaattcagatgaagaatgtaatgaaacagttaaggagaaacaaaaactatcagttccagtgagaaaaaaggataagcgtaattcttctgacagtgctatagataatcctaagcctaataaattgccaaaatctaagcaatcagagactgtggatcaaaattcagattctgatgaaatgctagcaatcctcaaagaggtgagcaggatgagtcacagttcttcttcagatactgatattaatgaaattcatacaaaccataagactttgtatgatttaaagactcaggcggggaaagatgataaaggaaaaaggaaacgaaaaagttctacatctggctcagattttgatactaaaaagggcaaatcagctaagagctctataatttctaaaaagaaacgacaaacccagtctgagtcttctaattatgactcagaattagaaaaagagataaagagcatgagtaaaattggtgctgccagaaccaccaaaaaaagaattccaaatacaaaagattttgactcttctgaagatgagaaacacagcaaaaaaggaatggataatcaagggcacaaaaatttgaagacctcacaagaaggatcatctgatgatgctgaaagaaaacaagagagagagactttctcttcagcagaaggcacagttgataaagacacgaccatcatggaattaagagatcgacttcctaagaagcagcaagcaagtgcttccactgatggtgtcgataagctttctgggaaagagcagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtcattttcctaagggcataaaacaaattaagaatggaacaactgatggagaaaagaaaagtaaaaaaataagagataaaacttctaaaaagaaggatgaattatctgattatgctgagaagtcaacagggaaaggagatagttgtgactcttcagaggataaaaagagtaagaatggagcatatggtagagagaagaaaaggtgcaagttgcttggaaagagttcaaggaagagacaagattgttcatcatctgatactgagaaatattccatgaaagaagatggttgtaactcttctgataagagactgaaaagaatagaattgagggaaagaagaaatttaagttcaaagagaaatactaaggaaatacaaagtggctcatcatcatctgatgctgaggaaagttctgaagataataaaaagaagaagcaaagaacttcatctaaaaagaaggcagtcattgtcaaggagaaaaagagaaactccctaagaacaagcactaaaaggaagcaagctgacattacatcctcatcttcttctgatatagaagatgatgatcagaattctataggtgagggaagcagcgatgaacagaaaattaagcctgtgactgaaaatttagtgctgtcttcacatactggattttgccaatcttcaggagatgaagccttatctaaatcagtgcctgtcacagtggatgatgatgatgacgacaatgatcctgagaatagaattgccaagaagatgcttttagaagaaattaaagccaatctttcctctgatgaggatggatcttcagatgatgagccagaagaagggaaaaaaagaactggaaaacaaaatgaagaaaacccaggagatgaggaagcaaaaaatcaagtcaattctgaatcagattcagattctgaagaatctaagaagccaagatacagacataggcttttgcggcacaaattgactgtgagtgacggagaatctggagaagaaaaaaagacaaagcctaaagagcataaagaagtcaaaggcagaaacagaagaaaggtgagcagtgaagattcagaagattctgattttcaggaatcaggagttagtgaagaagttagtgaatccgaagatgaacagcggcccagaacaaggtctgcaaagaaagcagagttggaagaaaatcagcggagctataaacagaaaaagaaaaggcgacgtattaaggttcaagaagattcatccagtgaaaacaagagtaattctgaggaagaagaggaggaaaaagaagaggaggaggaagaggaggaggaggaggaagaggaggaggaagatgaaaatgatgattccaagtctcctggaaaaggcagaaagaaaattcggaagattcttaaagatgataaactgagaacagaaacacaaaatgctcttaaggaagaggaagagagacgaaaacgtattgctgagagggagcgtgagcgagaaaaattgagagaggtgatagaaattgaagatgcttcacccaccaagtgtccaataacaaccaagttggttttagatgaagatgaagaaaccaaagaacctttagtgcaggttcatagaaatatggttatcaaattgaaaccccatcaagtagatggtgttcagtttatgtgggattgctgctgtgagtctgtgaaaaaaacaaagaaatctccaggttcaggatgcattcttgcccactgtatgggccttggtaagactttacaggtggtaagttttcttcatacagttcttttgtgtgacaaactggatttcagcacggcgttagtggtttgtcctcttaatactgctttgaattggatgaatgaatttgagaagtggcaagagggattaaaagatgatgagaagcttgaggtttctgaattagcaactgtgaaacgtcctcaggagagaagctacatgctgcagaggtggcaagaagatggtggtgttatgatcataggctatgagatgtatagaaatcttgctcaaggaaggaatgtgaagagtcggaaacttaaagaaatatttaacaaagctttggttgatccaggccctgattttgttgtttgtgatgaaggccatattctaaaaaatgaagcatctgctgtttctaaagctatgaattctatacgatcaaggaggaggattattttaacaggaacaccacttcaaaataacctaattgagtatcattgtatggttaattttatcaaggaaaatttacttggatccattaaggagttcaggaatagatttataaatccaattcaaaatggtcagtgtgcagattctaccatggtagatgtcagagtgatgaaaaaacgtgctcacattctctatgagatgttagctggatgtgttcagaggaaagattatacagcattaacaaaattcttgcctccaaaacacgaatatgtgttagctgtgagaatgacttctattcagtgcaagctctatcagtactacttagatcacttaacaggtgtgggcaataatagtgaaggtggaagaggaaaggcaggtgcaaagcttttccaagattttcagatgttaagtagaatatggactcatccttggtgtttgcagctagactacattagcaaagaaaataagggttattttgatgaagacagtatggatgaatttatagcctcagattctgatgaaacctccatgagtttaagctccgatgattatacaaaaaagaagaaaaaagggaaaaaggggaaaaaagatagtagctcaagtggaagtggcagtgacaatgatgttgaagtgattaaggtctggaattcaagatctcggggaggtggtgaaggaaatgtggatgaaacaggaaacaatccttctgtttctttaaaactggaagaaagtaaagctacttcttcttctaatccaagcagcccagctccagactggtacaaagattttgttacagatgctgatgctgaggttttagagcattctgggaaaatggtacttctctttgaaattcttcgaatggcagaggaaattggggataaagtccttgttttcagccagtccctcatatctctggacttgattgaagattttcttgaattagctagtagggagaagacagaagataaagataaaccccttatttataaaggtgaggggaagtggcttcgaaacattgactattaccgtttagatggttccactactgcacagtcaaggaagaagtgggctgaagaatttaatgatgaaactaatgtgagaggacgattatttatcatttctactaaagcaggatctctaggaattaatctggtagctgctaatcgagtaattatattcgacgcttcttggaatccatcttatgacatccagagtatattcagagtttatcgctttggacaaactaagcctgtttatgtatataggttcttagctcagggaaccatggaagataagatttatgatcggcaagtaactaagcagtcactgtcttttcgagttgttgatcagcagcaggtggagcgtcattttactatgaatgagcttactgaactttatacttttgagccagacttattagatgaccctaattcagaaaagaagaagaagagggatactcccatgctgccaaaggataccatacttgcagagctccttcagatacataaagaacacattgtaggataccatgaacatgattctcttttggaccacaaagaagaagaagagttgactgaagaagaaagaaaagcagcttgggctgagtatgaagcagagaagaagggactgaccatgcgtttcaacataccaactgggaccaatttaccccctgtcagtttcaactctcaaactccttatattcctttcaatttgggagccctgtcagcaatgagtaatcaacagctggaggacctcattaatcaaggaagagaaaaagttgtagaagcaacaaacagtgtgacagcagtgaggattcaacctcttgaggatataatttcagctgtatggaaggagaacatgaatctctcagaggcccaagtacaggcgttagcattaagtagacaagccagccaggagcttgatgttaaacgaagagaagcaatctacaatgatgtattgacaaaacaacagatgttaatcagctgtgttcagcgaatacttatgaacagaaggctccagcagcagtacaatcagcagcaacagcaacaaatgacttatcaacaagcaacactgggtcacctcatgatgccaaagcccccaaatttgatcatgaatccttctaactaccagcagattgatatgagaggaatgtatcagccagtggctggtggtatgcagccaccaccattacagcgtgcaccacccccaatgagaagcaaaaatccaggaccttcccaagggaaatcaatgtgattttgcactaaaagcttaatggattgttaaaatcatagaaagatcttttatttttttaggaatcaatgacttaacagaactcaactgtataaatagtttggtccccttaaatgccaatcttccatattagttttactttttttttttttaaatagggcataccatttcttcctgacatttgtcagtgatgttgcctagaatcttcttacacacgctgagtacagaagatatttcaaattgttttcagtgaaaacaagtccttccataatagtaacaactccacagatttcctctctaaatttttatgcctgcttttagcaaccataaaattgtcataaaattaataaatttaggaaagaataaagatttatatattcattctttacatataaaaacacacagctgagttcttagagttgattcctcaagttatgaaatacttttgtacttaatccatttcttgattaaagtgattgaaatggttttaatgttcttttgactgaagtctgaaactgggctcctgctttattgtctctgtgactgaaagttagaaactgagggttatctttgacacagaattgtgtgcaatattcttaaatactactgctctaaaagttggagaagtcttgcagttatcttagcattgtataaacagccttaagtatagcctaagaagagaattcctttttcttctttagtccttctgccattttttattttcagttatatgtgctgaaataattactggtaaaatttcagggttgtggattatcttccacacatgaattttctctctcctggcacgaatataaagcacatctcttaactgcatggtgccagtgctaatgcttcatcctgttgctggcagtgggatgtggacttagaaaatcaagttctagcattttagtaggttaacactgaagttgtggttgttaggttcacaccctgttttataaacaacatcaaaatggcagaaccattgctgactttaggttcacatgaggaatgtacttttaacaattcccagtactatcagtattgtgaaataattcctctgaaagataagaatcactggcttctatgcgcttcttttctctcatcatcatgttcttttaccccagtttccttacatttttttaaattgtttcagagtttgttttttttttagtttagattgtgaggcaattattaaatcaaaattaattcatccaatacccctttactagaagttttactagaaaatgtattacattttattttttcttaatccagttctgcaaaaatgacctataaatttattcatgtacaattttggttacttgaattgttaaagaaaacattgtttttgactatgggagtcaactcaacatggcagaaccatttttgagatgatgatacaacaggtagtgaaacagcttaagaattccaaaaaaaaaaaaaaaaaaaaaaaaaagaaaactgggtttgggctttgctttaggtatcactggattagaatgagtttaacattagctaaaactgctttgagttgtttggatgattaagagattgccatttttatcttggaagaactagtggtaaaacatccaagagcactaggattgtgatacagaatttgtgaggtttggtggatccacgcccctctcccccactttcccatgatgaaatatcactaataaatcctgtatatttagatattatgctagccatgtaatcagatttatttaattgggtggggcaggtgtgtatttactttagaaaaaatgaaaaagacaagatttatgagaaatatttgaaggcagtacactctggccaactgttaccagttggtatttctacaagttcagaatattttaaacctgatttactagacctgggaattttcaacatggtctaattatttactcaaagacatagatgtgaaaattttaggcaaccttctaaatctttttcaccatggatgaaactataacttaaagaataatacttagaagggttaattggaaatcagagtttgaaataaaacttggaccactttgtatacactcttctcacttgacattttagctatataatatgtactttgagtataacatcaagctttaacaaatatttaaagacaaaaaaatcacgtcagtaaaatactaaaaggctcatttttatatttgttttagatgttttaaatagttgcaatggattaaaaatgatgatttaaaatgttgcttgtaatacagttttgcctgctaaattctccacattttgtaacctgttttatttctttgggtgtaaagcgtttttgcttagtattgtgatattgtatatgttttgtcccagttgtatagtaatgtttcagtccatcatccagctttggctgctgaaatcatacagctgtgaagacttgcctttgtttctgttagactgcttttcagttctgtattgagtatcttaagtactgtagaaaagatgtcacttcttcctttaaggctgttttgtaatatatataaggactggaattgtgtttttaaagaaaagcattcaagtatgacaatatactatctgtgttttcaccattcaaagtgctgtttagtagttgaaacttaaactatttaatgtcatttaataaagtgaccaaaatgtgttgtgctctttattgtattttcacagctttgaaaatctgtgcacatactgtttcatagaaaatgtatagcttttgttgtcctatataatggtggttcttttgcacatttagttatttaatattgagaggtcacgaagtttggttattgaatctgttatatactaaattctgtaaagggagatctctcatctcaaaaagaatttacataccaggaagtccatgtgtgtttgtgttagttttggatgtctttgtgtaatccagccccatttcctgtttcccaacagctgtaacactcattttaagtcaagcagggctaccaacccacacttgatagaaaagctgcttaccattcagaagcttccttattacctggcctccaaatgagctgaatattttgtagccttcccttagctatgttcattttccctccattatcataaaatcagatcgatatttatgtgccccaaacaaaactttaagagcagttacattctgtcccagtagcccttgtttcctttgagagtagcatgttgtgaggctatagagacttattctaccagtaaaacaggtcaatccttttacatgtttattatactaaaaattatgttcagggtatttactactttatttcaccagactcagtctcaagtgacttggctatctccaaatcagatctacccttagagaataaacatttttctaccgttattttttttcaagtctataatctgagccagtcccaaaggagtgatcaagtttcagaaatgctttcatcttcacaacattttatatatactattatatggggtgaataaagttttaaatccgaaatataaaaaaaaaaaaaaaaaaa

SEQ ID NO: 7 - protein, 2454 aa, Homo sapiens
MTAEPMSESKLNTLVQKLHDFLAHSSEESEETSSPPRLAMNQNTDKISGSGSNSDMMENSKEEGTSSSEKSKSSGSSRSKRKPSIVTKYVESDDEKPLDDETVNEDASNENSENDITMQSLPKEDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKKIKVDSEKSNKVYEHTSRFSPKKTSSNCNGEEKKLDDSCSGSVTYSYSALIVPKEMIKKAKKLIETTANMNSSYVKFLKQATDNSEISSATKLRQLKAFKSVLADIKKAHLALEEDLNSEFRAMDAVNKEKNTKEHKVIDAKFETKARKGEKPCALEKKDISKSEAKLSRKQVDSEHMHQNVPTEEQRTNKSTGGEHKKSDRKEEPQYEPANTSEDLDMDIVSVPSSVPEDIFENLETAMEVQSSVDHQGDGSSGTEQEVESSSVKLNISSKDNRGGIKSKTTAKVTKELYVKLTPVSLSNSPIKGADCQEVPQDKDGYKSCGLNPKLEKCGLGQENSDNEHLVENEVSLLLEESDLRRSPRVKTTPLRRPTETNPVTSNSDEECNETVKEKQKLSVPVRKKDKRNSSDSAIDNPKPNKLPKSKQSETVDQNSDSDEMLAILKEVSRMSHSSSSDTDINEIHTNHKTLYDLKTQAGKDDKGKRKRKSSTSGSDFDTKKGKSAKSSIISKKKRQTQSESSNYDSELEKEIKSMSKIGAARTTKKRIPNTKDFDSSEDEKHSKKGMDNQGHKNLKTSQEGSSDDAERKQERETFSSAEGTVDKDTTIMELRDRLPKKQQASASTDGVDKLSGKEQSFTSLEVRKVAETKEKSKHLKTKTCKKVQDGLSDIAEKFLKKDQSDETSEDDKKQSKKGTEEKKKPSDFKKKVIKMEQQYESSSDGTEKLPEREEICHFPKGIKQIKNGTTDGEKKSKKIRDKTSKKKDELSDYAEKSTGKGDSCDSSEDKKSKNGAYGREKKRCKLLGKSSRKRQDCSSSDTEKYSMKEDGCNSSDKRLKRIELRERRNLSSKRNTKEIQSGSSSSDAEESSEDNKKKKQRTSSKKKAVIVKEKKRNSLRTSTKRKQADITSSSSSDIEDDDQNSIGEGSSDEQKIKPVTENLVLSSHTGFCQSSGDEALSKSVPVTVDDDDDDNDPENRIAKKMLLEEIKANLSSDEDGSSDDEPEEGKKRTGKQNEENPGDEEAKNQVNSESDSDSEESKKPRYRHRLLRHKLTVSDGESGEEKKTKPKEHKEVKGRNRRKVSSEDSEDSDFQESGVSEEVSESEDEQRPRTRSAKKAELEENQRSYKQKKKRRRIKVQEDSSSENKSNSEEEEEEKEEEEEEEEEEEEEEEDENDDSKSPGKGRKKIRKILKDDKLRTETQNALKEEEERRKRIAEREREREKLREVIEIEDASPTKCPITTKLVLDEDEETKEPLVQVHRNMVIKLKPHQVDGVQFMWDCCCESVKKTKKSPGSGCILAHCMGLGKTLQVVSFLHTVLLCDKLDFSTALVVCPLNTALNWMNEFEKWQEGLKDDEKLEVSELATVKRPQERSYMLQRWQEDGGVMIIGYEMYRNLAQGRNVKSRKLKEIFNKALVDPGPDFVVCDEGHILKNEASAVSKAMNSIRSRRRIILTGTPLQNNLIEYHCMVNFIKENLLGSIKEFRNRFINPIQNGQCADSTMVDVRVMKKRAHILYEMLAGCVQRKDYTALTKFLPPKHEYVLAVRMTSIQCKLYQYYLDHLTGVGNNSEGGRGKAGAKLFQDFQMLSRIWTHPWCLQLDYISKENKGYFDEDSMDEFIASDSDETSMSLSSDDYTKKKKKGKKGKKDSSSSGSGSDNDVEVIKVWNSRSRGGGEGNVDETGNNPSVSLKLEESKATSSSNPSSPAPDWYKDFVTDADAEVLEHSGKMVLLFEILRMAEEIGDKVLVFSQSLISLDLIEDFLELASREKTEDKDKPLIYKGEGKWLRNIDYYRLDGSTTAQSRKKWAEEFNDETNVRGRLFIISTKAGSLGINLVAANRVIIFDASWNPSYDIQSIFRVYRFGQTKPVYVYRFLAQGTMEDKIYDRQVTKQSLSFRVVDQQQVERHFTMNELTELYTFEPDLLDDPNSEKKKKRDTPMLPKDTILAELLQIHKEHIVGYHEHDSLLDHKEEEELTEEERKAAWAEYEAEKKGLTMRFNIPTGTNLPPVSFNSQTPYIPFNLGALSAMSNQQLEDLINQGREKVVEATNSVTAVRIQPLEDIISAVWKENMNLSEAQVQALALSRQASQELDVKRREAIYNDVLTKQQMLISCVQRILMNRRLQQQYNQQQQQQMTYQQATLGHLMMPKPPNLIMNPSNYQQIDMRGMYQPVAGGMQPPPLQRAPPPMRSKNPGPSQGKSM

SEQ ID NO: 8 - DNA, 11088 bp, Homo sapiens
  source 1..11088: mol_type = other DNA
aattctcctgcctgagcctcggcccaacaaaatggcggcggcagcggtgtcgctttgtttccgcggctcctgcggcggtggcagtggtagcggcctttgagctgtggggaggttccagcagcagctacagtgacgactaagactccagtgcatttctatcgtaaccgggcgcgggggagcgcagatcggcgcccagcaatcacagaagccgacaaggcgttcaagcgaaaacatgaccgctgagcccatgagtgaaagcaagttgaatacattggtgcagaagcttcatgacttccttgcacactcatcagaagaatctgaagaaacaagttctcctccacgacttgcaatgaatcaaaacacagataaaatcagtggttctggaagtaactctgatatgatggaaaacagcaaggaagagggaactagctcttcagaaaaatccaagtcttcaggatcgtcacgatcaaagaggaaaccttcaattgtaacaaagtatgtagaatcagatgatgaaaaacctttggatgatgaaactgtaaatgaagatgcgtctaatgaaaattcagaaaatgatattactatgcagagcttgccaaaagaagatgggcttcatgggattgtgagctgcactgcttgtggacaacaggtcaatcattttcaaaaagattccatttatagacacccttcattgcaagttcttatttgtaagaattgctttaagtattacatgagtgatgatattagccgtgactcagatggaatggatgaacaatgtaggtggtgtgcggaaggtggaaacttgatttgttgtgacttttgccataatgctttctgcaagaaatgcattctacgcaaccttggtcgaaaggagttgtccacaataatggatgaaaacaaccaatggtattgctacatttgtcacccagagcctttgttggacttggtcactgcatgtaacagcgtatttgagaatttagaacagttgttgcagcaaaataagaagaagataaaagttgacagtgaaaagagtaataaagtatatgaacatacatccagattttctccaaagaagactagttcaaattgtaatggagaagaaaagaaattagatgattcctgttctggctctgtaacctactcttattccgcactaattgtgcccaaagagatgattaagaaggcaaaaaaactgattgagaccacagccaacatgaactccagttatgttaaatttttaaagcaggcaacagataattcagaaatcagttctgctacaaaattacgtcagcttaaggcttttaagtctgtgttggctgatattaagaaggctcatcttgcattggaagaagacttaaattccgagtttcgagcgatggatgctgtaaacaaagagaaaaataccaaagagcataaagtcatagatgctaagtttgaaacaaaagcacgaaaaggagaaaaaccttgtgctttggaaaagaaggatatttcaaagtcagaagctaaactttcaagaaaacaggtagatagtgagcacatgcatcagaatgttccaacagaggaacaaagaacaaataaaagtaccggtggtgaacataagaaatctgatagaaaagaagaacctcaatatgaacctgccaacacttctgaagatttagacatggatattgtgtctgttccttcctcagttccagaagacatttttgagaatcttgagactgctatggaagttcagagttcagttgatcatcaaggggatggcagcagtggaactgaacaagaagtggagagttcatctgtaaaattaaatatttcttcaaaagacaacagaggaggtattaaatcaaaaactacagctaaagtaacaaaagaattatatgttaaactcactcctgtttccctttctaattccccaattaaaggtgctgattgtcaggaagttccacaagataaagatggctataaaagttgtggtctgaaccccaagttagagaaatgtggacttggacaggaaaacagtgataatgagcatttggttgaaaatgaagtttcattacttttagaggaatctgatcttcgaagatccccacgtgtaaagactacacccttgaggcgaccgacagaaactaaccctgtaacatctaattcagatgaagaatgtaatgaaacagttaaggagaaacaaaaactatcagttccagtgagaaaaaaggataagcgtaattcttctgacagtgctatagataatcctaagcctaataaattgccaaaatctaagcaatcagagactgtggatcaaaattcagattctgatgaaatgctagcaatcctcaaagaggtgagcaggatgagtcacagttcttcttcagatactgatattaatgaaattcatacaaaccataagactttgtatgatttaaagactcaggcggggaaagatgataaaggaaaaaggaaacgaaaaagttctacatctggctcagattttgatactaaaaagggcaaatcagctaagagctctataatttctaaaaagaaacgacaaacccagtctgagtcttctaattatgactcagaattagaaaaagagataaagagcatgagtaaaattggtgctgccagaaccaccaaaaaaagaattccaaatacaaaagattttgactcttctgaagatgagaaacacagcaaaaaaggaatggataatcaagggcacaaaaatttgaagacctcacaagaaggatcatctgatgatgctgaaagaaaacaagagagagagactttctcttcagcagaaggcacagttgataaagacacgaccatcatggaattaagagatcgacttcctaagaagcagcaagcaagtgcttccactgatggtgtcgataagctttctgggaaagagcagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtcattttcctaagggcataaaacaaattaagaatggaacaactgatggagaaaagaaaagtaaaaaaataagagataaaacttctaaaaagaaggatgaattatctgattatgctgagaagtcaacagggaaaggagatagttgtgactcttcagaggataaaaagagtaagaatggagcatatggtagagagaagaaaaggtgcaagttgcttggaaagagttcaaggaagagacaagattgttcatcatctgatactgagaaatattccatgaaagaagatggttgtaactcttctgataagagactgaaaagaatagaattgagggaaagaagaaatttaagttcaaagagaaatactaaggaaatacaaagtggctcatcatcatctgatgctgaggaaagttctgaagataataaaaagaagaagcaaagaacttcatctaaaaagaaggcagtcattgtcaaggagaaaaagagaaactccctaagaacaagcactaaaaggaagcaagctgacattacatcctcatcttcttctgatatagaagatgatgatcagaattctataggtgagggaagcagcgatgaacagaaaattaagcctgtgactgaaaatttagtgctgtcttcacatactggattttgccaatcttcaggagatgaagccttatctaaatcagtgcctgtcacagtggatgatgatgatgacgacaatgatcctgagaatagaattgccaagaagatgcttttagaagaaattaaagccaatctttcctctgatgaggatggatcttcagatgatgagccagaagaagggaaaaaaagaactggaaaacaaaatgaagaaaacccaggagatgaggaagcaaaaaatcaagtcaattctgaatcagattcagattctgaagaatctaagaagccaagatacagacataggcttttgcggcacaaattgactgtgagtgacggagaatctggagaagaaaaaaagacaaagcctaaagagcataaagaagtcaaaggcagaaacagaagaaaggtgagcagtgaagattcagaagattctgattttcaggaatcaggagttagtgaagaagttagtgaatccgaagatgaacagcggcccagaacaaggtctgcaaagaaagcagagttggaagaaaatcagcggagctataaacagaaaaagaaaaggcgacgtattaaggttcaagaagattcatccagtgaaaacaagagtaattctgaggaagaagaggaggaaaaagaagaggaggaggaagaggaggaggaggaggaagaggaggaggaagatgaaaatgatgattccaagtctcctggaaaaggcagaaagaaaattcggaagattcttaaagatgataaactgagaacagaaacacaaaatgctcttaaggaagaggaagagagacgaaaacgtattgctgagagggagcgtgagcgagaaaaattgagagaggtgatagaaattgaagatgcttcacccaccaagtgtccaataacaaccaagttggttttagatgaagatgaagaaaccaaagaacctttagtgcaggttcatagaaatatggttatcaaattgaaaccccatcaagtagatggtgttcagtttatgtgggattgctgctgtgagtctgtgaaaaaaacaaagaaatctccaggttcaggatgcattcttgcccactgtatgggccttggtaagactttacaggtggtaagttttcttcatacagttcttttgtgtgacaaactggatttcagcacggcgttagtggtttgtcctcttaatactgctttgaattggatgaatgaatttgagaagtggcaagagggattaaaagatgatgagaagcttgaggtttctgaattagcaactgtgaaacgtcctcaggagagaagctacatgctgcagaggtggcaagaagatggtggtgttatgatcataggctatgagatgtatagaaatcttgctcaaggaaggaatgtgaagagtcggaaacttaaagaaatatttaacaaagctttggttgatccaggccctgattttgttgtttgtgatgaaggccatattctaaaaaatgaagcatctgctgtttctaaagctatgaattctatacgatcaaggaggaggattattttaacaggaacaccacttcaaaataacctaattgagtatcattgtatggttaattttatcaaggaaaatttacttggatccattaaggagttcaggaatagatttataaatccaattcaaaatggtcagtgtgcagattctaccatggtagatgtcagagtgatgaaaaaacgtgctcacattctctatgagatgttagctggatgtgttcagaggaaagattatacagcattaacaaaattcttgcctccaaaacacgaatatgtgttagctgtgagaatgacttctattcagtgcaagctctatcagtactacttagatcacttaacaggtgtgggcaataatagtgaaggtggaagaggaaaggcaggtgcaaagcttttccaagattttcagatgttaagtagaatatggactcatccttggtgtttgcagctagactacattagcaaagaaaataagggttattttgatgaagacagtatggatgaatttatagcctcagattctgatgaaacctccatgagtttaagctccgatgattatacaaaaaagaagaaaaaagggaaaaaggggaaaaaagatagtagctcaagtggaagtggcagtgacaatgatgttgaagtgattaaggtctggaattcaagatctcggggaggtggtgaaggaaatgtggatgaaacaggaaacaatccttctgtttctttaaaactggaagaaagtaaagctacttcttcttctaatccaagcagcccagctccagactggtacaaagattttgttacagatgctgatgctgaggttttagagcattctgggaaaatggtacttctctttgaaattcttcgaatggcagaggaaattggggataaagtccttgttttcagccagtccctcatatctctggacttgattgaagattttcttgaattagctagtagggagaagacagaagataaagataaaccccttatttataaaggtgaggggaagtggcttcgaaacattgactattaccgtttagatggttccactactgcacagtcaaggaagaagtgggctgaagaatttaatgatgaaactaatgtgagaggacgattatttatcatttctactaaagcaggatctctaggaattaatctggtagctgctaatcgagtaattatattcgacgcttcttggaatccatcttatgacatccagagtatattcagagtttatcgctttggacaaactaagcctgtttatgtatataggttcttagctcagggaaccatggaagataagatttatgatcggcaagtaactaagcagtcactgtcttttcgagttgttgatcagcagcaggtggagcgtcattttactatgaatgagcttactgaactttatacttttgagccagacttattagatgaccctaattcagaaaagaagaagaagagggatactcccatgctgccaaaggataccatacttgcagagctccttcagatacataaagaacacattgtaggataccatgaacatgattctcttttggaccacaaagaagaagaagagttgactgaagaagaaagaaaagcagcttgggctgagtatgaagcagagaagaagggactgaccatgcgtttcaacataccaactgggaccaatttaccccctgtcagtttcaactctcaaactccttatattcctttcaatttgggagccctgtcagcaatgagtaatcaacagctggaggacctcattaatcaaggaagagaaaaagttgtagaagcaacaaacagtgtgacagcagtgaggattcaacctcttgaggatataatttcagctgtatggaaggagaacatgaatctctcagaggcccaagtacaggcgttagcattaagtagacaagccagccaggagcttgatgttaaacgaagagaagcaatctacaatgatgtattgacaaaacaacagatgttaatcagctgtgttcagcgaatacttatgaacagaaggctccagcagcagtacaatcagcagcaacagcaacaaatgacttatcaacaagcaacactgggtcacctcatgatgccaaagcccccaaatttgatcatgaatccttctaactaccagcagattgatatgagaggaatgtatcagccagtggctggtggtatgcagccaccaccattacagcgtgcaccacccccaatgagaagcaaaaatccaggaccttcccaagggaaatcaatgtgattttgcactaaaagcttaatggattgttaaaatcatagaaagatcttttatttttttaggaatcaatgacttaacagaactcaactgtataaatagtttggtccccttaaatgccaatcttccatattagttttactttttttttttttaaatagggcataccatttcttcctgacatttgtcagtgatgttgcctagaatcttcttacacacgctgagtacagaagatatttcaaattgttttcagtgaaaacaagtccttccataatagtaacaactccacagatttcctctctaaatttttatgcctgcttttagcaaccataaaattgtcataaaattaataaatttaggaaagaataaagatttatatattcattctttacatataaaaacacacagctgagttcttagagttgattcctcaagttatgaaatacttttgtacttaatccatttcttgattaaagtgattgaaatggttttaatgttcttttgactgaagtctgaaactgggctcctgctttattgtctctgtgactgaaagttagaaactgagggttatctttgacacagaattgtgtgcaatattcttaaatactactgctctaaaagttggagaagtcttgcagttatcttagcattgtataaacagccttaagtatagcctaagaagagaattcctttttcttctttagtccttctgccattttttattttcagttatatgtgctgaaataattactggtaaaatttcagggttgtggattatcttccacacatgaattttctctctcctggcacgaatataaagcacatctcttaactgcatggtgccagtgctaatgcttcatcctgttgctggcagtgggatgtggacttagaaaatcaagttctagcattttagtaggttaacactgaagttgtggttgttaggttcacaccctgttttataaacaacatcaaaatggcagaaccattgctgactttaggttcacatgaggaatgtacttttaacaattcccagtactatcagtattgtgaaataattcctctgaaagataagaatcactggcttctatgcgcttcttttctctcatcatcatgttcttttaccccagtttccttacatttttttaaattgtttcagagtttgttttttttttagtttagattgtgaggcaattattaaatcaaaattaattcatccaatacccctttactagaagttttactagaaaatgtattacattttattttttcttaatccagttctgcaaaaatgacctataaatttattcatgtacaattttggttacttgaattgttaaagaaaacattgtttttgactatgggagtcaactcaacatggcagaaccatttttgagatgatgatacaacaggtagtgaaacagcttaagaattccaaaaaaaaaaaaaaaaaaaaaaaaaagaaaactgggtttgggctttgctttaggtatcactggattagaatgagtttaacattagctaaaactgctttgagttgtttggatgattaagagattgccatttttatcttggaagaactagtggtaaaacatccaagagcactaggattgtgatacagaatttgtgaggtttggtggatccacgcccctctcccccactttcccatgatgaaatatcactaataaatcctgtatatttagatattatgctagccatgtaatcagatttatttaattgggtggggcaggtgtgtatttactttagaaaaaatgaaaaagacaagatttatgagaaatatttgaaggcagtacactctggccaactgttaccagttggtatttctacaagttcagaatattttaaacctgatttactagacctgggaattttcaacatggtctaattatttactcaaagacatagatgtgaaaattttaggcaaccttctaaatctttttcaccatggatgaaactataacttaaagaataatacttagaagggttaattggaaatcagagtttgaaataaaacttggaccactttgtatacactcttctcacttgacattttagctatataatatgtactttgagtataacatcaagctttaacaaatatttaaagacaaaaaaatcacgtcagtaaaatactaaaaggctcatttttatatttgttttagatgttttaaatagttgcaatggattaaaaatgatgatttaaaatgttgcttgtaatacagttttgcctgctaaattctccacattttgtaacctgttttatttctttgggtgtaaagcgtttttgcttagtattgtgatattgtatatgttttgtcccagttgtatagtaatgtttcagtccatcatccagctttggctgctgaaatcatacagctgtgaagacttgcctttgtttctgttagactgcttttcagttctgtattgagtatcttaagtactgtagaaaagatgtcacttcttcctttaaggctgttttgtaatatatataaggactggaattgtgtttttaaagaaaagcattcaagtatgacaatatactatctgtgttttcaccattcaaagtgctgtttagtagttgaaacttaaactatttaatgtcatttaataaagtgaccaaaatgtgttgtgctctttattgtattttcacagctttgaaaatctgtgcacatactgtttcatagaaaatgtatagcttttgttgtcctatataatggtggttcttttgcacatttagttatttaatattgagaggtcacgaagtttggttattgaatctgttatatactaaattctgtaaagggagatctctcatctcaaaaagaatttacataccaggaagtccatgtgtgtttgtgttagttttggatgtctttgtgtaatccagccccatttcctgtttcccaacagctgtaacactcattttaagtcaagcagggctaccaacccacacttgatagaaaagctgcttaccattcagaagcttccttattacctggcctccaaatgagctgaatattttgtagccttcccttagctatgttcattttccctccattatcataaaatcagatcgatatttatgtgccccaaacaaaactttaagagcagttacattctgtcccagtagcccttgtttcctttgagagtagcatgttgtgaggctatagagacttattctaccagtaaaacaggtcaatccttttacatgtttattatactaaaaattatgttcagggtatttactactttatttcaccagactcagtctcaagtgacttggctatctccaaatcagatctacccttagagaataaacatttttctaccgttattttttttcaagtctataatctgagccagtcccaaaggagtgatcaagtttcagaaatgctttcatcttcacaacattttatatatactattatatggggtgaataaagttttaaatccgaaatataaaaaaaaaaaaaaaaaaa

SEQ ID NO: 9 - protein, 2285 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 10 - DNA, 8585 bp, Homo sapiens
  source 1..8585: mol_type = other DNA
cagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcagaattacaagcggccaatggatggcacatatggccctcctgccaagcggcacgaaggggagatgtacagcgtgccatacagcactgggcaggggcagcctcagcagcagcagttgcccccagcccagccccagcctgccagccagcaacaagctgcccagccttcccctcagcaagatgtatacaaccagtatggcaatgcctatcctgccactgccacagctgctactgagcgccgaccagcaggcggcccccagaaccaatttccattccagtttggccgagaccgtgtctctgcaccccctggcaccaatgcccagcaaaacatgccaccacaaatgatgggcggccccatacaggcatcagctgaggttgctcagcaaggcaccatgtggcaggggcgtaatgacatgacctataattatgccaacaggcagagcacgggctctgccccccagggccccgcctatcatggcgtgaaccgaacagatgaaatgctgcacacagatcagagggccaaccacgaaggctcgtggccttcccatggcacacgccagcccccatatggtccctctgcccctgtgccccccatgacaaggccccctccatctaactaccagcccccaccaagcatgcagaatcacattcctcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaagacttt

SEQ ID NO: 11 - protein, 2068 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 12 - DNA, 7934 bp, Homo sapiens
  source 1..7934: mol_type = other DNA
cagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaagacttt

SEQ ID NO: 13 - protein, 26 aa, synthetic construct
  REGION 1..26: note = Synthesized peptide
  SITE 26: note = MISC_FEATURE - wherein the Lys is attached to a biotin and an amide
ATKAARKSAPATGGVKKPHRYRPGGK

SEQ ID NO: 14 - protein, 26 aa, synthetic construct
  REGION 1..26: note = Synthesized peptide
  SITE 7: note = MISC_FEATURE - wherein the Lys is trimethylated
  SITE 26: note = MISC_FEATURE - wherein the Lys is attached to a biotin and an amide
ATKAARKSAPATGGVKKPHRYRPGGK

(19) United States
(12) Patent Application Publication
(10) Pub. No.: US 2025/0163033 A1
(43) Pub. Date: May 22, 2025

(54) METHODS OF TREATING CANCER

(71) Applicant: Epizyme, Inc., Cambridge, MA (US)

(72) Inventors: Sarah K. Knutson, Lincoln, MA (US); Natalie Warholic, Cambridge, MA (US); Heike Keilhack, Belmont, MA (US)

(21) Appl. No.: 18/900,177

(22) Filed: Sep. 27, 2024

Related U.S. Application Data
(63) Continuation of application No. 17/689,704, filed on Mar. 8, 2022, now Pat. No. 12,162,865.
Application No. 17/689,704 is a continuation of application No. 16/749,073, filed on Jan. 22, 2020, now abandoned.
Application No. 16/749,073 is a continuation of application No. 15/979,916, filed on May 15, 2018, now abandoned.
Application No. 15/979,916 is a continuation of application No. 15/598,262, filed on May 17, 2017, now abandoned.
Application No. 15/598,262 is a continuation of application No. 14/054,646, filed on Oct. 15, 2013, now Pat. No. 9,688,665.
(60) Provisional application No. 61/786,277, filed on Mar. 14, 2013.
(60) Provisional application No. 61/780,703, filed on Mar. 13, 2013.
(60) Provisional application No. 61/758,972, filed on Jan. 31, 2013.
(60) Provisional application No. 61/714,045, filed on Oct. 15, 2012.
(60) Provisional application No. 61/714,140, filed on Oct. 15, 2012.
(60) Provisional application No. 61/714,145, filed on Oct. 15, 2012.

(51) Int. Cl. C07D 405/14; A61K 31/4412; A61K 31/4545; A61K 31/5377; C07D 211/86; C07D 405/12; C12Q 1/6886

(52) U.S. Cl. CPC C07D 405/14; A61K 31/4412; A61K 31/4545; A61K 31/5377; C07D 211/86; C07D 405/12; C12Q 1/6886

(57) ABSTRACT

The present invention relates to methods of treating cancer by administering the EZH2 inhibitor compounds and pharmaceutical compositions to subjects in need thereof. The present invention also relates to the use of such compounds for research or other non-therapeutic purposes.

### RELATED APPLICATIONS

This application is a continuation of U.S. application Ser. No. 17/689,704, filed Mar. 8, 2022, which is a continuation of U.S. application Ser. No. 16/749,073, filed Jan. 22, 2020, which is a continuation of U.S. application Ser. No. 15/979,916, filed May 15, 2018, which is a continuation application of U.S. application Ser. No. 15/598,262, filed May 17, 2017, which is a continuation application of U.S. application Ser. No. 14/054,646, filed Oct. 15, 2013 (now U.S. Pat. No. 9,688,665), which claims priority to, and the benefit of U.S. Provisional Application Nos. 61/714,045, filed Oct. 15, 2012, 61/758,972, filed Jan. 31, 2013, 61/714,140, filed Oct. 15, 2012, 61/714,145, filed Oct. 15, 2012, 61/780,703, filed Mar. 13, 2013, and 61/786,277, filed Mar. 14, 2013. The contents of each of these provisional applications are incorporated herein by reference in their entireties.

### INCORPORATION-BY-REFERENCE OF SEQUENCE LISTING

The contents of the text file named “41478-513001US_ST25.txt”, which was created on Jan. 10, 2014 and is 141 KB in size, are hereby incorporated by reference in their entireties.

### FIELD OF INVENTION

The present invention relates generally to the field of cancer treatment, and in particular, the treatment of cancer associated with the SWI/SNF complex (i.e., SWI/SNF mediated cancer). More particularly, the present invention provides methods and compositions which treat, alleviate, prevent, diminish or otherwise ameliorate the symptoms of cancer associated with the SWI/SNF complex.

### BACKGROUND OF THE INVENTION

Disease-associated chromatin-modifying enzymes (e.g., EZH2) play a role in diseases such as proliferative disorders, metabolic disorders, and blood disorders. Thus, there is a need for the development of small molecules that are capable of modulating the activity of EZH2.

### SUMMARY OF THE INVENTION

The present invention provides a method for treating or alleviating a symptom of a SWI/SNF-associated cancer in a subject by administering to a subject in need thereof a therapeutically effective amount of an EZH2 inhibitor, where the subject has a cancer selected from the group consisting of brain and central nervous system cancer, head and neck cancer, kidney cancer, ovarian cancer, pancreatic cancer, leukemia, lung cancer, lymphoma, myeloma, sarcoma, breast cancer, and prostate cancer. For example, the SWI/SNF-associated cancer is characterized by reduced expression and/or loss of function of the SWI/SNF complex or one or more components of the SWI/SNF complex.

For example, the subject has a cancer selected from the group consisting of medulloblastoma, malignant rhabdoid tumor, and atypical teratoid/rhabdoid tumor.

For example, the one or more components are selected from the group consisting of SNF5, ATRX, and ARID1A.

For example, the loss of function is caused by a loss of function mutation resulting from a point mutation, a deletion, and/or an insertion.

For example, the subject has a deletion of SNF5.

For example, the subject has a mutation of ATRX selected from the group consisting of a substitution of asparagine (N) for the wild type residue lysine (K) at amino acid position 688 of SEQ ID NO: 5 (K688N), and a substitution of isoleucine (I) for the wild type residue methionine (M) at amino acid position 366 of SEQ ID NO: 5 (M366I).

For example, subject has a mutation of ARID1A selected from the group consisting of a nonsense mutation for the wild type residue cysteine (C) at amino acid position 884 of SEQ ID NO: 11 (C884*), a substitution of lysine (K) for the wild type residue glutamic acid (E) at amino acid position 966 (E966K), a nonsense mutation for the wild type residue glutamine (Q) at amino acid position 1411 of SEQ ID NO: 11 (Q1411*), a frame shift mutation at the wild type residue phenylalanine (F) at amino acid position 1720 of SEQ ID NO: 11 (F1720fs), a frame shift mutation after the wild type residue glycine (G) at amino acid position 1847 of SEQ ID NO: 11 (G1847fs), a frame shift mutation at the wild type residue cysteine (C) at amino acid position 1874 of SEQ ID NO: 11 (C1874fs), a substitution of glutamic acid (E) for the wild type residue aspartic acid (D) at amino acid position 1957 (D1957E), a nonsense mutation for the wild type residue glutamine (Q) at amino acid position 1430 of SEQ ID NO: 11 (Q1430*), a frame shift mutation at the wild type residue arginine (R) at amino acid position 1721 of SEQ ID NO: 11 (R1721fs), a substitution of glutamic acid (E) for the wild type residue glycine (G) at amino acid position 1255 (G1255E), a frame shift mutation at the wild type residue glycine (G) at amino acid position 284 of SEQ ID NO: 11 (G284fs), a nonsense mutation for the wild type residue arginine (R) at amino acid position 1722 of SEQ ID NO: 11 (R1722*), a frame shift mutation at the wild type residue methionine (M) at amino acid position 274 of SEQ ID NO: 11 (M274fs), a frame shift mutation at the wild type residue glycine (G) at amino acid position 1847 of SEQ ID NO: 11 (G1847fs), a frame shift mutation at the wild type residue P at amino acid position 559 of SEQ ID NO: 11 (P559fs), a nonsense mutation for the wild type residue arginine (R) at amino acid position 1276 of SEQ ID NO: 11 (R1276*), a frame shift mutation at the wild type residue glutamine (Q) at amino acid position 2176 of SEQ ID NO: 11 (Q2176fs), a frame shift mutation at the wild type residue histidine (H) at amino acid position 203 of SEQ ID NO: 11 (H203fs), a frame shift mutation at the wild type residue alanine (A) at amino acid position 591 of SEQ ID NO: 11 (A591fs), a nonsense mutation for the wild type residue glutamine (Q) at amino acid position 1322 of SEQ ID NO: 11 (Q1322*), a nonsense mutation for the wild type residue serine(S) at amino acid position 2264 of SEQ ID NO: 11 (S2264*), a nonsense mutation for the wild type residue glutamine (Q) at amino acid position 586 of SEQ ID NO: 11 (Q586*), a frame shift mutation at the wild type residue glutamine (Q) at amino acid position 548 of SEQ ID NO: 11 (Q548fs), and a frame shift mutation at the wild type residue asparagine (N) at amino acid position 756 of SEQ ID NO: 11 (N756fs).

The present invention also provides a method of treating or alleviating a symptom of a SWI/SNF-associated cancer in a subject in need thereof by (a) determining the expression level of at least one gene selected from the group consisting of neuronal differentiation genes, cell cycle inhibition genes and tumor suppressor genes in a sample obtained from the subject; (b) selecting the subject having a decreased expression level of at least one gene in step a; and (c) administering to the subject selected in step b an effective amount of an EZH2 inhibitor, thereby treating or alleviating a symptom of cancer in the subject.

The present invention further provides a method of treating or alleviating a symptom of a SWI/SNF-associated cancer in a subject in need thereof by (a) determining the expression level of at least one gene selected from the group consisting of hedgehog pathway genes, myc pathway genes and histone methyltransferase genesin a sample obtained from the subject; (b) selecting the subject having an increased expression level of at least one gene in step a; and (c) administering to the subject selected in step b an effective amount of an EZH2 inhibitor, thereby treating or alleviating a symptom of cancer in the subject.

For example, the cancer can be medulloblastoma, malignant rhabdoid tumor or atypical teratoid rhabdoid tumor.

For example, the neuronal differentiation gene is CD133, DOCK4, or PTPRK.

For example, the cell cycle inhibition gene is CKDNIA or CDKN2A.

For example, the tumor suppressor gene is BIN1.

For example, the hedgehog pathway gene is GLI1 or PTCH1.

For example, the myc pathway gene is MYC.

For example, the histone methyltransferase gene is EZH2.

The present invention also provides a method of inducing neuronal differentiation, cell cycle inhibition or tumor suppression by contacting a cell with an EZH2 inhibitor. The EZH2 inhibitor may be in an amount sufficient to increase expression of at least one gene selected from the group consisting of CD133, DOCK4, PTPRK, CKDNIA, CDKN2A and BIN1.

The present invention also provides a method of inhibiting hedgehog signaling by contacting a cell with an EZH2 inhibitor. The EZH2 inhibitor can be in an amount sufficient to reduce expression of GLI1 and/or PTCH1.

The present invention also provides a method of inducing gene expression by contacting a cell with an EZH2 inhibitor. The EZH2 inhibitor can be in an amount sufficient to induce neuronal differentiation, cell cycle inhibition and/or tumor suppression. For example, the gene can be CD133, DOCK4, PTPRK, CKDNIA, CKDN2A or BIN1.

The present invention also provides a method of inhibiting gene expression by contacting a cell with an EZH2 inhibitor. The EZH2 inhibitor is in an amount sufficient to inhibit hedgehog signaling. For example, the gene can be GLI1 or PTCH1.

For example, the cell may have loss of function of SNF5, ARID1A, ATRX, and/or a component of the SWI/SNF complex.

For example, the loss of function is caused by a deletion of SNF5.

For example, the cell is a cancer cell. The cancer can be medulloblastoma, malignant rhabdoid tumor or atypical teratoid rhabdoid tumor.

For example, the EZH2 inhibitor is Compound A having the following formula:

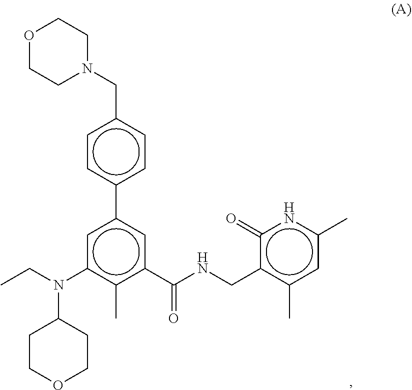

stereoisomers thereof, or pharmaceutically acceptable salts or solvates thereof.

For example, the EZH2 inhibitor is Compound B having the following formula:

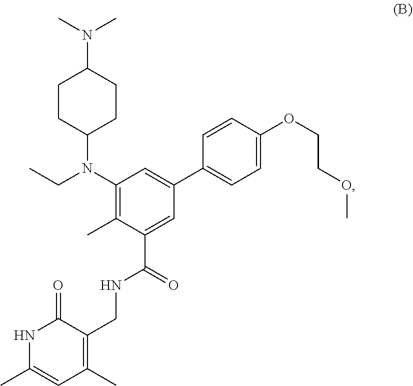

stereoisomers thereof, or pharmaceutically acceptable salts or solvates thereof.

For example, the EZH2 inhibitor is Compound C having the following formula:

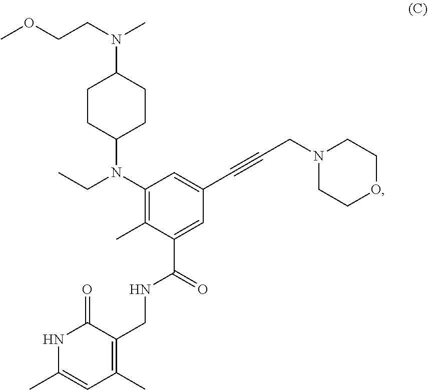 [SMILES: CCN(c1cc(C#CCN2CCOCC2)cc(C(=O)NCc2c(C)cc(C)[nH]c2=O)c1C)C1CCC(N(C)CCOC)CC1]

stereoisomers thereof, or pharmaceutically acceptable salts or solvates thereof.

For example, the EZH2 inhibitor is Compound D having the following formula:

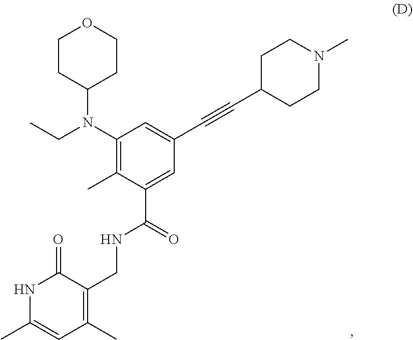

stereoisomers thereof, or pharmaceutically acceptable salts or solvates thereof.

For example, the EZH2 inhibitor is Compound E having the following formula:

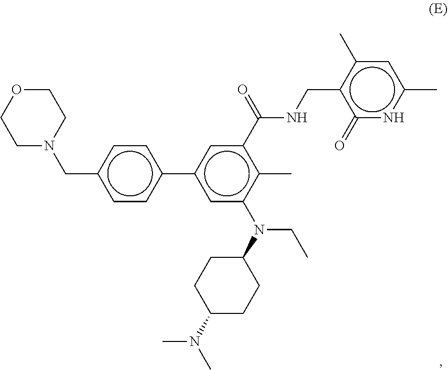

stereoisomers thereof, or pharmaceutically acceptable salts or solvates thereof.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTIONS OF FIGURES

FIGS. 1A and 1B are a series of Western blot analyses of cell lines with wild type (RD and SJCRH30) and mutant SNF5.

FIG. 2A-2E are a series of graphs establishing that SNF5 mutant cell lines A204 (FIG. 2C), G401 (FIG. 2D) and G402 (FIG. 2E) selectively respond to EZH2 compound (Compound E) compared to wild type cell lines RD (FIG. 2A) and SJCRH30 (FIG. 2B).

FIG. 3A-3D are a series of bar graphs showing that G401 SNF mutant cell line is responding to Compound E after 7 days in soft agar compared to wild type cells RD. FIG. 3A shows cell line RD (5,000 cells/well). FIG. 3B shows G401 cells (5,000 cells/well). FIG. 3C shows G401 cells in 2D growth. FIG. 3D shows G401 cells (10,000 cells/well).

FIG. 4A-4D are four graphs showing that G401 SNF5 mutant cell line is sensitive to Compound A in vitro. Wild type cell lines SJCRH30 (FIG. 4A) and RD (FIG. 4C) and SNF5 mutant cell lines G401 (FIG. 4B) and A204 (FIG. 4D) were pretreated for 7 days with indicated concentrations of Compound A and replated on day 0. Cell viability was determined by CellTiter-Glo® Luminescent Cell Viability Assay.

FIG. 5A-5E are a series of graphs showing durable regressions in G401 xenografts (malignant rhabdoid tumor model) with Compound A treatment. FIG. 5A shows tumor regressions induced by Compound A at the indicated doses. FIG. 5B shows tumor regressions induced by twice daily administration of Compound A at the indicated doses. Data represent the mean values±SEM (n=8). Compound administration was stopped on day 28. FIG. 5C shows the EZH2 target inhibition in G401 xenograft tumor tissue collected from a parallel cohort of mice on day 21. Each point shows the ratio of H3K27Me3 to total H3. Horizontal lines represent group mean values. BLLQ=below lower limit of quantification. FIGS. 5D and 5E show immunohistochemical staining of tumor histone methylation of tumor samples from the vehicle treated (FIG. 5D) and Compound A treated (FIG. 5E) (at 125 mg/kg) mice.

FIG. 6 is a graph showing the locations of ATRX mutations identified in SCLC cell lines.

FIG. 7A is a graph showing that LNCAP prostate cancer cells display dose-dependent cell growth inhibition with Compound E treatment in vitro.

FIG. 7B is a graph showing IC50 value of Compound E at day 11 and day 14 for WSU-DLCL2 and LNCAP cells.

FIG. 8A-8C are three graphs establishing that ATRX mutant SCLC lines NCI-H446 (FIG. 8A), SW1271 (FIG. 8B) and NCI-H841 (FIG. 8C) are responding to Compound E.

FIG. 9A-9C are three microscopy images showing that SCLC line NCI-H841 changes morphology after treatment with vehicle (FIG. 9A) or Compound E at concentration of 4.1E-02 uM (FIG. 9B) or 3.3 uM (FIG. 9C).

FIG. 10A-10F are a series of graphs showing effects of Compound A on cellular global histone methylation and cell viability. FIG. 10A shows the chemical structure of Compound A. FIG. 10B illustrates the concentration-dependent inhibition of cellular H3K27Me3 levels in G401 and RD cells. FIG. 10C through 10F illustrate the selective inhibition of proliferation of SMARCB1-deleted G401 cells by Compound A in vitro (measured by ATP content). G401 (FIGS. 10C and 10D) and RD cells (FIGS. 10E and 10F) were re-plated at the original seeding densities on day 7. Each point represents the mean for each concentration (n=3).

FIGS. 11A and 11B are a series of graphs showing biochemical mechanism of action studies. The IC₅₀ value of Compound A increases with increasing SAM concentration (FIG. 11A) and is minimally affected by increasing oligonucleosome concentration (FIG. 11B), indicating SAM-competitive and nucleosome-noncompetitive mechanism of action.

FIGS. 12A and 12B are a series of immunoblots demonstrating verification of SMARCB1 and EZH2 expression in cell lines and specificity of Compound A for inhibition of cellular histone methylation. FIG. 12A shows cell lysates analyzed by immunoblot with antibodies specific to SMARCB1, EZH2 and Actin (loading control). FIG. 12B illustrates selective inhibition of cellular H3K27 methylation in G401 and RD cells. Cells were incubated with Compound A for 4 days, and acid-extracted histones were analyzed by immunoblot.

FIGS. 13A and 13B are a series of bar graphs demonstrating that Compound A induces G1 arrest and apoptosis in SMARCB1-deleted MRT cells. Cell cycle analysis (by flow cytometry) and determination of apoptosis (by TUNEL assay) in RD (FIG. 13A) or G401 cells (FIG. 13B) during incubation with either vehicle or 1 μM Compound A for up to 14 days. G1 arrest was observed as of day 7 and apoptosis was induced as of day 11. Data are represented as mean values±SEM (n=2). The DMSO control values shown are the average±SEM from each time point. Cells were split and re-plated on days 4, 7 and 11 at the original seeding density.

FIG. 14A-14L are a series of graphs showing that Compound A induces changes in expression of SMARCB1 regulated genes and cell morphology. FIG. 14A shows the basal expression of SMARCB1 regulated genes in G401 SMARCB1-deleted cells, relative to RD control cells (measured by qPCR, n=2). FIG. 14B-14K show the expression of GLI1 (FIG. 14B), PTCh1 (FIG. 14C), DOCK4 (FIG. 14D), CD133 (FIG. 14E), PTPRK (FIG. 14F), BIN1 (FIG. 14G), CDKN1A (FIG. 14H), CDKN2A (FIG. 14I), EZH2 (FIG. 14J), and MYC (FIG. 14K) genes in G401 and RD cells. The cells were incubated with either DMSO or 1 μM Compound A for 2, 4 and 7 days. Gene expression was determined by qPCR (n=2) and is expressed relative to the DMSO control of each time point. FIG. 14L illustrates the morphology of G402 cells that were incubated with either DMSO (left panel) or 1 μM Compound A (right panel) for 14 days. Cells were split and re-plated to the original seeding density on day 7.

FIG. 15A-15D are a series of graphs demonstrating body weights, tumor regressions and plasma levels in G401 xenograft bearing mice treated with Compound A. FIG. 15A shows body weights that were determined twice a week for animals treated with Compound A on a BID schedule for 28 days. Data are presented as mean values±SEM (n=16 until day 21, n=8 from day 22 to 60). FIG. 15B shows tumor regressions induced by twice daily (BID) administration of Compound A for 21 days at the indicated doses (mean values±SEM, n=16). * p<0.05, ** p<0.01, repeated measures ANOVA, Dunnett's post-test vs. vehicle. FIG. 15C shows the tumor weights of 8 mice euthanized on day 21. **** p<0.0001, Fisher's exact test. FIG. 15D shows plasma levels of Compound A. Plasma was collected 5 min before and 3 h after dosing of Compound A on day 21, and compound levels were measured by LC-MS/MS. Animals were euthanized, and tumors were collected 3 h after dosing on day 21. Tumor homogenates were generated and subjected to LC-MS/MS analysis to determine Compound A concentrations. Note that tumor compound levels could not be determined from all animals especially in the higher dose groups because the xenografts were too small on day 21. Dots represent values for the individual animals; horizontal lines represent group mean values.

FIG. 16A-16F are a series of graphs showing that Compound A eradicates SMARCB1-deleted MRT xenografts in SCID mice. FIG. 16A illustrates tumor regressions induced by twice daily (BID) administration of Compound A for 28 days at the indicated doses. Compound administration was stopped on day 28 and tumors were allowed to re-grow until they reached 2000 mm³ (data shown as mean values±SEM, n=8). FIG. 16B shows the EZH2 target inhibition in G401 xenograft tumor tissue collected from mice euthanized on day 21. Each point shows the ratio of H3K27Me3 to total H3, measured by ELISA. Horizontal lines represent group mean values; grey symbols are values outside of the ELISA standard curve.

FIG. 16C-16F summarize the change in gene expression in G401 xenograft tumor tissue collected from mice treated with Compound A for 21 days. The graphs correspond to genes CD133 (FIG. 16C), PTPRK (FIG. 16D), DOCK4 (FIG. 16E), and GLI1 (FIG. 16F), respectively. Data are presented as fold change compared to vehicle±SEM (n=6, n=4 for 500 mg/kg group). * p<0.05, ** p<0.01, **** p<0.0001, vs. vehicle, Fisher's exact test.

### DETAILED DESCRIPTION OF THE INVENTION

The present invention is based in part upon the discovery that EZH2 inhibitors can effectively treat SWI/SNF-associated cancers that are characterized by altered expressions and/or loss of function of certain biomarkers or genes. Specifically, tumors or tumor cells having altered expressions and/or loss of function of selected biomarkers or genes are sensitive to the EZH2 inhibitors of the present invention. Accordingly, the present invention provides methods of treating or alleviating a symptom of cancers in a subject by administering a therapeutically effective amount of an EZH2 inhibitor to the subject, particular treating cancers associated with altered expression and/or loss of function of certain biomarkers or genes. For example, the biomarker is one component of the SWI/SNF complex. For example, the gene is selected from the group consisting of neuronal differentiation genes, cell cycle gene inhibition genes, tumor suppressor genes, hedgehog pathway genes, myc pathway genes and histone methyltransferase genes.

The SWI/SNF complex in human includes at least evolutionarily conserved core subunits and variant subunits. Evolutionarily conserved core subunits include SNF5 (also called SMARCB1, INI1 or BAF47), SMARCA4 (also known as BRM/SWI2-related gene 1, BRG1), BAF155, and BAF170. Variant subunits include BAF53 (A or B), BAF60 (A, B or C), BAF 57, BAF45 (A, B, C, or D). Other subunits include ARIDI1A (also known as SMARCF1), ARID1B, SMARCA2 (also known as brahma homologue, BRM), ATRX, BAF200, BAF180 (also known as PBRM1), and bromodomain-containing 7 (BRD7). The at least one component of the SWI/SNF complex can by any component of the complex, for example, the component/subunit described herein or known in the art.

In any methods presented herein, neuronal differentiation gene may be, but is not limited to, CD133 (also called PROM1), DOCK4, PTPRK, PROM2, LHX1, LHX6, LHX9, PAX6, PAX7, VEFGA, FZD3B, FYN, HIF1A, HTRA2, EVX1, CCDC64, or GFAP.

In any methods presented herein, cell cycle inhibition gene may be, but is not limited to, CKDNIA, CDKN2A, MEN1, CHEK1, IRF6, ALOX15B, CYP27B1, DBC1, NME6, GMNN, HEXIM1, LATS1, MYC, HRAS, TGFB1, IFNG, WNT1, TP53, THBS1, INHBA, IL8, IRF1, TPR, BMP2, BMP4, ETS1, HPGD, BMP7, GATA3, NR2F2, APC, PTPN3, CALR, IL12A, IL12B, PML, CDKN2B, CDKN2C, CDKN1B, SOX2, TAF6, DNA2, PLK1, TERF1, GAS1, CDKN2D, MLF1, PTEN, TGFB2, SMAD3, FOX04, CDK6, TFAP4, MAP2K1, NOTCH2, FOXC1, DLG1, MAD2L1, ATM, NAE1, DGKZ, FHL1, SCRIB, BTG3, PTPRK, RPS6KA2, STK11, CDKN3, TBRG1, CDC73, THAP5, CRLF3, DCUNID3, MYOCD, PAF1, LILRB1, UHMK1, PNPT1, USP47, HEXIM2, CDK5RAP1, NKX3-1, TIPIN, PCBP4, USP44, RBM38, CDT1, RGCC, RNF167, CLSPN, CHMP1A, WDR6, TCF7L2, LATS2, RASSF1, MLTK, MAD2L2, FBX05, ING4, or TRIM35.

In any methods presented herein, tumor suppressor gene may be, but is not limited to, BIN1. As used herein, the term “tumor suppressor gene” has its commonly understood meaning in the art, i.e. a gene whose expression and normal function act to suppress the neoplastic phenotype or induce apoptosis, or both. In some embodiments, tumor suppressor genes include cell cycle inhibition genes. Exemplary categories of tumor suppressors based on their functions include, but not limited to:
(1) genes that inhibit cell cycles;
(2) genes that are coupling the cell cycle to DNA damage. When there is damaged DNA in the cell, the cell should not divide. If the damage can be repaired, the cell cycle can continue. If the damage cannot be repaired, the cell should initiate apoptosis (programmed cell death);
(3) genes that prevent tumor cells from dispersing, block loss of contact inhibition, and inhibit metastasis. These genes and their encoded proteins are also known as metastasis suppressors; and
(4) DNA repair proteins. Mutations in these genes increase the risk of cancer.

In any methods presented herein, hedgehog signaling pathway gene may be, but is not limited to, GLI1, PTCH1, SUFU, KIF7, GLI2, BMP4, MAP3K10, SHH, TCTN3, DYRK2, PTCHD1, or SMO.

In any methods presented herein, myc pathway gene may be, but is not limited to, MYC NMI, NFYC, NFYB, Cyclin T1, RuvB-like 1, GTF2I, BRCA1, T-cell lymphoma invasion and metastasis-inducing protein 1, ACTL6A, PCAF, MYCBP2, MAPK8, Bcl-2, Transcription initiation protein SPT3 homolog, SAP130, DNMT3A, mothers against decapentaplegic homolog 3, MAX, mothers against decapentaplegic homolog 2, MYCBP, HTATIP, ZBTB17, Transformation/transcription domain-associated protein, TADA2L, PFDN5, MAPK1, TFAP2A, P73, TAF9, YY1, SMARCB1, SMARCA4, MLH1, EP400 or let-7.

In any methods presented herein, histone methyltransferase gene may be, but is not limited to, EZH2.

Compounds of the present invention inhibit the histone methyltransferase activity of EZH2 or a mutant thereof and, accordingly, in one aspect of the invention, compounds disclosed herein are candidates for treating or preventing certain conditions and diseases. The present invention provides methods for treating, preventing or alleviating a symptom of cancer or a precancerous condition. The method includes administering to a subject in need thereof, a therapeutically effective amount of a compound of the present invention, or a pharmaceutically acceptable salt, polymorph, solvate, or stereoisomeror thereof. Exemplary cancers that may be treated include medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epithelioid sarcoma, renal medullo carcinoma, diffuse large B-cell lymphoma, follicular lymphoma and not otherwise specified (NOS) sarcoma. Alternatively, cancers to be treated by the compounds of the present invention are non NHL cancers.

The present invention further provides the use of a compound of the present invention, or a pharmaceutically acceptable salt, polymorph or solvate thereof in the treatment of cancer or precancer, or, for the preparation of a medicament useful for the treatment of such cancer or pre-cancer. Exemplary cancers that may be treated include medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epithelioid sarcoma, renal medullo carcinoma, diffuse large B-cell lymphoma, follicular lymphoma and not otherwise specified (NOS) sarcoma. Alternatively, the compound of the present invention can be used for the treatment of non NHL cancers, or, for the preparation of a medicament useful for the treatment of non NHL cancers.

The compounds of this invention can be used to modulate protein (e.g., histone) methylation, e.g., to modulate histone methyltransferase or histone demethylase enzyme activity. The compounds of the invention can be used in vivo or in vitro for modulating protein methylation. Based upon the surprising discovery that methylation regulation by EZH2 involves in tumor formation, particular tumors bearing altered expression and/or loss of function of selected biomarkers/genes, the compounds described herein are suitable candidates for treating these diseases, i.e., to decrease methylation or restore methylation to roughly its level in counterpart normal cells.

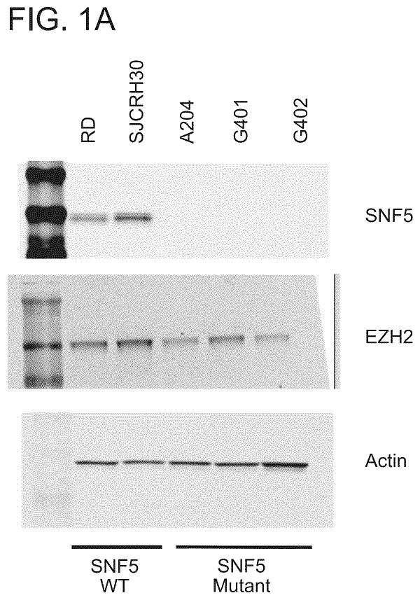

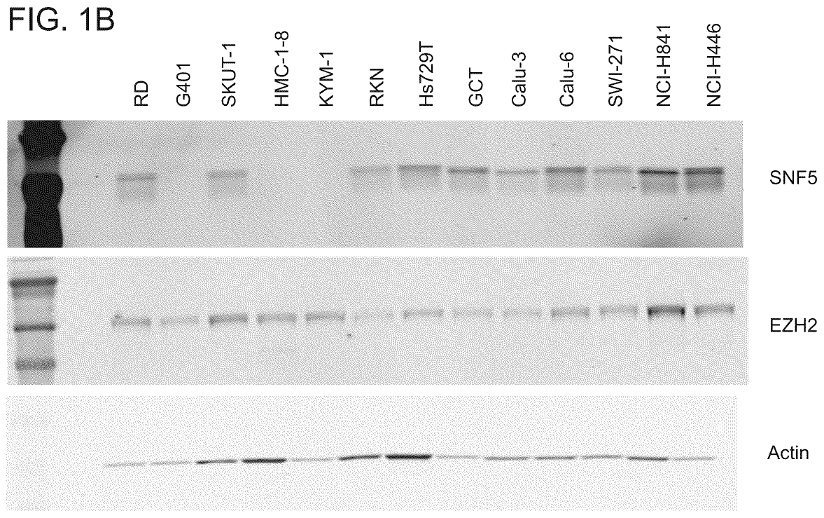

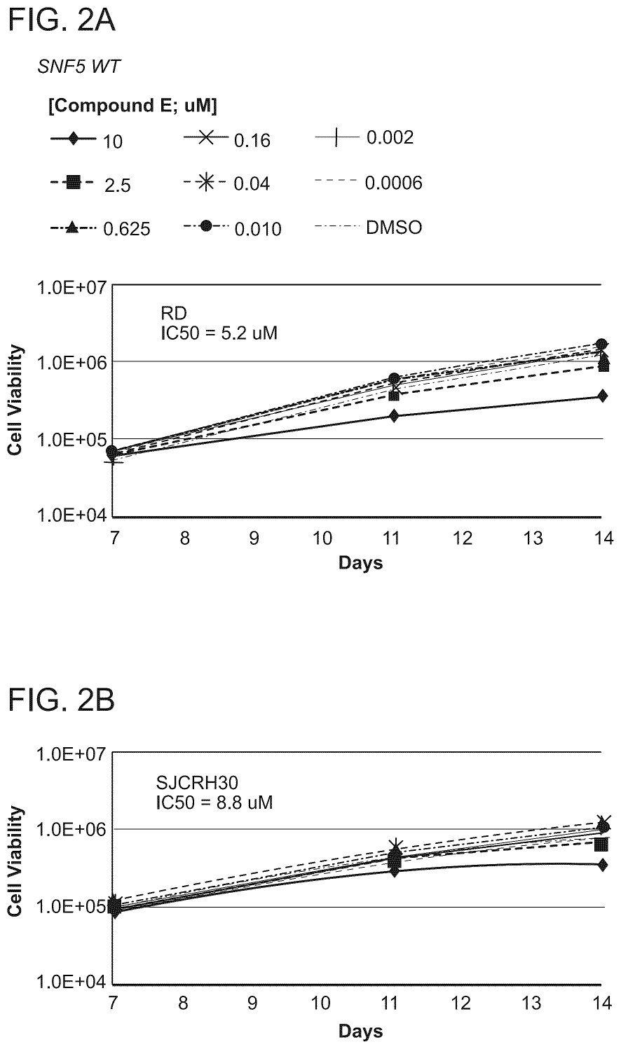

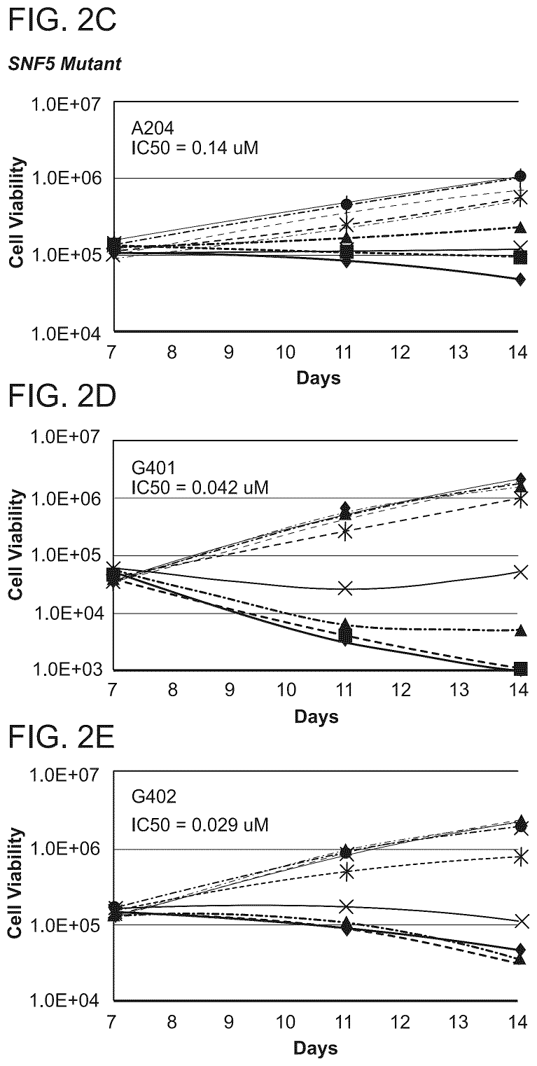

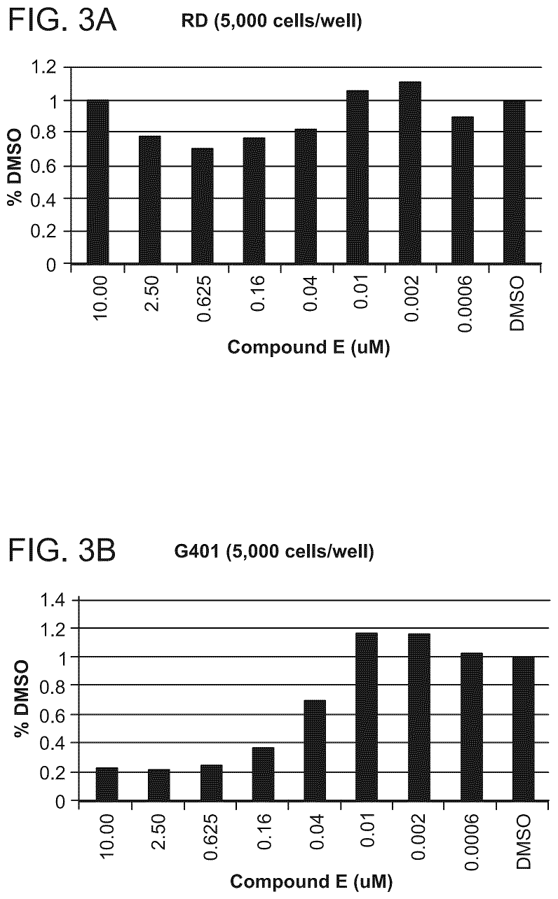

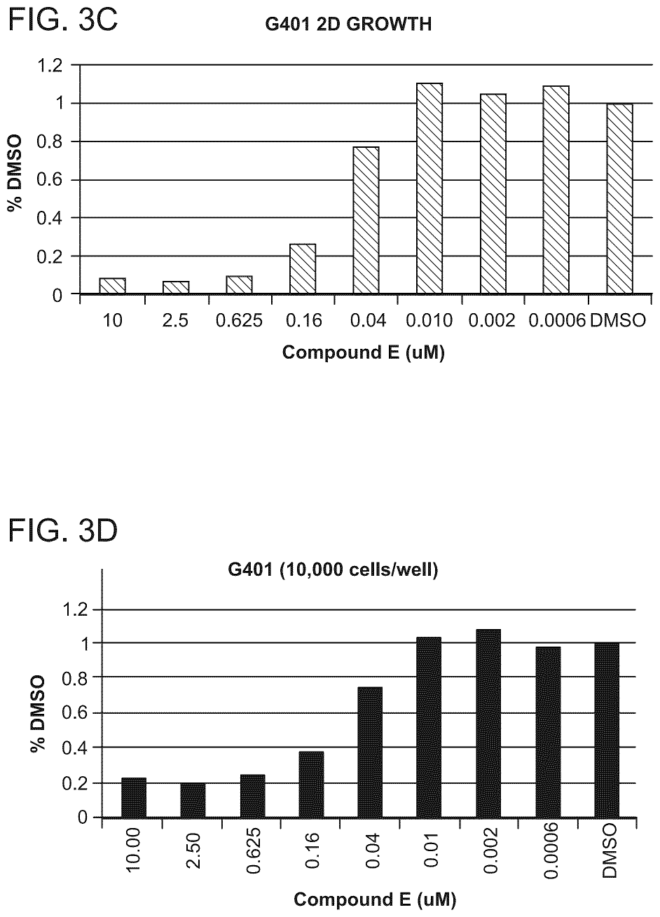

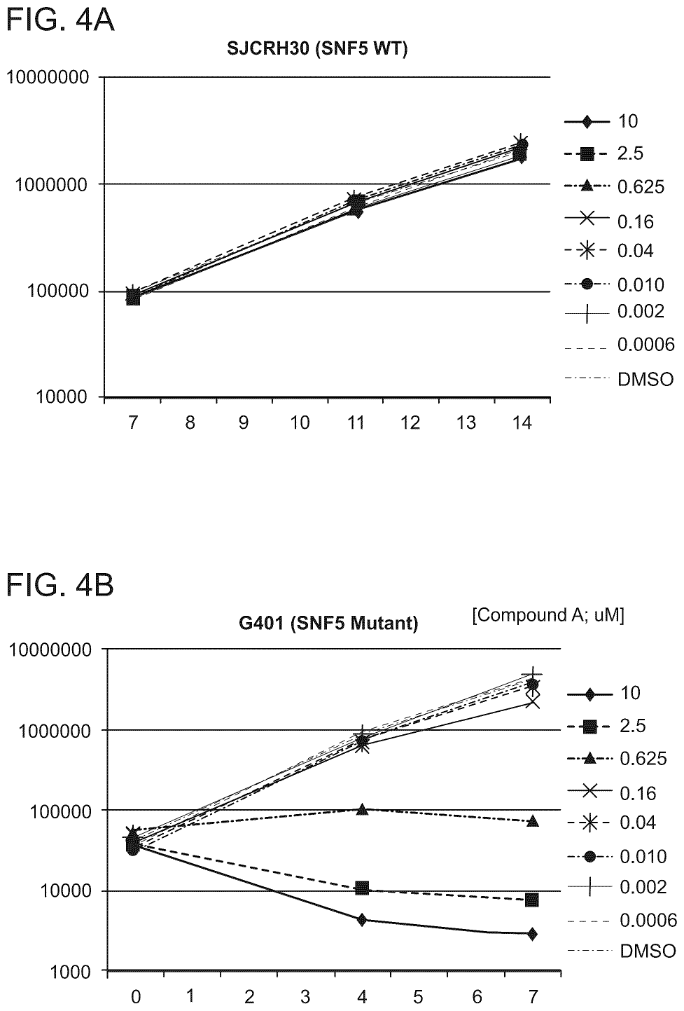

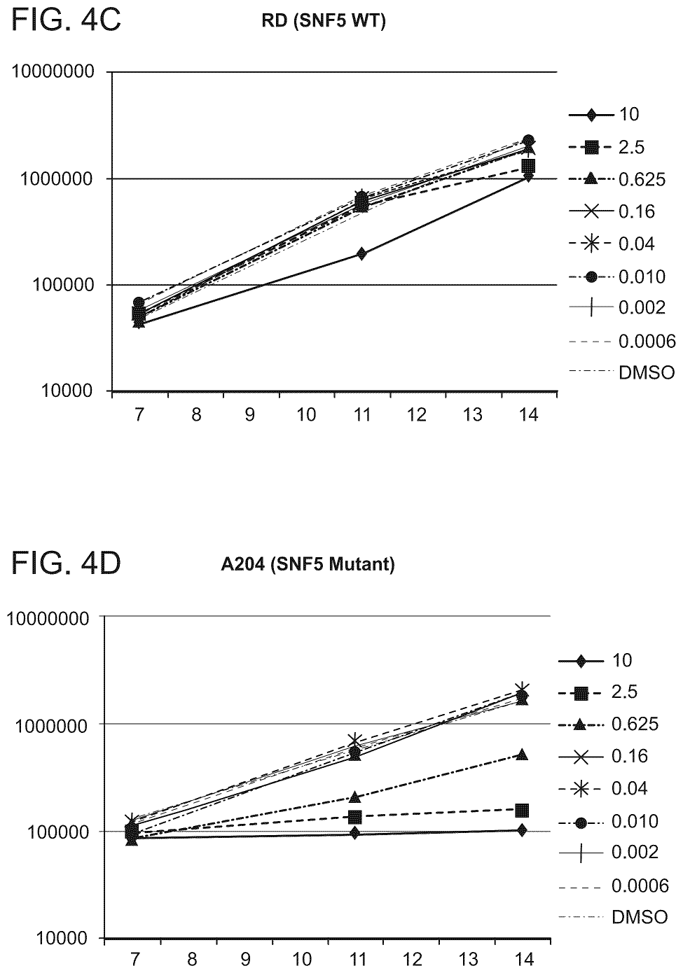

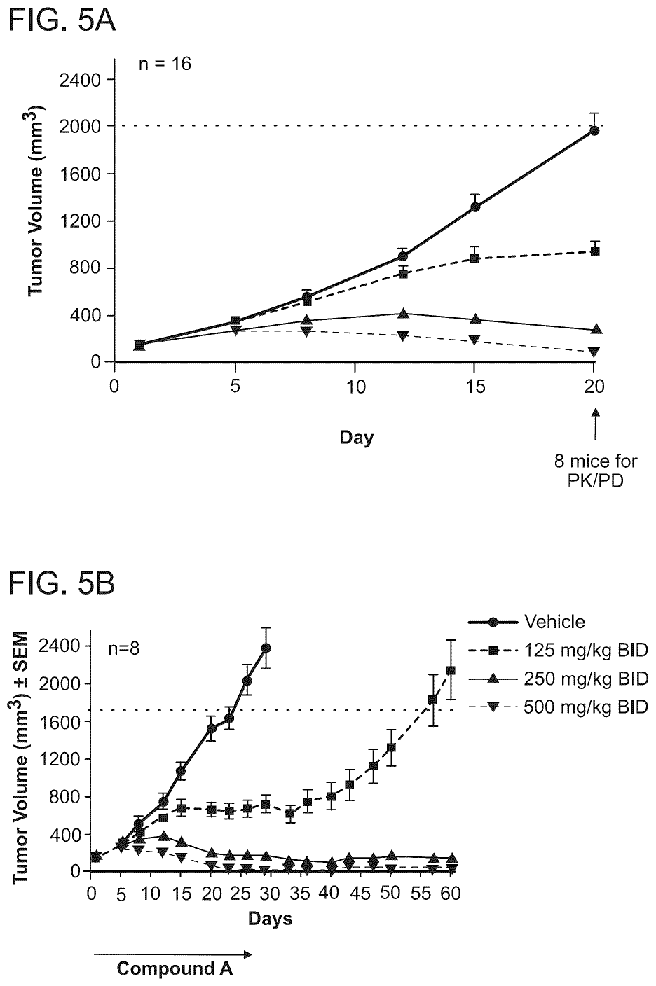

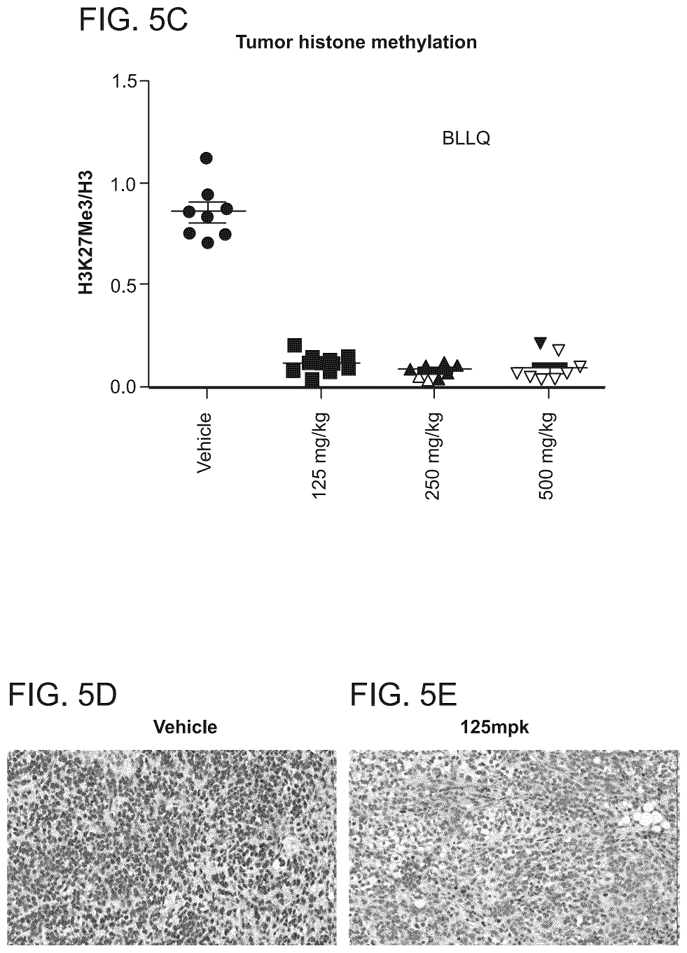

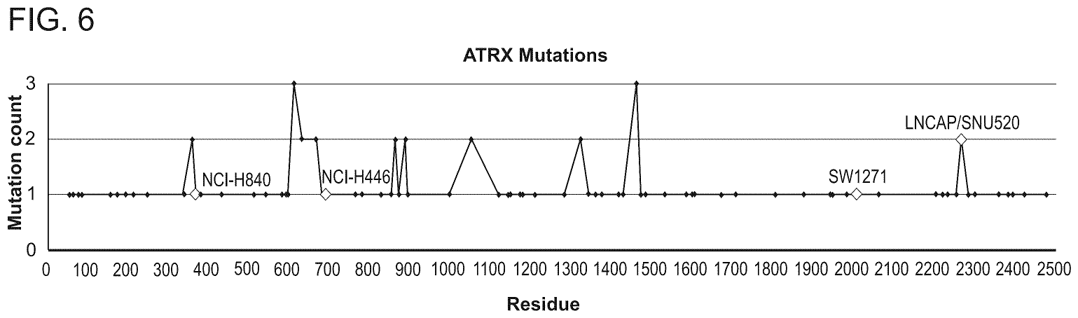

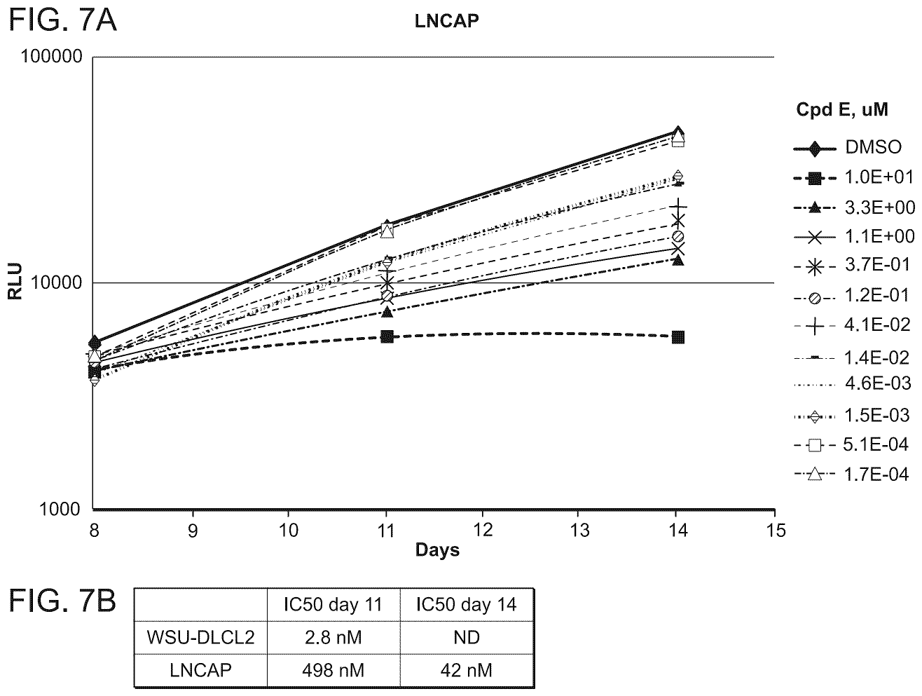

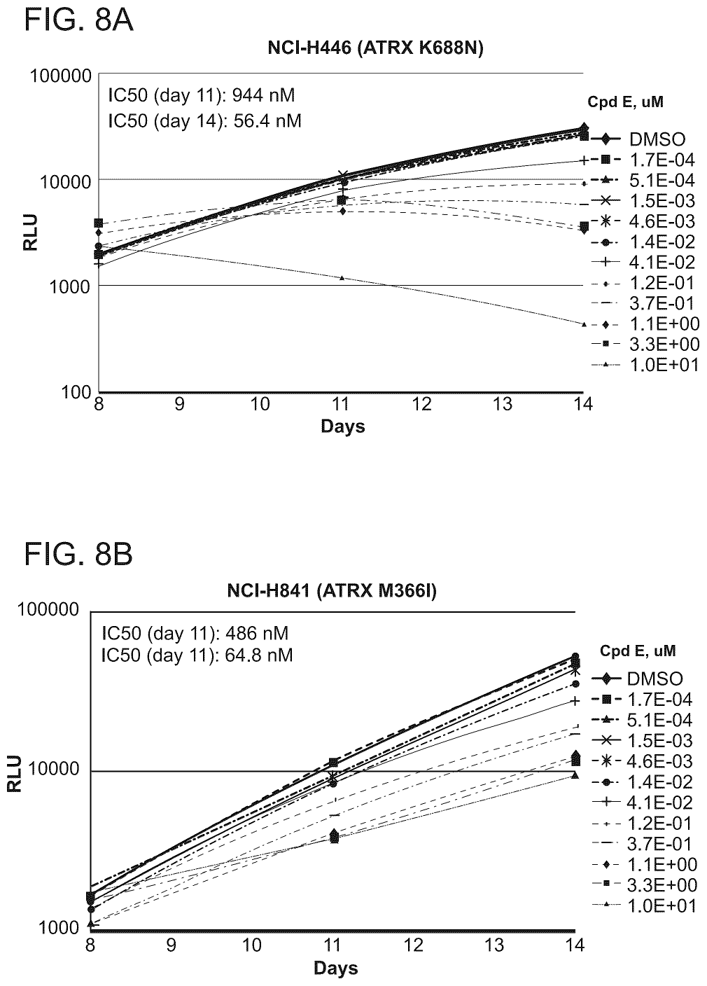

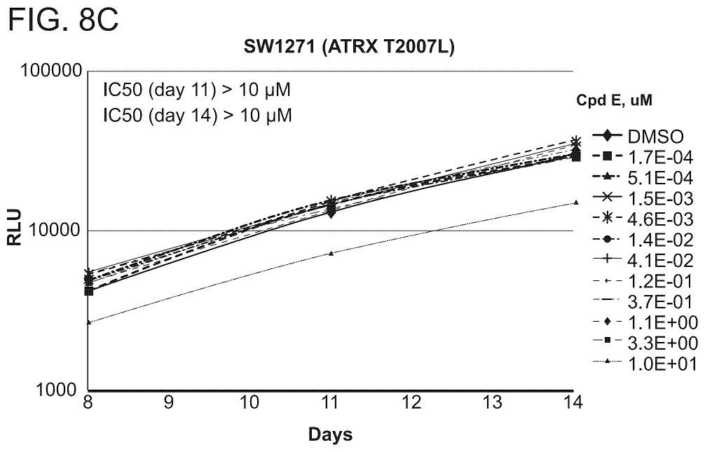

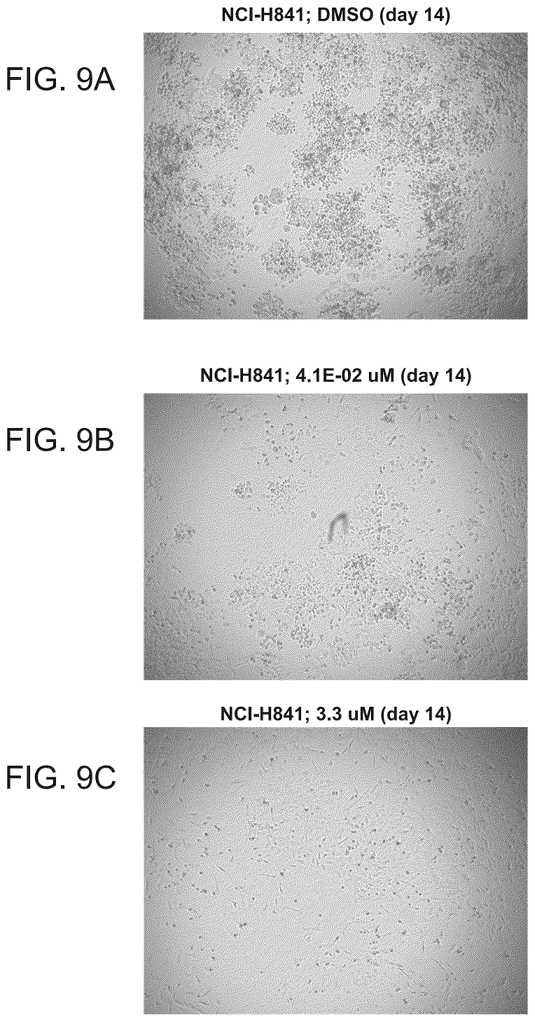

In some embodiments, compounds of the present invention can selectively inhibit proliferation of the SWI/SNF complex associated tumor or tumor cells (as shown in FIGS. 1-9). Accordingly, the present invention provides methods for treating, preventing or alleviating a symptom of the SWI/SNF complex associated cancer or a precancerous condition by a compound of the present invention, or a pharmaceutically acceptable salt, polymorph or solvate thereof. The present invention further provides the use of a compound of the present invention, or a pharmaceutically acceptable salt, polymorph or solvate thereof in the treatment of the SWI/SNF complex associated cancer or a precancer condition, or, for the preparation of a medicament useful for the treatment of such cancer or pre-cancer.

Also provided in the present invention are methods for determining responsiveness of a subject having a cancer to an EZH2 inhibitor. The method includes the steps of obtaining a sample (a nucleic acid sample or a protein sample) from the subject and detecting reduced expression, haploinsufficiency, and/or loss of function of at least one component of the SWI/SNF complex, detecting the expression and/or function of this component, and the presence of such reduced expression, haploinsufficiency, and/or loss of function indicates that the subject is responsive to the EZH2 inhibitor. The term “sample” means any biological sample derived from the subject, includes but is not limited to, cells, tissues samples, body fluids (including, but not limited to, mucus, blood, plasma, serum, urine, saliva, and semen), tumor cells, and tumor tissues. Samples can be provided by the subject under treatment or testing. Alternatively samples can be obtained by the physician according to routine practice in the art.

The present invention also provides methods for determining predisposition of a subject to a cancer or a precancerous condition by obtaining a sample from the subject and detecting reduced expression, haploinsufficiency, and/or loss of function of at least one component of the SWI/SNF complex, and the presence of such reduced expression, haploinsufficiency, and/or loss of function indicates that the subject is predisposed to (i.e., having higher risk of) developing the cancer or the precancerous condition compared to a subject without such loss of function of the at least one component of the SWI/SNF complex.

The term “predisposed” as used herein in relation to cancer or a precancerous condition is to be understood to mean the increased probability (e.g., at least 1%, 5%, 10%, 20%, 30%, 40%, 50%, 60%, 70%, 80%, 90%, 100%, 150%, 200%, or more increase in probability) that a subject with reduced expression, haploinsufficiency, and/or loss of function of at least one component of the SWI/SNF complex, will suffer cancer or a precancerous condition, as compared to the probability that another subject not having reduced expression, haploinsufficiency, and/or loss of function of at least one component of the SWI/SNF complex, will suffer cancer or a precancerous condition, under circumstances where other risk factors (e.g., chemical/environment, food, and smoking history, etc.) for having cancer or a precancerous condition between the subjects are the same.

“Risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion.

Accordingly, the present invention provides personalized medicine, treatment and/or cancer management for a subject by genetic screening of reduced expression, haploinsufficiency, and/or loss of function of at least one component of the SWI/SNF complex in the subject. For example, the present invention provides methods for treating, preventing or alleviating a symptom of cancer or a precancerous condition by determining responsiveness of the subject to an EZH2 inhibitor and when the subject is responsive to the EZH2 inhibitor, administering to the subject a therapeutically effective amount of the EZH2 inhibitor, or a pharmaceutically acceptable salt, solvate, or stereoisomeror thereof. The responsiveness is determined by obtaining a sample from the subject and detecting reduced expression, haploinsufficiency, and/or loss of function of at least one component of the SWI/SNF complex (such as SNF5, ARID1A or ATRX), and the presence of such loss of function indicates that the subject is responsive to the EZH2 inhibitor.

In other example, the present invention provides methods of cancer management in a subject by determining predisposition of the subject to a cancer or a precancerous condition periodically. The methods include steps of obtaining a sample from the subject and detecting reduced expression, haploinsufficiency, and/or loss of function of at least one component of the SWI/SNF complex, and the presence of such reduced expression, haploinsufficiency, and/or loss of function indicates that the subject is predisposed to developing the cancer or the precancerous condition compared to a subject without such reduced expression, haploinsufficiency, and/or loss of function of the at least one component of the SWI/SNF complex.

In merely illustrative embodiments, the methods of treatment presented herein include steps of (a) collecting a nucleic acid sample or a protein sample from a biological sample obtained from a subject, (b) measuring the expression level or function level of a component of the SWI/SNF complex in the sample, (c) measuring the expression level or function level of the component of the SWI/SNF in a control sample; (d) comparing the expression level or the function level of the component measured in step (b) in the tested sample to the expression level or the function level of the component measured in step (c) in the control sample (or a reference value); (e) identifying the subject as a candidate for treatment when the expression level or the function level of the component measured in step (b) is reduced or lost (e.g., haploinsufficiency or loss of function) compared to the expression level or the function level of the component measured in step (c); and (f) administering a therapeutically effective amount of an EZH2 inhibitor to the subject identified in step (e) or selecting a treatment regimen for the subject identified in step (e). The expression level or the function level of component in the subject sample is reduced, for example, 10%, 25%, 50% or 1-, 2-, 5- or more fold compared to the expression level or the function level of the component in the control sample. Any suitable methods known in the art can be utilized to measure the expression level or the function level of the component of the SWI/SNF complex. In some embodiments, the subject has malignant rhabdoid tumor, medulloblastoma or atypical teratoid rhabdoid tumor. In some embodiments, the component is SNF5, ARID1A or ATRX.

For example, the identified subject can be treated with the standard of care treatment as described in the most current National Comprehensive Cancer Network (NCCN) guidelines.

For example, a control sample is obtained from a healthy, normal subject. Alternatively, a control sample is obtained from a subject who is not suffering, has not been diagnosed, or is not at risk of developing cancer associated with the SWI/SNF complex.

In one preferred aspect, the present invention provides a method for treating or alleviating a symptom of cancer in a subject by determining responsiveness of the subject to an EZH2 inhibitor and administering to the subject a therapeutically effective amount of the EZH2 inhibitor if the subject is responsive to the EZH2 inhibitor and the subject has a cancer selected from the group consisting of brain and CNS cancer, kidney cancer, ovarian cancer, pancreatic cancer, leukemia, lymphoma, myeloma, and/or sarcoma. Such responsiveness is determined by obtaining a sample from the subject and detecting reduced expression, haploinsufficiency, and/or loss of function of SNF5, ARID1A, and/or ATRX, and the presence of the reduced expression, haploinsufficiency, and/or loss of function indicates the subject is responsive to the EZH2 inhibitor.

In another preferred aspect, the present invention provides a method for treating or alleviating a symptom of malignant rhabdoid tumor in a subject by determining responsiveness of the subject to an EZH2 inhibitor and administering to the subject a therapeutically effective amount of the EZH2 inhibitor if the subject is responsive to the EZH2 inhibitor. Such responsiveness is determined by obtaining a sample from the subject and detecting reduced expression, haploinsufficiency, and/or loss of function of SNF5, ARID1A, and/or ATRX, and the presence of the reduced expression, haploinsufficiency, and/or loss of function indicates the subject is responsive to the EZH2 inhibitor.

In another preferred aspect, the present invention provides a method for treating or alleviating a symptom of medulloblastoma in a subject by determining responsiveness of the subject to an EZH2 inhibitor and administering to the subject a therapeutically effective amount of the EZH2 inhibitor if the subject is responsive to the EZH2 inhibitor. Such responsiveness is determined by obtaining a sample from the subject and detecting reduced expression, haploinsufficiency, and/or loss of function of SNF5, ARID1A, and/or ATRX, and the presence of the reduced expression, haploinsufficiency, and/or loss of function indicates the subject is responsive to the EZH2 inhibitor.

In another preferred aspect, the present invention provides a method for treating or alleviating a symptom of atypical teratoid rhabdoid tumor in a subject by determining responsiveness of the subject to an EZH2 inhibitor and administering to the subject a therapeutically effective amount of the EZH2 inhibitor if the subject is responsive to the EZH2 inhibitor. Such responsiveness is determined by obtaining a sample from the subject and detecting reduced expression, haploinsufficiency, and/or loss of function of SNF5, ARID1A, and/or ATRX, and the presence of the reduced expression, haploinsufficiency, and/or loss of function indicates the subject is responsive to the EZH2 inhibitor.

Malignant rhabdoid tumors (MRTs) and atypical teratoid rhabdoid tumors (ATRTs) are extremely aggressive pediatric cancers of the brain, kidney, and soft tissues that are highly malignant, locally invasive, frequently metastatic, and particularly lethal. They are typically diploid and lack genomic aberrations; however, they are characterized by an almost complete penetrance of loss of SMARCB1 (also called SNF5, INI1 or BAF47), a core component of the SWI/SNF chromatin remodeling complex. The biallelic inactivation of SMARCB1 is in essence the sole genetic event in MRTs and ATRTs which suggests a driver role for this genetic aberration.

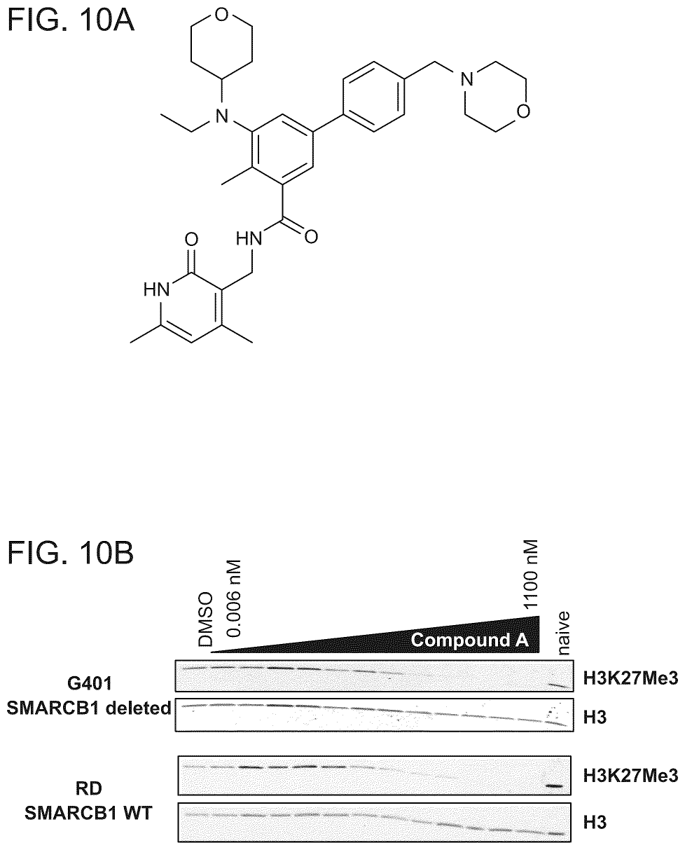

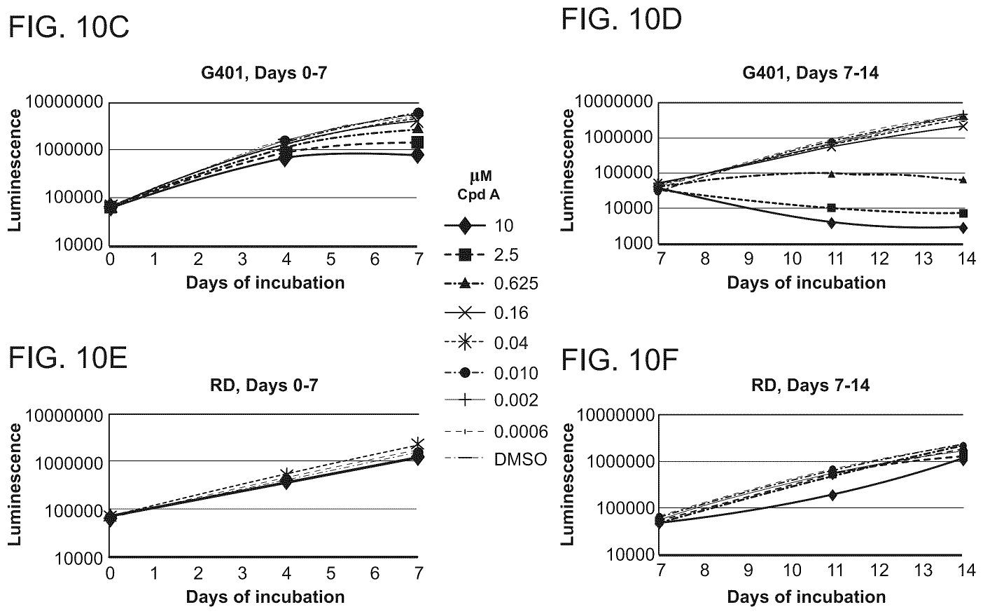

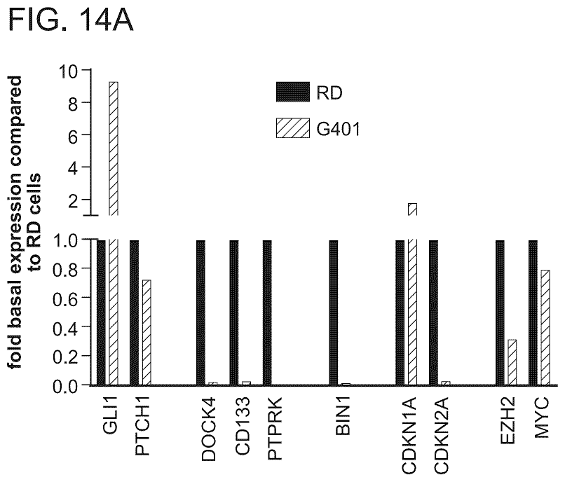

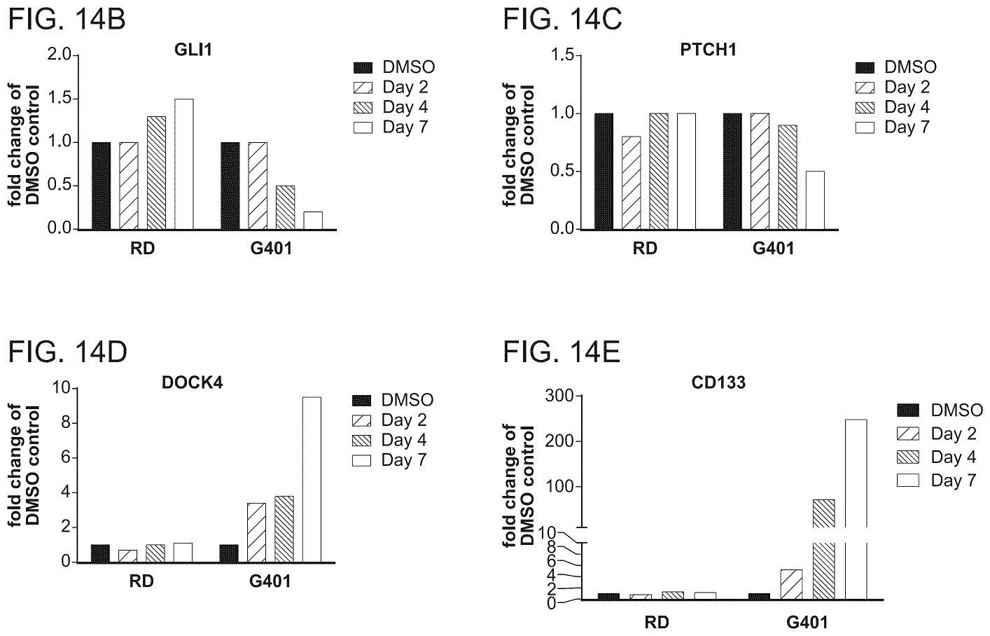

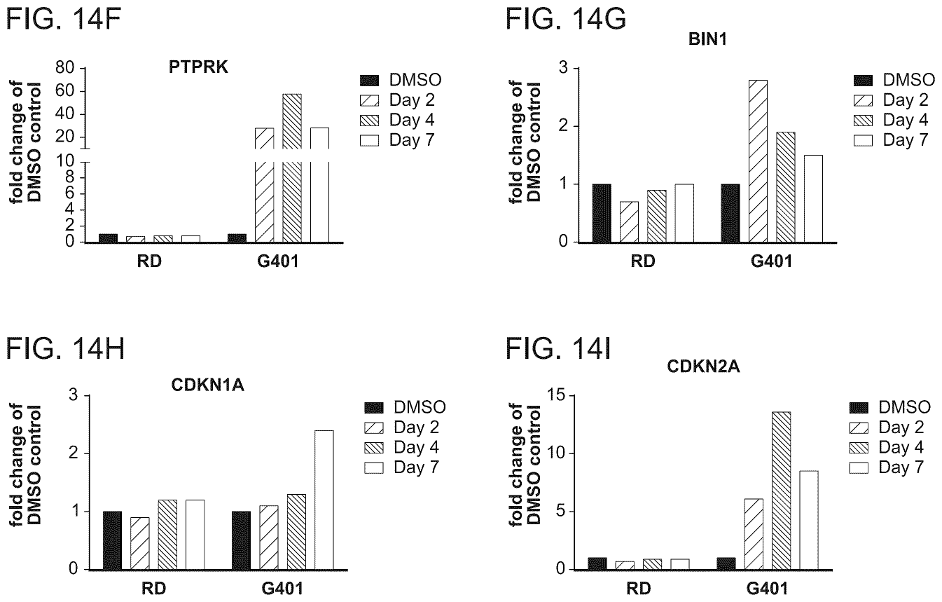

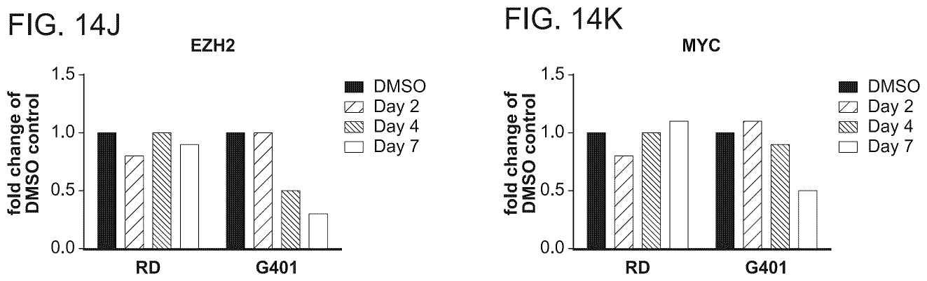

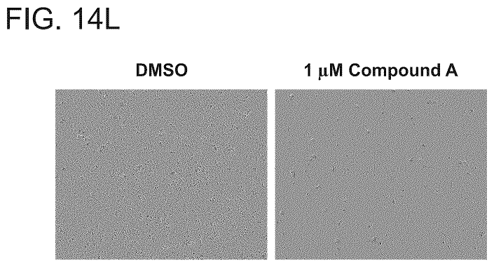

Without being bound by any theory, a compound of the present invention specifically inhibits cellular H3K27 methylation leading to selective apoptotic killing of SMARCB1 mutant MRT cells. For example, in vitro treatment of SMARCB1-deleted MRT cell lines with Compound A induced strong anti-proliferative effects with IC₅₀ values in the nM range; while the control (wild-type) cell lines were minimally affected (FIG. 10C and table 6). Furthermore, the compound of the present invention induces genes of neuronal differentiation, cell cycle inhibition and tumor suppression while suppressing expression of hedgehog pathway genes, MYC and EZH2. For example, Compound A treatment of G401 SMARCB1-deleted cells for up to 7 days strongly induced expression of CD133, DOCK4 and PTPRK and up-regulated cell cycle inhibitors CDKN1A and CDKN2A and tumor suppressor BIN1, all in a time-dependent manner (FIG. 14B). Simultaneously, the expression of hedgehog pathway genes, MYC and EZH2 were reduced. Notably, G402 SMARCB1-deleted cells exposed to Compound A for 14 days assumed a neuron-like morphology (FIG. 14C).

Accordingly, the present invention further provides methods of treating or alleviating a symptom of cancer in a subject in need thereof by (a) determining the expression level of at least one gene selected from the group consisting of neuronal differentiation genes, cell cycle inhibition genes and tumor suppressor genes in a sample obtained from the subject; (b) selecting a subject having a decreased expression level of at least one gene in step (a); and (c) administering to the subject selected in step (b) an effective amount of a compound of the invention, thus treating or alleviating a symptom of cancer in the subject.

The present invention also provides methods of treating or alleviating a symptom of cancer in a subject in need thereof by (a) determining the expression level of at least one gene selected from the group consisting of hedgehog pathway genes, myc pathway genes and histone methyltransferase genes in a sample obtained from the subject; (b) selecting a subject having an increased expression level of at least one gene in step (a); and (c) administering to the subject selected in step (b) an effective amount of a compound of the invention, thus treating or alleviating a symptom of cancer in the subject.

Also provided herein are methods of selecting a cancer therapy for a subject in need thereof by (a) determining the expression level of at least one gene selected from the group consisting of neuronal differentiation genes, cell cycle inhibition genes, and tumor suppressor genes in a sample obtained from the subject, and (b) selecting a cancer therapy when the subject has a decreased expression level of at least one gene in step (a), where the cancer therapy includes the administration of an effective amount of a compound of the invention to the subject.

The present invention further provides methods of selecting a cancer therapy for a subject in need thereof by (a) determining the expression level of at least one gene selected from the group consisting of hedgehog pathway genes, myc pathway genes and histone methyltransferase genes in a sample obtained from the subject, and (b) selecting a cancer therapy when the subject has an increased expression level of at least one gene in step (a), where the cancer therapy includes the administration of an effective amount of a compound of the invention to the subject.

In merely illustrative embodiments, the methods presented herein may include the steps of (a) collecting a nucleic acid or a protein sample from a biological sample obtained from a subject, (b) measuring the expression level of at least one gene selected from the group consisting of neuronal differentiation genes, cell cycle inhibition genes, and tumor suppressor genes in the sample, (c) measuring the expression level of the same gene(s) in a control sample; (d) comparing the expression level of the gene measured in step (b) in the tested sample to the expression level of the gene measured in step (c) in the control sample (or to a reference value); (e) identifying the subject as a candidate for treatment when the expression level of the component measured in step (b) is reduced compared to the expression level of the gene measured in step (c); and (f) administering a therapeutically effective amount of an EZH2 inhibitor to the subject identified in step (e) or selecting a treatment regimen for the subject identified in step (e). The expression level of the gene in the tested subject is reduced, for example, 10%, 25%, 50% or 1-, 2-, 5- or more fold compared to the expression level of the gene in the control sample.

In merely illustrative embodiments, the methods presented herein may include the steps of (a) collecting a nucleic acid or a protein sample from a biological sample obtained from a subject, (b) measuring the expression level of at least one gene selected from the group consisting of hedgehog pathway genes, myc pathway genes and histone methyltransferase genes in the sample, (c) measuring the expression level of the same gene(s) in a control sample; (d) comparing the expression level of the gene measured in step (b) in the tested sample to the expression level of the gene measured in step (c) in the control sample (or to a reference value); (e) identifying the subject as a candidate for treatment when the expression level of the component measured in step (b) is increased compared to the expression level of the gene measured in step (c); and (f) administering a therapeutically effective amount of an EZH2 inhibitor to the subject identified in step (e) or selecting a treatment regimen for the subject identified in step (e). The expression level of the gene in the tested subject is increased, for example, 10%, 25%, 50% or 1-, 2-, 5- or more fold compared to the expression level of the gene in the control sample.

The term “expression level” refers to protein, RNA, or mRNA level of a particular gene of interest. Any methods known in the art can be utilized to determine the expression level of a particular gene of interest. Examples include, but are not limited to, reverse transcription and amplification assays (such as PCR, ligation RT-PCR or quantitative RT-PCT), hybridization assays, Northern blotting, dot blotting, in situ hybridization, gel electrophoresis, capillary electrophoresis, column chromatography, Western blotting, immunohistochemistry, immunostaining, or mass spectrometry. Assays can be performed directly on biological samples or on protein/nucleic acids isolated from the samples. It is routine practice in the relevant art to carry out these assays. For example, the measuring step in any method described herein includes contacting the nucleic acid sample from the biological sample obtained from the subject with one or more primers that specifically hybridize to the gene of interest presented herein. Alternatively, the measuring step of any method described herein includes contacting the protein sample from the biological sample obtained from the subject with one or more antibodies that bind to the biomarker of the interest presented herein.

A decreased expression level of a particular gene means a decrease in its expression level by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level of this gene measured in a different (or previous) sample obtained from the same subject.

An increased expression level of a particular gene means an increase in its expression level by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level of this gene measured in a different (or previous) sample obtained from the same subject.

A “reference or baseline level/value” as used herein can be used interchangeably and is meant to be relative to a number or value derived from population studies, including without limitation, such subjects having similar age range, disease status (e.g., stage), subjects in the same or similar ethnic group, or relative to the starting sample of a subject undergoing treatment for cancer. Such reference values can be derived from statistical analyses and/or risk prediction data of populations obtained from mathematical algorithms and computed indices of cancer. Reference indices can also be constructed and used using algorithms and other methods of statistical and structural classification.

In some embodiments of the present invention, the reference or baseline value is the expression level of a particular gene of interest in a control sample derived from one or more healthy subjects or subjects who have not been diagnosed with any cancer.

In some embodiments of the present invention, the reference or baseline value is the expression level of a particular gene of interest in a sample obtained from the same subject prior to any cancer treatment. In other embodiments of the present invention, the reference or baseline value is the expression level of a particular gene of interest in a sample obtained from the same subject during a cancer treatment. Alternatively, the reference or baseline value is a prior measurement of the expression level of a particular gene of interest in a previously obtained sample from the same subject or from a subject having similar age range, disease status (e.g., stage) to the tested subject.

In some embodiments, an effective amount means an amount sufficient to increase the expression level of at least one gene which is decreased in the subject prior to the treatment or an amount sufficient to alleviate one or more symptoms of cancer. For example, an effective amount is an amount sufficient to increase the expression level of at least one gene selected from the group consisting of neuronal differentiation genes, cell cycle inhibition genes, and tumor suppressor genes by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

In some embodiments, an effective amount means an amount sufficient to decrease the expression level of at least one gene which is increased in the subject prior to the treatment or an amount sufficient to alleviate one or more symptoms of cancer. For example, an effective amount is an amount sufficient to decrease the expression level of at least one gene selected from the group consisting of hedgehog pathway genes, MYC and EZH2 by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

The precise effective amount for a subject will depend upon the subject's body weight, size, and health; the nature and extent of the condition; and the therapeutic selected for administration. An effective amount for a given situation can be determined by routine experimentation that is within the skill and judgment of the clinician.

The present invention further provides a method of determining efficacy of a cancer treatment in a subject in need thereof by (a) measuring the expression level of at least one gene selected from the group consisting of neuronal differentiation genes, cell cycle inhibition genes, and tumor suppressor genes in a sample obtained from the subject, (b) comparing the expression level of at least one gene in step (a) to a reference value or a prior measurement, and (c) determining the efficacy of the cancer treatment based on the comparison step. An exemplary cancer treatment is administering a compound of the invention to the tested subject.

The treatment is effective when the tested subject has an increased expression of at least one gene selected from the group consisting of neuronal differentiation genes, cell cycle inhibition genes and tumor suppressor genes 1) compared to a reference value or a prior measurement; or 2) over the period of time being monitored, such as 1, 2, 3, 4, 5, 6, or 7 days, or 1, 2, 3, 4, 5, 6, 7, 8, 9, 10 weeks, or 1, 2, 3, 4, 5, 6, 7, 8, 9, 10, 11, 12 months or longer. When the existing treatment is not effective, a new treatment or an increased dosage of the existing treatment (for example, increasing the dosage of the compound administered to the subject) should be sought for the tested subject.

The present invention also provides a method of determining efficacy of a cancer treatment in a subject in need thereof by (a) measuring the expression level of at least one gene selected from the group consisting of hedgehog pathway genes, myc pathway genes and histone methyltransferase genes in a sample obtained from the subject, (b) comparing the expression level of at least one gene in step (a) to a reference value or a prior measurement, and (c) determining the efficacy of the cancer treatment based on the comparison step. An exemplary cancer treatment is administering an EZH2 inhibitor of the invention to the tested subject.

For example, the treatment is effective when the tested subject has a decreased expression of at least one gene selected from the group consisting of hedgehog pathway genes, myc pathway genes and histone methyltransferase genes 1) compared to a reference value or a prior measurement; or 2) over the period of time being monitored, such as 1, 2, 3, 4, 5, 6, or 7 days, or 1, 2, 3, 4, 5, 6, 7, 8, 9, 10 weeks, or 1, 2, 3, 4, 5, 6, 7, 8, 9, 10, 11, 12 months or longer. When the existing treatment is not effective, a new treatment or an increased dosage of the existing treatment (for example, increasing the dosage of the compound administered to the subject) should be sought for the tested subject.

In any methods presented herein, cancer is selected from the group consisting of brain and central nervous system (CNS) cancer, head and neck cancer, kidney cancer, ovarian cancer, pancreatic cancer, leukemia, lung cancer, lymphoma, myeloma, sarcoma, breast cancer, and prostate cancer. Preferably, cancer is selected from the group consisting of medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epithelioid sarcoma, renal medullo carcinoma, diffuse large B-cell lymphoma, follicular lymphoma and not otherwise specified (NOS) sarcoma. More preferably, cancer is medulloblastoma, malignant rhabdoid tumor, or atypical teratoid rhabdoid tumor.

As used herein, the term “responsiveness” is interchangeable with terms “responsive”, “sensitive”, and “sensitivity”, and it is meant that a subject is showing therapeutic responses when administered an EZH inhibitor, e.g., tumor cells or tumor tissues of the subject undergo apoptosis and/or necrosis, and/or display reduced growing, dividing, or proliferation. This term is also meant that a subject will or has a higher probability, relative to the population at large, of showing therapeutic responses when administered an EZH inhibitor, e.g., tumor cells or tumor tissues of the subject undergo apoptosis and/or necrosis, and/or display reduced growing, dividing, or proliferation.

As used herein, a “subject” is interchangeable with a “subject in need thereof”, both of which refer to a subject having a disorder in which EZH2-mediated protein methylation plays a part, or a subject having an increased risk of developing such disorder relative to the population at large. A subject in need thereof may be a subject having a disorder associated with SWI/SNF complex. A subject in need thereof can have a precancerous condition. Preferably, a subject in need thereof has cancer. A subject in need thereof can have cancer associated with SWI/SNFcomplex. A subject in need thereof can have cancer associated with loss of function in at least one component of SWI/SNF complex. In a preferred aspect, a subject in need thereof has one or more cancers selected from the group consisting of brain and central nervous system (CNS) cancer, head and neck cancer, kidney cancer, ovarian cancer, pancreatic cancer, leukemia, lung cancer, lymphoma, myeloma, sarcoma, breast cancer, and prostate cancer. Preferably, a subject in need thereof has medulloblastoma, oligodendroglioma, ovarian clear cell adenocarcinoma, ovarian endometrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epithelioid sarcoma, renal medullo carcinoma, diffuse large B-cell lymphoma, follicular lymphoma and not otherwise specified (NOS) sarcoma. Alternatively, a subject in need thereof has a non NHL cancer.

As used herein, a “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject can be male or female.

A subject in need thereof can be one who has not been previously diagnosed or identified as having cancer or a precancerous condition. A subject in need thereof can be one who has been previously diagnosed or identified as having cancer or a precancerous condition. A subject in need thereof can also be one who is having (suffering from) cancer or a precancerous condition. Alternatively, a subject in need thereof can be one who has a risk of developing such disorder relative to the population at large (i.e., a subject who is predisposed to developing such disorder relative to the population at large).

Optionally a subject in need thereof has already undergone, is undergoing or will undergo, at least one therapeutic intervention for the cancer or precancerous condition.

A subject in need thereof may have refractory cancer on most recent therapy. “Refractory cancer” means cancer that does not respond to treatment. The cancer may be resistant at the beginning of treatment or it may become resistant during treatment. Refractory cancer is also called resistant cancer. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy.

A subject in need thereof may be one who had, is having or is predisposed to developing a cancer or a precancerous condition associated with the SWI/SNF complex. A subject in need thereof may be one who had, is having or is predisposed to developing cancer or a precancerous condition associated with loss of function of at least one component of the SWI/SNF complex. In a preferred aspect, a subject in need thereof is one who had, is having or is predisposed to developing one or more cancers selected from the group consisting of brain and central nervous system (CNS) cancer, head and neck cancer, kidney cancer, ovarian cancer, pancreatic cancer, leukemia, lung cancer, lymphoma, myeloma, sarcoma, breast cancer, and prostate cancer. Preferably, a subject in need thereof is one who had, is having or is predisposed to developing brain and CNS cancer, kidney cancer, ovarian cancer, pancreatic cancer, leukemia, lymphoma, myeloma, and/or sarcoma. Exemplary brain and central CNS cancer includes medulloblastoma, oligodendroglioma, atypical teratoid rhabdoid tumor, choroid plexus carcinoma, choroid plexus papilloma, ependymoma, glioblastoma, meningioma, neuroglial tumor, oligoastrocytoma, oligodendroglioma, and pineoblastoma. Exemplary ovarian cancer includes ovarian clear cell adenocarcinoma, ovarian endomethrioid adenocarcinoma, and ovarian serous adenocarcinoma. Exemplary pancreatic cancer includes pancreatic ductal adenocarcinoma and pancreatic endocrine tumor. Exemplary sarcoma includes chondrosarcoma, clear cell sarcoma of soft tissue, ewing sarcoma, gastrointestinal stromal tumor, osteosarcoma, rhabdomyosarcoma, and not otherwise specified (NOS) sarcoma. Alternatively, cancers to be treated by the compounds of the present invention are non NHL cancers.

Alternatively, a subject in need thereof is one who had, is having or is predisposed to developing one or more cancers selected from the group consisting of medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, and not otherwise specified (NOS) sarcoma. Preferably, a subject is one who had, is having or is predisposed to developing medulloblastoma, ovarian clear cell adenocarcinoma, ovarian endomethrioid adenocarcinoma, pancreatic ductal adenocarcinoma, malignant rhabdoid tumor, atypical teratoid rhabdoid tumor, choroid plexus carcinoma, choroid plexus papilloma, glioblastoma, meningioma, pineoblastoma, carcinosarcoma, extrarenal rhabdoid tumor, schwannoma, skin squamous cell carcinoma, chondrosarcoma, ewing sarcoma, epithelioid sarcoma, renal medullo carcinoma, diffuse large B-cell lymphoma, follicular lymphoma and/or NOS sarcoma. More preferably, a subject in need thereof is one who had, is having or is predisposed to developing malignant rhabdoid tumor, medulloblastoma and/or atypical teratoid rhabdoid tumor.

In some embodiments of the present invention, a subject in need thereof has a decreased expression level of at least one gene selected from the group consisting of neuronal differentiation genes, cell cycle inhibition genes, and tumor suppressor genes.

In some embodiments, a subject in need thereof has an increased expression level of at least one gene selected from the group consisting of hedgehog pathway genes, myc pathway genes and histone methyltransferase genes.

In some embodiments of the present invention, a subject in need thereof has loss of function of at least one component/subunit of the SWI/SNF complex. Alternatively, a subject in need thereof has reduced expression or haploinsufficiency of at least one component/subunit of the SWI/SNF complex. In certain embodiments, a subject in need thereof has loss of function of SNF5 subunit.

In any method of the present invention, a subject in need thereof may have reduced expression, haploinsufficiency or loss of function of at least one signaling component downstream of SWI/SNF complex. Such downstream component includes, but is not limited to, polycomb complex (PcG) and its targets.

As used herein, the term “loss of function” refers to less or no function of a gene product/protein compared to the wild type. Loss of function of a SWI/SNF complex component means the component/subunit or the entire SWI/SNF complex has less or no biological function compared to the wild type component/subunit or the entire SWI/SNF complex, respectively. Loss of function can be caused by transcriptional, post-transcription, or post translational mechanisms. In one aspect of the present invention, loss of function is caused by loss of function mutation resulted from a point mutation (e.g., a substitution, a missense mutation, or a nonsense mutation), an insertion, and/or a deletion in a polypeptide of a SWI/SNF complex component or a nucleic acid sequence encoding a polypeptide of a SWI/SNF complex component. The mutations referred herein are somatic mutations. The term “somatic mutation” refers to a deleterious alteration in at least one gene allele that is not found in every cell of the body, but is found only in isolated cells. A characteristic of the somatic mutations as used herein is, that they are restricted to particular tissues or even parts of tissues or cells within a tissue and are not present in the whole organism harboring the tissues or cells. The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene.

Accordingly, a loss of function mutation or a reduced expression can be detected using any suitable method available in the art. For example, a loss of function mutation can be detected by measuring the biological function of a gene product, such as the ATP-dependent chromatin remodeling activity of the SWI/SNF complex. Alternatively, a loss of function mutation can be determined by detecting any alternation in a nucleic acid sequence encoding a component of the SWI/SNF complex. For example, a nucleic acid sequence encoding a component of the SWI/SNF complex having a loss of function mutation can be detected by whole-genome resequencing or target region resequencing (the latter also known as targeted resequencing) using suitably selected sources of DNA and polymerase chain reaction (PCR) primers in accordance with methods well known in the art. The method typically and generally entails the steps of genomic DNA purification, PCR amplification to amplify the region of interest, cycle sequencing, sequencing reaction cleanup, capillary electrophoresis, and/or data analysis. Alternatively or in addition, the method may include the use of microarray-based targeted region genomic DNA capture and/or sequencing. Kits, reagents, and methods for selecting appropriate PCR primers and performing resequencing are commercially available, for example, from Applied Biosystems, Agilent, and NimbleGen (Roche Diagnostics GmbH). Alternatively or in addition, a nucleic acid sequence encoding a SWI/SNF polypeptide having a loss of function mutation may be detected using a Southern blot in accordance with methods well known in the art. Optionally, a loss of function mutation can be detected by measuring the absence of the expression of a component polypeptide or by measuring the expression of the mutant component polypeptide. Detection of (mutant) polypeptide expression can be carried out with any suitable immunoassay in the art, such as Western blot analysis.

Human nucleic acid and amino acid sequence of components of the SWI/SNF complex have previously been described. See, e.g., GenBank Accession Nos NP_003064.2, NM_003073.3, NP_001007469.1, and NM_001007468.1 for SNF5, GenBank Accession Nos NM_000489.3, NP_000480.2, NM_138270.2, and NP_612114.1 for ATRX, GenBank Accession Nos NP_006006.3, NM_006015.4, NP_624361.1, and NM_139135.2 for ARID1A, each of which is incorporated herein by reference in its entirety.

Spectrum of hSNF5 somatic mutations in human has also been described in Sevenet et al., Human Molecular Genetics, 8:2359-2368, 1999, which is incorporated herein by reference in its entirety.

A subject in need thereof may have reduced expression, haploinsufficiency, and/or loss of function of SNF5. For example, a subject can comprise a deletion of SNF5 in SNF5 polypeptide or a nucleic acid sequence encoding a SNF5 polypeptide.

| SWI/SNF-related matrix-associated actin-dependent regulator of chromatin | |
|---|---|
| subfamily B member 1 isoform a (SMARCB1, also called SNF5) [Homo sapiens] | |
| (SEQ ID NO: 1) | |
| 1 | mmmmalsktf gqkpvkfqle ddgefymigs evgnylrmfr gslykrypsl wrrlatveer |
| 61 | kkivasshgk ktkpntkdhg yttlatsvtl lkaseveeil dgndekykav sistepptyl |
| 121 | reqkakrnsq wvptlpnssh hldavpcstt inrnrmgrdk krtfplcfdd hdpavihena |
| 181 | sqpevlvpir ldmeidgqkl rdaftwnmne klmtpemfse ilcddldlnp ltfvpaiasa |
| 241 | irqqiesypt dsiledqsdq rviiklnihv gnislvdqfe wdmsekensp ekfalklcse |
| 301 | lglggefvtt iaysirgqls whqktyafse nplptveiai rntgdadqwc plletltdae |
| 361 | mekkirdqdr ntrrmrrlan tapaw |
| Homo sapiens SWI/SNF related, matrix associated, actin dependent | |
| regulator of chromatin, subfamily b, member 1 (SMARCB1, also called | |
| SNF5), transcript variant 1, mRNA | |
| (SEQ ID NO: 2) | |
| 1 | aacgccagcg cctgcgcact gagggcggcc tggtcgtcgt ctgcggcggc ggcggcggct |
| 61 | gaggagcccg gctgaggcgc cagtacccgg cccggtccgc atttcgcctt ccggcttcgg |
| 121 | tttccctcgg cccagcacgc cccggccccg ccccagccct cctgatccct cgcagcccgg |
| 181 | ctccggccgc ccgcctctgc cgccgcaatg atgatgatgg cgctgagcaa gaccttcggg |
| 241 | cagaagcccg tgaagttcca gctggaggac gacggcgagt tctacatgat cggctccgag |
| 301 | gtgggaaact acctccgtat gttccgaggt tctctgtaca agagataccc ctcactctgg |
| 361 | aggcgactag ccactgtgga agagaggaag aaaatagttg catcgtcaca tggtaaaaaa |
| 421 | acaaaaccta acactaagga tcacggatac acgactctag ccaccagtgt gaccctgtta |
| 481 | aaagcctcgg aagtggaaga gattctggat ggcaacgatg agaagtacaa ggctgtgtcc |
| 541 | atcagcacag agccccccac ctacctcagg gaacagaagg ccaagaggaa cagccagtgg |
| 601 | gtacccaccc tgcccaacag ctcccaccac ttagatgccg tgccatgctc cacaaccatc |
| 661 | aacaggaacc gcatgggccg agacaagaag agaaccttcc ccctttgctt tgatgaccat |
| 721 | gacccagctg tgatccatga gaacgcatct cagcccgagg tgctggtccc catccggctg |
| 781 | gacatggaga tcgatgggca gaagctgcga gacgccttca cctggaacat gaatgagaag |
| 841 | ttgatgacgc ctgagatgtt ttcagaaatc ctctgtgacg atctggattt gaacccgctg |
| 901 | acgtttgtgc cagccatcgc ctctgccatc agacagcaga tcgagtccta ccccacggac |
| 961 | agcatcctgg aggaccagtc agaccagcgc gtcatcatca agctgaacat ccatgtggga |
| 1021 | aacatttccc tggtggacca gtttgagtgg gacatgtcag agaaggagaa ctcaccagag |
| 1081 | aagtttgccc tgaagctgtg ctcggagctg gggttgggcg gggagtttgt caccaccatc |
| 1141 | gcatacagca tccggggaca gctgagctgg catcagaaga cctacgcctt cagcgagaac |
| 1201 | cctctgccca cagtggagat tgccatccgg aacacgggcg atgcggacca gtggtgccca |
| 1261 | ctgctggaga ctctgacaga cgctgagatg gagaagaaga tccgcgacca ggacaggaac |
| 1321 | acgaggcgga tgaggcgtct tgccaacacg gccccggcct ggtaaccagc ccatcagcac |
| 1381 | acggctccca cggagcatct cagaagattg ggccgcctct cctccatctt ctggcaagga |
| 1441 | cagaggcgag gggacagccc agcgccatcc tgaggatcgg gtgggggtgg agtgggggct |
| 1501 | tccaggtggc ccttcccggc acacattcca tttgttgagc cccagtcctg ccccccaccc |
| 1561 | caccctccct acccctcccc agtctctggg gtcaggaaga aaccttattt taggttgtgt |
| 1621 | tttgtttttg tataggagcc ccaggcaggg ctagtaacag tttttaaata aaaggcaaca |
| 1681 | ggtcatgttc aatttcttca acaaaaaaaa aaaaaaa |
| SWI/SNF-related matrix-associated actin-dependent regulator of | |
| chromatin subfamily B member 1 isoform b [Homo sapiens] (SMARCB1, | |
| also called SNF5) | |
| (SEQ ID NO: 3) | |
| 1 | mmmmalsktf gqkpvkfqle ddgefymigs evgnylrmfr gslykrypsl wrrlatveer |
| 61 | kkivasshdh gyttlatsvt llkaseveei ldgndekyka vsisteppty lreqkakrns |
| 121 | qwvptlpnss hhldavpcst tinrnrmgrd kkrtfplcfd dhdpavihen asqpevlvpi |
| 181 | rldmeidgqk lrdaftwnmn eklmtpemfs eilcddldln pltfvpaias airqqiesyp |
| 241 | tdsiledqsd qrviiklnih vgnislvdqf ewdmsekens pekfalklcs elglggefvt |
| 301 | tiaysirgql swhqktyafs enplptveia irntgdadqw cplletltda emekkirdqd |
| 361 | rntrrmrrla ntapaw |
| Homo sapiens SWI/SNF related, matrix associated, actin dependent | |
| regulator of chromatin, subfamily b, member 1 (SMARCB1, also called | |
| SNF5), transcript variant 2, mRNA | |
| (SEQ ID NO: 4) | |
| 1 | aacgccagcg cctgcgcact gagggcggcc tggtcgtcgt ctgcggcggc ggcggcggct |
| 61 | gaggagcccg gctgaggcgc cagtacccgg cccggtccgc atttcgcctt ccggcttcgg |
| 121 | tttccctcgg cccagcacgc cccggccccg ccccagccct cctgatccct cgcagcccgg |
| 181 | ctccggccgc ccgcctctgc cgccgcaatg atgatgatgg cgctgagcaa gaccttcggg |
| 241 | cagaagcccg tgaagttcca gctggaggac gacggcgagt tctacatgat cggctccgag |
| 301 | gtgggaaact acctccgtat gttccgaggt tctctgtaca agagataccc ctcactctgg |
| 361 | aggcgactag ccactgtgga agagaggaag aaaatagttg catcgtcaca tgatcacgga |
| 421 | tacacgactc tagccaccag tgtgaccctg ttaaaagcct cggaagtgga agagattctg |
| 481 | gatggcaacg atgagaagta caaggctgtg tccatcagca cagagccccc cacctacctc |
| 541 | agggaacaga aggccaagag gaacagccag tgggtaccca ccctgcccaa cagctcccac |
| 601 | cacttagatg ccgtgccatg ctccacaacc atcaacagga accgcatggg ccgagacaag |
| 661 | aagagaacct tccccctttg ctttgatgac catgacccag ctgtgatcca tgagaacgca |
| 721 | tctcagcccg aggtgctggt ccccatccgg ctggacatgg agatcgatgg gcagaagctg |
| 781 | cgagacgcct tcacctggaa catgaatgag aagttgatga cgcctgagat gttttcagaa |
| 841 | atcctctgtg acgatctgga tttgaacccg ctgacgtttg tgccagccat cgcctctgcc |
| 901 | atcagacagc agatcgagtc ctaccccacg gacagcatcc tggaggacca gtcagaccag |
| 961 | cgcgtcatca tcaagctgaa catccatgtg ggaaacattt ccctggtgga ccagtttgag |
| 1021 | tgggacatgt cagagaagga gaactcacca gagaagtttg ccctgaagct gtgctcggag |
| 1081 | ctggggttgg gcggggagtt tgtcaccacc atcgcataca gcatccgggg acagctgagc |
| 1141 | tggcatcaga agacctacgc cttcagcgag aaccctctgc ccacagtgga gattgccatc |
| 1201 | cggaacacgg gcgatgcgga ccagtggtgc ccactgctgg agactctgac agacgctgag |
| 1261 | atggagaaga agatccgcga ccaggacagg aacacgaggc ggatgaggcg tcttgccaac |
| 1321 | acggccccgg cctggtaacc agcccatcag cacacggctc ccacggagca tctcagaaga |
| 1381 | ttgggccgcc tctcctccat cttctggcaa ggacagaggc gaggggacag cccagcgcca |
| 1441 | tcctgaggat cgggtggggg tggagtgggg gcttccaggt ggcccttccc ggcacacatt |
| 1501 | ccatttgttg agccccagtc ctgcccccca ccccaccctc cctacccctc cccagtctct |
| 1561 | ggggtcagga agaaacctta ttttaggttg tgttttgttt ttgtatagga gccccaggca |
| 1621 | gggctagtaa cagtttttaa ataaaaggca acaggtcatg ttcaatttct tcaacaaaaa |
| 1681 | aaaaaaaaaa |

A subject in need thereof may have reduced expression, haploinsufficiency, and/or loss of function of ATRX. For example, a subject can comprise a mutation selected from the group consisting of a substitution of asparagine (N) for the wild type residue lysine (K) at amino acid position 688 of SEQ ID NO: 5 (K688N), and a substitution of isoleucine (I) for the wild type residue methionine (M) at amino acid position 366 of SEQ ID NO: 5 (M366I).

| Homo sapiens alpha thalassemia/mental retardation syndrome X-linked | |
|---|---|
| (ATRX) isoform 1 | |
| (SEQ ID NO: 5) | |
| 1 | mtaepmsesk lntlvqklhd flahsseese etsspprlam nqntdkisgs gsnsdmmens |
| 61 | keegtsssek skssgssrsk rkpsivtkyv esddekpldd etvnedasne nsenditmqs |
| 121 | lpkgtvivqp epvlnedkdd fkgpefrsrs kmktenlkkr gedglhgivs ctacgqqvnh |
| 181 | fqkdsiyrhp slqvlicknc fkyymsddis rdsdgmdeqc rwcaeggnli ccdfchnafc |
| 241 | kkcilrnlgr kelstimden nqwycyichp eplldlvtac nsvfenleql lqqnkkkikv |
| 301 | dseksnkvye htsrfspkkt ssncngeekk lddscsgsvt ysysalivpk emikkakkli |
| 361 | ettanmnssy vkflkqatdn seissatklr qlkafksvla dikkahlale edlnsefram |
| 421 | davnkekntk ehkvidakfe tkarkgekpc alekkdisks eaklsrkqvd sehmhqnvpt |
| 481 | eeqrtnkstg gehkksdrke epqyepants edldmdivsv pssvpedife nletamevqs |
| 541 | svdhqgdgss gteqevesss vklnisskdn rggiksktta kvtkelyvkl tpvslsnspi |
| 601 | kgadcqevpq dkdgykscgl npklekcglg qensdnehlv enevslllee sdlrrsprvk |
| 661 | ttplrrptet npvtsnsdee cnetvkekqk lsvpvrkkdk rnssdsaidn pkpnklpksk |
| 721 | qsetvdqnsd sdemlailke vsrmshssss dtdineihtn hktlydlktq agkddkgkrk |
| 781 | rksstsgsdf dtkkgksaks siiskkkrqt qsessnydse lekeiksmsk igaarttkkr |
| 841 | ipntkdfdss edekhskkgm dnqghknlkt sqegssddae rkqeretfss aegtvdkdtt |
| 901 | imelrdrlpk kqqasastdg vdklsgkeqs ftslevrkva etkekskhlk tktckkvgdg |
| 961 | lsdiaekflk kdqsdetsed dkkqskkgte ekkkpsdfkk kvikmeqqye sssdgteklp |
| 1021 | ereeichfpk gikqikngtt dgekkskkir dktskkkdel sdyaekstgk gdscdssedk |
| 1081 | kskngaygre kkrckllgks srkrqdcsss dtekysmked gcnssdkrlk rielrerrnl |
| 1141 | sskrntkeiq sgssssdaee ssednkkkkq rtsskkkavi vkekkrnslr tstkrkqadi |
| 1201 | tsssssdied ddqnsigegs sdeqkikpvt enlvlsshtg fcgssgdeal sksvpvtvdd |
| 1261 | ddddndpenr iakkmlleei kanlssdedg ssddepeegk krtgkqneen pgdeeaknqv |
| 1321 | nsesdsdsee skkpryrhrl lrhkltvsdg esgeekktkp kehkevkgrn rrkvssedse |
| 1381 | dsdfqesgvs eevsesedeq rprtrsakka eleenqrsyk qkkkrrrikv qedsssenks |
| 1441 | nseeeeeeke eeeeeeeeee eeeedendds kspgkgrkki rkilkddklr tetqnalkee |
| 1501 | eerrkriaer erereklrev ieiedasptk cpittklvld edeetkeplv qvhrnmvikl |
| 1561 | kphqvdgvqf mwdcccesvk ktkkspgsgc ilahcmglgk tlqvvsflht vllcdkldfs |
| 1621 | talvvcpint alnwmnefek wqeglkddek levselatvk rpgersymlq rwqedggvmi |
| 1681 | igyemyrnla qgrnvksrkl keifnkalvd pgpdfvvcde ghilkneasa vskamnsirs |
| 1741 | rrriiltgtp lqnnlieyhc mvnfikenll gsikefrnrf inpiqngqca dstmvdvrvm |
| 1801 | kkrahilyem lagcvqrkdy taltkflppk heyvlavrmt siqcklyqyy ldhltgvgnn |
| 1861 | seggrgkaga klfqdfqmls riwthpwclq ldyiskenkg yfdedsmdef iasdsdetsm |
| 1921 | slssddytkk kkkgkkgkkd ssssgsgsdn dvevikvwns rsrgggegnv detgnnpsys |
| 1981 | lkleeskats ssnpsspapd wykdfvtdad aevlehsgkm vllfeilrma eeigdkvlvf |
| 2041 | sqslisldli edflelasre ktedkdkpli ykgegkwlrn idyyrldgst taqsrkkwae |
| 2101 | efndetnvrg rlfiistkag slginlvaan rviifdaswn psydigsifr vyrfgqtkpv |
| 2161 | yvyrflaqgt medkiydrqv tkqslsfrvv dqqqverhft mneltelytf epdllddpns |
| 2221 | ekkkkrdtpm lpkdtilael lqihkehivg yhehdslldh keeeelteee rkaawaeyea |
| 2281 | ekkgltmrfn iptgtnlppv sfnsqtpyip fnlgalsams nqqledlinq grekvveatn |
| 2341 | svtavriqpl ediisavwke nmnlseaqvq alalsrqasq eldvkrreai yndvltkqqm |
| 2401 | liscvqrilm nrrlqqqynq qqqqqmtyqq atlghlmmpk ppnlimnpsn yqqidmrgmy |
| 2461 | qpvaggmqpp plqrapppmr sknpgpsqgk sm |
| Homo sapiens alpha thalassemia/mental retardation syndrome X-linked | |
| (ATRX), transcript variant 1, mRNA | |
| (SEQ ID NO: 6) | |
| 1 | aattctcctg cctgagcctc ggcccaacaa aatggcggcg gcagcggtgt cgctttgttt |
| 61 | ccgcggctcc tgcggcggtg gcagtggtag cggcctttga gctgtgggga ggttccagca |
| 121 | gcagctacag tgacgactaa gactccagtg catttctatc gtaaccgggc gcgggggagc |
| 181 | gcagatcggc gcccagcaat cacagaagcc gacaaggcgt tcaagcgaaa acatgaccgc |
| 241 | tgagcccatg agtgaaagca agttgaatac attggtgcag aagcttcatg acttccttgc |
| 301 | acactcatca gaagaatctg aagaaacaag ttctcctcca cgacttgcaa tgaatcaaaa |
| 361 | cacagataaa atcagtggtt ctggaagtaa ctctgatatg atggaaaaca gcaaggaaga |
| 421 | gggaactagc tcttcagaaa aatccaagtc ttcaggatcg tcacgatcaa agaggaaacc |
| 481 | ttcaattgta acaaagtatg tagaatcaga tgatgaaaaa cctttggatg atgaaactgt |
| 541 | aaatgaagat gcgtctaatg aaaattcaga aaatgatatt actatgcaga gcttgccaaa |
| 601 | aggtacagtg attgtacagc cagagccagt gctgaatgaa gacaaagatg attttaaagg |
| 661 | gcctgaattt agaagcagaa gtaaaatgaa aactgaaaat ctcaaaaaac gcggagaaga |
| 721 | tgggcttcat gggattgtga gctgcactgc ttgtggacaa caggtcaatc attttcaaaa |
| 781 | agattccatt tatagacacc cttcattgca agttcttatt tgtaagaatt gctttaagta |
| 841 | ttacatgagt gatgatatta gccgtgactc agatggaatg gatgaacaat gtaggtggtg |
| 901 | tgcggaaggt ggaaacttga tttgttgtga cttttgccat aatgctttct gcaagaaatg |
| 961 | cattctacgc aaccttggtc gaaaggagtt gtccacaata atggatgaaa acaaccaatg |
| 1021 | gtattgctac atttgtcacc cagagccttt gttggacttg gtcactgcat gtaacagcgt |
| 1081 | atttgagaat ttagaacagt tgttgcagca aaataagaag aagataaaag ttgacagtga |
| 1141 | aaagagtaat aaagtatatg aacatacatc cagattttct ccaaagaaga ctagttcaaa |
| 1201 | ttgtaatgga gaagaaaaga aattagatga ttcctgttct ggctctgtaa cctactctta |
| 1261 | ttccgcacta attgtgccca aagagatgat taagaaggca aaaaaactga ttgagaccac |
| 1321 | agccaacatg aactccagtt atgttaaatt tttaaagcag gcaacagata attcagaaat |
| 1381 | cagttctgct acaaaattac gtcagcttaa ggcttttaag tctgtgttgg ctgatattaa |
| 1441 | gaaggctcat cttgcattgg aagaagactt aaattccgag tttcgagcga tggatgctgt |
| 1501 | aaacaaagag aaaaatacca aagagcataa agtcatagat gctaagtttg aaacaaaagc |
| 1561 | acgaaaagga gaaaaacctt gtgctttgga aaagaaggat atttcaaagt cagaagctaa |
| 1621 | actttcaaga aaacaggtag atagtgagca catgcatcag aatgttccaa cagaggaaca |
| 1681 | aagaacaaat aaaagtaccg gtggtgaaca taagaaatct gatagaaaag aagaacctca |
| 1741 | atatgaacct gccaacactt ctgaagattt agacatggat attgtgtctg ttccttcctc |
| 1801 | agttccagaa gacatttttg agaatcttga gactgctatg gaagttcaga gttcagttga |
| 1861 | tcatcaaggg gatggcagca gtggaactga acaagaagtg gagagttcat ctgtaaaatt |
| 1921 | aaatatttct tcaaaagaca acagaggagg tattaaatca aaaactacag ctaaagtaac |
| 1981 | aaaagaatta tatgttaaac tcactcctgt ttccctttct aattccccaa ttaaaggtgc |
| 2041 | tgattgtcag gaagttccac aagataaaga tggctataaa agttgtggtc tgaaccccaa |
| 2101 | gttagagaaa tgtggacttg gacaggaaaa cagtgataat gagcatttgg ttgaaaatga |
| 2161 | agtttcatta cttttagagg aatctgatct tcgaagatcc ccacgtgtaa agactacacc |
| 2221 | cttgaggcga ccgacagaaa ctaaccctgt aacatctaat tcagatgaag aatgtaatga |
| 2281 | aacagttaag gagaaacaaa aactatcagt tccagtgaga aaaaaggata agcgtaattc |
| 2341 | ttctgacagt gctatagata atcctaagcc taataaattg ccaaaatcta agcaatcaga |
| 2401 | gactgtggat caaaattcag attctgatga aatgctagca atcctcaaag aggtgagcag |
| 2461 | gatgagtcac agttcttctt cagatactga tattaatgaa attcatacaa accataagac |
| 2521 | tttgtatgat ttaaagactc aggcggggaa agatgataaa ggaaaaagga aacgaaaaag |
| 2581 | ttctacatct ggctcagatt ttgatactaa aaagggcaaa tcagctaaga gctctataat |
| 2641 | ttctaaaaag aaacgacaaa cccagtctga gtcttctaat tatgactcag aattagaaaa |
| 2701 | agagataaag agcatgagta aaattggtgc tgccagaacc accaaaaaaa gaattccaaa |
| 2761 | tacaaaagat tttgactctt ctgaagatga gaaacacagc aaaaaaggaa tggataatca |
| 2821 | agggcacaaa aatttgaaga cctcacaaga aggatcatct gatgatgctg aaagaaaaca |
| 2881 | agagagagag actttctctt cagcagaagg cacagttgat aaagacacga ccatcatgga |
| 2941 | attaagagat cgacttccta agaagcagca agcaagtgct tccactgatg gtgtcgataa |
| 3001 | gctttctggg aaagagcaga gttttacttc tttggaagtt agaaaagttg ctgaaactaa |
| 3061 | agaaaagagc aagcatctca aaaccaaaac atgtaaaaaa gtacaggatg gcttatctga |
| 3121 | tattgcagag aaattcctaa agaaagacca gagcgatgaa acttctgaag atgataaaaa |
| 3181 | gcagagcaaa aagggaactg aagaaaaaaa gaaaccttca gactttaaga aaaaagtaat |
| 3241 | taaaatggaa caacagtatg aatcttcatc tgatggcact gaaaagttac ctgagcgaga |
| 3301 | agaaatttgt cattttccta agggcataaa acaaattaag aatggaacaa ctgatggaga |
| 3361 | aaagaaaagt aaaaaaataa gagataaaac ttctaaaaag aaggatgaat tatctgatta |
| 3421 | tgctgagaag tcaacaggga aaggagatag ttgtgactct tcagaggata aaaagagtaa |
| 3481 | gaatggagca tatggtagag agaagaaaag gtgcaagttg cttggaaaga gttcaaggaa |
| 3541 | gagacaagat tgttcatcat ctgatactga gaaatattcc atgaaagaag atggttgtaa |
| 3601 | ctcttctgat aagagactga aaagaataga attgagggaa agaagaaatt taagttcaaa |
| 3661 | gagaaatact aaggaaatac aaagtggctc atcatcatct gatgctgagg aaagttctga |
| 3721 | agataataaa aagaagaagc aaagaacttc atctaaaaag aaggcagtca ttgtcaagga |
| 3781 | gaaaaagaga aactccctaa gaacaagcac taaaaggaag caagctgaca ttacatcctc |
| 3841 | atcttcttct gatatagaag atgatgatca gaattctata ggtgagggaa gcagcgatga |
| 3901 | acagaaaatt aagcctgtga ctgaaaattt agtgctgtct tcacatactg gattttgcca |
| 3961 | atcttcagga gatgaagcct tatctaaatc agtgcctgtc acagtggatg atgatgatga |
| 4021 | cgacaatgat cctgagaata gaattgccaa gaagatgctt ttagaagaaa ttaaagccaa |
| 4081 | tctttcctct gatgaggatg gatcttcaga tgatgagcca gaagaaggga aaaaaagaac |
| 4141 | tggaaaacaa aatgaagaaa acccaggaga tgaggaagca aaaaatcaag tcaattctga |
| 4201 | atcagattca gattctgaag aatctaagaa gccaagatac agacataggc ttttgcggca |
| 4261 | caaattgact gtgagtgacg gagaatctgg agaagaaaaa aagacaaagc ctaaagagca |
| 4321 | taaagaagtc aaaggcagaa acagaagaaa ggtgagcagt gaagattcag aagattctga |
| 4381 | ttttcaggaa tcaggagtta gtgaagaagt tagtgaatcc gaagatgaac agcggcccag |
| 4441 | aacaaggtct gcaaagaaag cagagttgga agaaaatcag cggagctata aacagaaaaa |
| 4501 | gaaaaggcga cgtattaagg ttcaagaaga ttcatccagt gaaaacaaga gtaattctga |
| 4561 | ggaagaagag gaggaaaaag aagaggagga ggaagaggag gaggaggagg aagaggagga |
| 4621 | ggaagatgaa aatgatgatt ccaagtctcc tggaaaaggc agaaagaaaa ttcggaagat |
| 4681 | tcttaaagat gataaactga gaacagaaac acaaaatgct cttaaggaag aggaagagag |
| 4741 | acgaaaacgt attgctgaga gggagcgtga gcgagaaaaa ttgagagagg tgatagaaat |
| 4801 | tgaagatgct tcacccacca agtgtccaat aacaaccaag ttggttttag atgaagatga |
| 4861 | agaaaccaaa gaacctttag tgcaggttca tagaaatatg gttatcaaat tgaaacccca |
| 4921 | tcaagtagat ggtgttcagt ttatgtggga ttgctgctgt gagtctgtga aaaaaacaaa |
| 4981 | gaaatctcca ggttcaggat gcattcttgc ccactgtatg ggccttggta agactttaca |
| 5041 | ggtggtaagt tttcttcata cagttctttt gtgtgacaaa ctggatttca gcacggcgtt |
| 5101 | agtggtttgt cctcttaata ctgctttgaa ttggatgaat gaatttgaga agtggcaaga |
| 5161 | gggattaaaa gatgatgaga agcttgaggt ttctgaatta gcaactgtga aacgtcctca |
| 5221 | ggagagaagc tacatgctgc agaggtggca agaagatggt ggtgttatga tcataggcta |
| 5281 | tgagatgtat agaaatcttg ctcaaggaag gaatgtgaag agtcggaaac ttaaagaaat |
| 5341 | atttaacaaa gctttggttg atccaggccc tgattttgtt gtttgtgatg aaggccatat |
| 5401 | tctaaaaaat gaagcatctg ctgtttctaa agctatgaat tctatacgat caaggaggag |
| 5461 | gattatttta acaggaacac cacttcaaaa taacctaatt gagtatcatt gtatggttaa |
| 5521 | ttttatcaag gaaaatttac ttggatccat taaggagttc aggaatagat ttataaatcc |
| 5581 | aattcaaaat ggtcagtgtg cagattctac catggtagat gtcagagtga tgaaaaaacg |
| 5641 | tgctcacatt ctctatgaga tgttagctgg atgtgttcag aggaaagatt atacagcatt |
| 5701 | aacaaaattc ttgcctccaa aacacgaata tgtgttagct gtgagaatga cttctattca |
| 5761 | gtgcaagctc tatcagtact acttagatca cttaacaggt gtgggcaata atagtgaagg |
| 5821 | tggaagagga aaggcaggtg caaagctttt ccaagatttt cagatgttaa gtagaatatg |
| 5881 | gactcatcct tggtgtttgc agctagacta cattagcaaa gaaaataagg gttattttga |
| 5941 | tgaagacagt atggatgaat ttatagcctc agattctgat gaaacctcca tgagtttaag |
| 6001 | ctccgatgat tatacaaaaa agaagaaaaa agggaaaaag gggaaaaaag atagtagctc |
| 6061 | aagtggaagt ggcagtgaca atgatgttga agtgattaag gtctggaatt caagatctcg |
| 6121 | gggaggtggt gaaggaaatg tggatgaaac aggaaacaat ccttctgttt ctttaaaact |
| 6181 | ggaagaaagt aaagctactt cttcttctaa tccaagcagc ccagctccag actggtacaa |
| 6241 | agattttgtt acagatgctg atgctgaggt tttagagcat tctgggaaaa tggtacttct |
| 6301 | ctttgaaatt cttcgaatgg cagaggaaat tggggataaa gtccttgttt tcagccagtc |
| 6361 | cctcatatct ctggacttga ttgaagattt tcttgaatta gctagtaggg agaagacaga |
| 6421 | agataaagat aaacccctta tttataaagg tgaggggaag tggcttcgaa acattgacta |
| 6481 | ttaccgttta gatggttcca ctactgcaca gtcaaggaag aagtgggctg aagaatttaa |
| 6541 | tgatgaaact aatgtgagag gacgattatt tatcatttct actaaagcag gatctctagg |
| 6601 | aattaatctg gtagctgcta atcgagtaat tatattcgac gcttcttgga atccatctta |
| 6661 | tgacatccag agtatattca gagtttatcg ctttggacaa actaagcctg tttatgtata |
| 6721 | taggttctta gctcagggaa ccatggaaga taagatttat gatcggcaag taactaagca |
| 6781 | gtcactgtct tttcgagttg ttgatcagca gcaggtggag cgtcatttta ctatgaatga |
| 6841 | gcttactgaa ctttatactt ttgagccaga cttattagat gaccctaatt cagaaaagaa |
| 6901 | gaagaagagg gatactccca tgctgccaaa ggataccata cttgcagagc tccttcagat |
| 6961 | acataaagaa cacattgtag gataccatga acatgattct cttttggacc acaaagaaga |
| 7021 | agaagagttg actgaagaag aaagaaaagc agcttgggct gagtatgaag cagagaagaa |
| 7081 | gggactgacc atgcgtttca acataccaac tgggaccaat ttaccccctg tcagtttcaa |
| 7141 | ctctcaaact ccttatattc ctttcaattt gggagccctg tcagcaatga gtaatcaaca |
| 7201 | gctggaggac ctcattaatc aaggaagaga aaaagttgta gaagcaacaa acagtgtgac |
| 7261 | agcagtgagg attcaacctc ttgaggatat aatttcagct gtatggaagg agaacatgaa |
| 7321 | tctctcagag gcccaagtac aggcgttagc attaagtaga caagccagcc aggagcttga |
| 7381 | tgttaaacga agagaagcaa tctacaatga tgtattgaca aaacaacaga tgttaatcag |
| 7441 | ctgtgttcag cgaatactta tgaacagaag gctccagcag cagtacaatc agcagcaaca |
| 7501 | gcaacaaatg acttatcaac aagcaacact gggtcacctc atgatgccaa agcccccaaa |
| 7561 | tttgatcatg aatccttcta actaccagca gattgatatg agaggaatgt atcagccagt |
| 7621 | ggctggtggt atgcagccac caccattaca gcgtgcacca cccccaatga gaagcaaaaa |
| 7681 | tccaggacct tcccaaggga aatcaatgtg attttgcact aaaagcttaa tggattgtta |
| 7741 | aaatcataga aagatctttt atttttttag gaatcaatga cttaacagaa ctcaactgta |
| 7801 | taaatagttt ggtcccctta aatgccaatc ttccatatta gttttacttt tttttttttt |
| 7861 | aaatagggca taccatttct tcctgacatt tgtcagtgat gttgcctaga atcttcttac |
| 7921 | acacgctgag tacagaagat atttcaaatt gttttcagtg aaaacaagtc cttccataat |
| 7981 | agtaacaact ccacagattt cctctctaaa tttttatgcc tgcttttagc aaccataaaa |
| 8041 | ttgtcataaa attaataaat ttaggaaaga ataaagattt atatattcat tctttacata |
| 8101 | taaaaacaca cagctgagtt cttagagttg attcctcaag ttatgaaata cttttgtact |
| 8161 | taatccattt cttgattaaa gtgattgaaa tggttttaat gttcttttga ctgaagtctg |
| 8221 | aaactgggct cctgctttat tgtctctgtg actgaaagtt agaaactgag ggttatcttt |
| 8281 | gacacagaat tgtgtgcaat attcttaaat actactgctc taaaagttgg agaagtcttg |
| 8341 | cagttatctt agcattgtat aaacagcctt aagtatagcc taagaagaga attccttttt |
| 8401 | cttctttagt ccttctgcca ttttttattt tcagttatat gtgctgaaat aattactggt |
| 8461 | aaaatttcag ggttgtggat tatcttccac acatgaattt tctctctcct ggcacgaata |
| 8521 | taaagcacat ctcttaactg catggtgcca gtgctaatgc ttcatcctgt tgctggcagt |
| 8581 | gggatgtgga cttagaaaat caagttctag cattttagta ggttaacact gaagttgtgg |
| 8641 | ttgttaggtt cacaccctgt tttataaaca acatcaaaat ggcagaacca ttgctgactt |
| 8701 | taggttcaca tgaggaatgt acttttaaca attcccagta ctatcagtat tgtgaaataa |
| 8761 | ttcctctgaa agataagaat cactggcttc tatgcgcttc ttttctctca tcatcatgtt |
| 8821 | cttttacccc agtttcctta cattttttta aattgtttca gagtttgttt tttttttagt |
| 8881 | ttagattgtg aggcaattat taaatcaaaa ttaattcatc caatacccct ttactagaag |
| 8941 | ttttactaga aaatgtatta cattttattt tttcttaatc cagttctgca aaaatgacct |
| 9001 | ataaatttat tcatgtacaa ttttggttac ttgaattgtt aaagaaaaca ttgtttttga |
| 9061 | ctatgggagt caactcaaca tggcagaacc atttttgaga tgatgataca acaggtagtg |
| 9121 | aaacagctta agaattccaa aaaaaaaaaa aaaaaaaaaa aaaagaaaac tgggtttggg |
| 9181 | ctttgcttta ggtatcactg gattagaatg agtttaacat tagctaaaac tgctttgagt |
| 9241 | tgtttggatg attaagagat tgccattttt atcttggaag aactagtggt aaaacatcca |
| 9301 | agagcactag gattgtgata cagaatttgt gaggtttggt ggatccacgc ccctctcccc |
| 9361 | cactttccca tgatgaaata tcactaataa atcctgtata tttagatatt atgctagcca |
| 9421 | tgtaatcaga tttatttaat tgggtggggc aggtgtgtat ttactttaga aaaaatgaaa |
| 9481 | aagacaagat ttatgagaaa tatttgaagg cagtacactc tggccaactg ttaccagttg |
| 9541 | gtatttctac aagttcagaa tattttaaac ctgatttact agacctggga attttcaaca |
| 9601 | tggtctaatt atttactcaa agacatagat gtgaaaattt taggcaacct tctaaatctt |
| 9661 | tttcaccatg gatgaaacta taacttaaag aataatactt agaagggtta attggaaatc |
| 9721 | agagtttgaa ataaaacttg gaccactttg tatacactct tctcacttga cattttagct |
| 9781 | atataatatg tactttgagt ataacatcaa gctttaacaa atatttaaag acaaaaaaat |
| 9841 | cacgtcagta aaatactaaa aggctcattt ttatatttgt tttagatgtt ttaaatagtt |
| 9901 | gcaatggatt aaaaatgatg atttaaaatg ttgcttgtaa tacagttttg cctgctaaat |
| 9961 | tctccacatt ttgtaacctg ttttatttct ttgggtgtaa agcgtttttg cttagtattg |
| 10021 | tgatattgta tatgttttgt cccagttgta tagtaatgtt tcagtccatc atccagcttt |
| 10081 | ggctgctgaa atcatacagc tgtgaagact tgcctttgtt tctgttagac tgcttttcag |
| 10141 | ttctgtattg agtatcttaa gtactgtaga aaagatgtca cttcttcctt taaggctgtt |
| 10201 | ttgtaatata tataaggact ggaattgtgt ttttaaagaa aagcattcaa gtatgacaat |
| 10261 | atactatctg tgttttcacc attcaaagtg ctgtttagta gttgaaactt aaactattta |
| 10321 | atgtcattta ataaagtgac caaaatgtgt tgtgctcttt attgtatttt cacagctttg |
| 10381 | aaaatctgtg cacatactgt ttcatagaaa atgtatagct tttgttgtcc tatataatgg |
| 10441 | tggttctttt gcacatttag ttatttaata ttgagaggtc acgaagtttg gttattgaat |
| 10501 | ctgttatata ctaaattctg taaagggaga tctctcatct caaaaagaat ttacatacca |
| 10561 | ggaagtccat gtgtgtttgt gttagttttg gatgtctttg tgtaatccag ccccatttcc |
| 10621 | tgtttcccaa cagctgtaac actcatttta agtcaagcag ggctaccaac ccacacttga |
| 10681 | tagaaaagct gcttaccatt cagaagcttc cttattacct ggcctccaaa tgagctgaat |
| 10741 | attttgtagc cttcccttag ctatgttcat tttccctcca ttatcataaa atcagatcga |
| 10801 | tatttatgtg ccccaaacaa aactttaaga gcagttacat tctgtcccag tagcccttgt |
| 10861 | ttcctttgag agtagcatgt tgtgaggcta tagagactta ttctaccagt aaaacaggtc |
| 10921 | aatcctttta catgtttatt atactaaaaa ttatgttcag ggtatttact actttatttc |
| 10981 | accagactca gtctcaagtg acttggctat ctccaaatca gatctaccct tagagaataa |
| 11041 | acatttttct accgttattt tttttcaagt ctataatctg agccagtccc aaaggagtga |
| 11101 | tcaagtttca gaaatgcttt catcttcaca acattttata tatactatta tatggggtga |
| 11161 | ataaagtttt aaatccgaaa tataaaaaaa aaaaaaaaaa aa |
| Homo sapiens alpha thalassemia/mental retardation syndrome X-linked | |
| (ATRX) isoform 2 | |
| (SEQ ID NO: 7) | |
| 1 | mtaepmsesk lntlvqklhd flahsseese etsspprlam nqntdkisgs gsnsdmmens |
| 61 | keegtsssek skssgssrsk rkpsivtkyv esddekpldd etvnedasne nsenditmqs |
| 121 | lpkedglhgi vsctacgqqv nhfqkdsiyr hpslqvlick ncfkyymsdd isrdsdgmde |
| 181 | qcrwcaeggn liccdfchna fokkcilrnl grkelstimd ennqwycyic hpeplldlvt |
| 241 | acnsvfenle qllqqnkkki kvdseksnkv yehtsrfspk ktssncngee kklddscsgs |
| 301 | vtysysaliv pkemikkakk liettanmns syvkflkqat dnseissatk lrqlkafksv |
| 361 | ladikkahla leedlnsefr amdavnkekn tkehkvidak fetkarkgek pcalekkdis |
| 421 | kseaklsrkg vdsehmhqnv pteeqrtnks tggehkksdr keepqyepan tsedldmdiv |
| 481 | svpssvpedi fenletamev qssvdhqgdg ssgteqeves ssvklnissk dnrggikskt |
| 541 | takvtkelyv kltpvslsns pikgadcqev pqdkdgyksc glnpklekcg lggensdneh |
| 601 | lvenevslll eesdlrrspr vkttplrrpt etnpvtsnsd eecnetvkek qklsvpvrkk |
| 661 | dkrnssdsai dnpkpnklpk skqsetvdqn sdsdemlail kevsrmshss ssdtdineih |
| 721 | tnhktlydlk tqagkddkgk rkrksstsgs dfdtkkgksa kssiiskkkr qtqsessnyd |
| 781 | selekeiksm skigaarttk kripntkdfd ssedekhskk gmdnqghknl ktsgegssdd |
| 841 | aerkqeretf ssaegtvdkd ttimelrdrl pkkqqasast dgvdklsgke qsftslevrk |
| 901 | vaetkekskh lktktckkvg dglsdiaekf lkkdqsdets eddkkqskkg teekkkpsdf |
| 961 | kkkvikmegq yesssdgtek lpereeichf pkgikqikng ttdgekkskk irdktskkkd |
| 1021 | elsdyaekst gkgdscdsse dkkskngayg rekkrckllg kssrkrqdcs ssdtekysmk |
| 1081 | edgcnssdkr lkrielrerr nlsskrntke iqsgssssda eessednkkk kqrtsskkka |
| 1141 | vivkekkrns lrtstkrkqa ditsssssdi edddqnsige gssdeqkikp vtenlvlssh |
| 1201 | tgfcgssgde alsksvpvtv ddddddndpe nriakkmlle eikanlssde dgssddepee |
| 1261 | gkkrtgkqne enpgdeeakn qvnsesdsds eeskkpryrh rllrhkltvs dgesgeekkt |
| 1321 | kpkehkevkg rnrrkvssed sedsdfqesg vseevsesed eqrprtrsak kaeleenqrs |
| 1381 | ykqkkkrrri kvqedsssen ksnseeeeee keeeeeeeee eeeeeedend dskspgkgrk |
| 1441 | kirkilkddk lrtetqnalk eeeerrkria ererereklr evieiedasp tkcpittklv |
| 1501 | ldedeetkep lvqvhrnmvi klkphqvdgv qfmwdccces vkktkkspgs gcilahcmgl |
| 1561 | gktlqvvsfl htvllcdkld fstalvvcpl ntalnwmnef ekwqeglkdd eklevselat |
| 1621 | vkrpgersym lqrwqedggv miigyemyrn laqgrnvksr klkeifnkal vdpgpdfvvc |
| 1681 | deghilknea sayskamnsi rsrrriiltg tplqnnliey hcmvnfiken llgsikefrn |
| 1741 | rfinpiqngq cadstmvdvr vmkkrahily emlagcvqrk dytaltkflp pkheyvlavr |
| 1801 | mtsiqcklyq yyldhltgvg nnseggrgka gaklfqdfqm lsriwthpwc lqldyisken |
| 1861 | kgyfdedsmd efiasdsdet smslssddyt kkkkkgkkgk kdssssgsgs dndvevikvw |
| 1921 | nsrsrgggeg nvdetgnnps vslkleeska tsssnpsspa pdwykdfvtd adaevlehsg |
| 1981 | kmvllfeilr maeeigdkvl vfsqslisld liedflelas rektedkdkp liykgegkwl |
| 2041 | rnidyyrldg sttaqsrkkw aeefndetnv rgrlfiistk agslginlva anrviifdas |
| 2101 | wnpsydigsi frvyrfgqtk pvyvyrflaq gtmedkiydr qvtkqslsfr vvdqqqverh |
| 2161 | ftmneltely tfepdllddp nsekkkkrdt pmlpkdtila ellqihkehi vgyhehdsll |
| 2221 | dhkeeeelte eerkaawaey eaekkgltmr fniptgtnlp pvsfnsqtpy ipfnlgalsa |
| 2281 | msnqqledli nqgrekvvea tnsvtavriq plediisavw kenmnlseaq vgalalsrqa |
| 2341 | sqeldvkrre aiyndvltkq qmliscvgri lmnrrlqqqy nqqqqqqmty qqatlghlmm |
| 2401 | pkppnlimnp snyqqidmrg myqpvaggmq ppplqrappp mrsknpgpsq gksm |
| Homo sapiens alpha thalassemia/mental retardation syndrome X-linked | |
| (ATRX), transcript variant 2, mRNA | |
| (SEQ ID NO: 8) | |
| 1 | aattctcctg cctgagcctc ggcccaacaa aatggcggcg gcagcggtgt cgctttgttt |
| 61 | ccgcggctcc tgcggcggtg gcagtggtag cggcctttga gctgtgggga ggttccagca |
| 121 | gcagctacag tgacgactaa gactccagtg catttctatc gtaaccgggc gcgggggagc |
| 181 | gcagatcggc gcccagcaat cacagaagcc gacaaggcgt tcaagcgaaa acatgaccgc |
| 241 | tgagcccatg agtgaaagca agttgaatac attggtgcag aagcttcatg acttccttgc |
| 301 | acactcatca gaagaatctg aagaaacaag ttctcctcca cgacttgcaa tgaatcaaaa |
| 361 | cacagataaa atcagtggtt ctggaagtaa ctctgatatg atggaaaaca gcaaggaaga |
| 421 | gggaactagc tcttcagaaa aatccaagtc ttcaggatcg tcacgatcaa agaggaaacc |
| 481 | ttcaattgta acaaagtatg tagaatcaga tgatgaaaaa cctttggatg atgaaactgt |
| 541 | aaatgaagat gcgtctaatg aaaattcaga aaatgatatt actatgcaga gcttgccaaa |
| 601 | agaagatggg cttcatggga ttgtgagctg cactgcttgt ggacaacagg tcaatcattt |
| 661 | tcaaaaagat tccatttata gacacccttc attgcaagtt cttatttgta agaattgctt |
| 721 | taagtattac atgagtgatg atattagccg tgactcagat ggaatggatg aacaatgtag |
| 781 | gtggtgtgcg gaaggtggaa acttgatttg ttgtgacttt tgccataatg ctttctgcaa |
| 841 | gaaatgcatt ctacgcaacc ttggtcgaaa ggagttgtcc acaataatgg atgaaaacaa |
| 901 | ccaatggtat tgctacattt gtcacccaga gcctttgttg gacttggtca ctgcatgtaa |
| 961 | cagcgtattt gagaatttag aacagttgtt gcagcaaaat aagaagaaga taaaagttga |
| 1021 | cagtgaaaag agtaataaag tatatgaaca tacatccaga ttttctccaa agaagactag |
| 1081 | ttcaaattgt aatggagaag aaaagaaatt agatgattcc tgttctggct ctgtaaccta |
| 1141 | ctcttattcc gcactaattg tgcccaaaga gatgattaag aaggcaaaaa aactgattga |
| 1201 | gaccacagcc aacatgaact ccagttatgt taaattttta aagcaggcaa cagataattc |
| 1261 | agaaatcagt tctgctacaa aattacgtca gcttaaggct tttaagtctg tgttggctga |
| 1321 | tattaagaag gctcatcttg cattggaaga agacttaaat tccgagtttc gagcgatgga |
| 1381 | tgctgtaaac aaagagaaaa ataccaaaga gcataaagtc atagatgcta agtttgaaac |
| 1441 | aaaagcacga aaaggagaaa aaccttgtgc tttggaaaag aaggatattt caaagtcaga |
| 1501 | agctaaactt tcaagaaaac aggtagatag tgagcacatg catcagaatg ttccaacaga |
| 1561 | ggaacaaaga acaaataaaa gtaccggtgg tgaacataag aaatctgata gaaaagaaga |
| 1621 | acctcaatat gaacctgcca acacttctga agatttagac atggatattg tgtctgttcc |
| 1681 | ttcctcagtt ccagaagaca tttttgagaa tcttgagact gctatggaag ttcagagttc |
| 1741 | agttgatcat caaggggatg gcagcagtgg aactgaacaa gaagtggaga gttcatctgt |
| 1801 | aaaattaaat atttcttcaa aagacaacag aggaggtatt aaatcaaaaa ctacagctaa |
| 1861 | agtaacaaaa gaattatatg ttaaactcac tcctgtttcc ctttctaatt ccccaattaa |
| 1921 | aggtgctgat tgtcaggaag ttccacaaga taaagatggc tataaaagtt gtggtctgaa |
| 1981 | ccccaagtta gagaaatgtg gacttggaca ggaaaacagt gataatgagc atttggttga |
| 2041 | aaatgaagtt tcattacttt tagaggaatc tgatcttcga agatccccac gtgtaaagac |
| 2101 | tacacccttg aggcgaccga cagaaactaa ccctgtaaca tctaattcag atgaagaatg |
| 2161 | taatgaaaca gttaaggaga aacaaaaact atcagttcca gtgagaaaaa aggataagcg |
| 2221 | taattcttct gacagtgcta tagataatcc taagcctaat aaattgccaa aatctaagca |
| 2281 | atcagagact gtggatcaaa attcagattc tgatgaaatg ctagcaatcc tcaaagaggt |
| 2341 | gagcaggatg agtcacagtt cttcttcaga tactgatatt aatgaaattc atacaaacca |
| 2401 | taagactttg tatgatttaa agactcaggc ggggaaagat gataaaggaa aaaggaaacg |
| 2461 | aaaaagttct acatctggct cagattttga tactaaaaag ggcaaatcag ctaagagctc |
| 2521 | tataatttct aaaaagaaac gacaaaccca gtctgagtct tctaattatg actcagaatt |
| 2581 | agaaaaagag ataaagagca tgagtaaaat tggtgctgcc agaaccacca aaaaaagaat |
| 2641 | tccaaataca aaagattttg actcttctga agatgagaaa cacagcaaaa aaggaatgga |
| 2701 | taatcaaggg cacaaaaatt tgaagacctc acaagaagga tcatctgatg atgctgaaag |
| 2761 | aaaacaagag agagagactt tctcttcagc agaaggcaca gttgataaag acacgaccat |
| 2821 | catggaatta agagatcgac ttcctaagaa gcagcaagca agtgcttcca ctgatggtgt |
| 2881 | cgataagctt tctgggaaag agcagagttt tacttctttg gaagttagaa aagttgctga |
| 2941 | aactaaagaa aagagcaagc atctcaaaac caaaacatgt aaaaaagtac aggatggctt |
| 3001 | atctgatatt gcagagaaat tcctaaagaa agaccagagc gatgaaactt ctgaagatga |
| 3061 | taaaaagcag agcaaaaagg gaactgaaga aaaaaagaaa ccttcagact ttaagaaaaa |
| 3121 | agtaattaaa atggaacaac agtatgaatc ttcatctgat ggcactgaaa agttacctga |
| 3181 | gcgagaagaa atttgtcatt ttcctaaggg cataaaacaa attaagaatg gaacaactga |
| 3241 | tggagaaaag aaaagtaaaa aaataagaga taaaacttct aaaaagaagg atgaattatc |
| 3301 | tgattatgct gagaagtcaa cagggaaagg agatagttgt gactcttcag aggataaaaa |
| 3361 | gagtaagaat ggagcatatg gtagagagaa gaaaaggtgc aagttgcttg gaaagagttc |
| 3421 | aaggaagaga caagattgtt catcatctga tactgagaaa tattccatga aagaagatgg |
| 3481 | ttgtaactct tctgataaga gactgaaaag aatagaattg agggaaagaa gaaatttaag |
| 3541 | ttcaaagaga aatactaagg aaatacaaag tggctcatca tcatctgatg ctgaggaaag |
| 3601 | ttctgaagat aataaaaaga agaagcaaag aacttcatct aaaaagaagg cagtcattgt |
| 3661 | caaggagaaa aagagaaact ccctaagaac aagcactaaa aggaagcaag ctgacattac |
| 3721 | atcctcatct tcttctgata tagaagatga tgatcagaat tctataggtg agggaagcag |
| 3781 | cgatgaacag aaaattaagc ctgtgactga aaatttagtg ctgtcttcac atactggatt |
| 3841 | ttgccaatct tcaggagatg aagccttatc taaatcagtg cctgtcacag tggatgatga |
| 3901 | tgatgacgac aatgatcctg agaatagaat tgccaagaag atgcttttag aagaaattaa |
| 3961 | agccaatctt tcctctgatg aggatggatc ttcagatgat gagccagaag aagggaaaaa |
| 4021 | aagaactgga aaacaaaatg aagaaaaccc aggagatgag gaagcaaaaa atcaagtcaa |
| 4081 | ttctgaatca gattcagatt ctgaagaatc taagaagcca agatacagac ataggctttt |
| 4141 | gcggcacaaa ttgactgtga gtgacggaga atctggagaa gaaaaaaaga caaagcctaa |
| 4201 | agagcataaa gaagtcaaag gcagaaacag aagaaaggtg agcagtgaag attcagaaga |
| 4261 | ttctgatttt caggaatcag gagttagtga agaagttagt gaatccgaag atgaacagcg |
| 4321 | gcccagaaca aggtctgcaa agaaagcaga gttggaagaa aatcagcgga gctataaaca |
| 4381 | gaaaaagaaa aggcgacgta ttaaggttca agaagattca tccagtgaaa acaagagtaa |
| 4441 | ttctgaggaa gaagaggagg aaaaagaaga ggaggaggaa gaggaggagg aggaggaaga |
| 4501 | ggaggaggaa gatgaaaatg atgattccaa gtctcctgga aaaggcagaa agaaaattcg |
| 4561 | gaagattctt aaagatgata aactgagaac agaaacacaa aatgctctta aggaagagga |
| 4621 | agagagacga aaacgtattg ctgagaggga gcgtgagcga gaaaaattga gagaggtgat |
| 4681 | agaaattgaa gatgcttcac ccaccaagtg tccaataaca accaagttgg ttttagatga |
| 4741 | agatgaagaa accaaagaac ctttagtgca ggttcataga aatatggtta tcaaattgaa |
| 4801 | accccatcaa gtagatggtg ttcagtttat gtgggattgc tgctgtgagt ctgtgaaaaa |
| 4861 | aacaaagaaa tctccaggtt caggatgcat tcttgcccac tgtatgggcc ttggtaagac |
| 4921 | tttacaggtg gtaagttttc ttcatacagt tcttttgtgt gacaaactgg atttcagcac |
| 4981 | ggcgttagtg gtttgtcctc ttaatactgc tttgaattgg atgaatgaat ttgagaagtg |
| 5041 | gcaagaggga ttaaaagatg atgagaagct tgaggtttct gaattagcaa ctgtgaaacg |
| 5101 | tcctcaggag agaagctaca tgctgcagag gtggcaagaa gatggtggtg ttatgatcat |
| 5161 | aggctatgag atgtatagaa atcttgctca aggaaggaat gtgaagagtc ggaaacttaa |
| 5221 | agaaatattt aacaaagctt tggttgatcc aggccctgat tttgttgttt gtgatgaagg |
| 5281 | ccatattcta aaaaatgaag catctgctgt ttctaaagct atgaattcta tacgatcaag |
| 5341 | gaggaggatt attttaacag gaacaccact tcaaaataac ctaattgagt atcattgtat |
| 5401 | ggttaatttt atcaaggaaa atttacttgg atccattaag gagttcagga atagatttat |
| 5461 | aaatccaatt caaaatggtc agtgtgcaga ttctaccatg gtagatgtca gagtgatgaa |
| 5521 | aaaacgtgct cacattctct atgagatgtt agctggatgt gttcagagga aagattatac |
| 5581 | agcattaaca aaattcttgc ctccaaaaca cgaatatgtg ttagctgtga gaatgacttc |
| 5641 | tattcagtgc aagctctatc agtactactt agatcactta acaggtgtgg gcaataatag |
| 5701 | tgaaggtgga agaggaaagg caggtgcaaa gcttttccaa gattttcaga tgttaagtag |
| 5761 | aatatggact catccttggt gtttgcagct agactacatt agcaaagaaa ataagggtta |
| 5821 | ttttgatgaa gacagtatgg atgaatttat agcctcagat tctgatgaaa cctccatgag |
| 5881 | tttaagctcc gatgattata caaaaaagaa gaaaaaaggg aaaaagggga aaaaagatag |
| 5941 | tagctcaagt ggaagtggca gtgacaatga tgttgaagtg attaaggtct ggaattcaag |
| 6001 | atctcgggga ggtggtgaag gaaatgtgga tgaaacagga aacaatcctt ctgtttcttt |
| 6061 | aaaactggaa gaaagtaaag ctacttcttc ttctaatcca agcagcccag ctccagactg |
| 6121 | gtacaaagat tttgttacag atgctgatgc tgaggtttta gagcattctg ggaaaatggt |
| 6181 | acttctcttt gaaattcttc gaatggcaga ggaaattggg gataaagtcc ttgttttcag |
| 6241 | ccagtccctc atatctctgg acttgattga agattttctt gaattagcta gtagggagaa |
| 6301 | gacagaagat aaagataaac cccttattta taaaggtgag gggaagtggc ttcgaaacat |
| 6361 | tgactattac cgtttagatg gttccactac tgcacagtca aggaagaagt gggctgaaga |
| 6421 | atttaatgat gaaactaatg tgagaggacg attatttatc atttctacta aagcaggatc |
| 6481 | tctaggaatt aatctggtag ctgctaatcg agtaattata ttcgacgctt cttggaatcc |
| 6541 | atcttatgac atccagagta tattcagagt ttatcgcttt ggacaaacta agcctgttta |
| 6601 | tgtatatagg ttcttagctc agggaaccat ggaagataag atttatgatc ggcaagtaac |
| 6661 | taagcagtca ctgtcttttc gagttgttga tcagcagcag gtggagcgtc attttactat |
| 6721 | gaatgagctt actgaacttt atacttttga gccagactta ttagatgacc ctaattcaga |
| 6781 | aaagaagaag aagagggata ctcccatgct gccaaaggat accatacttg cagagctcct |
| 6841 | tcagatacat aaagaacaca ttgtaggata ccatgaacat gattctcttt tggaccacaa |
| 6901 | agaagaagaa gagttgactg aagaagaaag aaaagcagct tgggctgagt atgaagcaga |
| 6961 | gaagaaggga ctgaccatgc gtttcaacat accaactggg accaatttac cccctgtcag |
| 7021 | tttcaactct caaactcctt atattccttt caatttggga gccctgtcag caatgagtaa |
| 7081 | tcaacagctg gaggacctca ttaatcaagg aagagaaaaa gttgtagaag caacaaacag |
| 7141 | tgtgacagca gtgaggattc aacctcttga ggatataatt tcagctgtat ggaaggagaa |
| 7201 | catgaatctc tcagaggccc aagtacaggc gttagcatta agtagacaag ccagccagga |
| 7261 | gcttgatgtt aaacgaagag aagcaatcta caatgatgta ttgacaaaac aacagatgtt |
| 7321 | aatcagctgt gttcagcgaa tacttatgaa cagaaggctc cagcagcagt acaatcagca |
| 7381 | gcaacagcaa caaatgactt atcaacaagc aacactgggt cacctcatga tgccaaagcc |
| 7441 | cccaaatttg atcatgaatc cttctaacta ccagcagatt gatatgagag gaatgtatca |
| 7501 | gccagtggct ggtggtatgc agccaccacc attacagcgt gcaccacccc caatgagaag |
| 7561 | caaaaatcca ggaccttccc aagggaaatc aatgtgattt tgcactaaaa gcttaatgga |
| 7621 | ttgttaaaat catagaaaga tcttttattt ttttaggaat caatgactta acagaactca |
| 7681 | actgtataaa tagtttggtc cccttaaatg ccaatcttcc atattagttt tacttttttt |
| 7741 | ttttttaaat agggcatacc atttcttcct gacatttgtc agtgatgttg cctagaatct |
| 7801 | tcttacacac gctgagtaca gaagatattt caaattgttt tcagtgaaaa caagtccttc |
| 7861 | cataatagta acaactccac agatttcctc tctaaatttt tatgcctgct tttagcaacc |
| 7921 | ataaaattgt cataaaatta ataaatttag gaaagaataa agatttatat attcattctt |
| 7981 | tacatataaa aacacacagc tgagttctta gagttgattc ctcaagttat gaaatacttt |
| 8041 | tgtacttaat ccatttcttg attaaagtga ttgaaatggt tttaatgttc ttttgactga |
| 8101 | agtctgaaac tgggctcctg ctttattgtc tctgtgactg aaagttagaa actgagggtt |
| 8161 | atctttgaca cagaattgtg tgcaatattc ttaaatacta ctgctctaaa agttggagaa |
| 8221 | gtcttgcagt tatcttagca ttgtataaac agccttaagt atagcctaag aagagaattc |
| 8281 | ctttttcttc tttagtcctt ctgccatttt ttattttcag ttatatgtgc tgaaataatt |
| 8341 | actggtaaaa tttcagggtt gtggattatc ttccacacat gaattttctc tctcctggca |
| 8401 | cgaatataaa gcacatctct taactgcatg gtgccagtgc taatgcttca tcctgttgct |
| 8461 | ggcagtggga tgtggactta gaaaatcaag ttctagcatt ttagtaggtt aacactgaag |
| 8521 | ttgtggttgt taggttcaca ccctgtttta taaacaacat caaaatggca gaaccattgc |
| 8581 | tgactttagg ttcacatgag gaatgtactt ttaacaattc ccagtactat cagtattgtg |
| 8641 | aaataattcc tctgaaagat aagaatcact ggcttctatg cgcttctttt ctctcatcat |
| 8701 | catgttcttt taccccagtt tccttacatt tttttaaatt gtttcagagt ttgttttttt |
| 8761 | tttagtttag attgtgaggc aattattaaa tcaaaattaa ttcatccaat acccctttac |
| 8821 | tagaagtttt actagaaaat gtattacatt ttattttttc ttaatccagt tctgcaaaaa |
| 8881 | tgacctataa atttattcat gtacaatttt ggttacttga attgttaaag aaaacattgt |
| 8941 | ttttgactat gggagtcaac tcaacatggc agaaccattt ttgagatgat gatacaacag |
| 9001 | gtagtgaaac agcttaagaa ttccaaaaaa aaaaaaaaaa aaaaaaaaaa gaaaactggg |
| 9061 | tttgggcttt gctttaggta tcactggatt agaatgagtt taacattagc taaaactgct |
| 9121 | ttgagttgtt tggatgatta agagattgcc atttttatct tggaagaact agtggtaaaa |
| 9181 | catccaagag cactaggatt gtgatacaga atttgtgagg tttggtggat ccacgcccct |
| 9241 | ctcccccact ttcccatgat gaaatatcac taataaatcc tgtatattta gatattatgc |
| 9301 | tagccatgta atcagattta tttaattggg tggggcaggt gtgtatttac tttagaaaaa |
| 9361 | atgaaaaaga caagatttat gagaaatatt tgaaggcagt acactctggc caactgttac |
| 9421 | cagttggtat ttctacaagt tcagaatatt ttaaacctga tttactagac ctgggaattt |
| 9481 | tcaacatggt ctaattattt actcaaagac atagatgtga aaattttagg caaccttcta |
| 9541 | aatctttttc accatggatg aaactataac ttaaagaata atacttagaa gggttaattg |
| 9601 | gaaatcagag tttgaaataa aacttggacc actttgtata cactcttctc acttgacatt |
| 9661 | ttagctatat aatatgtact ttgagtataa catcaagctt taacaaatat ttaaagacaa |
| 9721 | aaaaatcacg tcagtaaaat actaaaaggc tcatttttat atttgtttta gatgttttaa |
| 9781 | atagttgcaa tggattaaaa atgatgattt aaaatgttgc ttgtaataca gttttgcctg |
| 9841 | ctaaattctc cacattttgt aacctgtttt atttctttgg gtgtaaagcg tttttgctta |
| 9901 | gtattgtgat attgtatatg ttttgtccca gttgtatagt aatgtttcag tccatcatcc |
| 9961 | agctttggct gctgaaatca tacagctgtg aagacttgcc tttgtttctg ttagactgct |
| 10021 | tttcagttct gtattgagta tcttaagtac tgtagaaaag atgtcacttc ttcctttaag |
| 10081 | gctgttttgt aatatatata aggactggaa ttgtgttttt aaagaaaagc attcaagtat |
| 10141 | gacaatatac tatctgtgtt ttcaccattc aaagtgctgt ttagtagttg aaacttaaac |
| 10201 | tatttaatgt catttaataa agtgaccaaa atgtgttgtg ctctttattg tattttcaca |
| 10261 | gctttgaaaa tctgtgcaca tactgtttca tagaaaatgt atagcttttg ttgtcctata |
| 10321 | taatggtggt tcttttgcac atttagttat ttaatattga gaggtcacga agtttggtta |
| 10381 | ttgaatctgt tatatactaa attctgtaaa gggagatctc tcatctcaaa aagaatttac |
| 10441 | ataccaggaa gtccatgtgt gtttgtgtta gttttggatg tctttgtgta atccagcccc |
| 10501 | atttcctgtt tcccaacagc tgtaacactc attttaagtc aagcagggct accaacccac |
| 10561 | acttgataga aaagctgctt accattcaga agcttcctta ttacctggcc tccaaatgag |
| 10621 | ctgaatattt tgtagccttc ccttagctat gttcattttc cctccattat cataaaatca |
| 10681 | gatcgatatt tatgtgcccc aaacaaaact ttaagagcag ttacattctg tcccagtagc |
| 10741 | ccttgtttcc tttgagagta gcatgttgtg aggctataga gacttattct accagtaaaa |
| 10801 | caggtcaatc cttttacatg tttattatac taaaaattat gttcagggta tttactactt |
| 10861 | tatttcacca gactcagtct caagtgactt ggctatctcc aaatcagatc tacccttaga |
| 10921 | gaataaacat ttttctaccg ttattttttt tcaagtctat aatctgagcc agtcccaaag |
| 10981 | gagtgatcaa gtttcagaaa tgctttcatc ttcacaacat tttatatata ctattatatg |
| 11041 | gggtgaataa agttttaaat ccgaaatata aaaaaaaaaa aaaaaaaa |

A subject in need thereof may have reduced expression, haploinsufficiency, and/or loss of function of ARID1A. For example, a subject may comprise a mutation selected from the group consisting of a nonsense mutation for the wild type residue cysteine (C) at amino acid position 884 of SEQ ID NO: 11 (C884*), a substitution of lysine (K) for the wild type residue glutamic acid (E) at amino acid position 966 (E966K), a nonsense mutation for the wild type residue glutamine (Q) at amino acid position 1411 of SEQ ID NO: 11 (Q1411*), a frame shift mutation at the wild type residue phenylalanine (F) at amino acid position 1720 of SEQ ID NO: 11 (F1720fs), a frame shift mutation after the wild type residue glycine (G) at amino acid position 1847 of SEQ ID NO: 11 (G1847fs), a frame shift mutation at the wild type residue cysteine (C) at amino acid position 1874 of SEQ ID NO: 11 (C1874fs), a substitution of glutamic acid (E) for the wild type residue aspartic acid (D) at amino acid position 1957 (D1957E), a nonsense mutation for the wild type residue glutamine (Q) at amino acid position 1430 of SEQ ID NO: 11 (Q1430*), a frame shift mutation at the wild type residue arginine (R) at amino acid position 1721 of SEQ ID NO: 11 (R1721fs), a substitution of glutamic acid (E) for the wild type residue glycine (G) at amino acid position 1255 (G1255E), a frame shift mutation at the wild type residue glycine (G) at amino acid position 284 of SEQ ID NO: 11 (G284fs), a nonsense mutation for the wild type residue arginine (R) at amino acid position 1722 of SEQ ID NO: 11 (R1722*), a frame shift mutation at the wild type residue methionine (M) at amino acid position 274 of SEQ ID NO: 11 (M274fs), a frame shift mutation at the wild type residue glycine (G) at amino acid position 1847 of SEQ ID NO: 11 (G1847fs), a frame shift mutation at the wild type residue P at amino acid position 559 of SEQ ID NO: 11 (P559fs), a nonsense mutation for the wild type residue arginine (R) at amino acid position 1276 of SEQ ID NO: 11 (R1276*), a frame shift mutation at the wild type residue glutamine (Q) at amino acid position 2176 of SEQ ID NO: 11 (Q2176fs), a frame shift mutation at the wild type residue histidine (H) at amino acid position 203 of SEQ ID NO: 11 (H203fs), a frame shift mutation at the wild type residue alanine (A) at amino acid position 591 of SEQ ID NO: 11 (A591fs), a nonsense mutation for the wild type residue glutamine (Q) at amino acid position 1322 of SEQ ID NO: 11 (Q1322*), a nonsense mutation for the wild type residue serine(S) at amino acid position 2264 of SEQ ID NO: 11 (S2264*), a nonsense mutation for the wild type residue glutamine (Q) at amino acid position 586 of SEQ ID NO: 11 (Q586*), a frame shift mutation at the wild type residue glutamine (Q) at amino acid position 548 of SEQ ID NO: 11 (Q548fs), and a frame shift mutation at the wild type residue asparagine (N) at amino acid position 756 of SEQ ID NO: 11 (N756fs). “*” used herein refers to a stop codon. “fs” used herein refers to a frame shift.

| AT-rich interactive domain-containing protein 1A (ARID1A) isoform a | |
|---|---|
| [Homo sapiens] | |
| (SEQ ID NO: 9) | |
| 1 | maaqvapaaa sslgnppppp pselkkaeqq qreeaggeaa aaaaaergem kaaagqeseg |
| 61 | pavgppqplg kelqdgaesn gggggggags gggpgaepdl knsngnagpr palnnnltep |
| 121 | pggggggssd gvgapphsaa aalpppaygf gqpygrspsa vaaaaaavfh qqhggqqspg |
| 181 | laalqsgggg glepyagpqq nshdhgfpnh qynsyypnrs aypppapaya lssprggtpg |
| 241 | sgaaaaagsk pppsssasas sssssfaqqr fgamggggps aagggtpqpt atptlnqllt |
| 301 | spssargyqg ypggdysggp qdggagkgpa dmasqcwgaa aaaaaaaaas ggaqqrshha |
| 361 | pmspgssggg gqplartpqp sspmdqmgkm rpqpyggtnp ysqqqgppsg pqqghgypgq |
| 421 | pygsqtpqry pmtmggraqs amgglsytqq ippygqqgps gygqqgqtpy ynggsphpqq |
| 481 | qqppysqqpp sqtphaqpsy qqqpqsqppq lqssqppysq qpsqpphqqs papypsqqst |
| 541 | tqqhpqsqpp ysqpqaqspy qqqqpqqpap stlsqqaayp qpqsqqsqqt aysqqrfppp |
| 601 | qelsqdsfgs qassapsmts skggqedmnl slqsrpsslp dlsgsiddlp mgtegalspg |
| 661 | vstsgisssq gegsnpagsp fsphtsphlp girgpspspv gspasvaqsr sgplspaavp |
| 721 | gnqmpprpps gqsdsimhps mngssiaqdr gymqrnpqmp qysspqpgsa lsprqpsggq |
| 781 | ihtgmgsyqq nsmgsygpqg gqygpqggyp rqpnynalpn anypsagmag ginpmgaggq |
| 841 | mhgqpgippy gtlppgrmsh asmgnrpygp nmanmppqvg sgmcpppggm nrktqetava |
| 901 | mhvaansiqn rppgypnmnq ggmmgtgppy gqginsmagm inpqgppysm ggtmannsag |
| 961 | maaspemmgl gdvkltpatk mnnkadgtpk teskskksss stttnekitk lyelggeper |
| 1021 | kmwvdrylaf teekamgmtn lpavgrkpld lyrlyvsvke iggltqvnkn kkwrelatnl |
| 1081 | nvgtsssaas slkkqyiqcl yafeckierg edpppdifaa adskksqpki qppspagsgs |
| 1141 | mqgpqtpqst sssmaeggdl kpptpastph sqipplpgms rsnsvgigda fndgsdstfq |
| 1201 | krnsmtpnpg yqpsmntsdm mgrmsyepnk dpygsmrkap gsdpfmssgq gpnggmgdpy |
| 1261 | sraagpglgn vamgprqhyp yggpydrvrt epgigpegnm stgapqpnlm psnpdsgmys |
| 1321 | psryppqqqq qqqqrhdsyg nqfstqgtps gspfpsqqtt myqqqqqnyk rpmdgtygpp |
| 1381 | akrhegemys vpystgqgqp qqqqlppaqp qpasqqqaaq pspqqdvynq ygnaypatat |
| 1441 | aaterrpagg pqnqfpfqfg rdrvsappgt naqqnmppqm mggpigasae vaqqgtmwqg |
| 1501 | rndmtynyan rqstgsapqg payhgvnrtd emlhtdqran hegswpshgt rqppygpsap |
| 1561 | vppmtrppps nyqpppsmqn hipqvsspap lprpmenrts pskspflhsg mkmqkagppv |
| 1621 | pashiapapv qppmirrdit fppgsveatq pvlkqrrrlt mkdigtpeaw rvmmslksgl |
| 1681 | laestwaldt inillyddns imtfnlsqlp gllellveyf rrclieifgi lkeyevgdpg |
| 1741 | qrtlldpgrf skvsspapme ggeeeeellg pkleeeeeee vvendeeiaf sgkdkpasen |
| 1801 | seekliskfd klpvkivqkn dpfvvdcsdk lgrvqefdsg llhwrigggd ttehiqthfe |
| 1861 | sktellpsrp hapcppaprk hvttaegtpg ttdqegpppd gppekritat mddmlstrss |
| 1921 | tltedgakss eaikesskfp fgispaqshr nikiledeph skdetplctl ldwqdslakr |
| 1981 | cvcvsntirs lsfvpgndfe mskhpgllli lgklillhhk hperkqaplt yekeeeqdqg |
| 2041 | vscnkvewww dclemlrent lvtlanisgq ldlspypesi clpvldgllh wavcpsaeaq |
| 2101 | dpfstlgpna vlspqrlvle tlsklsiqdn nvdlilatpp fsrleklyst mvrflsdrkn |
| 2161 | pvcremavvl lanlaggdsl aaraiavqkg signllgfle dslaatqfqq sgasllhmqn |
| 2221 | ppfeptsvdm mrraaralla lakvdenhse ftlyesrlld isysplmnsl vsqvicdvlf |
| 2281 | ligqs |
| Homo sapiens AT rich interactive domain 1A (SWI-like) (ARID1A), | |
| transcript variant 1, mRNA | |
| (SEQ ID NO: 10) | |
| 1 | cagaaagcgg agagtcacag cggggccagg ccctggggag cggagcctcc accgcccccc |
| 61 | tcattcccag gcaagggctt ggggggaatg agccgggaga gccgggtccc gagcctacag |
| 121 | agccgggagc agctgagccg ccggcgcctc ggccgccgcc gccgcctcct cctcctccgc |
| 181 | cgccgccagc ccggagcctg agccggcggg gcggggggga gaggagcgag cgcagcgcag |
| 241 | cagcggagcc ccgcgaggcc cgcccgggcg ggtggggagg gcagcccggg ggactgggcc |
| 301 | ccggggcggg gtgggagggg gggagaagac gaagacaggg ccgggtctct ccgcggacga |
| 361 | gacagcgggg atcatggccg cgcaggtcgc ccccgccgcc gccagcagcc tgggcaaccc |
| 421 | gccgccgccg ccgccctcgg agctgaagaa agccgagcag cagcagcggg aggaggcggg |
| 481 | gggcgaggcg gcggcggcgg cagcggccga gcgcggggaa atgaaggcag ccgccgggca |
| 541 | ggaaagcgag ggccccgccg tggggccgcc gcagccgctg ggaaaggagc tgcaggacgg |
| 601 | ggccgagagc aatgggggtg gcggcggcgg cggagccggc agcggcggcg ggcccggcgc |
| 661 | ggagccggac ctgaagaact cgaacgggaa cgcgggccct aggcccgccc tgaacaataa |
| 721 | cctcacggag ccgcccggcg gcggcggtgg cggcagcagc gatggggtgg gggcgcctcc |
| 781 | tcactcagcc gcggccgcct tgccgccccc agcctacggc ttcgggcaac cctacggccg |
| 841 | gagcccgtct gccgtcgccg ccgccgcggc cgccgtcttc caccaacaac atggcggaca |
| 901 | acaaagccct ggcctggcag cgctgcagag cggcggcggc gggggcctgg agccctacgc |
| 961 | ggggccccag cagaactctc acgaccacgg cttccccaac caccagtaca actcctacta |
| 1021 | ccccaaccgc agcgcctacc ccccgcccgc cccggcctac gcgctgagct ccccgagagg |
| 1081 | tggcactccg ggctccggcg cggcggcggc tgccggctcc aagccgcctc cctcctccag |
| 1141 | cgcctccgcc tcctcgtcgt cttcgtcctt cgctcagcag cgcttcgggg ccatgggggg |
| 1201 | aggcggcccc tccgcggccg gcgggggaac tccccagccc accgccaccc ccaccctcaa |
| 1261 | ccaactgctc acgtcgccca gctcggcccg gggctaccag ggctaccccg ggggcgacta |
| 1321 | cagtggcggg ccccaggacg ggggcgccgg caagggcccg gcggacatgg cctcgcagtg |
| 1381 | ttggggggct gcggcggcgg cagctgcggc ggcggccgcc tcgggagggg cccaacaaag |
| 1441 | gagccaccac gcgcccatga gccccgggag cagcggcggc ggggggcagc cgctcgcccg |
| 1501 | gacccctcag ccatccagtc caatggatca gatgggcaag atgagacctc agccatatgg |
| 1561 | cgggactaac ccatactcgc agcaacaggg acctccgtca ggaccgcagc aaggacatgg |
| 1621 | gtacccaggg cagccatacg ggtcccagac cccgcagcgg tacccgatga ccatgcaggg |
| 1681 | ccgggcgcag agtgccatgg gcggcctctc ttatacacag cagattcctc cttatggaca |
| 1741 | acaaggcccc agcgggtatg gtcaacaggg ccagactcca tattacaacc agcaaagtcc |
| 1801 | tcaccctcag cagcagcagc caccctactc ccagcaacca ccgtcccaga cccctcatgc |
| 1861 | ccaaccttcg tatcagcagc agccacagtc tcaaccacca cagctccagt cctctcagcc |
| 1921 | tccatactcc cagcagccat cccagcctcc acatcagcag tccccggctc catacccctc |
| 1981 | ccagcagtcg acgacacagc agcaccccca gagccagccc ccctactcac agccacaggc |
| 2041 | tcagtctcct taccagcagc agcaacctca gcagccagca ccctcgacgc tctcccagca |
| 2101 | ggctgcgtat cctcagcccc agtctcagca gtcccagcaa actgcctatt cccagcagcg |
| 2161 | cttccctcca ccgcaggagc tatctcaaga ttcatttggg tctcaggcat cctcagcccc |
| 2221 | ctcaatgacc tccagtaagg gagggcaaga agatatgaac ctgagccttc agtcaagacc |
| 2281 | ctccagcttg cctgatctat ctggttcaat agatgacctc cccatgggga cagaaggagc |
| 2341 | tctgagtcct ggagtgagca catcagggat ttccagcagc caaggagagc agagtaatcc |
| 2401 | agctcagtct cctttctctc ctcatacctc ccctcacctg cctggcatcc gaggcccttc |
| 2461 | cccgtcccct gttggctctc ccgccagtgt tgctcagtct cgctcaggac cactctcgcc |
| 2521 | tgctgcagtg ccaggcaacc agatgccacc tcggccaccc agtggccagt cggacagcat |
| 2581 | catgcatcct tccatgaacc aatcaagcat tgcccaagat cgaggttata tgcagaggaa |
| 2641 | cccccagatg ccccagtaca gttcccccca gcccggctca gccttatctc cgcgtcagcc |
| 2701 | ttccggagga cagatacaca caggcatggg ctcctaccag cagaactcca tggggagcta |
| 2761 | tggtccccag gggggtcagt atggcccaca aggtggctac cccaggcagc caaactataa |
| 2821 | tgccttgccc aatgccaact accccagtgc aggcatggct ggaggcataa accccatggg |
| 2881 | tgccggaggt caaatgcatg gacagcctgg catcccacct tatggcacac tccctccagg |
| 2941 | gaggatgagt cacgcctcca tgggcaaccg gccttatggc cctaacatgg ccaatatgcc |
| 3001 | acctcaggtt gggtcaggga tgtgtccccc accagggggc atgaaccgga aaacccaaga |
| 3061 | aactgctgtc gccatgcatg ttgctgccaa ctctatccaa aacaggccgc caggctaccc |
| 3121 | caatatgaat caagggggca tgatgggaac tggacctcct tatggacaag ggattaatag |
| 3181 | tatggctggc atgatcaacc ctcagggacc cccatattcc atgggtggaa ccatggccaa |
| 3241 | caattctgca gggatggcag ccagcccaga gatgatgggc cttggggatg taaagttaac |
| 3301 | tccagccacc aaaatgaaca acaaggcaga tgggacaccc aagacagaat ccaaatccaa |
| 3361 | gaaatccagt tcttctacta caaccaatga gaagatcacc aagttgtatg agctgggtgg |
| 3421 | tgagcctgag aggaagatgt gggtggaccg ttatctggcc ttcactgagg agaaggccat |
| 3481 | gggcatgaca aatctgcctg ctgtgggtag gaaacctctg gacctctatc gcctctatgt |
| 3541 | gtctgtgaag gagattggtg gattgactca ggtcaacaag aacaaaaaat ggcgggaact |
| 3601 | tgcaaccaac ctcaatgtgg gcacatcaag cagtgctgcc agctccttga aaaagcagta |
| 3661 | tatccagtgt ctctatgcct ttgaatgcaa gattgaacgg ggagaagacc ctcccccaga |
| 3721 | catctttgca gctgctgatt ccaagaagtc ccagcccaag atccagcctc cctctcctgc |
| 3781 | gggatcagga tctatgcagg ggccccagac tccccagtca accagcagtt ccatggcaga |
| 3841 | aggaggagac ttaaagccac caactccagc atccacacca cacagtcaga tccccccatt |
| 3901 | gccaggcatg agcaggagca attcagttgg gatccaggat gcctttaatg atggaagtga |
| 3961 | ctccacattc cagaagcgga attccatgac tccaaaccct gggtatcagc ccagtatgaa |
| 4021 | tacctctgac atgatggggc gcatgtccta tgagccaaat aaggatcctt atggcagcat |
| 4081 | gaggaaagct ccagggagtg atcccttcat gtcctcaggg cagggcccca acggcgggat |
| 4141 | gggtgacccc tacagtcgtg ctgccggccc tgggctagga aatgtggcga tgggaccacg |
| 4201 | acagcactat ccctatggag gtccttatga cagagtgagg acggagcctg gaatagggcc |
| 4261 | tgagggaaac atgagcactg gggccccaca gccgaatctc atgccttcca acccagactc |
| 4321 | ggggatgtat tctcctagcc gctacccccc gcagcagcag cagcagcagc agcaacgaca |
| 4381 | tgattcctat ggcaatcagt tctccaccca aggcacccct tctggcagcc ccttccccag |
| 4441 | ccagcagact acaatgtatc aacagcaaca gcagaattac aagcggccaa tggatggcac |
| 4501 | atatggccct cctgccaagc ggcacgaagg ggagatgtac agcgtgccat acagcactgg |
| 4561 | gcaggggcag cctcagcagc agcagttgcc cccagcccag ccccagcctg ccagccagca |
| 4621 | acaagctgcc cagccttccc ctcagcaaga tgtatacaac cagtatggca atgcctatcc |
| 4681 | tgccactgcc acagctgcta ctgagcgccg accagcaggc ggcccccaga accaatttcc |
| 4741 | attccagttt ggccgagacc gtgtctctgc accccctggc accaatgccc agcaaaacat |
| 4801 | gccaccacaa atgatgggcg gccccataca ggcatcagct gaggttgctc agcaaggcac |
| 4861 | catgtggcag gggcgtaatg acatgaccta taattatgcc aacaggcaga gcacgggctc |
| 4921 | tgccccccag ggccccgcct atcatggcgt gaaccgaaca gatgaaatgc tgcacacaga |
| 4981 | tcagagggcc aaccacgaag gctcgtggcc ttcccatggc acacgccagc ccccatatgg |
| 5041 | tccctctgcc cctgtgcccc ccatgacaag gccccctcca tctaactacc agcccccacc |
| 5101 | aagcatgcag aatcacattc ctcaggtatc cagccctgct cccctgcccc ggccaatgga |
| 5161 | gaaccgcacc tctcctagca agtctccatt cctgcactct gggatgaaaa tgcagaaggc |
| 5221 | aggtccccca gtacctgcct cgcacatagc acctgcccct gtgcagcccc ccatgattcg |
| 5281 | gcgggatatc accttcccac ctggctctgt tgaagccaca cagcctgtgt tgaagcagag |
| 5341 | gaggcggctc acaatgaaag acattggaac cccggaggca tggcgggtaa tgatgtccct |
| 5401 | caagtctggt ctcctggcag agagcacatg ggcattagat accatcaaca tcctgctgta |
| 5461 | tgatgacaac agcatcatga ccttcaacct cagtcagctc ccagggttgc tagagctcct |
| 5521 | tgtagaatat ttccgacgat gcctgattga gatctttggc attttaaagg agtatgaggt |
| 5581 | gggtgaccca ggacagagaa cgctactgga tcctgggagg ttcagcaagg tgtctagtcc |
| 5641 | agctcccatg gagggtgggg aagaagaaga agaacttcta ggtcctaaac tagaagagga |
| 5701 | agaagaagag gaagtagttg aaaatgatga ggagatagcc ttttcaggca aggacaagcc |
| 5761 | agcttcagag aatagtgagg agaagctgat cagtaagttt gacaagcttc cagtaaagat |
| 5821 | cgtacagaag aatgatccat ttgtggtgga ctgctcagat aagcttgggc gtgtgcagga |
| 5881 | gtttgacagt ggcctgctgc actggcggat tggtgggggg gacaccactg agcatatcca |
| 5941 | gacccacttc gagagcaaga cagagctgct gccttcccgg cctcacgcac cctgcccacc |
| 6001 | agcccctcgg aagcatgtga caacagcaga gggtacacca gggacaacag accaggaggg |
| 6061 | gcccccacct gatggacctc cagaaaaacg gatcacagcc actatggatg acatgttgtc |
| 6121 | tactcggtct agcaccttga ccgaggatgg agctaagagt tcagaggcca tcaaggagag |
| 6181 | cagcaagttt ccatttggca ttagcccagc acagagccac cggaacatca agatcctaga |
| 6241 | ggacgaaccc cacagtaagg atgagacccc actgtgtacc cttctggact ggcaggattc |
| 6301 | tcttgccaag cgctgcgtct gtgtgtccaa taccattcga agcctgtcat ttgtgccagg |
| 6361 | caatgacttt gagatgtcca aacacccagg gctgctgctc atcctgggca agctgatcct |
| 6421 | gctgcaccac aagcacccag aacggaagca ggcaccacta acttatgaaa aggaggagga |
| 6481 | acaggaccaa ggggtgagct gcaacaaagt ggagtggtgg tgggactgct tggagatgct |
| 6541 | ccgggaaaac accttggtta cactcgccaa catctcgggg cagttggacc tatctccata |
| 6601 | ccccgagagc atttgcctgc ctgtcctgga cggactccta cactgggcag tttgcccttc |
| 6661 | agctgaagcc caggacccct tttccaccct gggccccaat gccgtccttt ccccgcagag |
| 6721 | actggtcttg gaaaccctca gcaaactcag catccaggac aacaatgtgg acctgattct |
| 6781 | ggccacaccc cccttcagcc gcctggagaa gttgtatagc actatggtgc gcttcctcag |
| 6841 | tgaccgaaag aacccggtgt gccgggagat ggctgtggta ctgctggcca acctggctca |
| 6901 | gggggacagc ctggcagctc gtgccattgc agtgcagaag ggcagtatcg gcaacctcct |
| 6961 | gggcttccta gaggacagcc ttgccgccac acagttccag cagagccagg ccagcctcct |
| 7021 | ccacatgcag aacccaccct ttgagccaac tagtgtggac atgatgcggc gggctgcccg |
| 7081 | cgcgctgctt gccttggcca aggtggacga gaaccactca gagtttactc tgtacgaatc |
| 7141 | acggctgttg gacatctcgg tatcaccgtt gatgaactca ttggtttcac aagtcatttg |
| 7201 | tgatgtactg tttttgattg gccagtcatg acagccgtgg gacacctccc ccccccgtgt |
| 7261 | gtgtgtgcgt gtgtggagaa cttagaaact gactgttgcc ctttatttat gcaaaaccac |
| 7321 | ctcagaatcc agtttaccct gtgctgtcca gcttctccct tgggaaaaag tctctcctgt |
| 7381 | ttctctctcc tccttccacc tcccctccct ccatcacctc acgcctttct gttccttgtc |
| 7441 | ctcaccttac tcccctcagg accctacccc accctctttg aaaagacaaa gctctgccta |
| 7501 | catagaagac tttttttatt ttaaccaaag ttactgttgt ttacagtgag tttggggaaa |
| 7561 | aaaaataaaa taaaaatggc tttcccagtc cttgcatcaa cgggatgcca catttcataa |
| 7621 | ctgtttttaa tggtaaaaaa aaaaaaaaaa aatacaaaaa aaaattctga aggacaaaaa |
| 7681 | aggtgactgc tgaactgtgt gtggtttatt gttgtacatt cacaatcttg caggagccaa |
| 7741 | gaagttcgca gttgtgaaca gaccctgttc actggagagg cctgtgcagt agagtgtaga |
| 7801 | ccctttcatg tactgtactg tacacctgat actgtaaaca tactgtaata ataatgtctc |
| 7861 | acatggaaac agaaaacgct gggtcagcag caagctgtag tttttaaaaa tgtttttagt |
| 7921 | taaacgttga ggagaaaaaa aaaaaaggct tttcccccaa agtatcatgt gtgaacctac |
| 7981 | aacaccctga cctctttctc tcctccttga ttgtatgaat aaccctgaga tcacctctta |
| 8041 | gaactggttt taacctttag ctgcagcggc tacgctgcca cgtgtgtata tatatgacgt |
| 8101 | tgtacattgc acataccctt ggatccccac agtttggtcc tcctcccagc taccccttta |
| 8161 | tagtatgacg agttaacaag ttggtgacct gcacaaagcg agacacagct atttaatctc |
| 8221 | ttgccagata tcgcccctct tggtgcgatg ctgtacaggt ctctgtaaaa agtccttgct |
| 8281 | gtctcagcag ccaatcaact tatagtttat ttttttctgg gtttttgttt tgttttgttt |
| 8341 | tctttctaat cgaggtgtga aaaagttcta ggttcagttg aagttctgat gaagaaacac |
| 8401 | aattgagatt ttttcagtga taaaatctgc atatttgtat ttcaacaatg tagctaaaac |
| 8461 | ttgatgtaaa ttcctccttt ttttcctttt ttggcttaat gaatatcatt tattcagtat |
| 8521 | gaaatcttta tactatatgt tccacgtgtt aagaataaat gtacattaaa tcttggtaag |
| 8581 | acttt |
| AT-rich interactive domain-containing protein 1A (ARID1A) isoform b | |
| (SEQ ID NO: 11) | |
| 1 | maaqvapaaa sslgnppppp pselkkaeqq qreeaggeaa aaaaaergem kaaagqeseg |
| 61 | pavgppqplg kelqdgaesn gggggggags gggpgaepdl knsngnagpr palnnnltep |
| 121 | pggggggssd gvgapphsaa aalpppaygf gqpygrspsa vaaaaaavfh qqhggqqspg |
| 181 | laalqsgggg glepyagpqq nshdhgfpnh qynsyypnrs aypppapaya lssprggtpg |
| 241 | sgaaaaagsk pppsssasas sssssfaqqr fgamggggps aagggtpqpt atptlnqllt |
| 301 | spssargyqg ypggdysggp qdggagkgpa dmasqcwgaa aaaaaaaaas ggaqqrshha |
| 361 | pmspgssggg gqplartpqp sspmdqmgkm rpqpyggtnp ysqqqgppsg pqqghgypgq |
| 421 | pygsqtpqry pmtmggraqs amgglsytqq ippygqqgps gygqqgqtpy ynggsphpqq |
| 481 | qqppysqqpp sqtphaqpsy qqqpqsqppq lqssqppysq qpsqpphqqs papypsqqst |
| 541 | tqqhpqsqpp ysqpqaqspy qqqqpqqpap stlsqqaayp qpqsqqsqqt aysqqrfppp |
| 601 | gelsqdsfgs qassapsmts skggqedmnl slqsrpsslp dlsgsiddlp mgtegalspg |
| 661 | vstsgisssq gegsnpaqsp fsphtsphlp girgpspspv gspasvaqsr sgplspaavp |
| 721 | gnqmpprpps gqsdsimhps mnqssiaqdr gymqrnpqmp qysspqpgsa lsprqpsggq |
| 781 | ihtgmgsyqq nsmgsygpqg gqygpqggyp rqpnynalpn anypsagmag ginpmgaggq |
| 841 | mhgqpgippy gtlppgrmsh asmgnrpygp nmanmppqvg sgmcpppggm nrktqetava |
| 901 | mhvaansiqn rppgypnmnq ggmmgtgppy gqginsmagm inpqgppysm ggtmannsag |
| 961 | maaspemmgl gdvkltpatk mnnkadgtpk teskskksss stttnekitk lyelggeper |
| 1021 | kmwvdrylaf teekamgmtn lpavgrkpld lyrlyvsvke iggltqvnkn kkwrelatnl |
| 1081 | nvgtsssaas slkkqyiqcl yafeckierg edpppdifaa adskksqpki qppspagsgs |
| 1141 | mqgpqtpqst sssmaeggdl kpptpastph sqipplpgms rsnsvgiqda fndgsdstfq |
| 1201 | krnsmtpnpg yqpsmntsdm mgrmsyepnk dpygsmrkap gsdpfmssgq gpnggmgdpy |
| 1261 | sraagpglgn vamgprqhyp yggpydrvrt epgigpegnm stgapqpnlm psnpdsgmys |
| 1321 | psryppqqqq qqqqrhdsyg nqfstqgtps gspfpsqqtt myqqqqqvss paplprpmen |
| 1381 | rtspskspfl hsgmkmqkag ppvpashiap apvqppmirr ditfppgsve atqpvlkqrr |
| 1441 | rltmkdigtp eawrvmmslk sgllaestwa ldtinillyd dnsimtfnls qlpgllellv |
| 1501 | eyfrrcliei fgilkeyevg dpgqrtlldp grfskvsspa pmeggeeeee llgpkleeee |
| 1561 | eeevvendee iafsgkdkpa senseeklis kfdklpvkiv qkndpfvvdc sdklgrvqef |
| 1621 | dsgllhwrig ggdttehiqt hfesktellp srphapcppa prkhvttaeg tpgttdqegp |
| 1681 | ppdgppekri tatmddmlst rsstltedga ksseaikess kfpfgispaq shrnikiled |
| 1741 | ephskdetpl ctlldwgdsl akrcvcvsnt irslsfvpgn dfemskhpgl llilgklill |
| 1801 | hhkhperkqa pltyekeeeq dqgvscnkve wwwdclemlr entivtlani sgqldlspyp |
| 1861 | esiclpvldg llhwavcpsa eaqdpfstlg pnavlspqrl vletlsklsi qdnnvdlila |
| 1921 | tppfsrlekl ystmvrflsd rknpvcrema vvllanlaqg dslaaraiav qkgsignllg |
| 1981 | fledslaatq fqqsqasllh mqnppfepts vdmmrraara llalakvden hseftlyesr |
| 2041 | lldisvsplm nslvsqvicd vlfligqs |
| Homo sapiens AT rich interactive domain 1A (SWI-like) (ARID1A), | |
| transcript variant 2, mRNA | |
| (SEQ ID NO: 12) | |
| 1 | cagaaagcgg agagtcacag cggggccagg ccctggggag cggagcctcc accgcccccc |
| 61 | tcattcccag gcaagggctt ggggggaatg agccgggaga gccgggtccc gagcctacag |
| 121 | agccgggagc agctgagccg ccggcgcctc ggccgccgcc gccgcctcct cctcctccgc |
| 181 | cgccgccagc ccggagcctg agccggcggg gcggggggga gaggagcgag cgcagcgcag |
| 241 | cagcggagcc ccgcgaggcc cgcccgggcg ggtggggagg gcagcccggg ggactgggcc |
| 301 | ccggggcggg gtgggagggg gggagaagac gaagacaggg ccgggtctct ccgcggacga |
| 361 | gacagcgggg atcatggccg cgcaggtcgc ccccgccgcc gccagcagcc tgggcaaccc |
| 421 | gccgccgccg ccgccctcgg agctgaagaa agccgagcag cagcagcggg aggaggcggg |
| 481 | gggcgaggcg gcggcggcgg cagcggccga gcgcggggaa atgaaggcag ccgccgggca |
| 541 | ggaaagcgag ggccccgccg tggggccgcc gcagccgctg ggaaaggagc tgcaggacgg |
| 601 | ggccgagagc aatgggggtg gcggcggcgg cggagccggc agcggcggcg ggcccggcgc |
| 661 | ggagccggac ctgaagaact cgaacgggaa cgcgggccct aggcccgccc tgaacaataa |
| 721 | cctcacggag ccgcccggcg gcggcggtgg cggcagcagc gatggggtgg gggcgcctcc |
| 781 | tcactcagcc gcggccgcct tgccgccccc agcctacggc ttcgggcaac cctacggccg |
| 841 | gagcccgtct gccgtcgccg ccgccgcggc cgccgtcttc caccaacaac atggcggaca |
| 901 | acaaagccct ggcctggcag cgctgcagag cggcggcggc gggggcctgg agccctacgc |
| 961 | ggggccccag cagaactctc acgaccacgg cttccccaac caccagtaca actcctacta |
| 1021 | ccccaaccgc agcgcctacc ccccgcccgc cccggcctac gcgctgagct ccccgagagg |
| 1081 | tggcactccg ggctccggcg cggcggcggc tgccggctcc aagccgcctc cctcctccag |
| 1141 | cgcctccgcc tcctcgtcgt cttcgtcctt cgctcagcag cgcttcgggg ccatgggggg |
| 1201 | aggcggcccc tccgcggccg gcgggggaac tccccagccc accgccaccc ccaccctcaa |
| 1261 | ccaactgctc acgtcgccca gctcggcccg gggctaccag ggctaccccg ggggcgacta |
| 1321 | cagtggcggg ccccaggacg ggggcgccgg caagggcccg gcggacatgg cctcgcagtg |
| 1381 | ttggggggct gcggcggcgg cagctgcggc ggcggccgcc tcgggagggg cccaacaaag |
| 1441 | gagccaccac gcgcccatga gccccgggag cagcggcggc ggggggcagc cgctcgcccg |
| 1501 | gacccctcag ccatccagtc caatggatca gatgggcaag atgagacctc agccatatgg |
| 1561 | cgggactaac ccatactcgc agcaacaggg acctccgtca ggaccgcagc aaggacatgg |
| 1621 | gtacccaggg cagccatacg ggtcccagac cccgcagcgg tacccgatga ccatgcaggg |
| 1681 | ccgggcgcag agtgccatgg gcggcctctc ttatacacag cagattcctc cttatggaca |
| 1741 | acaaggcccc agcgggtatg gtcaacaggg ccagactcca tattacaacc agcaaagtcc |
| 1801 | tcaccctcag cagcagcagc caccctactc ccagcaacca ccgtcccaga cccctcatgc |
| 1861 | ccaaccttcg tatcagcagc agccacagtc tcaaccacca cagctccagt cctctcagcc |
| 1921 | tccatactcc cagcagccat cccagcctcc acatcagcag tccccggctc catacccctc |
| 1981 | ccagcagtcg acgacacagc agcaccccca gagccagccc ccctactcac agccacaggc |
| 2041 | tcagtctcct taccagcagc agcaacctca gcagccagca ccctcgacgc tctcccagca |
| 2101 | ggctgcgtat cctcagcccc agtctcagca gtcccagcaa actgcctatt cccagcagcg |
| 2161 | cttccctcca ccgcaggagc tatctcaaga ttcatttggg tctcaggcat cctcagcccc |
| 2221 | ctcaatgacc tccagtaagg gagggcaaga agatatgaac ctgagccttc agtcaagacc |
| 2281 | ctccagcttg cctgatctat ctggttcaat agatgacctc cccatgggga cagaaggagc |
| 2341 | tctgagtcct ggagtgagca catcagggat ttccagcagc caaggagagc agagtaatcc |
| 2401 | agctcagtct cctttctctc ctcatacctc ccctcacctg cctggcatcc gaggcccttc |
| 2461 | cccgtcccct gttggctctc ccgccagtgt tgctcagtct cgctcaggac cactctcgcc |
| 2521 | tgctgcagtg ccaggcaacc agatgccacc tcggccaccc agtggccagt cggacagcat |
| 2581 | catgcatcct tccatgaacc aatcaagcat tgcccaagat cgaggttata tgcagaggaa |
| 2641 | cccccagatg ccccagtaca gttcccccca gcccggctca gccttatctc cgcgtcagcc |
| 2701 | ttccggagga cagatacaca caggcatggg ctcctaccag cagaactcca tggggagcta |
| 2761 | tggtccccag gggggtcagt atggcccaca aggtggctac cccaggcagc caaactataa |
| 2821 | tgccttgccc aatgccaact accccagtgc aggcatggct ggaggcataa accccatggg |
| 2881 | tgccggaggt caaatgcatg gacagcctgg catcccacct tatggcacac tccctccagg |
| 2941 | gaggatgagt cacgcctcca tgggcaaccg gccttatggc cctaacatgg ccaatatgcc |
| 3001 | acctcaggtt gggtcaggga tgtgtccccc accagggggc atgaaccgga aaacccaaga |
| 3061 | aactgctgtc gccatgcatg ttgctgccaa ctctatccaa aacaggccgc caggctaccc |
| 3121 | caatatgaat caagggggca tgatgggaac tggacctcct tatggacaag ggattaatag |
| 3181 | tatggctggc atgatcaacc ctcagggacc cccatattcc atgggtggaa ccatggccaa |
| 3241 | caattctgca gggatggcag ccagcccaga gatgatgggc cttggggatg taaagttaac |
| 3301 | tccagccacc aaaatgaaca acaaggcaga tgggacaccc aagacagaat ccaaatccaa |
| 3361 | gaaatccagt tcttctacta caaccaatga gaagatcacc aagttgtatg agctgggtgg |
| 3421 | tgagcctgag aggaagatgt gggtggaccg ttatctggcc ttcactgagg agaaggccat |
| 3481 | gggcatgaca aatctgcctg ctgtgggtag gaaacctctg gacctctatc gcctctatgt |
| 3541 | gtctgtgaag gagattggtg gattgactca ggtcaacaag aacaaaaaat ggcgggaact |
| 3601 | tgcaaccaac ctcaatgtgg gcacatcaag cagtgctgcc agctccttga aaaagcagta |
| 3661 | tatccagtgt ctctatgcct ttgaatgcaa gattgaacgg ggagaagacc ctcccccaga |
| 3721 | catctttgca gctgctgatt ccaagaagtc ccagcccaag atccagcctc cctctcctgc |
| 3781 | gggatcagga tctatgcagg ggccccagac tccccagtca accagcagtt ccatggcaga |
| 3841 | aggaggagac ttaaagccac caactccagc atccacacca cacagtcaga tccccccatt |
| 3901 | gccaggcatg agcaggagca attcagttgg gatccaggat gcctttaatg atggaagtga |
| 3961 | ctccacattc cagaagcgga attccatgac tccaaaccct gggtatcagc ccagtatgaa |
| 4021 | tacctctgac atgatggggc gcatgtccta tgagccaaat aaggatcctt atggcagcat |
| 4081 | gaggaaagct ccagggagtg atcccttcat gtcctcaggg cagggcccca acggcgggat |
| 4141 | gggtgacccc tacagtcgtg ctgccggccc tgggctagga aatgtggcga tgggaccacg |
| 4201 | acagcactat ccctatggag gtccttatga cagagtgagg acggagcctg gaatagggcc |
| 4261 | tgagggaaac atgagcactg gggccccaca gccgaatctc atgccttcca acccagactc |
| 4321 | ggggatgtat tctcctagcc gctacccccc gcagcagcag cagcagcagc agcaacgaca |
| 4381 | tgattcctat ggcaatcagt tctccaccca aggcacccct tctggcagcc ccttccccag |
| 4441 | ccagcagact acaatgtatc aacagcaaca gcaggtatcc agccctgctc ccctgccccg |
| 4501 | gccaatggag aaccgcacct ctcctagcaa gtctccattc ctgcactctg ggatgaaaat |
| 4561 | gcagaaggca ggtcccccag tacctgcctc gcacatagca cctgcccctg tgcagccccc |
| 4621 | catgattcgg cgggatatca ccttcccacc tggctctgtt gaagccacac agcctgtgtt |
| 4681 | gaagcagagg aggcggctca caatgaaaga cattggaacc ccggaggcat ggcgggtaat |
| 4741 | gatgtccctc aagtctggtc tcctggcaga gagcacatgg gcattagata ccatcaacat |
| 4801 | cctgctgtat gatgacaaca gcatcatgac cttcaacctc agtcagctcc cagggttgct |
| 4861 | agagctcctt gtagaatatt tccgacgatg cctgattgag atctttggca ttttaaagga |
| 4921 | gtatgaggtg ggtgacccag gacagagaac gctactggat cctgggaggt tcagcaaggt |
| 4981 | gtctagtcca gctcccatgg agggtgggga agaagaagaa gaacttctag gtcctaaact |
| 5041 | agaagaggaa gaagaagagg aagtagttga aaatgatgag gagatagcct tttcaggcaa |
| 5101 | ggacaagcca gcttcagaga atagtgagga gaagctgatc agtaagtttg acaagcttcc |
| 5161 | agtaaagatc gtacagaaga atgatccatt tgtggtggac tgctcagata agcttgggcg |
| 5221 | tgtgcaggag tttgacagtg gcctgctgca ctggcggatt ggtggggggg acaccactga |
| 5281 | gcatatccag acccacttcg agagcaagac agagctgctg ccttcccggc ctcacgcacc |
| 5341 | ctgcccacca gcccctcgga agcatgtgac aacagcagag ggtacaccag ggacaacaga |
| 5401 | ccaggagggg cccccacctg atggacctcc agaaaaacgg atcacagcca ctatggatga |
| 5461 | catgttgtct actcggtcta gcaccttgac cgaggatgga gctaagagtt cagaggccat |
| 5521 | caaggagagc agcaagtttc catttggcat tagcccagca cagagccacc ggaacatcaa |
| 5581 | gatcctagag gacgaacccc acagtaagga tgagacccca ctgtgtaccc ttctggactg |
| 5641 | gcaggattct cttgccaagc gctgcgtctg tgtgtccaat accattcgaa gcctgtcatt |
| 5701 | tgtgccaggc aatgactttg agatgtccaa acacccaggg ctgctgctca tcctgggcaa |
| 5761 | gctgatcctg ctgcaccaca agcacccaga acggaagcag gcaccactaa cttatgaaaa |
| 5821 | ggaggaggaa caggaccaag gggtgagctg caacaaagtg gagtggtggt gggactgctt |
| 5881 | ggagatgctc cgggaaaaca ccttggttac actcgccaac atctcggggc agttggacct |
| 5941 | atctccatac cccgagagca tttgcctgcc tgtcctggac ggactcctac actgggcagt |
| 6001 | ttgcccttca gctgaagccc aggacccctt ttccaccctg ggccccaatg ccgtcctttc |
| 6061 | cccgcagaga ctggtcttgg aaaccctcag caaactcagc atccaggaca acaatgtgga |
| 6121 | cctgattctg gccacacccc ccttcagccg cctggagaag ttgtatagca ctatggtgcg |
| 6181 | cttcctcagt gaccgaaaga acccggtgtg ccgggagatg gctgtggtac tgctggccaa |
| 6241 | cctggctcag ggggacagcc tggcagctcg tgccattgca gtgcagaagg gcagtatcgg |
| 6301 | caacctcctg ggcttcctag aggacagcct tgccgccaca cagttccagc agagccaggc |
| 6361 | cagcctcctc cacatgcaga acccaccctt tgagccaact agtgtggaca tgatgcggcg |
| 6421 | ggctgcccgc gcgctgcttg ccttggccaa ggtggacgag aaccactcag agtttactct |
| 6481 | gtacgaatca cggctgttgg acatctcggt atcaccgttg atgaactcat tggtttcaca |
| 6541 | agtcatttgt gatgtactgt ttttgattgg ccagtcatga cagccgtggg acacctcccc |
| 6601 | cccccgtgtg tgtgtgcgtg tgtggagaac ttagaaactg actgttgccc tttatttatg |
| 6661 | caaaaccacc tcagaatcca gtttaccctg tgctgtccag cttctccctt gggaaaaagt |
| 6721 | ctctcctgtt tctctctcct ccttccacct cccctccctc catcacctca cgcctttctg |
| 6781 | ttccttgtcc tcaccttact cccctcagga ccctacccca ccctctttga aaagacaaag |
| 6841 | ctctgcctac atagaagact ttttttattt taaccaaagt tactgttgtt tacagtgagt |
| 6901 | ttggggaaaa aaaataaaat aaaaatggct ttcccagtcc ttgcatcaac gggatgccac |
| 6961 | atttcataac tgtttttaat ggtaaaaaaa aaaaaaaaaa atacaaaaaa aaattctgaa |
| 7021 | ggacaaaaaa ggtgactgct gaactgtgtg tggtttattg ttgtacattc acaatcttgc |
| 7081 | aggagccaag aagttcgcag ttgtgaacag accctgttca ctggagaggc ctgtgcagta |
| 7141 | gagtgtagac cctttcatgt actgtactgt acacctgata ctgtaaacat actgtaataa |
| 7201 | taatgtctca catggaaaca gaaaacgctg ggtcagcagc aagctgtagt ttttaaaaat |
| 7261 | gtttttagtt aaacgttgag gagaaaaaaa aaaaaggctt ttcccccaaa gtatcatgtg |
| 7321 | tgaacctaca acaccctgac ctctttctct cctccttgat tgtatgaata accctgagat |
| 7381 | cacctcttag aactggtttt aacctttagc tgcagcggct acgctgccac gtgtgtatat |
| 7441 | atatgacgtt gtacattgca catacccttg gatccccaca gtttggtcct cctcccagct |
| 7501 | acccctttat agtatgacga gttaacaagt tggtgacctg cacaaagcga gacacagcta |
| 7561 | tttaatctct tgccagatat cgcccctctt ggtgcgatgc tgtacaggtc tctgtaaaaa |
| 7621 | gtccttgctg tctcagcagc caatcaactt atagtttatt tttttctggg tttttgtttt |
| 7681 | gttttgtttt ctttctaatc gaggtgtgaa aaagttctag gttcagttga agttctgatg |
| 7741 | aagaaacaca attgagattt tttcagtgat aaaatctgca tatttgtatt tcaacaatgt |
| 7801 | agctaaaact tgatgtaaat tcctcctttt tttccttttt tggcttaatg aatatcattt |
| 7861 | attcagtatg aaatctttat actatatgtt ccacgtgtta agaataaatg tacattaaat |
| 7921 | cttggtaaga cttt |

The present invention also provides methods of inducing neuronal differentiation by contacting a cell with a compound (i.e., an EZH2 inhibitor) of the invention. Preferably, the compound is in an amount sufficient to increase expression of at least one gene selected from the group consisting of CD133 (also called PROM1), DOCK4, PTPRK, PROM2, LHX1, LHX6, LHX9, PAX6, PAX7, VEFGA, FZD3B, FYN, HIF1A, HTRA2, EVX1, CCDC64, and GFAP.

The term “inducing neuronal differentiation” used herein refers to causing a cell to develop into a cell of the neuronal lineage as a result of a direct or intentional effect on the cell.

The present invention also provides methods of inducing cell cycle inhibition by contacting a cell with a compound of the invention. Preferably, the compound is in an amount sufficient to increase expression of at least one gene selected from the group consisting of CKDNIA, CDKN2A, MEN1, CHEK1, IRF6, ALOX15B, CYP27B1, DBC1, NME6, GMNN, HEXIM1, LATS1, MYC, HRAS, TGFB1, IFNG, WNT1, TP53, THBS1, INHBA, IL8, IRF1, TPR, BMP2, BMP4, ETS1, HPGD, BMP7, GATA3, NR2F2, APC, PTPN3, CALR, IL12A, IL12B, PML, CDKN2B, CDKN2C, CDKN1B, SOX2, TAF6, DNA2, PLK1, TERF1, GAS1, CDKN2D, MLF1, PTEN, TGFB2, SMAD3, FOX04, CDK6, TFAP4, MAP2K1, NOTCH2, FOXC1, DLG1, MAD2L1, ATM, NAE1, DGKZ, FHL1, SCRIB, BTG3, PTPRK, RPS6KA2, STK11, CDKN3, TBRG1, CDC73, THAP5, CRLF3, DCUNID3, MYOCD, PAF1, LILRB1, UHMK1, PNPT1, USP47, HEXIM2, CDK5RAP1, NKX3-1, TIPIN, PCBP4, USP44, RBM38, CDT1, RGCC, RNF167, CLSPN, CHMP1A, WDR6, TCF7L2, LATS2, RASSF1, MLTK, MAD2L2, FBX05, ING4, and TRIM35.

The term “inducing cell cycle inhibition” used herein refers to causing an accumulation or an arrest at any phase during cell division and/or duplication.

The present invention also provides methods of inducing tumor suppression by contacting a cell with a compound of the invention. Preferably, the compound is in an amount sufficient to increase expression of BIN1 or any tumor suppressors.

The term “inducing tumor suppression” may include, but is not limited to, a reduction in size of a tumor, a reduction in tumor volume, a decrease in number of tumors, a decrease in number of metastatic lesions in other tissues or organs distant from the primary tumor site, an increase in average survival time of a population of treated subjects in comparison to a population receiving carrier alone, an increase in average survival time of a population of treated subjects in comparison to a population of untreated subjects, an increase in average survival time of a population of treated subjects in comparison to a population receiving monotherapy with a drug that is not a compound of the present invention, a decrease in the mortality rate of a population of treated subjects in comparison to a population receiving carrier alone, a decrease in tumor growth rate, or a decrease in tumor regrowth rate.

The present invention also provides methods of inhibiting hedgehog signaling by contacting a cell with a compound of the invention. Preferably, the compound is in an amount sufficient to reduce expression of at least one gene selected from the group consisting of GLI1, PTCH1, SUFU, KIF7, GLI2, BMP4, MAP3K10, SHH, TCTN3, DYRK2, PTCHD1, and SMO.

The phrase “inhibiting hedgehog signaling” means the hedgehog signaling strength (intensity) with a compound treatment is reduced by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to the hedgehog signaling strength (intensity) without any compound treatment.

The present invention also provides methods of inducing a gene expression by contacting a cell with a compound of the invention. Preferably, the compound is in an amount sufficient to induce neuronal differentiation, cell cycle inhibition and/or tumor suppression. Such gene is selected from the group consisting of CD133 (also called PROM1), DOCK4, PTPRK, PROM2, LHX1, LHX6, LHX9, PAX6, PAX7, VEFGA, FZD3B, FYN, HIF1A, HTRA2, EVX1, CCDC64, GFAP, CKDNIA, CDKN2A, MEN1, CHEK1, IRF6, ALOX15B, CYP27B1, DBC1, NME6, GMNN, HEXIM1, LATS1, MYC, HRAS, TGFB1, IFNG, WNT1, TP53, THBS1, INHBA, IL8, IRF1, TPR, BMP2, BMP4, ETS1, HPGD, BMP7, GATA3, NR2F2, APC, PTPN3, CALR, IL12A, IL12B, PML, CDKN2B, CDKN2C, CDKN1B, SOX2, TAF6, DNA2, PLK1, TERF1, GAS1, CDKN2D, MLF1, PTEN, TGFB2, SMAD3, FOXO4, CDK6, TFAP4, MAP2K1, NOTCH2, FOXC1, DLG1, MAD2L1, ATM, NAE1, DGKZ, FHL1, SCRIB, BTG3, PTPRK, RPS6KA2, STK11, CDKN3, TBRG1, CDC73, THAP5, CRLF3, DCUNID3, MYOCD, PAF1, LILRB1, UHMK1, PNPT1, USP47, HEXIM2, CDK5RAP1, NKX3-1, TIPIN, PCBP4, USP44, RBM38, CDT1, RGCC, RNF167, CLSPN, CHMP1A, WDR6, TCF7L2, LATS2, RASSF1, MLTK, MAD2L2, FBX05, ING4, TRIM35, BIN1 and any tumor suppressors.

The phrase “inducing a gene expression” means the expression level of a particular gene of interest is increased by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to the expression level of this gene without any compound treatment.

The present invention also provides methods of inhibiting a gene expression comprising contacting a cell with a compound of the invention. Preferably, the compound is in an amount sufficient to inhibit hedgehog signaling. Such gene is GLI1, PTCH1, SUFU, KIF7, GLI2, BMP4, MAP3K10, SHH, TCTN3, DYRK2, PTCHD1, or SMO.

The phrase “inhibiting a gene expression” means the expression level of a particular gene of interest is reduced by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to the expression level of this gene without any compound treatment.

Neuronal differentiation, cell cycle inhibition, tumor suppression and hedgehog signaling inhibition can be determined by any methods known in the art.

As used herein, a cell refers to any cell that can be obtained and used by a method described herein. For example, a cell may be obtained from a cell culture. Alternatively, a cell may be isolated from a subject. A cell may also refer to a cell of a subject.

A cell may comprise loss of function of SNF5, ARID1A, ATRX, and/or a component of the SWI/SNF complex. Preferably, a cell may comprise a deletion of SNF5.

A cell may be a cancer cell, where the cancer is selected from the group consisting of medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epithelioid sarcoma, renal medullo carcinoma, diffuse large B-cell lymphoma, follicular lymphoma and not otherwise specified (NOS) sarcoma. More preferably a cell is a cancer cell of medulloblastoma, malignant rhabdoid tumor, or atypical teratoid rhabdoid tumor.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, NO, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1 (mi), PN1a, PN1b, PN1c, pN2, pN2a, pN2b, pN3, pN3a, pN3b, or pN3c.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “monotherapy” refers to the administration of a single active or therapeutic compound to a subject in need thereof. Preferably, monotherapy will involve administration of a therapeutically effective amount of an active compound. For example, cancer monotherapy with one of the compound of the present invention, or a pharmaceutically acceptable salt, polymorph, solvate, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the present invention, or a pharmaceutically acceptable salt, polymorph or solvate thereof, is more effective than combination therapy in inducing a desired biological effect.

As used herein, “treating” or “treat” describes the management and care of a patient for the purpose of combating a disease, condition, or disorder and includes the administration of a compound of the present invention, or a pharmaceutically acceptable salt, polymorph or solvate thereof, to alleviate one or more symptoms or complications of a disease, condition or disorder, or to eliminate the disease, condition or disorder. The term “treat” can also include treatment of a cell in vitro or an animal model.

A compound of the present invention, or a pharmaceutically acceptable salt, polymorph or solvate thereof, can also be used to prevent a disease, condition or disorder, or used to identify suitable candidates for such purposes. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, polymorph, solvate, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, polymorph, solvate, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

Treating cancer can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100 (5): 2674-8, 2003. In an aspect, cell death occurs by apoptosis.

As used herein, the term “selectively” means tending to occur at a higher frequency in one population than in another population. The compared populations can be cell populations. Preferably, a compound of the present invention, or a pharmaceutically acceptable salt, polymorph or solvate thereof, acts selectively on a cancer or precancerous cell but not on a normal cell. Preferably, a compound of the present invention, or a pharmaceutically acceptable salt, polymorph or solvate thereof, acts selectively to modulate one molecular target (e.g., a target protein methyltransferase) but does not significantly modulate another molecular target (e.g., a non-target protein methyltransferase). The invention also provides a method for selectively inhibiting the activity of an enzyme, such as a protein methyltransferase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A compound of the present invention, or a pharmaceutically acceptable salt, polymorph or solvate thereof, can modulate the activity of a molecular target (e.g., a target protein methyltransferase). Modulating refers to stimulating or inhibiting an activity of a molecular target. Preferably, a compound of the present invention, or a pharmaceutically acceptable salt, polymorph or solvate thereof, modulates the activity of a molecular target if it stimulates or inhibits the activity of the molecular target by at least 2-fold relative to the activity of the molecular target under the same conditions but lacking only the presence of said compound. More preferably, a compound of the present invention, or a pharmaceutically acceptable salt, polymorph or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A compound of the present invention, or a pharmaceutically acceptable salt, polymorph or solvate thereof, does not significantly modulate the activity of a molecular target if the addition of the compound does not stimulate or inhibit the activity of the molecular target by greater than 10% relative to the activity of the molecular target under the same conditions but lacking only the presence of said compound.

As used herein, the term “isozyme selective” means preferential inhibition or stimulation of a first isoform of an enzyme in comparison to a second isoform of an enzyme (e.g., preferential inhibition or stimulation of a protein methyltransferase isozyme alpha in comparison to a protein methyltransferase isozyme beta). Preferably, a compound of the present invention, or a pharmaceutically acceptable salt, polymorph or solvate thereof, demonstrates a minimum of a fourfold differential, preferably a tenfold differential, more preferably a fifty fold differential, in the dosage required to achieve a biological effect. Preferably, a compound of the present invention, or a pharmaceutically acceptable salt, polymorph or solvate thereof, demonstrates this differential across the range of inhibition, and the differential is exemplified at the IC₅₀, i.e., a 50% inhibition, for a molecular target of interest.

Administering a compound of the present invention, or a pharmaceutically acceptable salt, polymorph or solvate thereof, to a cell or a subject in need thereof can result in modulation (i.e., stimulation or inhibition) of an activity of a protein methyltransferase of interest.

Detection of methylation of H3-K27, formation of trimethylated H3-K27, conversion of monomethylated H3-K27 to dimethylated H3-K27, or conversion of dimethylated H3-K27 to trimethylated H3-K27 can be accomplished using any suitable method. Exemplary methods can be found in US20120071418, the contents of which are incorporated herein by reference.

Administering a compound of the present invention, or a pharmaceutically acceptable salt, polymorph or solvate thereof, to a cell or a subject in need thereof results in modulation (i.e., stimulation or inhibition) of an activity of an intracellular target (e.g., substrate). Several intracellular targets can be modulated with the compounds of the present invention, including, but not limited to, protein methyltrasferase.

Preferably, an effective amount of a compound of the present invention, or a pharmaceutically acceptable salt, polymorph or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present invention, or a pharmaceutically acceptable salt, polymorph or solvate thereof, can induce or activate cell death selectively in cancer cells. Administering to a subject in need thereof a compound of the present invention, or a pharmaceutically acceptable salt, polymorph or solvate thereof, can induce or activate cell death selectively in cancer cells. Contacting a cell with a compound of the present invention, or a pharmaceutically acceptable salt, polymorph or solvate thereof, can induce cell death selectively in one or more cells affected by a cell proliferative disorder. Preferably, administering to a subject in need thereof a compound of the present invention, or a pharmaceutically acceptable salt, polymorph or solvate thereof, induces cell death selectively in one or more cells affected by a cell proliferative disorder.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the invention.

A compound (i.e., an EZH2 inhibitor) that can be used in any methods described herein may have the following Formula I:

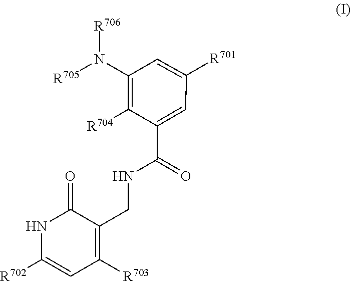

or a pharmaceutically acceptable salt thereof; wherein
R⁷⁰¹ is H, F, OR⁷⁰⁷, NHR⁷⁰⁷, —(C═C)—(CH₂)ₙ₇—R⁷⁰⁸, phenyl, 5- or 6-membered heteroaryl, C3-8 cycloalkyl, or 4-7 membered heterocycloalkyl containing 1-3 heteroatoms, wherein the phenyl, 5- or 6-membered heteroaryl, C3-8 cycloalkyl or 4-7 membered heterocycloalkyl each independently is optionally substituted with one or more groups selected from halo, C₁₋₃ alkyl, OH, O—C₁₋₆ alkyl, NH—C₁₋₆ alkyl, and, C₁₋₃ alkyl substituted with C₃₋₈ cycloalkyl or 4-7 membered heterocycloalkyl containing 1-3 heteroatoms, wherein each of the O—C₁₋₆ alkyl and NH—C₁₋₆ alkyl is optionally substituted with hydroxyl, O—C₁₋₃ alkyl or NH—C₁₋₃ alkyl, each of the O—C₁₋₃ alkyl and NH—C₁₋₃ alkyl being optionally further substituted with O—C₁₋₃ alkyl or NH—C₁₋₃ alkyl;
each of R⁷⁰² and R⁷⁰³, independently is H, halo, C₁₋₄ alkyl, C₁₋₆ alkoxyl or C₆-C₁₀ aryloxy, each optionally substituted with one or more halo;
each of R⁷⁰⁴ and R⁷⁰⁵, independently is C₁₋₄ alkyl;
R⁷⁰⁶ is cyclohexyl substituted by N(C₁₋₄ alkyl) 2 wherein one or both of the C₁₋₄ alkyl is substituted with C₁₋₆ alkoxy; or R⁷⁰⁶ is tetrahydropyranyl;
R⁷⁰⁷ is C₁₋₄ alkyl optionally substituted with one or more groups selected from hydroxyl, C₁₋₄ alkoxy, amino, mono- or di-C₁₋₄ alkylamino, C₃₋₈ cycloalkyl, and 4-7 membered heterocycloalkyl containing 1-3 heteroatoms, wherein the C₃₋₈ cycloalkyl or 4-7 membered heterocycloalkyl each independently is further optionally substituted with C₁₋₃ alkyl;
R⁷⁰⁸ is C₁₋₄ alkyl optionally substituted with one or more groups selected from OH, halo, and C₁₋₄ alkoxy, 4-7 membered heterocycloalkyl containing 1-3 heteroatoms, or O—C₁₋₆ alkyl, wherein the 4-7 membered heterocycloalkyl can be optionally further substituted with OH or C₁₋₆ alkyl; and n₇ is 0, 1 or 2.

For example, R⁷⁰⁶ is cyclohexyl substituted by N(C₁₋₄ alkyl)₂ wherein one of the C₁₋₄ alkyl is unsubstituted and the other is substituted with methoxy.

For example, R⁷⁰⁶ is

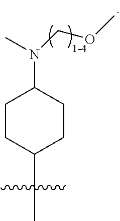

For example, the compound is of Formula II:

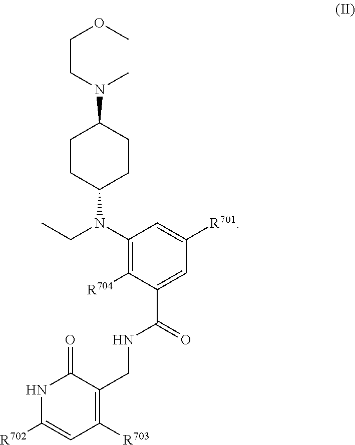

For example, R⁷⁰² is methyl or isopropyl and R⁷⁰³ is methyl or methoxyl.

For example, R⁷⁰⁴ is methyl.

For example, R⁷⁰¹ is OR⁷⁰⁷ and R⁷⁰⁷ is C₁₋₃ alkyl optionally substituted with OCH₃ or morpholine.

For example, R⁷⁰¹ is H or F.

For example, R⁷⁰¹ is tetrahydropyranyl, phenyl, pyridyl, pyrimidyl, pyrazinyl, imidazolyl, or pyrazolyl, each of which is optionally substituted with methyl, methoxy, ethyl substituted with morpholine, or —OCH₂CH₂OCH₃.

For example, R⁷⁰⁸ is morpholine, piperidine, piperazine, pyrrolidine, diazepane, or azetidine, each of which is optionally substituted with OH or C₁₋₆ alkyl.

For example, R⁷⁰⁸ is morpholine

For example, R⁷⁰⁸ is piperazine substituted with C₁₋₆ alkyl.

For example, R⁷⁰⁸ is methyl, t-butyl or C(CH₃)₂OH.

A compound (i.e., an EZH2 inhibitor) that can be used in any methods described herein may have the following Formula III:

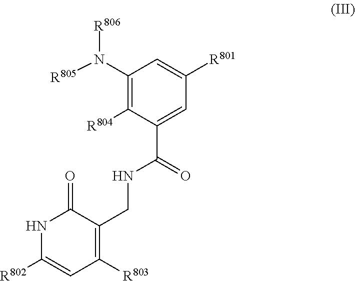

or a pharmaceutically acceptable salt thereof.

In this formula:
R⁸⁰¹ is C₁₋₆ alkyl, C₂₋₆ alkenyl, C₂₋₆ alkynyl, C₃₋₈ cycloalkyl, 4-7 membered heterocycloalkyl containing 1-3 heteroatoms, phenyl or 5- or 6-membered heteroaryl, each of which is substituted with O—C₁₋₆ alkyl-Rₓ or NH—C₁₋₆ alkyl-Rₓ, wherein Rₓ is hydroxyl, O—C₁₋₃ alkyl or NH—C₁₋₃ alkyl, and Rₓ is optionally further substituted with O—C₁₋₃ alkyl or NH—C₁₋₃ alkyl except when Rₓ is hydroxyl; or R⁸⁰¹ is phenyl substituted with -Q2-T2, wherein Q₂ is a bond or C₁-C₃ alkyl linker optionally substituted with halo, cyano, hydroxyl or C₁-C₆ alkoxy, and T2 is optionally substituted 4- to 12-membered heterocycloalkyl; and R⁸⁰¹ is optionally further substituted;
each of R⁸⁰² and R⁸⁰³, independently is H, halo, C₁₋₄ alkyl, C₁₋₆ alkoxyl or C₆-C₁₀ aryloxy, each optionally substituted with one or more halo;
each of R⁸⁰⁴ and R⁸⁰⁵, independently is C₁₋₄ alkyl; and
R⁸⁰⁶ is -Qₓ-Tₓ, wherein Qₓ is a bond or C₁₋₄ alkyl linker, Tₓ is H, optionally substituted C₁₋₄ alkyl, optionally substituted C₃-C₈ cycloalkyl or optionally substituted 4- to 14-membered heterocycloalkyl.

For example, each of Qₓ and Q₂ independently is a bond or methyl linker, and each of Tₓ and T₂ independently is tetrahydropyranyl, piperidinyl substituted by 1, 2, or 3 C₁₋₄ alkyl groups, or cyclohexyl substituted by N(C₁₋₄ alkyl)₂ wherein one or both of the C₁₋₄ alkyl is optionally substituted with C₁₋₆ alkoxy;

For example, R⁸⁰⁶ is cyclohexyl substituted by N(C₁₋₄ alkyl)₂ or R⁸⁰⁶ is tetrahydropyranyl.

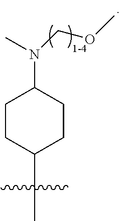

For example, R⁸⁰⁶ is

For example, R⁸⁰¹ is phenyl or 5- or 6-membered heteroaryl substituted with O—C₁₋₆ alkyl-Rₓ, or R⁸⁰¹ is phenyl substituted with CH₂-tetrahydropyranyl.

For example, a compound of the present invention is of Formula IVa or IVb:

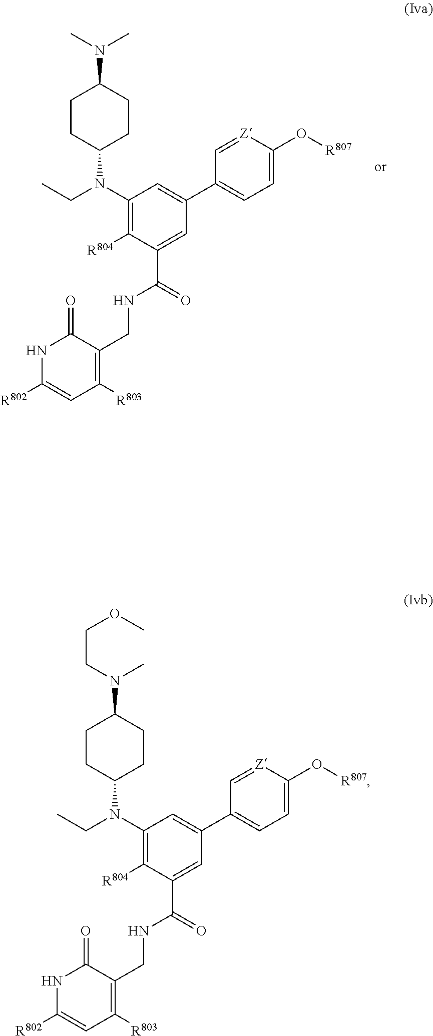

wherein Z′ is CH or N, and R⁸⁰⁷ is C₂₋₃ alkyl-Rₓ.

For example, R⁸⁰⁷ is —CH₂CH₂OH, —CH₂CH₂OCH₃, or —CH₂CH₂OCH₂CH₂OCH₃.

For example, R⁸⁰² is methyl or isopropyl and R⁸⁰³ is methyl or methoxyl.

For example, R⁸⁰⁴ is methyl.

A compound of the present invention may have the following Formula (V):

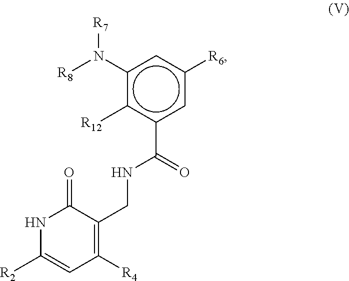

or a pharmaceutically acceptable salt or ester thereof.

In this formula:
R₂, R₄ and R₁₂ are each independently C₁₋₆ alkyl;
R₆ is C₆-C10 aryl or 5- or 6-membered heteroaryl, each of which is optionally substituted with one or more -Q₂-T2, wherein Q₂ is a bond or C₁-C₃ alkyl linker optionally substituted with halo, cyano, hydroxyl or C₁-C₆ alkoxy, and T2 is H, halo, cyano, —ORₐ, —NRₐR_{b}, —(NRₐR_{b}R_{c})⁺A⁻, —C(O)Rₐ, —C(O)ORₐ, —C(O)NRₐR_{b}, —NR_{b}C(O)Rₐ, —NR_{b}C(O)ORₐ, —S(O)₂Rₐ, —S(O)₂NRₐR_{b}, or R_{S2}, in which each of Rₐ, R_{b}, and R_{c}, independently is H or R_{S3}, A⁻ is a pharmaceutically acceptable anion, each of R_{S2} and R_{S3}, independently, is C₁-C₆ alkyl, C₃-C₈ cycloalkyl, C₆-C₁₀ aryl, 4 to 12-membered heterocycloalkyl, or 5- or 6-membered heteroaryl, or Rₐ and R_{b}, together with the N atom to which they are attached, form a 4 to 12-membered heterocycloalkyl ring having 0 or 1 additional heteroatom, and each of R_{S2}, R_{S3}, and the 4 to 12-membered heterocycloalkyl ring formed by Rₐ and R_{b}, is optionally substituted with one or more -Q₃-T3, wherein Q₃ is a bond or C₁-C₃ alkyl linker each optionally substituted with halo, cyano, hydroxyl or C₁-C₆ alkoxy, and T3 is selected from the group consisting of halo, cyano, C₁-C₆ alkyl, C₃-C₈ cycloalkyl, C6-C10 aryl, 4 to 12-membered heterocycloalkyl, 5- or 6-membered heteroaryl, OR_{d}, COOR_{d}, —S(O)₂R_{d}, —NR_{d}Rₑ, and —C(O)NR_{d}Rₑ, each of R_{d} and Rₑ independently being H or C₁-C₆ alkyl, or -Q₃-T3 is oxo; or any two neighboring-Q₂-T2, together with the atoms to which they are attached form a 5- or 6-membered ring optionally containing 1-4 heteroatoms selected from N, O and S and optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C10 aryl, 4 to 12-membered heterocycloalkyl, and 5- or 6-membered heteroaryl;
R₇ is -Q₄-T4, in which Q₄ is a bond, C₁-C₄ alkyl linker, or C2-C4 alkenyl linker, each linker optionally substituted with halo, cyano, hydroxyl or C₁-C₆ alkoxy, and T4 is H, halo, cyano, NR_{f}R_{g}, —OR_{f}, —C(O)R_{f}, —C(O)OR_{f}, —C(O)NR_{f}R_{g}, —C(O)NR_{f}OR_{g}, —NR(C(O)R_{g}, —S(O)₂R_{f}, or R_{S4}, in which each of R_{f} and R_{g}, independently is H or R_{S5}, each of R_{S4} and R_{S5}, independently is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and each of R_{S4} and R_{S5} is optionally substituted with one or more -Q₅-T5, wherein Q₅ is a bond, C(O), C(O)NRₖ, NRₖC(O), S(O)₂, or C₁-C₃ alkyl linker, Rₖ being H or C₁-C₆ alkyl, and T₅ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)_{q}R_{q} in which q is 0, 1, or 2 and R_{q} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T₅ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl except when T5 is H, halo, hydroxyl, or cyano; or -Q₅-T₅ is oxo; and
R₈ is H, halo, hydroxyl, COOH, cyano, R_{S6}, OR_{S6}, or COOR_{S6}, in which R_{S6} is C₁-C₆ alkyl, C₂-C₆ alkenyl, C₂-C₆ alkynyl, C₃-C₈ cycloalkyl, 4 to 12-membered heterocycloalkyl, amino, mono-C₁-C₆ alkylamino, or di-C₁-C₆ alkylamino, and R_{S6} is optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino; or R₇ and R₈, together with the N atom to which they are attached, form a 4 to 11-membered heterocycloalkyl ring having 0 to 2 additional heteroatoms, and the 4 to 11-membered heterocycloalkyl ring formed by R₇ and R₈ is optionally substituted with one or more -Q₆-T₆, wherein Q₆ is a bond, C(O), C(O)NRₘ, NRₘC(O), S(O)₂, or C₁-C₃ alkyl linker, Rₘ being H or C₁-C₆ alkyl, and T6 is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)ₚRₚ in which p is 0, 1, or 2 and Rₚ is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T6 is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl except when T6 is H, halo, hydroxyl, or cyano; or -Q₆-T6 is oxo.

For example, R₆ is C₆-C₁₀ aryl or 5- or 6-membered heteroaryl, each of which is optionally, independently substituted with one or more -Q₂-T2, wherein Q₂ is a bond or C₁-C₃ alkyl linker, and T2 is H, halo, cyano, —ORₐ, —NRₐR_{b},

(NRₐR_{b}R_{c})⁺A⁻, —C(O)NRₐR_{b}, —NR_{b}C(O)Rₐ, —S(O)₂Rₐ, or R_{S2}, in which each of Rₐ and R_{b}, independently is H or R_{S3}, each of R_{S2} and R_{S3}, independently, is C₁-C₆ alkyl, or Rₐ and R_{b}, together with the N atom to which they are attached, form a 4 to 7-membered heterocycloalkyl ring having 0 or 1 additional heteroatom, and each of R_{S2}, R_{S3}, and the 4 to 7-membered heterocycloalkyl ring formed by Rₐ and R_{b}, is optionally, independently substituted with one or more -Q₃-T3, wherein Q₃ is a bond or C₁-C₃ alkyl linker and T3 is selected from the group consisting of halo, C₁-C₆ alkyl, 4 to 7-membered heterocycloalkyl, OR_{d}, —S(O)₂Rₐ, and —NR_{d}Rₑ, each of Rₐ and Rₑ independently being H or C₁-C₆ alkyl, or -Q₃-T3 is oxo; or any two neighboring -Q₂-T2, together with the atoms to which they are attached form a 5- or 6-membered ring optionally containing 1-4 heteroatoms selected from N, O and S.

For example, the compound of the present invention is of Formula (VI):

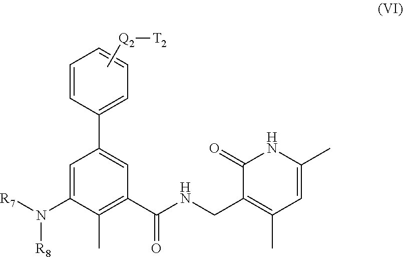

or a pharmaceutically acceptable salt thereof, wherein Q₂ is a bond or methyl linker, T2 is H, halo, —ORₐ, —NRₐR_{b}, —(NRₐR_{b}R_{c}) A, or —S(O)₂NRₐR_{b}, R₇ is piperidinyl, tetrahydropyran, cyclopentyl, or cyclohexyl, each optionally substituted with one-Q₅-T5 and R₈ is ethyl.

A compound of the present invention may have the following Formula (VIa):

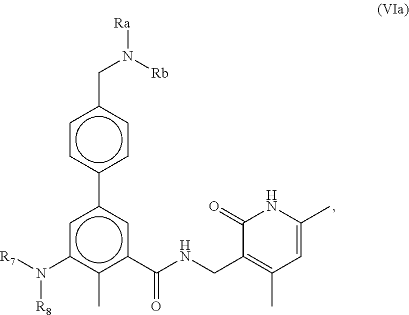

wherein
each of Rₐ and R_{b}, independently is H or R_{S3}, R_{S3} being C₁-C₆ alkyl, C₃-C₈ cycloalkyl, C₆-C₁₀ aryl, 4 to 12-membered heterocycloalkyl, or 5- or 6-membered heteroaryl, or Rₐ and R_{b}, together with the N atom to which they are attached, form a 4 to 12-membered heterocycloalkyl ring having 0 or 1 additional heteroatom, and each of R_{S3} and the 4 to 12-membered heterocycloalkyl ring formed by Rₐ and R_{b}, is optionally substituted with one or more -Q₃-T3, wherein Q₃ is a bond or C₁-C₃ alkyl linker each optionally substituted with halo, cyano, hydroxyl or C₁-C₆ alkoxy, and T3 is selected from the group consisting of halo, cyano, C₁-C₆ alkyl, C₃-C₈ cycloalkyl, C₆-C₁₀ aryl, 4 to 12-membered heterocycloalkyl, 5- or 6-membered heteroaryl, OR_{d}, COOR_{d}, —S(O)₂Rd, —NR_{d}Rₑ, and —C(O)NR_{d}Rₑ, each of Rₐ and Rₑ independently being H or C₁-C₆ alkyl, or -Q₃-T3 is oxo;
R₇ is -Q₄-T4, in which Q₄ is a bond, C₁-C₄ alkyl linker, or C₂-C₄ alkenyl linker, each linker optionally substituted with halo, cyano, hydroxyl or C₁-C₆ alkoxy, and T4 is H, halo, cyano, NR_{f}R_{g}, —OR_{f}, —C(O)R_{f}, —C(O)OR_{f}, —C(O)NR_{f}R_{g}, —C(O)NR_{f}OR_{g}, —NR(C(O)R_{g}, —S(O)₂R_{f}, or R_{S4}, in which each of R_{f} and R_{g}, independently is H or R_{S5}, each of R_{S4} and R_{S5}, independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, or 5- or 6-membered heteroaryl, and each of R_{S4} and R_{S5} is optionally substituted with one or more -Q₅-T5, wherein Q₅ is a bond, C(O), C(O)NRₖ, NRₖC(O), S(O)₂, or C₁-C₃ alkyl linker, Rₖ being H or C₁-C₆ alkyl, and T₅ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 7-membered heterocycloalkyl, 5- or 6-membered heteroaryl, or S(O)_{q}R_{q} in which q is 0, 1, or 2 and R_{q} is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, or 5- or 6-membered heteroaryl, and T₅ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 7-membered heterocycloalkyl, and 5- or 6-membered heteroaryl except when T5 is H, halo, hydroxyl, or cyano; or -Q₅-T5 is oxo; provided that R₇ is not H; and
R₈ is H, halo, hydroxyl, COOH, cyano, R_{S6}, OR_{S6}, or COOR_{S6}, in which R_{S6} is C₁-C₆ alkyl, C₂-C₆ alkenyl, C₂-C₆ alkynyl, amino, mono-C₁-C₆ alkylamino, or di-C₁-C₆ alkylamino, and R_{S6} is optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino; or R₇ and R₈, together with the N atom to which they are attached, form a 4 to 11-membered heterocycloalkyl ring which has 0 to 2 additional heteroatoms and is optionally substituted with one or more -Q₆-T6, wherein Q₆ is a bond, C(O), C(O)NRₘ, NRₘC(O), S(O)₂, or C₁-C₃ alkyl linker, Rₘ being H or C₁-C₆ alkyl, and T6 is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 7-membered heterocycloalkyl, 5- or 6-membered heteroaryl, or S(O)ₚRₚ in which p is 0, 1, or 2 and Rₚ is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, or 5- or 6-membered heteroaryl, and T6 is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 7-membered heterocycloalkyl, and 5- or 6-membered heteroaryl except when T6 is H, halo, hydroxyl, or cyano; or -Q₆-T6 is oxo.

For example, Rₐ and R_{b}, together with the N atom to which they are attached, form a 4 to 7-membered heterocycloalkyl ring having 0 or 1 additional heteroatoms to the N atom and the ring is optionally substituted with one or more -Q₃-T3, wherein the heterocycloalkyl is azetidinyl, pyrrolidinyl, imidazolidinyl, pyrazolidinyl, oxazolidinyl, isoxazolidinyl, triazolidinyl, piperidinyl, 1,2,3,6-tetrahydropyridinyl, piperazinyl, or morpholinyl.

For example, R₇ is C₃-C₈ cycloalkyl or 4 to 7-membered heterocycloalkyl, each optionally substituted with one or more -Q₅-T5.

For example, R₇ is piperidinyl, tetrahydropyran, tetrahydro-2H-thiopyranyl, cyclopentyl, cyclohexyl, pyrrolidinyl, or cycloheptyl, each optionally substituted with one or more -Q₅-T5.

For example, R₈ is H or C₁-C₆ alkyl which is optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino.

In some embodiments, a compound that can be used in any methods presented here is:

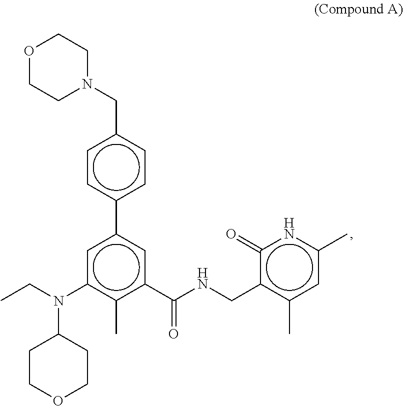

stereoisomers thereof or pharmaceutically acceptable salt or solvate thereof.

In some embodiments, a compound that can be used in any methods presented here is:

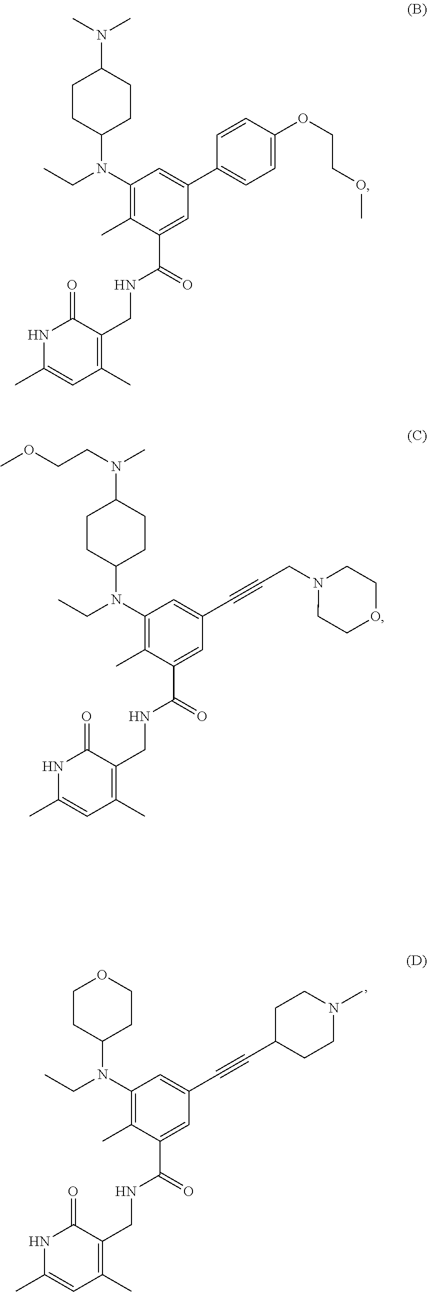

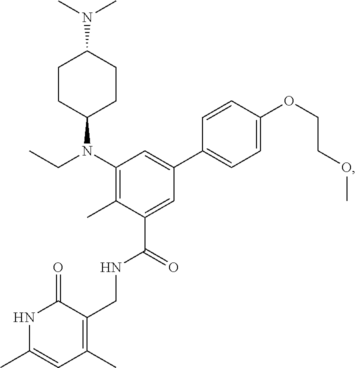

stereoisomers thereof or pharmaceutically acceptable salts and solvates thereof.

In some embodiments, a compound that can be used in any methods presented here is:

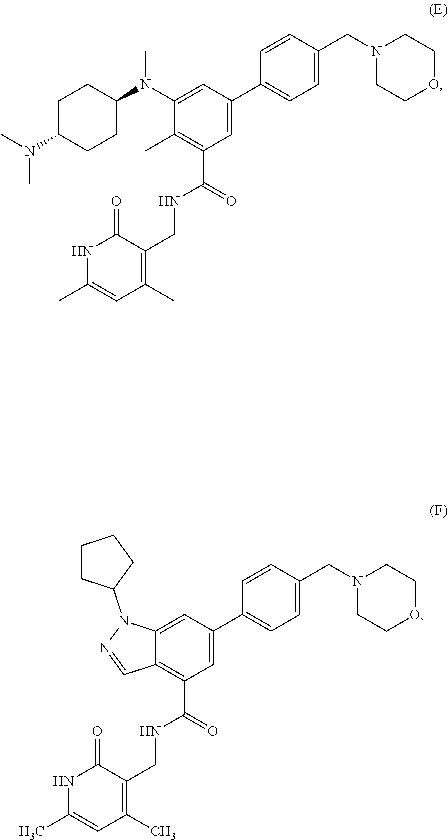 [SMILES: Cc1cc(C)c(CNC(=O)c2cc(-c3ccc(CN4CCOCC4)cc3)cc(N(C)[C@H]3CC[C@H](N(C)C)CC3)c2C)c(=O)[nH]1 ; Cc1cc(C)c(CNC(=O)c2cc(-c3ccc(CN4CCOCC4)cc3)cc3c2cnn3C2CCCC2)c(=O)[nH]1]

stereoisomers thereof or pharmaceutically acceptable salts and solvates thereof.

In some embodiments, the compounds suitable for use in the method of this invention include compounds of Formula (VII):

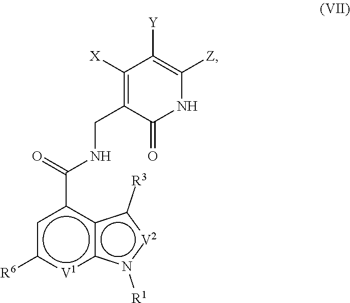

wherein,
V¹ is N or CR⁷,
V² is N or CR², provided when V¹ is N, V² is N,
X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀) bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂Rₐ, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R_{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
Y is H or halo;
R₁ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀) bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, —CONR^{a}NR^{a}R^{b};
R₂ is hydrogen, (C₁-C₈)alkyl, trifluoromethyl, alkoxy, or halo, in which said (C₁-C₅)alkyl is optionally substituted with one to two groups selected from amino and (C₁-C₃)alkylamino;
R⁷ is hydrogen, (C₁-C₃)alkyl, or alkoxy;
R³ is hydrogen, (C₁-C₈)alkyl, cyano, trifluoromethyl, —NR^{a}R^{b}, or halo;
R⁶ is selected from the group consisting of hydrogen, halo, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀) bicycloalkyl, unsubstituted or substituted heterocycloalkyl, unsubstituted or substituted heterocycloalkyl-(C₁—C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, —OC(O)NR^{a}R^{b};
wherein any (C₁-C₅)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, —(C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, OC(O)NR^{a}R^{b}, heterocycloalkyl, aryl, heteroaryl, aryl(C₁-C₄)alkyl, and heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a},
   —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};
R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀) bicycloalkyl, heterocycloalkyl, aryl, or heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂ (C₁-C₄)alkyl, —CONH₂, —CONH(C₁-C₄)alkyl,
   —CON((C₁-C₄)alkyl)((C₁-C₄)alkyl), —SO₂ (C₁-C₄)alkyl, —SO₂NH₂, —SO₂NH(C₁-C₄)alkyl, and SO₂N((C₁-C₄)alkyl)((C₁-C₄)alkyl);
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy (C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;
each R^{c} is independently (C₁-C₄)alkylamino, —NR^{a}SO₂R, —SOR^{a}, —SO₂R^{a}, —NR^{a}C(O)OR^{a}, —NR^{a}R^{b}, or —CO₂R^{a};
or a salt thereof.

Subgroups of the compounds encompassed by the general structure of Formula (I) are represented as follows:

### Subgroup A of Formula (VII)

X and Z are selected from the group consisting of (C₁-C₅)alkyl, (C₃-C₈)cycloalkyl, heterocycloalkyl, aryl, heteroaryl, —NR^{a}R^{b}, and —OR^{a};
Y is H or F;
R¹ is selected from the group consisting of (C₁-C₅)alkyl, (C₃-C₈)cycloalkyl, heterocycloalkyl, aryl, and heteroaryl;
R² is hydrogen, (C₁-C₈)alkyl, trifluoromethyl, alkoxy, or halo, in which said (C₁-C₈)alkyl is optionally substituted with one to two groups selected from amino and (C₁-C₃)alkylamino;
R⁷ is hydrogen, (C₁-C₃)alkyl, or alkoxy;
R³ is selected from the group consisting of hydrogen, (C₁-C₈)alkyl, cyano, trifluoromethyl, —NR^{a}R^{b}, and halo;
R⁶ is selected from the group consisting of hydrogen, halo, cyano, trifluoromethyl, amino, (C₁-C₈)alkyl, (C₃-C₈)cycloalkyl, aryl, heteroaryl, acylamino; (C₂-C₈)alkynyl, arylalkynyl, heteroarylalkynyl; —SO₂R^{a}; —SO₂NR^{a}R^{b} and —NR^{a}SO₂R^{b};
   wherein any (C₁-C₈)alkyl, (C₃-C₈)cycloalkyl, (C₂-C₈)alkynyl, arylalkynyl, heteroarylalkynyl group is optionally substituted by 1, 2 or 3 groups independently selected from —O(C₁-C₆)alkyl(R^{c})₁₋₂, —S(C₁-C₆)alkyl(R^{c})₁₋₂, —(C₁-C₆)alkyl(R^{c})₁₋₂, —(C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀) bicycloalkyl, heterocycloalkyl, aryl, or heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂ (C₁-C₄)alkyl, —CONH₂, —CONH(C₁-C₄)alkyl, —CON((C₁-C₄)alkyl)((C₁-C₄)alkyl), —SO₂ (C₁-C₄)alkyl, —SO₂NH₂, —SO₂NH(C₁-C₄)alkyl, and —SO₂N((C₁-C₄)alkyl)((C₁-C₄)alkyl);
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy (C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring. An aryl or heteroaryl group in this particular subgroup A is selected independently from the group consisting of furan, thiophene, pyrrole, oxazole, thiazole, imidazole, pyrazole, oxadiazole, thiadiazole, triazole, tetrazole, benzofuran, benzothiophene, benzoxazole, benzothiazole, phenyl, pyridine, pyridazine, pyrimidine, pyrazine, triazine, tetrazine, quinoline, cinnoline, quinazoline, quinoxaline, and naphthyridine or another aryl or heteroaryl group as follows:

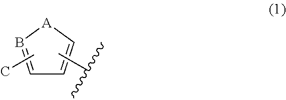

wherein in (1),
A is O, NH, or S; B is CH or N, and C is hydrogen or C₁-C₈ alkyl; or

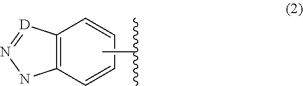

wherein in (2),
D is N or C optionally substituted by hydrogen or C₁-C₈ alkyl; or

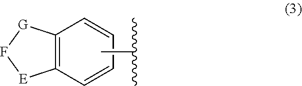

wherein in (3),
E is NH or CH₂; F is O or CO; and G is NH or CH₂; or

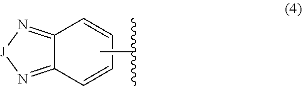

wherein in (4),
J is O, S or CO; or

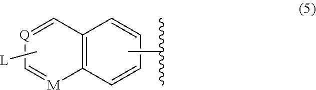

wherein in (5),
Q is CH or N;
M is CH or N; and
L/(5) is hydrogen, halo, amino, cyano, (C₁-C₈)alkyl, (C₃-C₈)cycloalkyl, —COR^{a}, —CO₂R^{a}, —CONR^{a}R^{b}, —CONR^{a}NR^{a}R^{b}, —SO₂R^{a}, —SO₂NR^{a}R^{b}, —NRₐR^{b}, —NR^{a}C(O)R^{b}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, or —OR^{a},
   wherein any (C₁-C₈)alkyl or (C₃-C₈)cycloalkyl group is optionally substituted by 1, 2 or 3 groups independently selected from (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b}; wherein R^{a} and R^{b} are defined as above; or

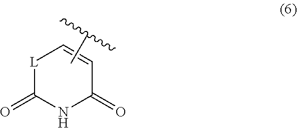

wherein in (6),
L/(6) is NH or CH₂; or

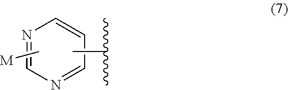

wherein in 7,
   M/(7) is hydrogen, halo, amino, cyano, (C₁-C₈)alkyl, (C₃-C₈)cycloalkyl, heterocycloalkyl, —COR^{a}, —CO₂R^{a}, —CONR^{a}R^{b}, —CONR^{a}NR^{a}R^{b}, —SO₂R^{a}, —SO₂NR^{a}R^{b}, —NR^{a}R^{b}, —NR^{a}C(O)R^{b}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, or —OR^{a},
   wherein any (C₁-C₈)alkyl, (C₃-C₈)cycloalkyl, or heterocycloalkyl group is optionally substituted by 1, 2 or 3 groups independently selected from (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b}; wherein R^{a} and R^{b} are defined as above; or

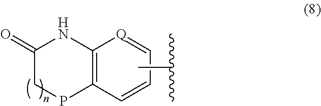

wherein in (8),
P is CH₂, NH, O, or S; Q/(8) is CH or N; and n is 0-2; or

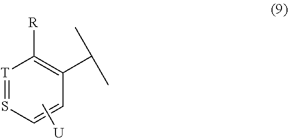

wherein in (9),
S/(9) and T/(9) is C, or S/(9) is C and T/(9) is N, or S/(9) is N and T/(9) is C;
R is hydrogen, amino, methyl, trifluoromethyl, or halo;
U is hydrogen, halo, amino, cyano, nitro, trifluoromethyl, (C₁-C₈)alkyl, (C₃-C₈)cycloalkyl, —COR^{a}, —CO₂R^{a}, —CONR^{a}R^{b}, —SO₂R^{a}, —SO₂NR^{a}R^{b}, —NR^{a}R^{b}, —NR^{a}C(O)R^{b}, —NR^{a}SO₂R^{b}, —NR^{a}SO₂NR^{a}R^{b}, —NR^{a}NR^{a}R^{b}, —NR^{a}NR^{a}C(O)R^{b}, —OR^{a}, or 4-(1H-pyrazol-4-yl),
   wherein any (C₁-C₈)alkyl or (C₃-C₈)cycloalkyl group is optionally substituted by 1, 2 or 3 groups independently selected from (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a}, SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)ORₐ, —NR^{a}SO₂R^{b}, —NR^{a}SO₂NR^{a}R^{b}, —OR^{a}, —OC(O)R^{a}, and —OC(O)NR^{a}R^{b}; wherein R^{a} and R^{b} are defined as above.

### Subgroup B of Formula (VII)

X and Z are selected independently from the group consisting of (C₁-C₈)alkyl, (C₃-C₈)cycloalkyl, heterocycloalkyl, aryl, heteroaryl, —NR^{a}R^{b}, and —OR^{a};
Y is H;
R¹ is (C₁-C₈)alkyl, (C₃-C₈)cycloalkyl, or heterocycloalkyl;
R² is hydrogen, (C₁-C₃)alkyl, or halo, in which said (C₁-C₃)alkyl is optionally substituted with one to two groups selected from amino and (C₁-C₃)alkylamino;
R⁷ is hydrogen, (C₁-C₃)alkyl, or alkoxy;
R³ is hydrogen, (C₁-C₈)alkyl or halo;
R⁶ is hydrogen, halo, cyano, trifluoromethyl, amino, (C₁-C₈)alkyl, (C₃-C₈)cycloalkyl, aryl, heteroaryl, acylamino; (C₂-C₈)alkynyl, arylalkynyl, heteroarylalkynyl, —SO₂R^{a}, —SO₂NR^{a}R^{b}, or
—NR^{a}SO₂R^{b};
   wherein any (C₁-C₈)alkyl, (C₃-C₈)cycloalkyl, (C₂-C₈)alkynyl, arylalkynyl, or heteroarylalkynyl group is optionally substituted by 1, 2 or 3 groups independently selected from halo, (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀) bicycloalkyl, heterocycloalkyl, aryl, or heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂ (C₁-C₄)alkyl, —CONH₂, —CONH(C₁-C₄)alkyl,
   —CON((C₁-C₄)alkyl)((C₁-C₄)alkyl), —SO₂ (C₁-C₄)alkyl, —SO₂NH₂, —SO₂NH(C₁-C₄)alkyl, and —SO₂N((C₁-C₄)alkyl)((C₁-C₄)alkyl);
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy (C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring. Aryl and heteroaryl in this definition are selected from the group consisting of furan, thiophene, pyrrole, oxazole, thiazole, imidazole, pyrazole, oxadiazole, thiadiazole, triazole, tetrazole, benzofuran, benzothiophene, benzoxazole, benzothiazole, phenyl, pyridine, pyridazine, pyrimidine, pyrazine, triazine, tetrazine, quinoline, cinnoline, quinazoline, quinoxaline, and naphthyridine or a compound of another aryl or heteroaryl group as follows:

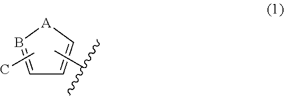

wherein in (1),
A is O, NH, or S; B is CH or N, and C is hydrogen or C₁-C₈ alkyl; or

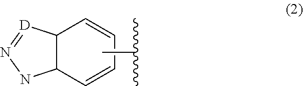

wherein in (2),
D is N or C optionally substituted by hydrogen or C₁-C₈ alkyl; or

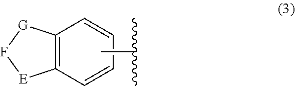

wherein in (3),
E is NH or CH₂; F is O or CO; and G is NH or CH₂; or

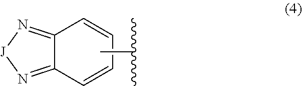

wherein in (4),
J is O, S or CO; or

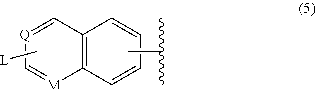

wherein in (5),
Q is CH or N;
M is CH or N; and
L/(5) is hydrogen, halo, amino, cyano, (C₁-C₈)alkyl, (C₃-C₈)cycloalkyl, —COR^{a}, —CO₂R^{a}, —CONR^{a}R^{b}, —CONR^{a}NR^{a}R^{b}, —SO₂R^{a}, —SO₂NR^{a}R^{b}, —NRₐR^{b}, —NR^{a}C(O)R^{b}, —NR^{a}SO₂R^{b}, —NR^{a}SO₂NR^{a}R^{b}, —NR^{a}NRₐR^{b}, —NR^{a}NR^{a}C(O)R^{b}, —NR^{a}NR^{a}C(O)NR^{a}R^{b}, or —OR^{a},
   wherein any (C₁-C₈)alkyl, (C₃-C₈)cycloalkyl, group is optionally substituted by 1,2 or 3 groups independently selected from (C₁-C₆)alkyl, (C₃-C₈)cycloalkyl, (C₅-C₈)cycloalkenyl, (C₁-C₆) haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, NR^{a}SO₂R^{b}, —NR^{a}SO₂NR^{a}R^{b}, —OR^{a}, —OC(O)R^{a}, and —OC(O)NR^{a}R^{b},
      wherein R^{a} and R^{b} are defined as above; or

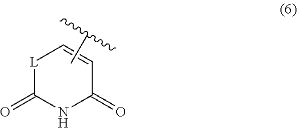

wherein in (6),
L/(6) is NH or CH₂; or

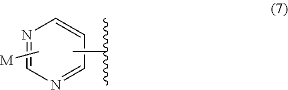

wherein in (7),
   M/(7) is hydrogen, halo, amino, cyano, (C₁-C₈)alkyl, (C₃-C₈)cycloalkyl, heterocycloalkyl, —COR^{a}, —CO₂R^{a}, —CONR^{a}R^{b}, —CONR^{a}NR^{a}R^{b}, —SO₂R^{a}, —SO₂NR^{a}R^{b}, —NR^{a}R^{b}, —NR^{a}C(O)R^{b}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, or —OR^{a},
   wherein any (C₁-C₈)alkyl, (C₃-C₈)cycloalkyl, heterocycloalkyl group is optionally substituted by 1, 2 or 3 groups independently selected from (C₁-C₆)alkyl, (C₃-C₈)cycloalkyl, (C₃-C₈)cycloalkenyl, (C₁-C₆) haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}; wherein R^{a} and R^{b} are defined as above; or

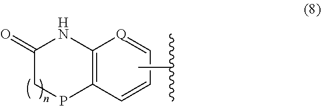

wherein in (8),
P is CH₂, NH, O, or S; Q/(8) is CH or N; and n is 0-2; or

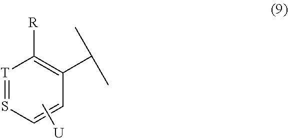

wherein in (9),
S/(9) and T/(9) is C, or S/(9) is C and T/(9) is N, or S/(9) is N and T/(9) is C;
R is hydrogen, amino, methyl, trifluoromethyl, halo;
U is hydrogen, halo, amino, cyano, nitro, trifluoromethyl, (C₁-C₈)alkyl, (C₃-C₈)cycloalkyl, —COR^{a}, —CO₂R^{a}, —CONR^{a}R^{b}, —SO₂R^{a}, —SO₂NR^{a}R^{b}, —NR^{a}R^{b}, —NR^{a}C(O)R^{b}, —NR^{a}SO₂R^{b},
—NR^{a}SO₂NR^{a}R^{b}, —NR^{a}NR^{a}R^{b}, —NR^{a}NR^{a}C(O)R^{b}, —OR^{a}, or 4-(1H-pyrazol-4-yl),
   wherein any (C₁-C₈)alkyl, or (C₃-C₈)cycloalkyl group is optionally substituted by 1, 2 or 3 groups independently selected from (C₁-C₆)alkyl, (C₃-C₈)cycloalkyl, (C₅-C₅)cycloalkenyl, (C₁-C₆) haloalkyl, cyano, —COR^{a}, —CO₂R^{a}, —CONR^{a}R^{b}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b}, wherein R^{a} and R^{b} are defined as above.

In some embodiments, the EZH2 inhibitor is:

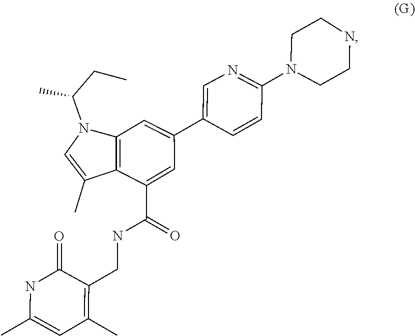

stereoisomers thereof or pharmaceutically acceptable salt or solvate thereof.

In some embodiments, the EZH2 inhibitor is

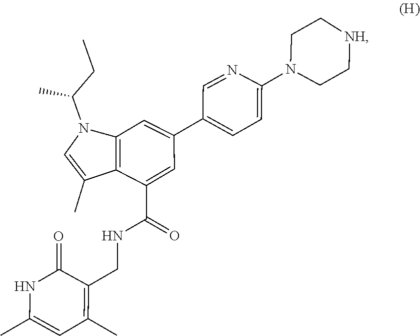 [SMILES: CC[C@@H](C)n1cc(C)c2c(C(=O)NCc3c(C)cc(C)[nH]c3=O)cc(-c3ccc(N4CCNCC4)nc3)cc21]

stereoisomers thereof or pharmaceutically acceptable salt or solvate thereof.

The compounds described herein can be synthesized according to any method known in the art. For example, the compounds having the Formula (VII) can be synthesized according to the method described in WO 2011/140325; WO 2011/140324; and WO 2012/005805, each of which is incorporated by reference in its entirety.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon mono- or multi-ring (e.g., fused, bridged, or spiro rings) system having 3 to 30 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, and adamantyl. The term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

The term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl(benzyl)). An “alkylaryl” moiety is an aryl substituted with an alkyl (e.g., methylphenyl).

As used herein, “alkyl linker” is intended to include C₁, C₂, C₃, C₄, C₅ or C₆ straight chain (linear) saturated divalent aliphatic hydrocarbon groups and C₃, C₄, C₅ or C₆ branched saturated aliphatic hydrocarbon groups. For example, C₁-C₆ alkyl linker is intended to include C₁, C₂, C₃, C₄, C₅ and C₆ alkyl linker groups. Examples of alkyl linker include, moieties having from one to six carbon atoms, such as, but not limited to, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂—), n-pentyl (—CH₂CH₂CH₂CH₂CH₂—), s-pentyl (—CHCH₃CH₂CH₂CH₂—) or n-hexyl (—CH₂CH₂CH₂CH₂CH₂CH₂—).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

As used herein, “carbocycle” or “carbocyclic ring” is intended to include any stable monocyclic, bicyclic or tricyclic ring having the specified number of carbons, any of which may be saturated, unsaturated, or aromatic. Carbocycle includes cycloalkyl and aryl. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” or “heterocyclic group” includes any ring structure (saturated, unsaturated, or aromatic) which contains at least one ring heteroatom (e.g., N, O or S). Heterocycle includes heterocycloalkyl and heteroaryl. Examples of heterocycles include, but are not limited to, morpholine, pyrrolidine, tetrahydrothiophene, piperidine, piperazine, oxetane, pyran, tetrahydropyran, azetidine, and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1/-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5 (4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R₁) occurs more than one time in any constituent or formula for a compound, its definition at each occurrence is independent of its definition at every other occurrence. Thus, for example, if a group is shown to be substituted with 0-2 R₁ moieties, then the group may optionally be substituted with up to two R₁ moieties and R₁ at each occurrence is selected independently from the definition of R₁. Also, combinations of substituents and/or variables are permissible, but only if such combinations result in stable compounds.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —COOH or its C₁-C₆ alkyl ester.

“Acyl” includes moieties that contain the acyl radical (R—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen bonded to two carbon atoms or heteroatoms. For example, the term includes “alkoxyalkyl,” which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls, and alkthioalkynyls. The term “alkthioalkyls” include moieties with an alkyl, alkenyl, or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” refers to unsubstituted or substituted —NH₂. “Alkylamino” includes groups of compounds wherein nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

In the present specification, the structural formula of the compound represents a certain isomer for convenience in some cases, but the present invention includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present invention. Furthermore, so-called metabolite which is produced by degradation of the present compound in vivo is included in the scope of the present invention.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present invention may be depicted as different chiral isomers or geometric isomers. It should also be understood that when compounds have chiral isomeric or geometric isomeric forms, all isomeric forms are intended to be included in the scope of the present invention, and the naming of the compounds does not exclude any isomeric forms, it being understood that not all isomers may have the same level of activity.

Furthermore, the structures and other compounds discussed in this invention include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between pyridin-2 (1H)-ones and the corresponding pyridin-2-ols, as shown below.

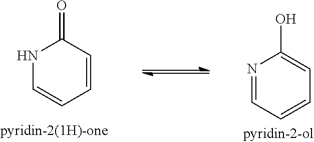 [SMILES: O=c1cccc[nH]1 ; Oc1ccccn1]

It is to be understood that the compounds of the present invention may be depicted as different tautomers. It should also be understood that when compounds have tautomeric forms, all tautomeric forms are intended to be included in the scope of the present invention, and the naming of the compounds does not exclude any tautomer form. It will be understood that certain tautomers may have a higher level of activity than others.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of any of Formulae disclosed herein include the compounds themselves, as well as their salts or their solvates, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms.

Additionally, the compounds of the present invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are aryl- or heteroaryl-substituted benzene compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present invention is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The present invention provides methods for the synthesis of the compounds of any Formula disclosed herein. The present invention also provides detailed methods for the synthesis of various disclosed compounds of the present invention according to the following schemes as shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, polymorph or solvate thereof.

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present invention.

Compounds of the present invention can be conveniently prepared by a variety of methods familiar to those skilled in the art. The compounds of this invention with any Formula disclosed herein may be prepared according to the procedures illustrated in Schemes 1-10 below, from commercially available starting materials or starting materials which can be prepared using literature procedures. The Z and R groups (such as R₂, R₃, R₄, R₆, R₇, R₈, and R₁₂) in Schemes 1-10 are as defined in any of Formulae disclosed herein, unless otherwise specified.

One of ordinary skill in the art will note that, during the reaction sequences and synthetic schemes described herein, the order of certain steps may be changed, such as the introduction and removal of protecting groups.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3rd edition, John Wiley & Sons: New York, 1999.

Preferred protecting groups include, but are not limited to:
For a hydroxyl moiety: TBS, benzyl, THP, Ac
For carboxylic acids: benzyl ester, methyl ester, ethyl ester, allyl ester
For amines: Cbz, BOC, DMB
For diols: Ac (×2) TBS (×2), or when taken together acetonides
For thiols: Ac
For benzimidazoles: SEM, benzyl, PMB, DMB
For aldehydes: di-alkyl acetals such as dimethoxy acetal or diethyl acetyl.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

The following abbreviations are used throughout the specification and are defined below:
Ac acetyl
AcOH acetic acid
aq. aqueous
BID or b.i.d. bis in die (twice a day)
BOC tert-butoxy carbonyl
Cbz benzyloxy carbonyl
CDCl₃ deuterated chloroform
CH₂C₁₂ dichloromethane
DCM dichloromethane
DMB 2,4 dimethoxy benzyl
DMF N,N-Dimethylformamide
DMSO Dimethyl sulfoxide
EA or EtOAc Ethyl acetate
EDC or EDCI N-(3-Dimethylaminopropyl)-N′-ethylcarbodiimide
ESI− Electrospray negative mode
ESI+ Electrospray positive mode
EtOH ethanol
h hours
H₂O water
HOBt 1-Hydroxybenzotriazole
HCl hydrogen chloride or hydrochloric acid
HPLC High performance liquid chromatography
K₂CO₃ potassium carbonate
LC/MS or LC-MS Liquid chromatography mass spectrum
M Molar
MeCN Acetonitrile
min minutes
Na₂CO₃ sodium carbonate
Na₂SO₄ sodium sulfate
NaHCO₃ sodium bicarbonate
NaHMDs Sodium hexamethyldisilazide
NaOH sodium hydroxide
NaHCO₃ sodium bicarbonate
Na₂SO₄ sodium sulfate
NMR Nuclear Magnetic Resonance
Pd(OH)₂ Palladium dihydroxide
PMB para methoxybenzyl
p.o. per os (oral administration)
ppm parts per million
prep HPLC preparative High Performance Liquid Chromatography
PYBOP (Benzotriazol-1-yloxy)tripyrrolidinophosphonium hexafluorophosphate
Rt or RT Room temperature
TBME tert-Butyl methyl ether
TFA trifluoroacetic acid
THF tetrahydrofuran
THP tetrahydropyran

The present invention also provides pharmaceutical compositions comprising a compound of any Formula disclosed herein in combination with at least one pharmaceutically acceptable excipient or carrier.

A “pharmaceutical composition” is a formulation containing the compounds of the present invention in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present invention are capable of further forming salts. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds, or pharmaceutically acceptable salts or solvates thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

In the synthetic schemes described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

An EZH2 inhibitor of the present invention may, if desired, be presented in a kit (e.g., a pack or dispenser device) which may contain one or more unit dosage forms containing the EZH2 inhibitor. The pack may for example comprise metal or plastic foil, such as a blister pack. The pack or dispenser device may be accompanied by instructions for administration. Compositions comprising an EZH2 inhibitor of the invention formulated in a compatible pharmaceutical carrier may also be prepared, placed in an appropriate container, and labeled for treatment of an indicated condition. Instructions for use may also be provided.

Also provided herein are kits comprising a plurality of methylation detection reagents that detect the methylated H3-K27. For example, the kit includes mono-methylated H3-K27, di-methylated H3-K27 and tri-methylated H3-K27 detection reagents. The detection reagent is for example antibodies or fragments thereof, polypeptide or aptamers.

A kit may also include reagents for detecting loss of function of at least one component of the SWI/SNF complex, e.g., nucleic acids that specifically identify a mutant component nucleic acid sequence by having homologous nucleic acid sequences, such as oligonucleotide sequences, complementary to a portion of the mutant component nucleic acid sequence or antibodies to proteins encoded by the wild type and/or mutant component nucleic acids packaged together in the form of a kit. The oligonucleotides can be fragments of the component gene. For example the oligonucleotides can be 200, 150, 100, 50, 25, 10 or less nucleotides in length. The kit may contain in separate containers an aptamer or an antibody, control formulations (positive and/or negative), and/or a detectable label such as fluorescein, green fluorescent protein, rhodamine, cyanine dyes, Alexa dyes, luciferase, radiolabels, among others. In addition, reagents for detecting the biological activity of the SWI/SNF complex (such as its chromatin remodeling activity) may be included in the kit.

Instructions (e.g., written, tape, VCR, CD-ROM, etc.) for carrying out the assay may be included in the kit. The assay may for example be in the form of a Western Blot analysis, Immunohistochemistry (IHC), immunofluorescence (IF), sequencing and Mass spectrometry (MS) as known in the art.

### Example 1: Durable Tumor Regression in Genetically Altered Lymphomas and Malignant Rhabdoid Tumors by Inhibition of EZH2

Compound A is a potent and selective inhibitor of EZH2: Cell free biochemical assays that included radiolabeled SAM and either chicken erythrocyte oligonucleosomes or peptides corresponding to H3K27 as substrates showed that Compound A selectively inhibited the activity of human PRC2 containing wild-type EZH2 with an inhibition constant (Ki) value of 2.5±0.5 nmol/L and IC₅₀ values of 11±5 nM (nucleosome assay) or 16±12 nM (peptide assay). The IC₅₀ values were similar for human and rat EZH2 enzymes as well as for EZH2 proteins bearing all known lymphoma change-of-function mutations. The IC₅₀ value of Compound A increased with increasing concentration of SAM, but was minimally affected by increasing the amount of oligonucleosome which is consistent with a SAM-competitive and nucleosome-noncompetitive modality of inhibition. In order to demonstrate HMT selectivity, inhibition by Compound A against a panel of HMTs other than EZH2 encompassing both lysine and arginine HMTs was assessed. Compound A displayed a 35-fold selectivity versus EZH1 and greater than 4500-fold selectivity relative to the 14 other HMTs tested.

Compound A specifically inhibits cellular H3K27 methylation in cells: When WSU-DLCL2 EZH2 Y641F mutant lymphoma cells were incubated with Compound A for 4 days, a concentration-dependent reduction in global H3K27Me3 levels was observed with an average IC₅₀ value of 0.26 μM (H3K27Me3 levels determined by ELISA). When studying the kinetics of methylation inhibition, the half-life of H3K27Me3 was approximately 1 day as 90% inhibition was only achieved after 3 to 4 days of incubation. When OCI-LY19 EZH2 wild-type lymphoma cells were incubated with 2.7 μM Compound A for 4 days, the only methyl marks affected were the H3K27Me1, H3K27Me2 and H3K27Me3, the three known products of PRC2 catalysis. Incubation with Compound A also resulted in an increase in H3K27 acetylation. The ability of Compound A to reduce global H3K27 trimethylation levels was further tested in several other human lymphoma cell lines including lines expressing either wild-type or mutant EZH2. Compound A reduced H3K27Me3 with similar potency in all cell lines independent of the EZH2 status (Table 1).

Compound A leads to selective killing of lymphoma cell lines bearing EZH2 point mutations: Incubation of WSU-DLCL2 EZH2 Y641F mutant cells with Compound A lead to anti-proliferative effects with an average IC₅₀ value of 0.28±0.14 μM in a 6 day proliferation assay. The kinetics of the effect of Compound A on viable cell number was further tested over an extended period of 11 days. The antiproliferative effect of Compound A was apparent after WSU-DLCL2 cells had been exposed to compound for longer than 4 days, consistent with the kinetics of Compound A-mediated cellular H3K27 methylation inhibition. The IC₅₀ value for Compound A inhibition of proliferation of WSU-DLCL2 cells in the 11-day assay (0.0086 μM, Table 1) was lower when compared with results obtained with a 6-day proliferation assay, suggesting increased sensitivity with longer incubation periods. In contrast to the WSU-DLCL2 cells, the growth of OCI-LY19 human lymphoma cells (EZH2 wild type for residue Y641) over 11 days was not significantly affected, despite comparable IC₅₀ values for H3K27Me3 inhibition for both cell lines (Table 1). In order to identify a concentration at which cells stop proliferating considering the entire incubation period of 11 days, the lowest cytotoxic concentration (LCC) for a particular cell line was calculated. The LCC value for WSU-DLCL2 EZH2 Y641F mutant human lymphoma cells was significantly lower when compared with OCI-LY19 cells that are wild type for EZH2 (Table 1). This context specific cell killing was further supported by results from 11-day proliferation assays with an extended lymphoma cell line panel. All cell lines harboring an EZH2 mutation, with the exception of the RL cell line (EZH2 Y641N), were more sensitive to the antiproliferative effects of Compound A when compared with cell lines with wild-type EZH2 (Table 1). The Pfeiffer cell line (EZH2 A677G) showed a 20 to 300 fold increase in sensitivity to Compound A, as measured by IC₅₀ value and LCC, respectively, over the Y641 mutant cell lines. Next the minimum time of compound exposure necessary for sustained cell killing was investigated by washout experiments. The LCC values on day 11 or 14 for WSU-DLCL2cells that were either incubated with Compound A for 7 days (followed by 7 days of compound washout) or continuously for 14 days were similar (Table 2). Drug exposure for only 4 days, however, was not sufficient to induce LCC values similar to continuous incubation.

Compound A induces G₁ arrest and apoptosis in EZH2 mutant lymphoma cells: Next, the effects of incubation with Compound A (1 μM) for 7 days on cell cycle progression and apoptosis in WSU-DLCL2 cells were assessed. An increase in the percentage of cells in G₁ phase, and a decrease in the percentage of cells in S phase and G2/M phase was apparent after 2 days of Compound A incubation. The maximum effect was achieved after 4 days. There was no apparent increase in the sub-G1 fraction suggesting that apoptosis was not induced by Compound A incubation for 7 days. This is in agreement with the growth curves of WSU-DLCL2 cells in the presence of Compound A indicating that cytotoxic effects were observed only after 7 days of incubation. Following incubation of WSU-DLCL2 cells with Compound A for up to 14 days, the fraction of apoptotic cells determined by TUNEL assay was significantly increased on day 14 compared to vehicle, indicating that Compound A-mediated cell death occurred through the induction of apoptosis.

Oral administration of Compound A leads to EZH2 target inhibition in EZH2 mutant xenograft models in mice: The effect of oral dosing of Compound A on systemic compound exposure and in vivo target inhibition in mice bearing EZH2 mutant lymphoma xenografts was investigated. First, SCID mice implanted subcutaneously with WSU-DLCL2 xenografts were orally dosed with Compound A for 4 or 7 days. Measuring Compound A plasma levels either 5 minutes before or 3 hours after the last dose revealed a clear dose dependent increase in exposure. Only animals dosed at 160 mg/kg TID or 213 mg/kg BID maintained mean compound levels in plasma above the LCC for WSU-DLCL2 cells throughout a dosing cycle (1652 ng/mL, with mouse plasma protein binding considered). Compound determination in homogenates from tumors collected 3 hours after the last dose revealed that only for the highest dose groups compound levels in the 2 compartments were similar. When H3K27Me3 levels in tumors were analyzed, dose dependent EZH2 target inhibition was observed. H3K27Me3 inhibition was less in tumors from mice dosed at 213 mg/kg QD, suggesting that maintaining a plasma concentration above LCC throughout a dosing cycle is required for optimal target inhibition. Dosing for 4 days at 160 mg/kg TID resulted in slightly lower target inhibition than dosing for 7 days at the same dose and schedule, indicating that prolonged dosing increased the degree of target inhibition in WSU-DLCL2 tumors. A similar 7-day study in nude mice implanted subcutaneously with KARAPS-422 xenografts assessing both BID and QD schedules was performed. Compound A induced a dose-dependent reduction of tumor H3K27Me3 levels at both regimens.

Compound A induces significant antitumor effects in several EZH2 mutant lymphoma xenografts: When WSU-DLCL2 EZH2 Y641F mutant xenograft tumor bearing SCID mice were treated with Compound A for 28 days, dose-dependent tumor growth inhibition, 58% at the highest dose of 150 mg/kg TID, was observed. Only animals administered the highest dose maintained mean Compound A plasma levels above LCC for WSU-DLCL2 cells throughout the dosing cycle. Dosing of Compound A for 28 days led to a relative compound accumulation in tumor tissue compared with plasma, in contrast to what was observed with 7-day dosing. ELISA analysis of histones from tumors collected on day 28 indicated dose-dependent target inhibition. H3K27Me3 levels in WSU-DLCL2 xenografts were lower in mice dosed for 28 days compared with 7 days indicating that prolonged administration of Compound A increased the degree of target inhibition. In KARPAS-422 EZH2 Y461N mutant xenografts, 28-day dosing of Compound A on a BID schedule had much more dramatic effects. Tumor growth inhibition was observed at doses as low as 80.5 mg/kg BID, but higher doses eradicated the xenografts, and no re-growth was observed for up to 90 days after cessation of dosing. When intermittent dosing schedules were investigated in KARPAS-422 xenograft bearing mice, Compound A again showed significant dose-dependent antitumor effects with two cycles of 7-day on/7-day off and 21 day on/7 day off schedules. For all dosing schedules, tumor growth inhibition and complete regressions were observed at 90 and 361 mg/kg BID, respectively. The Pfeiffer EZH2 A677G mutant xenograft model was the most sensitive tumor model, as suggested by the potent anti-proliferative effects of Compound A on this cell line in vitro. All Compound A dose groups (QD schedule) except the lowest one (30 mg/kg QD) showed complete tumor regressions in all animals. Again, tumor re-growth was not observed until the end of the study (36 days after stopping Compound A administration). Although tumor re-growth was observed at 30 mg/kg QD, this very low dose induced tumor stasis during the administration period. Due to tolerability issues dosing was stopped on day 12 for mice administered 1140 mg/kg QD; still, durable complete regressions were observed in this group that were only exposed to Compound A for 12 days.

Compound A selectively kills SMARCB1 mutant MRT cells in vitro and in vivo: Whether EZH2 inhibition had any effects on the growth and survival of SMARCB1-deleted MRT cells was tested. Incubating SMARCB1-deleted MRT cell lines G401 and A204 with Compound A in a 14-day proliferation assay in vitro induced strong anti-proliferative effects with IC₅₀ values in the nM range while the control cell lines RD and SJCRH30 which expressed SMARCB1 were minimally affected (Table 3). Dosing of SCID mice bearing subcutaneous G401 xenografts with Compound A at 266 or 532 mg/kg BID for 28 days eliminated those extremely fast growing tumors. Similar to the KARPAS-422 and Pfeiffer EZH2 mutant NHL xenograft models re-growth was not observed at study end, 32 days after dosing stop. Compound A dosed at 133 mg/kg induced stasis during the administration period, and produced a significant tumor growth delay compared to vehicle after dosing stop. Tumors that were harvested from subsets of mice from each group on day 21 showed strong EZH2 target inhibition at all doses.

Compound A inhibits H3K27 methylation in nontumor tissues in a dose dependent manner: The data described above demonstrate that Compound A represents a new treatment modality for SWI/SNF driven cancers and MRTs. Measuring pharmacodynamic biomarker modulation post-dose is often performed in early clinical trials to assess the degree of target inhibition that is predicted to produce a response based on data from preclinical models. Since the collection of post-dose tumor biopsies is often not possible, easier accessible surrogate tissues such as peripheral blood mononuclear cells (PBMCs), skin or bone marrow are often collected instead. To test EZH2 target inhibition in surrogate tissues male and female Sprague Dawley rats were orally administered 100, 300, or 1000 mg/kg Compound A for 28 days, and PBMCs, bone marrow and skin samples were collected at study end. Plasma levels of Compound A increased dose-dependently in both male and female rats, and the plasma levels were generally higher in females compared with those in males. Due to tolerability issues, females in the 1000 mg/kg group had to be euthanized on day 23. Dose-dependent target inhibition was observed in PBMCs and bone marrow from rats dosed with Compound A, as measured by ELISA. The degree of target inhibition was less pronounced for PBMCs from females that were dosed for 22 days compared with males that were dosed for 28 days (same dose of 1000 mg/kg). A dose dependent reduction in H3K27Me3 positive cells was observed in the epidermis of skin of Compound A-dosed rats, as assessed by an IHC assay. The maximum effect was observed at the highest dose, and was already evident after 22 days of Compound A administration.

Compound A displayed similar properties as other EZH2 inhibitors in vitro, such as very high specificity for EZH2 in biochemical assays when compared with other HMTs and specific inhibition of cellular H3K27 methylation leading to context specific killing of EZH2 mutated NHL cell lines. However, this compound achieved an approximately 10-fold increase in potency, reflected by decreased Ki and IC₅₀ values determined in biochemical and cell-functional assays. In addition, Compound A showed excellent oral bioavailability when administered to rodents which lead to dose dependent EZH2 target inhibition in xenograft tumor and nontumor tissues. Importantly, dosing of Compound A induced significant antitumor effects in mice bearing EZH2 mutant lymphoma xenografts. The responses ranged from tumor eradication (no regrowth after dosing cessation) to dose-dependent tumor growth inhibition. The delayed onset of antitumor activity (after 4 to 7 days) was consistent with the kinetics of methylation inhibition and antiproliferative activity induced by incubation of cells with Compound A in vitro. Keeping Compound A plasma levels above LCC throughout a dosing cycle was necessary for the WSU-DLCL2 xenograft model to induce maximal target inhibition and antitumor response. The other two lymphoma xenograft models (KARPAS-422 and Pfeiffer), however, were extremely sensitive to Compound A administration, and keeping plasma levels above LCC was not necessary. Pfeiffer EZH2 A677G mutant xenograft tumors disappeared permanently with very low doses or short dosing periods, suggesting that patients with this type of genetically defined NHL would have a significant treatment effect with Compound A.

MRTs are extremely aggressive pediatric cancers of the brain, kidney, and soft tissues that are highly malignant, locally invasive, frequently metastatic, and particularly lethal, but they are typically diploid and lack genomic aberrations. They are, however, characterized by an almost complete penetrance of loss of expression of the SMARCB1, a core component of the SWI/SNF chromatin remodeling complex. The biallelic inactivation of SMARCB1, for instance induced by mutations, is in essence the sole genetic event in MRTs which suggests a driver role for this genetic aberration. Through genetic studies it has been suggested that PRC2 and SWI/SNF antagonistically regulate gene expression around the RB, Cyclin D1 and MYC pathways. Here, it has been demonstrated pharmacological EZH2 inhibition induced antiproliferative effects in SMARCB1 deleted MRT cell lines and permanently eradicated MRT xenografts in mice. This confirms the dependency of such cancers, in which EZH2 itself is not genetically altered, on PRC2 activity.

Compound A represents a new treatment modality for genetically defined subsets of NHL and for MRTs. The ability to measure dose-dependent changes in H3K27Me3 levels in skin, PBMCs and bone marrow portends the use of signal from these surrogate tissues as a non-invasive pharmacodynamics biomarker in human clinical trials.

TABLE 1
IC₅₀ Values for Methylation and Proliferation as well as
LCC Values for Compound A in Human Lymphoma Cell Lines

| | | Methylation | Proliferation | |
|---|---|---|---|---|
| | | IC₅₀ | IC₅₀ | LCC |
| Cell Line | EZH2 Status | (nmol/L)^{a} | (μmol/L)^{b} | (μmol/L)^{b} |
| DOHH-2 | Wild Type | ND | 1.7 | >10 |
| Farage | Wild Type | ND | 0.099 | >10 |
| OCI-LY19 | Wild Type | 8 | 6.2 | 10-25 |
| Toledo | Wild Type | ND | 7.6 | >10 |
| Karpas-422 | Y641N | 90 | 0.0018 | 0.12 |
| Pfeiffer | A677G | 2 | 0.00049 | 0.0005 |
| RL | Y641N | 22 | 5.8 | >25 |
| SU-DHL-10 | Y641F | ND | 0.0058 | 0.14 |
| SU-DHL-6 | Y641N | 20 | 0.0047 | 0.21 |
| WSU-DLCL2 | Y641F | 9 | 0.0086 | 0.17 |

^{a}Derived after incubation for 4 days by immunoblot. Values represent the result from one experiment.
^{b}Derived after incubation for 11 days. Compound incubations for each experiment were performed in triplicate, and values represent one experiment for all cell lines except OCI-LY19, Pfeiffer, and WSU-DLCL2. For the remaining three cell lines, values represent the mean from the following number of experiments: OCI-LY19 n = 9; Pfeiffer n = 2 and WSU-DLCL2 n = 15.

TABLE 2
LCC Values for Compound A for WSU-DLCL2 Human Lymphoma Cells
Dosed Either Continuously or After Compound Washout

| | Day 11 | Day 14 |
|---|---|---|
| | LCC | LCC |
| WSU-DLCL2 Washout | (μM) | (μM) |
| No Washout | 0.17 | 0.11 |
| 4-day Compound A; | 0.36 | 0.42 |
| 11-day Washout | | |
| 7-day Compound A; | 0.19 | 0.075 |
| 7-day Washout | | |

Values represent the mean of duplicate experiments with three replicates per incubation concentration within the experiments.

TABLE 3
IC₅₀ Values for Compound A for SMARCB1 Negative MRT
Cell Lines and SMARCB1 Positive Control Cell Lines

| | | Proliferation | Proliferation |
|---|---|---|---|
| | SMARCB1 | IC₅₀ (μM), | IC₅₀ (μM), |
| Cell Line | Status | day 7 | day 14 |
| RD | Wild Type | 9.2 | 5.2 |
| SJCRH30 | Wild Type | 6.1 | 8.8 |
| G401 | Mutant | 0.087 | 0.042 |
| A204 | Mutant | 3.2 | 0.14 |

Values represent the mean of duplicate experiments with three replicates per incubation concentration within the experiments.

### Example 2: Durable Tumor Regression in Genetically Altered Malignant Rhabdoid Tumors by Inhibition of EZH2

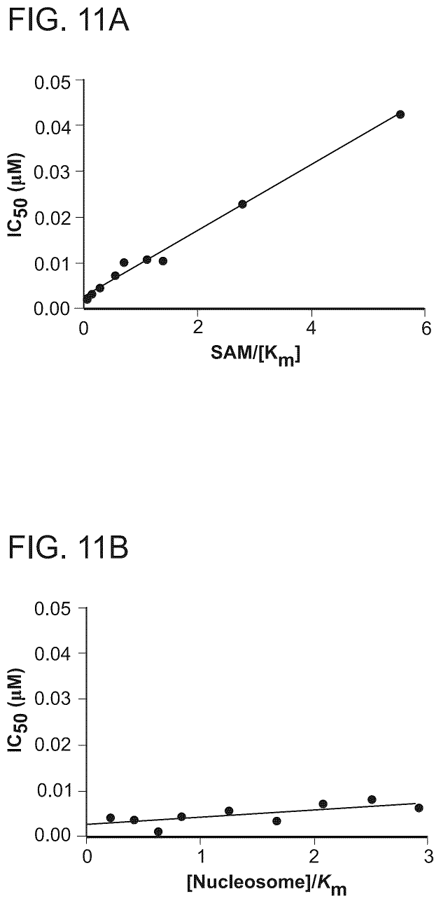

Compound A is a potent and selective inhibitor of EZH2: Compound A was developed through iterative medicinal chemistry (FIG. 10A). Compound A inhibited the activity of human PRC2 containing wild-type EZH2 with an inhibition constant (Ki) value of 2.5±0.5 nM, and similar potency was observed for EZH2 proteins bearing all known lymphoma change-of-function mutations (Table 5). The compound was found to be SAM-competitive and nucleosome-noncompetitive by steady state kinetic studies (FIG. 11). Inhibition by Compound A against a panel of HMTs other than EZH2 encompassing both lysine and arginine HMTs was also assessed. Compound A displayed a 35-fold selectivity versus EZH1 and >4500-fold selectivity relative to 14 other HMTs tested (Table 5).

TABLE 4
Histone Methyltransferase Inhibition by Compound A

| | IC₅₀ | % Inhibition at |
|---|---|---|
| Enzyme Assay | (nM) | 1 μM Compound A^{a} |
| CARM1 | >50,000^{b} | 5 ± 3 |
| DOT1L | >50,000^{c} | 2 ± 8 |
| EHMT1 | >50,000^{c} | 6 ± 6 |
| EHMT2 | >50,000^{c} | 7 ± 3 |
| EZH1^{d,e} | 392 ± 72^{f} | 98 ± 1 |
| EZH2 Peptide | 11 ± 5^{f} | ND |
| Assay^{d,e} | | |
| EZH2 Nucleosome | 16 ± 12^{f} | 100 ± 1 |
| Assay^{d} | | |
| A677G EZH2^{d,e} | 2^{b} | ND |
| A687V EZH2^{d,e} | 2^{b} | ND |
| Y641F EZH2^{d,e} | 14 ± 5^{f} | ND |
| Y641C EZH2^{d,e} | 16^{c} | ND |
| Y641H EZH2^{d,e} | 6^{c} | ND |
| Y641N EZH2^{d,e} | 38^{b} | ND |
| Y641S EZH2^{d,e} | 6^{c} | ND |
| rat EZH2^{d,e} | 4^{c} | ND |
| PRMT1 | >50,000^{c} | 5 ± 4 |
| PRMT3 | ND | 2 ± 2 |
| PRMT5/MEP50 | >50,000^{c} | 2 ± 6 |
| PRMT6 | ND | 3 ± 3 |
| PRMT8 | >50,000^{c} | 7 ± 3 |
| SETD7 | ND | 4 ± 3 |
| SMYD2 | >50,000^{c} | 1 ± 2 |
| SMYD3 | ND | 0 ± 5 |
| WHSC1 | >100,000^{c} | 8 ± 3 |
| WHSC1L1 | >100,000^{c} | 9 ± 8 |

^{a}Values represent the mean and standard deviation of duplicate experiments determined at 10 μmol/L Compound A.
^{b}Values represent the mean of duplicate experiments with two replicates per experiment.
^{c}Values represent one experiment with two replicates per experiment.
^{d}All EZH1 and EZH2 proteins were assayed in the context of 4 PRC2 components (EZH1/2, SUZ12, RBAP48, EED).
^{e}Assayed with H3K27 peptides as substrates.

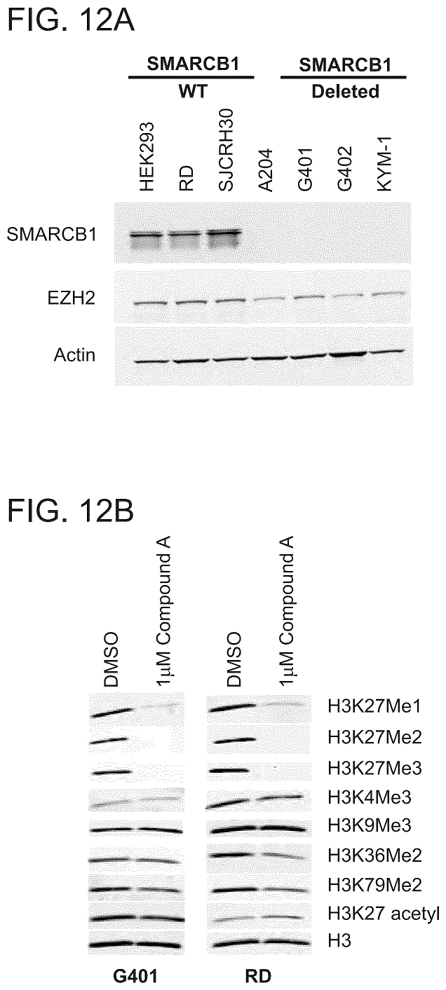

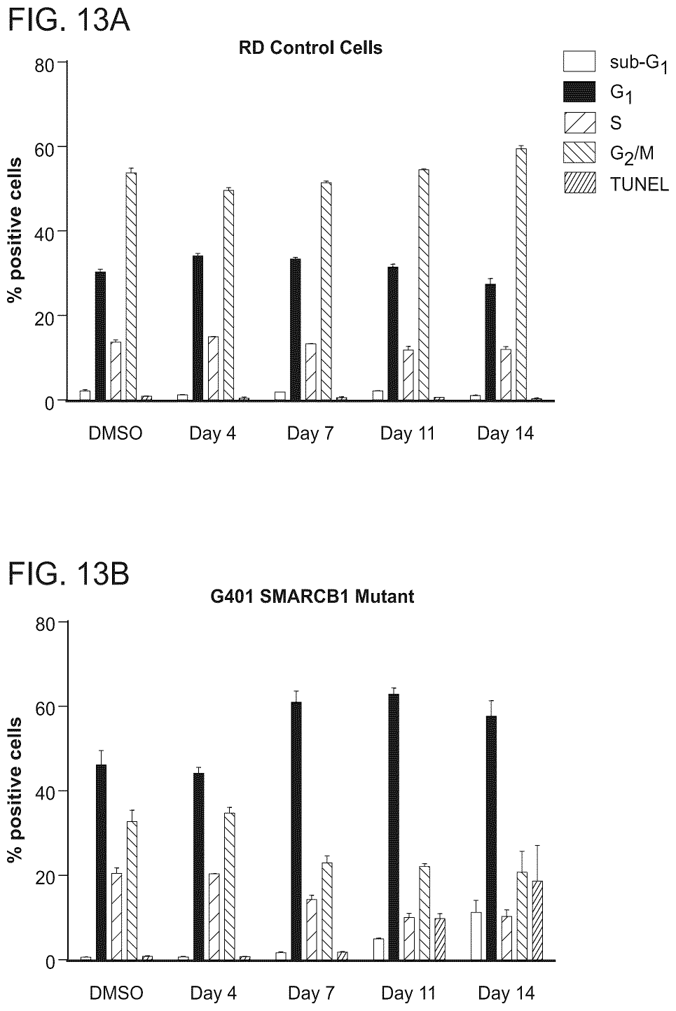

Compound A specifically inhibits cellular H3K27 methylation leading to selective apoptotic killing of SMARCB1 mutant MRT cells: A panel of SMARCB1 deficient MRT cells and SMARCB1 wild-type control cells (confirmed by immunoblot, FIG. 12A) were treated with Compound A for 4 days, resulting in concentration-dependent reductions in global H3K27Me3 levels (FIG. 10B and table 6). Treatment of either wild-type or mutant cells resulted in diminution only of methyl marks on H3K27, with no other histone methyl marks being affected (FIG. 12B). In vitro treatment of SMARCB1-deleted MRT cell lines with Compound A induced strong anti-proliferative effects with IC₅₀ values in the nM range; while the control (wild-type) cell lines were minimally affected (FIG. 10C and table 6). Antiproliferative effects were apparent in SMARCB1-deleted MRT cells after 7 days of compound exposure, but required 14 days of exposure for maximal activity. The effects of incubation with Compound A (1 μM) for 14 days on cell cycle progression and apoptosis in G401 and RD cells were also assessed. Compound A incubation of RD SMARCB1 wild-type cells showed no changes in cell cycle or apoptosis compared to the DMSO control (FIG. 13A). In contrast, G401 SMARCB1-deleted cells showed an increase in the percentage of cells in G1 phase, and a concomitant decrease in S phase and G2/M phase after 7 days (FIG. 13B). There was no apparent increase in the sub-G₁ fraction through day 7, suggesting that apoptosis was not yet induced by that time. This coincides with the growth curves of G401 cells in the presence of Compound A that display cytotoxicity only after 7 days of incubation (FIG. 10C). Following Compound A treatment of G401 cells for up to 14 days, the fraction of cells in sub-G₁ as well as apoptotic cells determined by TUNEL assay increased in a time dependent manner through days 11 and 14, indicating that Compound A-mediated cell death occurred through the induction of apoptosis (FIG. 13B).

TABLE 6

| | | | Proliferation |
|---|---|---|---|
| | SMARCB1 | Methylation | IC₅₀ on Day |
| Cell Line | Status | IC₅₀ (nM)^{a} | 14 (nM)^{b} |
| G401 | mutant | 2.7 | 135 |
| A204 | mutant | 1.4 | 590 |
| G402 | mutant | 1.7 | 144 |
| KYM-1 | mutant | 4.3 | 32 |
| RD | wild-type | 5.6 | 6100, >10000^{c} |
| 293 | wild-type | 2.4 | >10000 |
| SJCRH30 | wild-type | 4.9 | 5100, >10000^{c} |

^{a}Derived after incubation for 4 days, extraction of histones, immunoblot and densitometry. Values represent the mean from two experiments.
^{b}Compound incubations for each experiment were performed in triplicate, and values represent the mean of 2 experiments for all cell lines.
^{c}Mean calculation of duplicate experiment not possible.

Compound A induces genes of neuronal differentiation and cell cycle inhibition while suppressing expression of hedgehog pathway genes, MYC and EZH2: It has been suggested that SMARCB1 loss drives cancer formation through simultaneous epigenetic perturbation of key cancer pathways. The present data confirmed the previously described reduced expression of genes important for neuronal differentiation (CD133, DOCK4, PTPRK), cell cycle inhibition (CDKN2A) and tumor suppression (BIN1), as well as increased expression of the hedgehog pathway gene GLI1 in SMARCB1-deleted G401 cells compared to control cells (FIG. 14A). Compound A treatment of G401 cells for up to 7 days strongly induced expression of CD133, DOCK4 and PTPRK and up-regulated cell cycle inhibitors CDKN1A and CDKN2A and tumor suppressor BIN1, all in a time-dependent manner (FIG. 14B). Simultaneously, the expression of hedgehog pathway genes, MYC and EZH2 were reduced. Notably, G402 SMARCB1-deleted cells exposed to Compound A for 14 days assumed a neuron-like morphology (FIG. 14C). In contrast, Compound A incubation of RD control cells had minimal effect on expression of the above-mentioned genes.

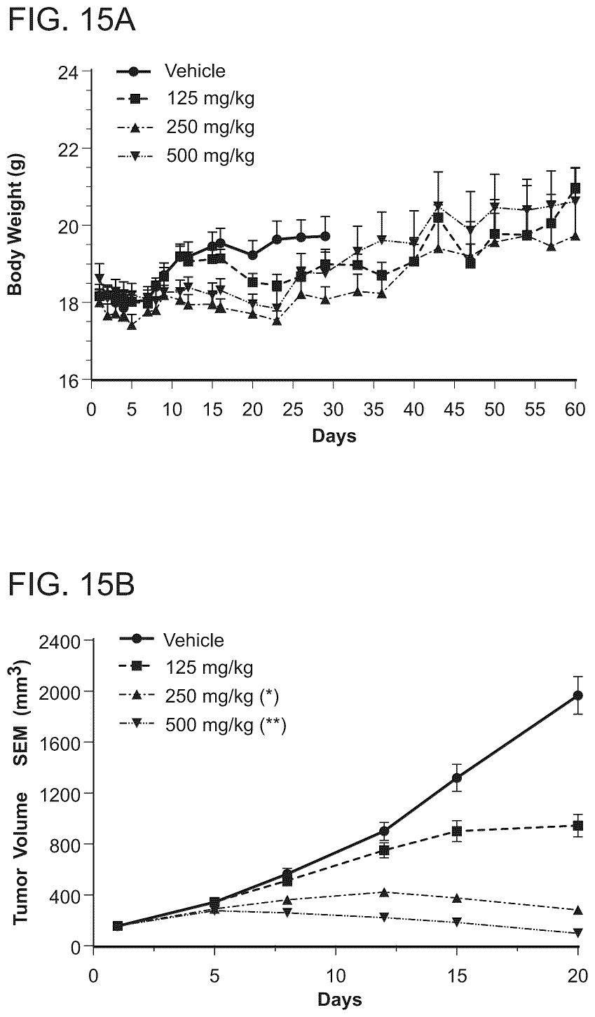

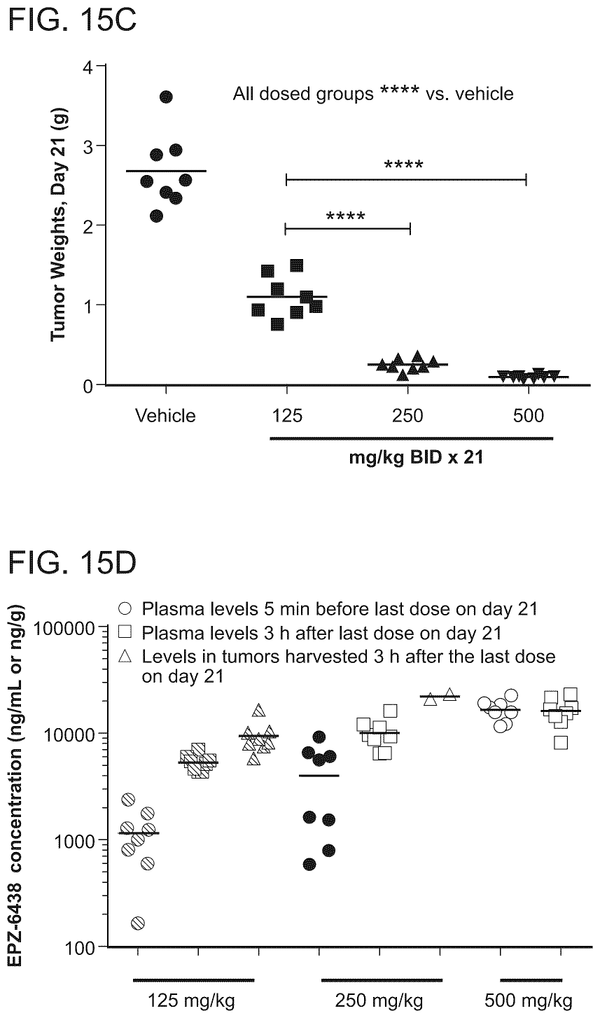

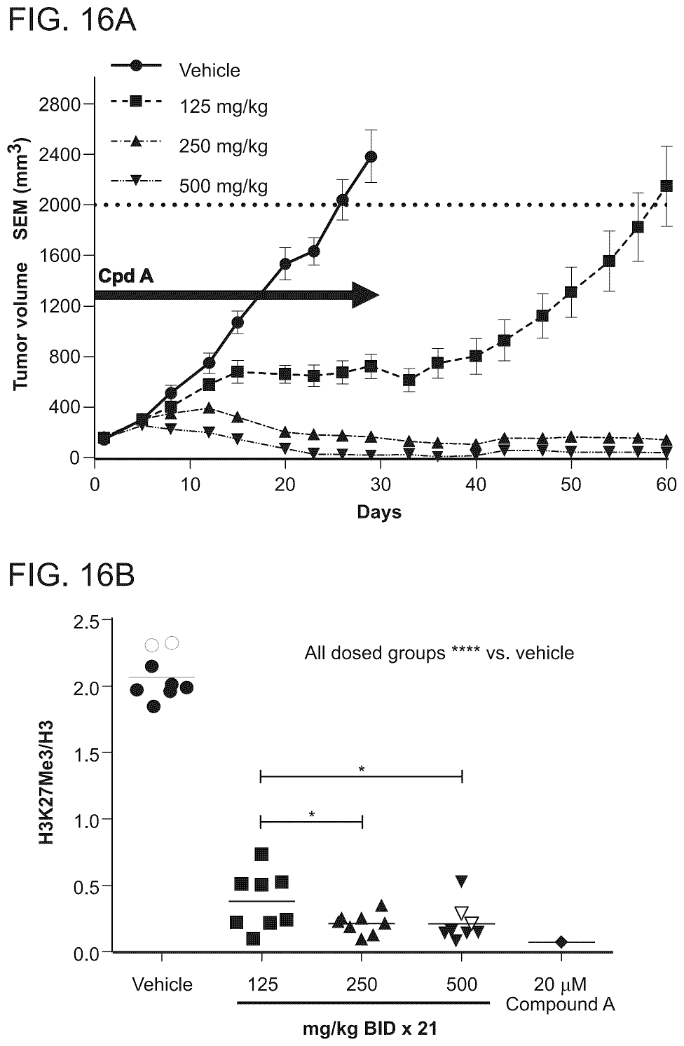

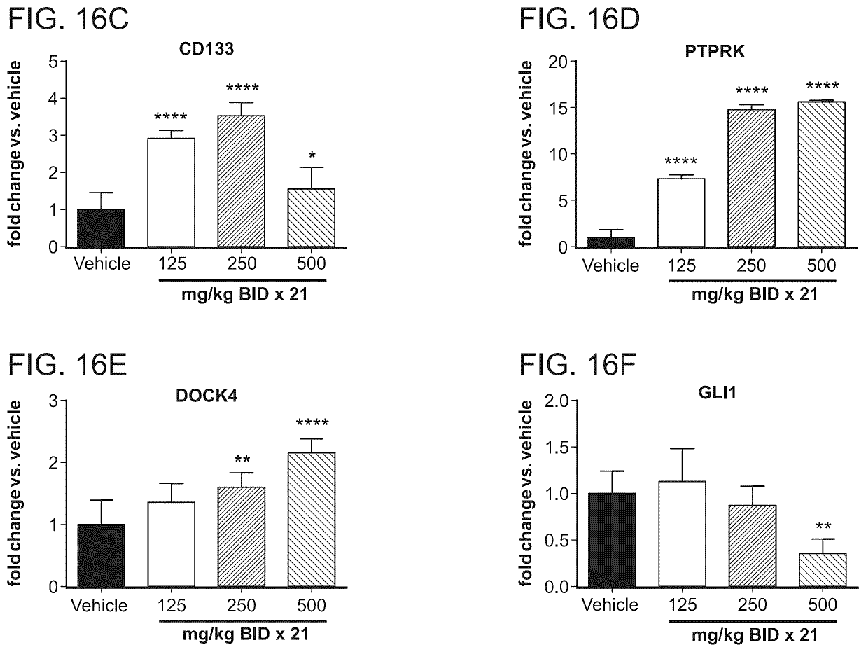

Compound A eradicates SMARCB1 mutant MRT xenografts: Oral dosing of Compound A led to systemic compound exposure, in vivo target inhibition and antitumor activity in mice bearing SMARCB1-deleted MRT xenografts. A study in SCID mice bearing subcutaneous G401 xenografts was performed where animals were dosed for 21 days with Compound A. Half of the mice per group were euthanized on day 21 to collect blood and tissues, while the remaining animals were treated for an additional 7 days and then left without dosing for another 32 days. Compound A was well tolerated at all doses with minimal effect on body weight (FIG. 15A). Dosing at 250 or 500 mg/kg twice daily (BID) for 21 to 28 days practically eliminated the fast-growing G401 tumors (FIGS. 15B, 14C and 16A). Re-growth was not observed for 32 days after dose cessation. Compound A dosed at 125 mg/kg induced tumor stasis during the administration period, and produced a significant tumor growth delay compared to vehicle after the dosing period. Measuring Compound A plasma levels either 5 min before or 3 h after dosing on day 21 revealed a clear dose-dependent increase in systemic exposure (FIG. 15D). Tumors that were harvested from subsets of mice from each group on day 21 showed strong inhibition of H3K27me3, correlating with the antitumor activity (maximum effect achieved at 250 mg/kg, FIG. 16B). In addition, dose-dependent changes in the expression of CD133, PTPRK, DOCK4 and GLI1 were detected in the G401 xenograft tumors (FIG. 16C).

The present data demonstrate that pharmacological inhibition of EZH2 induced antiproliferative effects specifically in SMARCB1-deleted MRT cell lines and permanently eradicated MRT xenografts in mice. This confirms the dependency of such cancers on PRC2 activity, despite the fact that EZH2 itself is not genetically altered in this context. Data presented herein show that in the context of SMARCB1-deleted MRT, inhibition of EZH2 functions as a SMARCB1 surrogate and de-represses neuronal differentiation genes, cell cycle inhibitors and tumor suppressors while reducing GLI1, PTCH1, MYC and EZH2. The sum of the effects of Compound A mediated EZH2 inhibition on several cancer pathways is the cause for the dramatic and permanent anti-tumor activity seen in MRT models. Thus, Compound A represents a new treatment modality for these lethal childhood tumors.

Furthermore, since several members of the SWI/SNF complex are genetically altered in other cancer types besides MRT, it is conceivable that EZH2 also plays a role in tumor maintenance and survival in a spectrum of cancer types. Combined with recent reports demonstrating the effectiveness of EZH2 inhibitors in selective killing of EZH2 mutant bearing non-Hodgkin lymphomas, the present data demonstrate that small molecule-based inhibition of EZH2 is an effective mechanism of therapeutic intervention in a variety of hematologic and solid tumors for which genetic alterations-either target-directed or indirect-confer a proliferative dependency on EZH2 enzymatic activity.

### Example 3: Material and Methods

Cell Culture: Cell lines 293T, RD, SJCRH30, A204, G401, G402, and KYM-1. 293T (CRL-11268), RD (CRL-136), SJCRH30 (CRL-2061), A204 (HTB-82), G401 (CRL-1441), and G402 (CRL-1440) were obtained from ATCC. KYM-1 (JCRB0627) was obtained from JCRB. 293T and RD cells were cultured in DMEM+10% FBS. SJCRH30 cells were cultured in RPMI+10% FBS. A204, G401, and G402 cells were cultured in McCoys 5a+10% FBS. KYM-1 cells were cultured in DMEM/Ham's F12+10% FBS.

Western blots analysis: Histones were acid extracted as previously described (Daigle et al., Blood. 2013 Aug. 8; 122 (6): 1017-25). Western blots for acid extracted histones were performed as previously described (Knutson et al., Proc Natl Acad Sci USA. 2013 May 7; 110 (19): 7922-7). Whole cell lysates (WCL) were prepared using a modified RIPA buffer (10× RIPA Lysis Buffer (Millipore #20-188), 0.1% SDS (Invitrogen AM9823), protease mini-tablet (Roche #1836153)). Cells were pelleted, washed with ice cold PBS, resuspended in ice cold RIPA buffer, and incubated on ice for 5 minutes. Lysates were sonicated 3× for 10 sec at 50% power, then incubated on ice for 10 minutes. Lysates were then centrifuged at max speed for 15 minutes at 4 degrees in a table top centrifuge. Clarified lysates were aliquoted to a fresh tube, and protein concentrations for WCL were determined by BCA assay (Pierce). Ten micrograms of each lysate was fractionated on 10-20% Tris-Glycine gel (Biorad), transferred using iBlot (7 minutes on program 3, using Nitrocellulose transfer stacks), and probed with the following antibodies in Odyssey blocking buffer: SNF5 (CST #8745), EZH2 (CST #5246), and Beta-actin (CST #3700).

In vitro cell assays: For the adherent cell line proliferation assays (all cell lines except KYM-1, which was analyzed as previously described for suspension cell lines (Daigle et al., Blood. 2013 Aug. 8; 122 (6): 1017-25), plating densities for each cell line were determined based on growth curves (measured by ATP viability) and density over a 7 day timecourse. On the day before compound treatment, cells were plated in either 96-well plates in triplicate (for the day 0-7 timecourse) or 6-well plates (for replating on day 7 for the remainder of the timecourse). On Day 0, cells were either untreated, DMSO-treated, or treated with Compound A starting at 10 μM and decreasing in either 3- or 4-fold dilutions. Plates were read on Day 0, Day 4, and Day 7 using CellTiter-Glo® (Promega), with compound/media being replenished on Day 4. On Day 7, the 6-well plates were trypsinized, centrifuged, and resuspended in fresh media for counting by Vi-Cell. Cells from each treatment were replated at the original density in 96-well plates in triplicate. Cells were allowed to adhere to the plate overnight, and cells were treated as on Day 0. On Day 7, 11 and 14, plates were read using CellTiter-Glo®, with compound/media being replenished on Day 11. Averages of triplicates were used to plot proliferation over the timecourse, and calculate IC₅₀ values. For cell cycle and apoptosis, G401 and RD cells were plated in 15 cm dishes in duplicate at a density of 1×10⁶ cells per plate. Cells were incubated with Compound A at 1 μM, in a total of 25 mL, over a course of 14 days, with cells being split back to original plating density on day 4, 7, and 11. Cell cycle analysis and TUNEL assay were performed using a Guava® flow cytometer, following the manufacturer's protocol.

Gene Expression Analysis: G401 and RD cells were plated in T-75 flasks at 175,000 cells/flask and 117,000 cells/flask respectively and allowed to adhere overnight. On Day 0, cells were treated in duplicates with DMSO or 1 μM Compound A. Cells were harvested and pelleted on Day 2, 4, and 7 with media and compound being replenished on Day 4. Tumor tissue from the G401 xenograft animals dosed for 21 days (vehicle, 125 mg/kg, and 250 mg/kg (6 animals each) and 500 mg/kg (4 animals) Compound A dose groups) were used for gene expression analysis. Total mRNA was extracted from cell pellets and tumor tissue using the RNeasy Mini Kit (Qiagen #74106) and reverse transcribed by the High Capacity cDNA Reverse Transcription Kit (Applied Biosystems (AB) #4368813). RT-PCR was performed by ViiA™ 7 Real-Time PCR Systems (AB) using TaqMan Fast Advanced Master Mix (AB #4444964) and TaqMan primer/probe sets in table below. Gene expression was normalized to 18S (AB #Hs99999901_s1) and fold change was calculated using the AACt method. For the in vivo samples, the average Ct value+/−SD was determined for each dose group and fold change compared to vehicle dose group was calculated using the AACt method.

| Gene | AB# |
|---|---|
| MYC | Hs00153408_m1 |
| EZH2 | Hs00172783_m1 |
| PTCH1 | Hs00181117_m1 |
| PROM1 (CD133) | Hs01009250_m1 |
| GLI1 | Hs01110766_m1 |
| DOCK4 | Hs00206807_m1 |
| PTPRK | Hs00267788_m1 |
| BIN1 | Hs00184913_m1 |

ELISA: Histones were isolated from tumors as previously described (Daigle et al) and were prepared in equivalent concentrations (0.5 ng/ul for H3 and 4 ng/ul for H3K27Me3) in coating buffer (PBS with 0.05% BSA). Sample or standard (100 μL) was added in duplicate to two 96-well ELISA plates (Thermo Labsystems, Immulon 4HBX #3885). Histones isolated from G401 cells that were treated with DMSO or 10 μmol/L Compound A for 4 days were added to control wells at the same histone concentration as the tumor histone samples. The plates were sealed and incubated overnight at 4° C. The following day, plates were washed 3 times with 300 μL/well PBST (PBS with 0.05% Tween 20; 10× PBST, KPL #51-14-02) on a Bio Tek plate washer. Plates were blocked with 300 μL/well of diluent (PBS+2% BSA+0.05% Tween 20), incubated at room temperature for 2 hours, and washed 3 times with PBST. All antibodies were diluted in diluent. 100 μL/well of anti-H3K27Me3 (CST #9733, 50% glycerol stock 1:1000) or anti-total H3 (Abcam #ab1791, 50% glycerol stock 1:10,000) was added to each plate. Plates were incubated for 90 minutes at room temperature and washed 3 times with PBST. 100 μL/well of anti-R_{b}-IgG-HRP (Cell Signaling Technology, 7074) was added 1:2000 to the H3K27Me3 plate and 1:6000 to the H3 plate and incubated for 90 minutes at room temperature. Plates were washed 4 times with PBST. For detection, 100 L/well of TMB substrate (BioFx Laboratories, #TMBS) was added and plates incubated in the dark at room temperature for 5 minutes. Reaction was stopped with 100 μL/well 1N H₂SO₄. Absorbance at 450 nm was read on SpectraMax M5 Microplate reader.

Xenograft study: All the procedures related to animal handling, care and the treatment in this study were performed according to the guidelines approved by the Institutional Animal Care and Use Committee (IACUC) of Shanghai Chemparner following the guidance of the Association for Assessment and Accreditation of Laboratory Animal Care (AAALAC). For the in vivo study, mice were inoculated subcutaneously at the right flank with G-401 tumor cells (5×10⁶/mouse) in 0.2 ml mixture of base media and Matrigel (McCoy's 5A: Matrigel=1:1) for tumor development. The treatments were started when the tumor size reached approximately 157 mm³ for the tumor efficacy study (n=16 mice per group). Compound A or vehicle (0.5% NaCMC+0.1% Tween-80 in water) was administered orally BID at a dose volume of 10 μL/g for either 21 or 28 days. Animal body weights were measured every day during the first week, then twice weekly for the remainder of the study. Tumor size was measured twice weekly in two dimensions using a caliper, and the volume was expressed in mm³. For PK/PD analysis, 8 mice with the largest tumor burden were euthanized for tumor and blood collection after 21 days of dosing. The remaining mice continued dosing for one more week, and from day 29, treatment was stopped and the mice were enrolled in a tumor growth delay study. Mice were observed as individuals until they reached the tumor weight endpoint (2000 mm³) or until day 60 (whichever came first).

Pharmacokinetic analyses: Dexamethasone was used as internal standard. An aliquot of 30 μL plasma sample was added with 30 μL IS (Dexamethasone, 1000 ng/mL) and 150 μL ACN. The mixture was vortexed for 5 min and centrifuged at 14000 rpm for 5 min. An aliquot of 2 μL supernatant was injected for LC-MS/MS analysis (Q-trap 3200). For 10-fold diluted plasma samples an aliquot of 3 μL plasma sample was added with 27 μL blank plasma, the dilution factor was 10, then added with 30 μL IS (Dexamethasone, 1000 ng/ml) and 150 μL ACN. The mixture was vortexed for 5 min and centrifuged at 14000 rpm for 5 min. An aliquot of 2 μL supernatant was injected for LC-MS/MS analysis. Tumor samples were homogenized on Beadbeater® for 30 seconds with 3× PBS (w/v) to obtain a tumor homogenate. An aliquot of 30 μL tumor homogenate sample was added with 30 μL IS (Dexamethasone, 1000 ng/mL) and 150 μL ACN. The mixture was vortexed for 5 min and centrifuged at 14000 rpm for 5 min. An aliquot of 2 μL supernatant was injected for LC-MS/MS analysis.

### Example 4: General Experimental Procedures

### NMR

1H-NMR spectra were taken using CDCl₃ unless otherwise stated and were recorded at 400 or 500 MHz using a Varian or Oxford instruments magnet (500 MHz) instruments. Multiplicities indicated are s=singlet, d=doublet, t=triplet, q=quartet, quint=quintet, sxt=sextet, m=multiplet, dd-doublet of doublets, dt=doublet of triplets; br indicates a broad signal.

### LCMS and HPLC

Shimadzu LC-Q, Shimadzu LCMS-2010EV or Waters Acquity Ultra Performance LC. HPLC: Products were analyzed by Shimadzu SPD-20A with 150×4.5 mm YMC ODS-M80 column or 150×4.6 mm YMC-Pack Pro C18 column at 1.0 ml/min.

Mobile phase was MeCN:H2O=3:2 (containing 0.3% SDS and 0.05% H₃PO₄),

0.05% TFA in water, 0.05% TFA in acetonitrile (gradient Initial 20%, then 0.05% TFA/MeCN to conc. to 95% in 3 min. holds for 0.5 min. at 3.51 to 4.50 min then 0.05% TFA/MeCN conc. 20%).

Alternatively the LCMS, 2 different methods were used; the one we use the most is the high pH (METCR1600) and the other one for more standard compounds (METCR1416).

0.1% Formic acid in water-Mobile phase “A” 0.1% Formic acid in acetonitrile-Mobile phase “B” utilizing Waters Atlantis dC18, 2.1 mm×100 mm, 3 μm column, with a flow rate=0.6 ml/min Column temperature=40° C.; Time (mins) % B 0.00 min 5% B. 5.0 mins 100% B, 5.4 mins 100% B and 0.42 mins 5% B

3.5 minute method refers to Atlantis dC18, 2.1 mm×50 mm, 3 μm column, flow rate of 1 ml/min at 40C. Mobile phase A Formic acid (aq.) 0.1% mobile phase B formic acid (MeCN) 0.1%, injection 3 μL, gradient 0 mins (5% organic), 2.5 min (100% organic), 2.7 mins (100% organic), 2.71 min (5% organic), 3.5 min (5% organic)

7.0 minute method refers to Atlantis dC18, 2.1 mm×100 mm, 3 μm column, flow rate of 0.6 ml/min at 40C. Mobile phase A Formic acid (aq.) 0.1% mobile phase B formic acid (MeCN) 0.1%, injection 3 μL, gradient 0 mins (5% organic), 5 min (100% organic), 5.4 mins (100% organic), 5.42 min (5% organic), 7 min (5% organic)

Both the 3. 5 and 7 minute methods were performed on a MS18 Shimadzu LCMS-2010EV or a MS19 Shimadzu LCMS-2010EV system utilizing LC-20AB pumps and SPD-M20A PDA detectors.

Products were purified by HPLC/MS using Waters AutoPurification System with 3100 Mass Detector.

HPLC analyses may also be performed on a Shimdazu LC-2010CHT using an YMC ODS-A, C₁₈, (150×4.6×5 μm) column at ambient temperature with a flow Rate of 1.4 ml/min. An injection volume of 10 μl is utilized and detection occurs via UV/PDA. Mobile Phase A is 0.05% TFA in water and Mobile Phase B is 0.05% TFA in acetonitrile with a gradient program of Initial 5% B to 95% B in 8 min, hold for 1.5 min, at 9.51 to 12 min B. conc. 0.5%. The diluent is the mobile phase

### Other

Automated flash column chromatography was performed on a Biotage Isolera version 4. 10 g SNAP cartridge running at 12 ml/min or a 25 g SNAP cartridge running at 25 ml/min and detecting at 254 nm and 280 nm.

Select Nitrile reductions may be performed on a ThalesNano H-Cube® according to the conditions described in the experimental procedure.

Other related general procedures can also be found in PCT publication No. WO12/118812, PCT application No. PCT/US2012/033648 and PCT application No. PCT/US2012/033662, each of which is incorporated herein by reference in its entirety.

### Example 5: Synthesis of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide

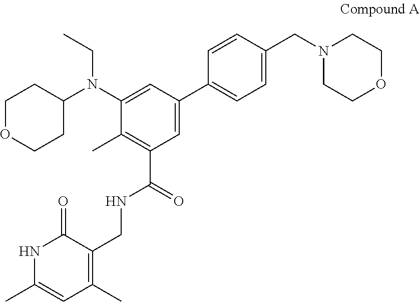 [SMILES: CCN(c1cc(-c2ccc(CN3CCOCC3)cc2)cc(C(=O)NCc2c(C)cc(C)[nH]c2=O)c1C)C1CCOCC1]

### Step 1: Synthesis of 5-bromo-2-methyl-3-nitrobenzoic acid

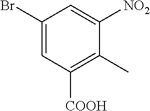

To stirred solution of 2-methyl-3-nitrobenzoic acid (100 g, 552 mmol) in conc. H₂SO₄ (400 mL), 1,3-dibromo-5,5-dimethyl-2,4-imidazolidinedione (88 g, 308 mmol) was added in a portion wise manner at room temperature and the reaction mixture was then stirred at room temperature for 5 h. The reaction mixture was poured onto ice cold water, the precipitated solid was filtered off, washed with water and dried under vacuum to afford the desired compound as a solid (140 g, 98%). The isolated compound was taken directly into the next step. ¹H NMR (DMSO-d₆, 400 MHZ) δ 8.31 (s, 1H), 8.17 (s, 1H), 2.43 (s, 3H).

### Step 2: Synthesis of methyl 5-bromo-2-methyl-3-nitrobenzoate

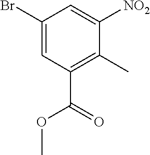

To a stirred solution of 5-bromo-2-methyl-3-nitrobenzoic acid (285 g, 1105 mmol) in DMF (2.8 L) at room temperature was added sodium carbonate (468 g, 4415 mmol) followed by addition of methyl iodide (626.6 g, 4415 mmol). The resulting reaction mixture was heated at 60° C. for 8 h. After completion (monitored by TLC), the reaction mixture was filtered (to remove sodium carbonate) and washed with ethyl acetate (1L×3). The combined filtrate was washed with water (3L×5) and the aqueous phase was back extracted with ethyl acetate (1L×3). The combined organic layers were dried over anhydrous sodium sulfate, filtered and concentrated under reduced pressure to afford the title compound as a solid (290 g, 97% yield). The isolated compound was taken directly into the next step. ¹H NMR (CDCl₃, 400 MHZ) δ 8.17 (s, 1H), 7.91 (s, 1H), 3.96 (s, 3H), 2.59 (s, 3H).

### Step 3: Synthesis of methyl 3-amino-5-bromo-2-methylbenzoate

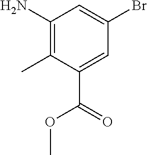

To a stirred solution of methyl 5-bromo-2-methyl-3-nitrobenzoate (290 g, 1058 mmol) in ethanol (1.5 L) was added aqueous ammonium chloride (283 g, 5290 mmol dissolved in 1.5 L water). The resulting mixture was stirred at 80° C. to which iron powder (472 g, 8451 mmol) was added in a portion wise manner. The resulting reaction mixture was heated at 80° C. for 12 h. Upon completion as determined by TLC, the reaction mixture was hot filtered over Celite® and the celite bed was washed with methanol (5 L) followed by washing with 30% MeOH in DCM (5 L). The combined filtrate was concentrated in-vacuo, the residue obtained was diluted with aqueous sodium bicarbonate solution (2 L) and extracted with ethyl acetate (5L×3). The combined organic layers were dried over anhydrous sodium sulfate, filtered and concentrated under reduced pressure to afford the title compound as a solid (220 g, 85%). The compound was taken directly into the next step. ¹H NMR (CDCl₃, 400 MHZ) δ 7.37 (s, 1H), 6.92 (s, 1H), 3.94 (s, 3H), 3.80 (bs, 2H), 2.31 (s, 3H).

### Step 4: Synthesis of methyl 5-bromo-2-methyl-3-((tetrahydro-2H-pyran-4-yl) amino)benzoate

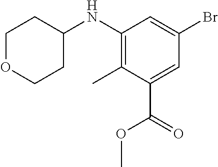 [SMILES: COC(=O)c1cc(Br)cc(NC2CCOCC2)c1C]

To a stirred solution of methyl 3-amino-5-bromo-2-methylbenzoate (15 g, 61.5 mmol) and dihydro-2H-pyran-4 (3)-one (9.2 g, 92 mmol) in dichloroethane (300 mL) was added acetic acid (22 g, 369 mmol) and the reaction mixture stirred at room temperature for 15 minutes, then the reaction mixture was cooled to 0° C. and sodium triacetoxyborohydride (39 g, 184 mmol) was added. The reaction mixture was stirred overnight at room temperature. Upon completion of the reaction as determined by TLC, aqueous sodium bicarbonate solution was added to the reaction mixture until a pH of 7-8 was obtained. The organic phase was separated and the aqueous phase was extracted with ethyl acetate. The combined organic layers were dried over anhydrous sodium sulfate, filtered and concentrated under reduced pressure. The crude compound was purified by column chromatography (100-200 mesh silica gel) eluting with ethyl acetate: hexane to afford the desired compound as a solid (14 g, 69%). ¹H NMR (DMSO-d₆, 400 MHZ) δ 7.01 (s, 1H), 6.98 (s, 1H), 5.00 (d, 1H, J=7.6 Hz), 3.84-3.87 (m, 2H), 3.79 (s, 3H), 3.54-3.56 (m, 1H), 3.43 (t, 2H, J=12 Hz), 2.14 (s, 3H), 1.81-1.84 (m, 2H), 1.47-1.55 (m, 2H).

### Step 5: Synthesis of methyl 5-bromo-3-(ethyl(tetrahydro-2H-pyran-4-yl)amino)-2-methylbenzoate

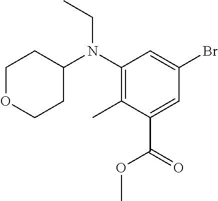 [SMILES: CCN(c1cc(Br)cc(C(=O)OC)c1C)C1CCOCC1]

To a stirred solution of methyl 5-bromo-2-methyl-3-((tetrahydro-2H-pyran-4-yl) amino)benzoate (14 g, 42.7 mmol) in dichloroethane (150 mL) was added acetaldehyde (3.75 g, 85.2 mmol) and acetic acid (15.3 g, 256 mmol). The resulting reaction mixture was stirred at room temperature for 15 minutes. The mixture was cooled to 0° C. and sodium triacetoxyborohydride (27 g, 128 mmol) was added. The reaction mixture was stirred at room temperature for 3 hours. Upon completion of the reaction as determined by TLC, aqueous sodium bicarbonate solution was added to the reaction mixture until a pH 7-8 was obtained, the organic phase was separated and the aqueous phase was extracted with ethyl acetate. The combined organic layers were dried over anhydrous sodium sulfate, filtered and concentrated under reduced pressure. The crude compound was purified by column chromatography (100-200 mesh silica gel) eluting with ethyl acetate: hexane to afford the desired compound as a viscous liquid (14 g, 93%). ¹H NMR (DMSO-d₆, 400 MHZ) δ 7.62 (s, 1H), 7.52 (s, 1H), 3.80 (bs, 5H), 3.31 (t, 2H), 2.97-3.05 (m, 2H), 2.87-2.96 (m, 1H), 2.38 (s, 3H), 1.52-1.61 (m, 2H), 1.37-1.50 (m, 2H), 0.87 (t, 3H, J=6.8 Hz).

### Step 6: Synthesis of 5-bromo-N-((4,6-dimethyl-2-oxo-1,2-dihydropyridin-3-yl)methyl)-3-(ethyl (tetrahydro-2H-pyran-4-yl)amino)-2-methylbenzamide

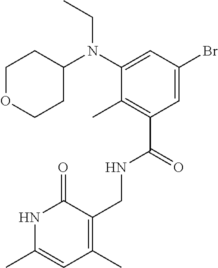 [SMILES: CCN(c1cc(Br)cc(C(=O)NCc2c(C)cc(C)[nH]c2=O)c1C)C1CCOCC1]

To a stirred solution of 5-bromo-3-(ethyl (tetrahydro-2H-pyran-4-yl)amino)-2-methylbenzoate (14 g, 39.4 mmol) in ethanol (100 mL) was added aqueous NaOH (2.36 g, 59.2 mmol in 25 mL water) and the resulting mixture was stirred at 60° C. for 1 h. Upon completion of the reaction as determined by TLC, the solvent was removed under reduced pressure and the residue obtained was acidified with 1N HCl until a pH 7 was obtained and then aqueous citric acid solution was added until a pH 5-6 was obtained. The aqueous layer was extracted with 10% MeOH in DCM (200 mL×3), the combined organic layers were dried over anhydrous sodium sulfate, filtered and concentrated under reduced pressure to give the respective acid (14 g, 100%).

The above acid (14 g, 40.9 mmol) was then dissolved in DMSO (70 mL) and 3-(amino methyl)-4,6-dimethylpyridin-2 (1H)-one (12.4 g, 81.9 mmol) was added to it. The reaction mixture was stirred at room temperature for 15 minutes, then PYBOP (31.9 g, 61.4 mmol) was added and stirring was continued for overnight at room temperature. Upon completion of the reaction as determined by TLC, the reaction mixture was poured onto ice-cold water (700 mL), stirred for 30 minutes and the precipitated solid was collected by filtration, washed with water (500 mL) and air dried. The solid obtained was stirred with acetonitrile (75 mL×2), filtered and air dried. The solid obtained was again stirred with 5% MeOH in DCM (100 mL), filtered and dried completely under vacuum to afford the title compound as a solid (14 g, 74%). ¹H NMR (DMSO-d₆, 400 MHZ) δ 11.47 (s, 1H), 8.23 (t, 1H), 7.30 (s, 1H), 7.08 (s, 1H), 5.85 (s, 1H), 4.23 (d, 2H, J=4.4 Hz), 3.81 (d, 2H, J=10.4 Hz), 3.20-3.26 (m, 2H), 3.00-3.07 (m, 1H), 2.91-2.96 (m, 2H), 2.18 (s, 3H), 2.14 (s, 3H), 2.10 (s, 3H), 1.58-1.60 (m, 2H), 1.45-1.50 (m, 2H), 0.78 (t, 3H, J=6.8 Hz).

### Step 7: Synthesis of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide

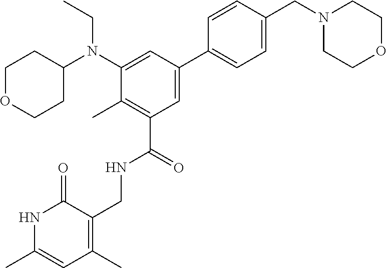 [SMILES: CCN(c1cc(-c2ccc(CN3CCOCC3)cc2)cc(C(=O)NCc2c(C)cc(C)[nH]c2=O)c1C)C1CCOCC1]

To a stirred solution of 5-bromo-N-((4,6-dimethyl-2-oxo-1,2-dihydropyridin-3-yl)methyl)-3-(ethyl (tetrahydro-2H-pyran-4-yl)amino)-2-methylbenzamide (14 g, 29.5 mmol) in dioxane/water mixture (70 mL/14 mL) was added 4-(4-(4,4,5,5-tetramethyl-1,3,2-dioxaborolan-2-yl) benzyl) morpholine (13.4 g, 44.2 mmol) followed by addition of Na₂CO₃ (11.2 g, 106.1 mmol). The solution was purged with argon for 15 minutes and then Pd(PPh₃)₄ (3.40 g, 2.94 mmol) was added and the solution was again purged with argon for a further 10 min. The reaction mixture was heated at 100° C. for 4 h. After completion (monitored by TLC), the reaction mixture was diluted with water and extracted with 10% MeOH/DCM. The combined organic layers were dried over anhydrous sodium sulphate, filtered and concentrated under reduced pressure. The crude compound was purified by column chromatography (100-200 mesh silica gel) eluting with methanol: DCM to the title compound as a solid (12 g, 71%). Analytical Data: LCMS: 573.35 (M+1)⁺; HPLC: 99.5% (@254 nm) (R; 3.999; Method: Column: YMC ODS-A 150 mm×4.6 mm×5μ; Mobile Phase: A; 0.05% TFA in water/B; 0.05% TFA in acetonitrile; Inj. Vol: 10 μL, Col. Temp.: 30° C.; Flow rate: 1.4 mL/min.; Gradient: 5% B to 95% B in 8 min, Hold for 1.5 min, 9.51-12 min 5% B); ¹H NMR (DMSO-d₆, 400 MHZ) δ 11.46 (s, 1H), 8.19 (t, 1H), 7.57 (d, 2H, J=7.2 Hz), 7.36-7.39 (m, 3H), 7.21 (s, 1H), 5.85 (s, 1H), 4.28 (d, 2H, J=2.8 Hz), 3.82 (d, 2H, J=9.6 Hz), 3.57 (bs, 4H), 3.48 (s, 2H), 3.24 (t, 2H, J=10.8 Hz), 3.07-3.09 (m, 2H), 3.01 (m, 1H), 2.36 (m, 4H), 2.24 (s, 3H), 2.20 (s, 3H), 2.10 (s, 3H), 1.64-1.67 (m, 2H), 1.51-1.53 (m, 2H), 0.83 (t, 3H, J=6.4 Hz).

### Step 8: Synthesis of N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide trihydrochloride

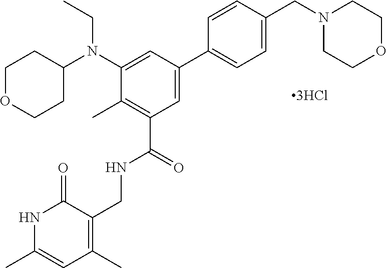 [SMILES: CCN(c1cc(-c2ccc(CN3CCOCC3)cc2)cc(C(=O)NCc2c(C)cc(C)[nH]c2=O)c1C)C1CCOCC1]

N-((4,6-dimethyl-2-oxo-1,2-dihydropyridin-3-yl)methyl)-5-(ethyl (tetrahydro-2H-pyran-4-yl)amino)-4-methyl-4′-(morpholinomethyl)-[1,1′-biphenyl]-3-carboxamide (12 g, 21.0 mmol) was dissolved in methanolic HCl (200 mL) and stirred at room temperature for 3 h. After three hours of stirring, the reaction mixture was concentrated under reduced pressure. The solid obtained was stirred with ether (100 mL×2) to afford the desired salt as a solid (11 g, 77%). Analytical Data of the tri-HCl salt: LCMS: 573.40 (M+1)+; HPLC: 99.1% (@ 254 nm) (Rr; 3.961; Method: Column: YMC ODS-A 150 mm×4.6 mm×5μ; Mobile Phase: A; 0.05% TFA in water/B; 0.05% TFA in acetonitrile; Inj. Vol: 10 μL, Col. Temp.: 30° C.; Flow rate: 1.4 mL/min.; Gradient: 5% B to 95% B in 8 min, Hold for 1.5 min, 9.51-12 min 5% B); ¹H NMR (D₂O 400 MHZ) δ 7.92 (bs, 1H) 7.80 (s, 1H), 7.77 (d, 2H, J=8 Hz), 7.63 (s, 1H), 7.61 (s, 1H), 6.30 (s, 1H), 4.48 (s, 2H), 4.42 (s, 2H), 4.09-4.11 (m, 4H), 3.95-3.97 (m, 2H), 3.77 (t, 3H, J=10.4 Hz), 3.44-3.47 (m, 3H), 3.24-3.32 (m, 3H), 2.42 (s, 3H), 2.35 (s, 3H), 2.26 (s, 3H), 2.01 (m, 2H), 1.76 (m, 2H), 1.04 (t, 3H, J=6.8 Hz).

### Example 6: N-((4,6-dimethyl-2-oxo-1,2-dihydropyridin-3-yl)methyl)-5-(((1r,4r)-4-(dimethylamino)cyclohexyl)(ethyl)amino)-4-methyl-4′-(morpholinomethyl)-[1,1′-biphenyl]-3-carboxamide

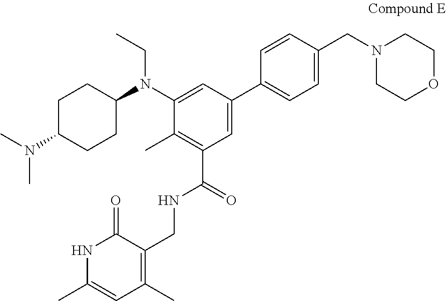 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

### Step 1:5-bromo-2-methyl-3-nitrobenzoic acid

To stirred solution of 2-methyl-3-nitrobenzoic acid (100 g, 552.48 mmol) in conc. H₂SO₄ (400 mL), 1,3-dibromo-5,5-dimethyl-2,4-imidazolidinedione (87.98 g, 307.70 mmol) was added in a portion-wise manner at room temperature. The reaction mixture was then stirred at room temperature for 5 h. The reaction mixture was poured into ice cold water, the precipitated solid collected by filtration, washed with water and dried under vacuum to afford desired 5-bromo-2-methyl-3-nitrobenzoic acid as off-white solid (140 g, 97.90% yield). 1H NMR (DMSO-d₆, 400 MHZ) δ 8.31 (s, 1H), 8.17 (s, 1H), 2.43 (s, 3H).

### Step 2: methyl 5-bromo-2-methyl-3-nitrobenzoate

To a stirred solution of 5-bromo-2-methyl-3-nitrobenzoic acid (285 g, 1104.65 mmol) in DMF (2.8 L) was added sodium carbonate (468 g, 4415.09 mmol) followed by addition of methyl iodide (626.63 g, 4415 mmol) at room temperature. The resulting reaction mixture was stirred at 60° C. for 8 h. The reaction mixture was then filtered to remove suspended solids which were washed well with ethyl acetate (3×1 L). The combined filtrates were washed well with water (5×3 L) and the aqueous phase back extracted with ethyl acetate (3×1 L). The combined organic extracts dried over anhydrous sodium sulfate, filtered and concentrated under reduced pressure to afford methyl 5-bromo-2-methyl-3-nitrobenzoate as an off-white solid (290 g, 97% yield). ¹H NMR (CDCl₃, 400 MHZ) δ 8.17 (s, 1H), 7.91 (s, 1H), 3.96 (s, 3H), 2.59 (s, 3H).

### Step 3: methyl 3-amino-5-bromo-2-methylbenzoate

To a stirred solution of methyl 5-bromo-2-methyl-3-nitrobenzoate (290 g, 1058.39 mmol) in ethanol (1.5 L) was added aqueous ammonium chloride (283 g, 5290 mmol dissolved in 1.5 L water). The resulting mixture was stirred and heated at 80° C. followed by addition of iron powder (472 g, 8451 mmol) in portions at 80° C. The resulting reaction mixture was heated at 80° C. for 12 h. The reaction mixture was then hot filtered through Celite® and the Celite® bed washed well methanol (5 L) and then with 30% MeOH in DCM (5 L). The combined filtrates were concentrated in vacuo and the residue obtained was diluted with aqueous bicarbonate (2 L) and extracted with ethyl acetate (3×5 L). The combined organic layers were dried over anhydrous sodium sulfate, filtered and concentrated under reduced pressure to afford methyl 3-amino-5-bromo-2-methylbenzoate as a brown solid (220 g, 89.41% yield).

A portion of the product (5 g) was dissolved in hot ethanol (20 mL), insoluble residue filtered off and mother liquor concentrated to obtain methyl 3-amino-5-bromo-2-methylbenzoate (3.5 g, 70% yield) with HPLC purity 93.81% as light brown solid. 1H NMR (CDCl₃, 400 MHZ) δ 7.37 (s, 1H), 6.92 (s, 1H), 3.94 (s, 3H), 3.80 (bs, 2H), 2.31 (s, 3H).

### Step 4: methyl 5-bromo-3-(((1r,4r)-4-((tert-butoxycarbonyl)amino)cyclohexyl)amino)-2-methylbenzoate

To a stirred solution of methyl 3-amino-5-bromo-2-methylbenzoate (5 g, 20.5 mmol) and tert-butyl (4-oxocyclohexyl)carbamate (5.69 g, 26.7 mmol) in dichloroethane (50 mL), acetic acid (7.4 g, 123 mmol) was added and the reaction was stirred at room temperature for 10 minutes. Sodium triacetoxyborohydride (13.1 g, 61.7 mmol) was then added at 0° C. and reaction was stirred at room temperature for 16 hours. The reaction was quenched with aqueous sodium bicarbonate, the organic phase separated and the aqueous phase extracted with dichloromethane. The combined organic layers were dried over anhydrous sodium sulfate and concentrated in vacuo. The crude product was purified by silica gel column chromatography (100-200 mesh size) eluting with 10% ethyl acetate in hexane to afford 3.5 g of the more polar (trans) isomer, methyl 5-bromo-3-(((1r,4r)-4-((tert-butoxycarbonyl)amino)cyclohexyl)amino)-2-methylbenzoate, as solid (38.46%). ¹H NMR (CDCl₃, 400 MHZ) δ 7.21 (s, 1H), 6.80 (s, 1H), 4.41 (bs, 1H), 3.85 (s, 3H), 3.60 (m, 1H), 3.45 (m, 1H), 3.20 (m, 1H), 2.22 (s, 3H), 2.15 (bs, 2H), 2.05 (bs, 2H), 1.45 (s, 9H), 1.30 (m, 4H).

### Step 5: methyl 5-bromo-3-(((1r,4r)-4-((tert-butoxycarbonyl)amino)cyclohexyl)-(ethyl)amino)-2-methylbenzoate

To a stirred solution of methyl 5-bromo-3-(((1r,4r)-4-((tert-butoxycarbonyl)amino)-cyclohexyl)(ethyl)amino)-2-methylbenzoate (55 g, 0.124 mol) and acetaldehyde (11 g, 0.25 mol) in dichloroethane (550 mL), acetic acid (44.64 g, 0.744 mol) was added and the reaction mixture stirred at room temperature for 10 minutes. Sodium triacetoxyborohydride (79 g, 0.372 mol) was then added at 0° C. and the reaction mixture was stirred at room temperature for 16 hours. The reaction was quenched with aqueous sodium bicarbonate, the organic phase separated and the aqueous phase extracted with dichloromethane. The combined extracts were dried over anhydrous sodium sulfate and concentrated in-vacuo. The crude compound was purified by silica gel column chromatography (100-200 mesh size) eluting with 10% ethyl acetate in hexane to afford 44 g of methyl 5-bromo-3-(((1r,4r)-4-((tert-butoxycarbonyl)amino)cyclohexyl)-(ethyl)amino)-2-methylbenzoate (75.2%) as solid. ¹H NMR (DMSO-d₆, 400 MHZ) δ 7.55 (s, 1H), 7.45 (s, 1H), 6.65 (d, 1H), 3.80 (s, 3H), 3.15 (bs, 1H), 3.05 (q, 2H), 2.60 (m, 1H), 2.30 (s, 3H), 1.75 (m, 4H), 1.40 (m, 2H), 1.35 (s, 9H), 1.10 (m, 2H), 0.80 (t, 3H).

### Step 6: tert-butyl ((1r,4r)-4-((5-bromo-3-(((4,6-dimethyl-2-oxo-1,2-dihydropyridin-3-yl)methyl)carbamoyl)-2-methylphenyl)(ethyl)amino)cyclohexyl)carbamate

Aqueous NaOH (3.5 g, 0.08 mol in 10 mL H₂O) was added to a solution of methyl 5-bromo-3-(((1r,4r)-4-((tert-butoxycarbonyl)amino)cyclohexyl)-(ethyl)amino)-2-methylbenzoate (25 g, 0.053 mol) in EtOH (100 mL) and stirred at 60° C. for 1 h. The ethanol was then removed under reduced pressure and acidified to pH 8 with dilute HCl and to pH 6 with citric acid. The mixture was extracted with 10% methanol in DCM (3×200 mL). The combined organic layers were dried and concentrated giving the respective acid (24.2 g, 99.0%). ¹H NMR (DMSO-d₆, 400 MHZ) δ 13.13 (s, 1H), 7.54 (s, 1H), 7.43 (s, 1H), 6.68 (d, 1H), 3.14 (bs, 1H), 3.03 (q, 2H), 2.56 (m, 1H), 2.33 (s, 3H), 1.80-1.65 (m, 4H), 1.40 (m, 2H), 1.35 (s, 9H), 1.10 (m, 2H), 0.77 (t, 3H).

The acid (24 g, 0.053 mol) was dissolved in DMSO (100 mL) and 3-(aminomethyl)-4,6-dimethylpyridin-2 (1H)-one (16 g, 0.106 mol) and triethylamine (5.3 g, 0.053 mol) was added. The reaction mixture was stirred at room temperature for 15 min before PyBop (41 g, 0.079 mmol) was added and stirring was then continued for overnight at room temperature. The reaction mixture was poured into ice water (IL). The resulting precipitate was collected by filtration, washed well with water (2×1 L) and dried. The product obtained was further purified by washings with acetonitrile (3×200 mL) and DCM (100 mL) to afford tert-butyl ((1r,4r)-4-((5-bromo-3-(((4,6-dimethyl-2-oxo-1,2-dihydropyridin-3-yl)methyl)carbamoyl)-2-methylphenyl)(ethyl)amino)cyclohexyl)-carbamate (24 g, 77%). ¹H NMR (DMSO-d₆, 400 MHZ) δ 11.47 (s, 1H), 8.24 (t, 1H), 7.25 (s, 1H), 7.04 (s, 1H), 6.67 (d, 1H), 5.85 (s, 1H), 4.24 (d, 2H), 3.13 (bs, 1H), 3.01 (q, 2H), 2.53 (m, 1H), 2.18 (s, 3H), 2.10 (s, 6H), 1.80-1.65 (m, 4H), 1.40 (m, 2H), 1.35 (s, 9H), 1.10 (m, 2H), 0.77 (t, 3H).

### Step 7: tert-butyl ((1r,4r)-4-((5-(((4,6-dimethyl-2-oxo-1,2-dihydropyridin-3-yl)methyl)carbamoyl)-4-methyl-4′-(morpholinomethyl)-[1,1′-biphenyl]-3-yl)(ethyl)amino)cyclohexyl)carbamate

To a stirred solution of tert-butyl ((1r,4r)-4-((5-bromo-3-(((4,6-dimethyl-2-oxo-1,2-dihydropyridin-3-yl)methyl)carbamoyl)-2-methylphenyl)(ethyl)amino)cyclohexyl)-carbamate (24 g, 0.041 mol) and 4-(4-(4,4,5,5-tetramethyl-1,3,2-dioxaborolan-2-yl) benzyl) morpholine (18 g, 0.061 mol) in dioxane/water mixture (160 mL+40 mL), Na₂CO₃ (15 g, 0.15 mol) was added and solution purged with argon for 15 min. Pd(PPh₃)₄ (4.7 g, 0.041 mol) was then added and the reaction mixture again purged with argon for 10 min. The reaction mixture was heated at 100° C. for 4 h. The reaction mixture was then diluted with 10% MeOH/DCM (500 mL) and filtered. The filtrate was concentrated, diluted with water (500 mL) and extracted with 10% MeOH in DCM (3×500 mL). The combined organic layers were dried over Na₂SO₄ and solvent removed under reduced pressure. The crude product was purified by silica gel column chromatography (100-200 mesh) eluting with 7% MeOH in DCM to afford tert-butyl ((1r,4r)-4-((5-(((4,6-dimethyl-2-oxo-1,2-dihydropyridin-3-yl)methyl)carbamoyl)-4-methyl-4′-(morpholinomethyl)-[1,1′-biphenyl]-3-yl)(ethyl)amino)cyclohexyl)carbamate (20 g, 71.43%). 1H NMR (DMSO-d₆, 400 MHz) δ 11.46 (s, 1H), 8.20 (t, 1H), 7.56 (d, 2H), 7.36 (m, 3H), 7.17 (s, 1H), 6.66 (d, 1H), 5.85 (s, 1H), 4.28 (d, 2H), 3.57 (bs, 4H), 3.48 (s, 2H), 3.20-3.05 (m, 3H), 2.62 (m, 1H), 2.36 (bs, 4H), 2.20 (s, 6H), 2.10 (s, 3H), 1.75 (m, 4H), 1.42 (m, 2H), 1.35 (s, 9H), 1.10 (m, 2H), 0.82 (t, 3H).

### Step 8:5-(((1r,4r)-4-aminocyclohexyl)(ethyl)amino)-N-((4,6-dimethyl-2-oxo-1,2-dihydropyridin-3-yl)methyl)-4-methyl-4′-(morpholinomethyl)-[1,1′-biphenyl]-3-carboxamide

To a stirred solution of tert-butyl ((1r,4r)-4-((5-(((4,6-dimethyl-2-oxo-1,2-dihydropyridin-3-yl)methyl)carbamoyl)-4-methyl-4′-(morpholinomethyl)-[1,1′-biphenyl]-3-yl)(ethyl)amino)cyclohexyl)carbamate (20 g, 0.03 mol) in DCM (200 mL) at 0° C., TFA (75 mL) was added and reaction was stirred for 2 h at room temperature. The reaction mixture was then concentrated to dryness and the residue basified with aqueous saturated bicarbonate solution (300 mL) to pH 8. The mixture was extracted with 20% methanol in DCM (4×200 m). The combined extracts were dried over Na₂SO₄ and the solvent removed under reduced pressure to afford 5-(((1r,4r)-4-aminocyclohexyl)(ethyl)amino)-N-((4,6-dimethyl-2-oxo-1,2-dihydropyridin-3-yl)methyl)-4-methyl-4′-(morpholinomethyl)-[1,1′-biphenyl]-3-carboxamide (15.5 g, 91%) which was used as is in the next reaction. ¹H NMR (DMSO-d₆, 400 MHZ) δ 8.18 (bs, 1H), 7.57 (d, 2H), 7.38 (m, 3H), 7.20 (s, 1H), 5.85 (s, 1H), 4.29 (d, 2H), 3.57 (bs, 4H), 3.48 (s, 2H), 3.31 (bs, 2H), 3.10 (m, 2H), 2.91 (m, 1H), 2.67 (m, 1H), 2.36 (bs, 4H), 2.21 (s, 3H), 2.20 (s, 3H), 2.10 (s, 3H), 1.90 (m, 2H), 1.83 (m, 2H), 1.45 (m, 2H), 1.23 (m, 2H), 0.83 (t, 3H).

### Step 9: N-((4,6-dimethyl-2-oxo-1,2-dihydropyridin-3-yl)methyl)-5-(((1r,4r)-4-(dimethylamino)cyclohexyl)(ethyl)amino)-4-methyl-4′-(morpholinomethyl)-[1,1′-biphenyl]-3-carboxamide

To a stirred solution of 5-(((1r,4r)-4-aminocyclohexyl)(ethyl)amino)-N-((4,6-dimethyl-2-oxo-1,2-dihydropyridin-3-yl)methyl)-4-methyl-4′-(morpholinomethyl)-[1,1′-biphenyl]-3-carboxamide (14 g, 0.023 mol) in dichloromethane (150 mL) was added aqueous 35% formaldehyde solution (2.4 g, 0.080 mol) at 0° C. After stirring for 20 min, Na(OAc)₃BH (12.2 g, 0.057 mol) was added and stirring continued for 2 h at 0° C. Water (100 mL) was then added to the reaction mixture and the mixture extracted with 20% methanol in DCM (3×200 mL). The combined extracts were dried over Na₂SO₄ and the solvent removed under reduced pressure. The crude product was purified by basic alumina column chromatography eluting with 6-7% MeOH in DCM to afford the title compound (10 g, 63.6%). LCMS: 614.65 (M+1)⁺; HPLC: 98.88% (@ 210-370 nm) (Rr; 3.724; Method: Column: YMC ODS-A 150 mm×4.6 mm×5μ; Mobile Phase: A; 0.05% TFA in water/B; 0.05% TFA in acetonitrile; Inj. Vol: 10 μL, Col. Temp.: 30° C.; Flow rate: 1.4 mL/min.; Gradient: 5% B to 95% B in 8 min, Hold for 1.5 min, 9.51-12 min 5% B); ¹H NMR (DMSO-d₆, 400 MHz) δ 11.45 (s, 1H), 8.17 (t, 1H), 7.56 (d, 2H, J=8 Hz), 7.36 (m, 3H), 7.17 (s, 1H), 5.85 (s, 1H), 4.29 (d, 2H, J=4.4 Hz), 3.57 (bs, 4H), 3.48 (s, 2H), 3.09 (q, 2H), 2.66 (m, 1H), 2.36 (bs, 4H), 2.21 (s, 3H), 2.20 (s, 3H), 2.11 (s, 9H), 1.79 (m, 4H), 1.36 (m, 2H), 1.11 (m, 2H), 0.82 (t, 3H, J=6.4&6.8 Hz).

### Example 7: Bioassay Protocol and General Methods

### Protocol for Wild-Type and Mutant PRC2 Enzyme Assays

General Materials.S-adenosylmethionine (SAM), S-adenosylhomocyteine (SAH), bicine, KCl, Tween20, dimethylsulfoxide (DMSO) and bovine skin gelatin (BSG) were purchased from Sigma-Aldrich at the highest level of purity possible. Dithiothreitol (DTT) was purchased from EMD. ³H-SAM was purchased from American Radiolabeled Chemicals with a specific activity of 80 Ci/mmol. 384-well streptavidin Flashplates were purchased from PerkinElmer.

Substrates. Peptides representative of human histone H3 residues 21-44 containing either an unmodified lysine 27 (H3K27me0) or dimethylated lysine 27 (H3K27me2) were synthesized with a C-terminal G (K-biotin) linker-affinity tag motif and a C-terminal amide cap by 21^{st} Century Biochemicals. The peptides were high-performance liquid chromatography (HPLC) purified to greater than 95% purity and confirmed by liquid chromatography mass spectrometry (LC-MS). The sequences are listed below.

H3K27me0:
(SEQ ID NO: 13)
ATKAARKSAPATGGVKKPHRYRPGGK(biotin)-amide
H3K27me2:
(SEQ ID NO: 14)
ATKAARK(me2)SAPATGGVKKPHRYRPGGK(biotin)-amide

Chicken erythrocyte oligonucleosomes were purified from chicken blood according to established procedures.

Recombinant PRC2 Complexes. Human PRC2 complexes were purified as 4-component enzyme complexes co-expressed in Spodoptera frugiperda (sf9) cells using a baculovirus expression system. The subunits expressed were wild-type EZH2 (NM_004456) or EZH2 Y641F, N, H, S or C mutants generated from the wild-type EZH2 construct, EED (NM_003797), Suz12 (NM_015355) and RbAp48 (NM_005610). The EED subunit contained an N-terminal FLAG tag that was used to purify the entire 4-component complex from sf9 cell lysates. The purity of the complexes met or exceeded 95% as determined by SDS-PAGE and Agilent Bioanalyzer analysis. Concentrations of enzyme stock concentrations (generally 0.3-1.0 mg/mL) was determined using a Bradford assay against a bovine serum albumin (BSA) standard.

General Procedure for PRC2 Enzyme Assays on Peptide Substrates. The assays were all performed in a buffer consisting of 20 mM bicine (pH=7.6), 0.5 mM DTT, 0.005% BSG and 0.002% Tween20, prepared on the day of use. Compounds in 100% DMSO (1 μL) were spotted into polypropylene 384-well V-bottom plates (Greiner) using a Platemate 2×3 outfitted with a 384-channel pipet head (Thermo). DMSO (1 μL) was added to columns 11, 12, 23, 24, rows A-H for the maximum signal control, and SAH, a known product and inhibitor of PRC2 (1 μL) was added to columns 11, 12, 23, 24, rows I-P for the minimum signal control. A cocktail (40 μL) containing the wild-type PRC2 enzyme and H3K27me0 peptide or any of the Y641 mutant enzymes and H3K27me2 peptide was added by Multidrop Combi (Thermo). The compounds were allowed to incubate with PRC2 for 30 min at 25° C., then a cocktail (10 μL) containing a mixture of non-radioactive and 3H-SAM was added to initiate the reaction (final volume=51 μL). In all cases, the final concentrations were as follows: wild-type or mutant PRC2 enzyme was 4 nM, SAH in the minimum signal control wells was 1 mM and the DMSO concentration was 1%. The final concentrations of the rest of the components are indicated in Table 7, below. The assays were stopped by the addition of non-radioactive SAM (10 μL) to a final concentration of 600 μM, which dilutes the ³H-SAM to a level where its incorporation into the peptide substrate is no longer detectable. 50 μL of the reaction in the 384-well polypropylene plate was then transferred to a 384-well Flashplate and the biotinylated peptides were allowed to bind to the streptavidin surface for at least 1 h before being washed three times with 0.1% Tween20 in a Biotek ELx405 plate washer. The plates were then read in a PerkinElmer TopCount platereader to measure the quantity of ³H-labeled peptide bound to the Flashplate surface, measured as disintegrations per minute (dpm) or alternatively, referred to as counts per minute (cpm).

TABLE 7
Final concentrations of components for each assay variation
based upon EZH2 identity (wild-type or Y641 mutant EZH2)

| PRC2 Enzyme | | | |
|---|---|---|---|
| (denoted by EZH2 | Peptide | Non-radioactive SAM | ³H-SAM |
| identity) | (nM) | (nM) | (nM) |
| Wild-type | 185 | 1800 | 150 |
| Y641F | 200 | 850 | 150 |
| Y641N | 200 | 850 | 150 |
| Y641H | 200 | 1750 | 250 |
| Y641S | 200 | 1300 | 200 |
| Y641C | 200 | 3750 | 250 |

General Procedure for Wild-Type PRC2 Enzyme Assay on Oligonucleosome Substrate. The assays was performed in a buffer consisting of 20 mM bicine (pH=7.6), 0.5 mM DTT, 0.005% BSG, 100 mM KCl and 0.002% Tween20, prepared on the day of use. Compounds in 100% DMSO (1 μL) were spotted into polypropylene 384-well V-bottom plates (Greiner) using a Platemate 2×3 outfitted with a 384-channel pipet head (Thermo). DMSO (1 μL) was added to columns 11, 12, 23, 24, rows A-H for the maximum signal control, and SAH, a known product and inhibitor of PRC2 (1 μL) was added to columns 11, 12, 23, 24, rows I-P for the minimum signal control. A cocktail (40 μL) containing the wild-type PRC2 enzyme and chicken erythrocyte oligonucleosome was added by Multidrop Combi (Thermo). The compounds were allowed to incubate with PRC2 for 30 min at 25° C., then a cocktail (10 μL) containing a mixture of non-radioactive and 3H-SAM was added to initiate the reaction (final volume=51 μL). The final concentrations were as follows: wild-type PRC2 enzyme was 4 nM, non-radioactive SAM was 430 nM, 3H-SAM was 120 nM, chicken erythrocyte olignonucleosome was 120 nM, SAH in the minimum signal control wells was 1 mM and the DMSO concentration was 1%. The assay was stopped by the addition of non-radioactive SAM (10 μL) to a final concentration of 600 μM, which dilutes the 3H-SAM to a level where its incorporation into the chicken erythrocyte olignonucleosome substrate is no longer detectable. 50 μL of the reaction in the 384-well polypropylene plate was then transferred to a 384-well Flashplate and the chicken erythrocyte nucleosomes were immobilized to the surface of the plate, which was then washed three times with 0.1% Tween20 in a Biotek ELx405 plate washer. The plates were then read in a PerkinElmer TopCount platereader to measure the quantity of 3H-labeled chicken erythrocyte oligonucleosome bound to the Flashplate surface, measured as disintegrations per minute (dpm) or alternatively, referred to as counts per minute (cpm).

$$\% Inhibition Calculation$$

$$\% inh = 100 - ( \frac{{dpm}_{{cmpd}^{-}} {dpm}_{min}}{{dpm}_{{max}^{-}} {dpm}_{min}} ) \times 100$$

Where dpm=disintegrations per minute, cmpd=signal in assay well, and min and max are the respective minimum and maximum signal controls.

$$Four - parameter {IC}_{50} fit$$

$$Y = Bottom + \frac{( Top - Bottom )}{1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

IC₅₀ values for the PRC2 enzyme assays on peptide substrates (e.g., EZH2 wild type andY641F) are presented in Table 8 below.

### WSU-DLCL2 Methylation Assay

WSU-DLCL2 suspension cells were purchased from DSMZ (German Collection of Microorganisms and Cell Cultures, Braunschweig, Germany). RPMI/Glutamax Medium, Penicillin-Streptomycin, Heat Inactivated Fetal Bovine Serum, and D-PBS were purchased from Life Technologies, Grand Island, NY, USA. Extraction Buffer and Neutralization Buffer (5×) were purchased from Active Motif, Carlsbad, CA, USA. Rabbit anti-Histone H3 antibody was purchased from Abcam, Cambridge, MA, USA. Rabbit anti-H3K27me3 and HRP-conjugated anti-rabbit-IgG were purchased from Cell Signaling Technology, Danvers, MA, USA. TMB “Super Sensitive” substrate was sourced from BioFX Laboratories, Owings Mills, MD, USA. IgG-free Bovine Serum Albumin was purchased from Jackson ImmunoResearch, West Grove, PA, USA. PBS with Tween (10× PBST) was purchased from KPL, Gaithersburg, MD, USA. Sulfuric Acid was purchased from Ricca Chemical, Arlington, TX, USA. Immulon ELISA plates were purchased from Thermo, Rochester, NY, USA. V-bottom cell culture plates were purchased from Corning Inc., Corning, NY, USA. V-bottom polypropylene plates were purchased from Greiner Bio-One, Monroe, NC, USA.

WSU-DLCL2 suspension cells were maintained in growth medium (RPMI 1640 supplemented with 10% v/v heat inactivated fetal bovine serum and 100 units/mL penicillin-streptomycin) and cultured at 37° C. under 5% CO₂. Under assay conditions, cells were incubated in Assay Medium (RPMI 1640 supplemented with 20% v/v heat inactivated fetal bovine serum and 100 units/mL penicillin-streptomycin) at 37° C. under 5% CO₂ on a plate shaker.

WSU-DLCL2 cells were seeded in assay medium at a concentration of 50,000 cells per mL to a 96-well V-bottom cell culture plate with 200 μL per well. Compound (1 μL) from 96 well source plates was added directly to V-bottom cell plate. Plates were incubated on a titer-plate shaker at 37° C., 5% CO₂ for 96 hours. After four days of incubation, plates were spun at 241×g for five minutes and medium was aspirated gently from each well of cell plate without disturbing cell pellet. Pellet was resuspended in 200 μL DPBS and plates were spun again at 241×g for five minutes. The supernatant was aspirated and cold (4° C.) Extraction buffer (100 μL) was added per well. Plates were incubated at 4° C. on orbital shaker for two hours. Plates were spun at 3427×g×10 minutes. Supernatant (80 μL per well) was transferred to its respective well in 96 well V-bottom polypropylene plate. Neutralization Buffer 5× (20 μL per well) was added to V-bottom polypropylene plate containing supernatant. V-bottom polypropylene plates containing crude histone preparation (CHP) were incubated on orbital shaker×five minutes. Crude Histone Preparations were added (2 μL per well) to each respective well into duplicate 96 well ELISA plates containing 100 μL Coating Buffer (1× PBS+BSA 0.05% w/v). Plates were sealed and incubated overnight at 4° C. The following day, plates were washed three times with 300 μL per well 1× PBST. Wells were blocked for two hours with 300 μL per well ELISA Diluent ((PBS (1×) BSA (2% w/v) and Tween20 (0.05% v/v)). Plates were washed three times with 1×PBST. For the Histone H3 detection plate, 100 μL per well were added of anti-Histone-H3 antibody (Abcam, ab1791) diluted 1:10,000 in ELISA Diluent. For H3K27 trimethylation detection plate, 100 μL per well were added of anti-H3K27me3 diluted 1:2000 in ELISA diluent. Plates were incubated for 90 minutes at room temperature. Plates were washed three times with 300 μL 1× PBST per well. For Histone H3 detection, 100 μL of HRP-conjugated anti-rabbit IgG antibody diluted to 1:6000 in ELISA diluent was added per well. For H3K27me3 detection, 100 μL of HRP conjugated anti-rabbit IgG antibody diluted to 1:4000 in ELISA diluent was added per well. Plates were incubated at room temperature for 90 minutes. Plates were washed four times with 1×PBST 300 μL per well. TMB substrate 100 μL was added per well. Histone H3 plates were incubated for five minutes at room temperature. H3K27me3 plates were incubated for 10 minutes at room temperature. The reaction was stopped with sulfuric acid 1N (100 μL per well). Absorbance for each plate was read at 450 nm.

First, the ratio for each well was determined by:

$$( \frac{H 3 K 27 me 3 OD 450 value}{Histone H 3 OD 450 value} )$$

Each plate included eight control wells of DMSO only treatment (Minimum Inhibition) as well as eight control wells for maximum inhibition (Background wells).

The average of the ratio values for each control type was calculated and used to determine the percent inhibition for each test well in the plate. Test compound was serially diluted three-fold in DMSO for a total of ten test concentrations, beginning at 25 μM. Percent inhibition was determined and IC₅₀ curves were generated using duplicate wells per concentration of compound. IC₅₀ values for this assay are presented in Table 8 below.

$$Perfect Inhibition = 100 - ( ( \frac{( Individual Test Sample Ratio ) - ( Back g round Avg Ratio )}{( Minimum Inhibition Ratio ) - ( Back g round Average Ratio )} ) * 100 )$$

### Cell Proliferation Analysis

WSU-DLCL2 suspension cells were purchased from DSMZ (German Collection of Microorganisms and Cell Cultures, Braunschweig, Germany). RPMI/Glutamax Medium, Penicillin-Streptomycin, Heat Inactivated Fetal Bovine Serum were purchased from Life Technologies, Grand Island, NY, USA. V-bottom polypropylene 384-well plates were purchased from Greiner Bio-One, Monroe, NC, USA. Cell culture 384-well white opaque plates were purchased from Perkin Elmer, Waltham, MA, USA. Cell-Titer Glo® was purchased from Promega Corporation, Madison, WI, USA. SpectraMax M5 plate reader was purchased from Molecular Devices LLC, Sunnyvale, CA, USA.

WSU-DLCL2 suspension cells were maintained in growth medium (RPMI 1640 supplemented with 10% v/v heat inactivated fetal bovine serum and cultured at 37° C. under 5% CO₂. Under assay conditions, cells were incubated in Assay Medium (RPMI 1640 supplemented with 20% v/v heat inactivated fetal bovine serum and 100 units/mL penicillin-streptomycin) at 37° C. under 5% CO₂.

For the assessment of the effect of compounds on the proliferation of the WSU-DLCL2 cell line, exponentially growing cells were plated in 384-well white opaque plates at a density of 1250 cell/ml in a final volume of 50 μl of assay medium. A compound source plate was prepared by performing triplicate nine-point 3-fold serial dilutions in DMSO, beginning at 10 mM (final top concentration of compound in the assay was 20 μM and the DMSO was 0.2%). A 100 nL aliquot from the compound stock plate was added to its respective well in the cell plate. The 100% inhibition control consisted of cells treated with 200 nM final concentration of staurosporine and the 0% inhibition control consisted of DMSO treated cells. After addition of compounds, assay plates were incubated for 6 days at 37° C., 5% CO₂, relative humidity >90% for 6 days. Cell viability was measured by quantization of ATP present in the cell cultures, adding 35 μl of CellTiter-Glo®® reagent to the cell plates. Luminescence was read in the SpectraMax M5. The concentration inhibiting cell viability by 50% was determined using a 4-parametric fit of the normalized dose response curves.

### INCORPORATION BY REFERENCE

The entire disclosure of each of the patent documents and scientific articles referred to herein is incorporated by reference for all purposes.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples above are for purposes of illustration and not limitation of the claims that follow.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method for treating or alleviating a symptom of a SWI/SNF-associated cancer in a subject comprising administering to a subject in need thereof a therapeutically effective amount of an EZH2 inhibitor, wherein the subject has a cancer selected from the group consisting of brain and central nervous system cancer, head and neck cancer, kidney cancer, ovarian cancer, pancreatic cancer, leukemia, lung cancer, lymphoma, myeloma, sarcoma, breast cancer, and prostate cancer.

2. The method of claim 1, wherein the SWI/SNF-associated cancer is characterized by reduced expression or loss of function of the SWI/SNF complex or one or more components of the SWI/SNF complex.

3. The method of claim 1, wherein the subject has a cancer selected from the group consisting of medulloblastoma, malignant rhabdoid tumor and atypical teratoid/rhabdoid tumor.

4. The method of claim 2, wherein the one or more components are selected from the group consisting of SNF5, ATRX and ARID1A.

5. The method of claim 2, wherein the loss of function is caused by a loss of function mutation resulting from a point mutation, a deletion and/or an insertion.

6. The method of claim 1, wherein the subject has a deletion of SNF5.

7. The method of claim 1, wherein the subject has a mutation of ATRX selected from the group consisting of a substitution of asparagine (N) for the wild type residue lysine (K) at amino acid position 688 of SEQ ID NO: 5 (K688N), and a substitution of isoleucine (I) for the wild type residue methionine (M) at amino acid position 366 of SEQ ID NO: 5 (M366I).

8. The method of claim 1, wherein said subject has a mutation of ARID1A selected from the group consisting of a nonsense mutation for the wild type residue cysteine (C) at amino acid position 884 of SEQ ID NO: 11 (C884*), a substitution of lysine (K) for the wild type residue glutamic acid (E) at amino acid position 966 (E966K), a nonsense mutation for the wild type residue glutamine (Q) at amino acid position 1411 of SEQ ID NO: 11 (Q1411*), a frame shift mutation at the wild type residue phenylalanine (F) at amino acid position 1720 of SEQ ID NO: 11 (F1720fs), a frame shift mutation after the wild type residue glycine (G) at amino acid position 1847 of SEQ ID NO: 11 (G1847fs), a frame shift mutation at the wild type residue cysteine (C) at amino acid position 1874 of SEQ ID NO: 11 (C1874fs), a substitution of glutamic acid (E) for the wild type residue aspartic acid (D) at amino acid position 1957 (D1957E), a nonsense mutation for the wild type residue glutamine (Q) at amino acid position 1430 of SEQ ID NO: 11 (Q1430*), a frame shift mutation at the wild type residue arginine (R) at amino acid position 1721 of SEQ ID NO: 11 (R₁₇₂₁fs), a substitution of glutamic acid (E) for the wild type residue glycine (G) at amino acid position 1255 (G1255E), a frame shift mutation at the wild type residue glycine (G) at amino acid position 284 of SEQ ID NO: 11 (G284fs), a nonsense mutation for the wild type residue arginine (R) at amino acid position 1722 of SEQ ID NO: 11 (R1722*), a frame shift mutation at the wild type residue methionine (M) at amino acid position 274 of SEQ ID NO: 11 (M274fs), a frame shift mutation at the wild type residue glycine (G) at amino acid position 1847 of SEQ ID NO: 11 (G1847fs), a frame shift mutation at the wild type residue P at amino acid position 559 of SEQ ID NO: 11 (P559fs), a nonsense mutation for the wild type residue arginine (R) at amino acid position 1276 of SEQ ID NO: 11 (R1276*), a frame shift mutation at the wild type residue glutamine (Q) at amino acid position 2176 of SEQ ID NO: 11 (Q2176fs), a frame shift mutation at the wild type residue histidine (H) at amino acid position 203 of SEQ ID NO: 11 (H203fs), a frame shift mutation at the wild type residue alanine (A) at amino acid position 591 of SEQ ID NO: 11 (A591fs), a nonsense mutation for the wild type residue glutamine (Q) at amino acid position 1322 of SEQ ID NO: 11 (Q1322*), a nonsense mutation for the wild type residue serine(S) at amino acid position 2264 of SEQ ID NO: 11 (S2264*), a nonsense mutation for the wild type residue glutamine (Q) at amino acid position 586 of SEQ ID NO: 11 (Q586*), a frame shift mutation at the wild type residue glutamine (Q) at amino acid position 548 of SEQ ID NO: 11 (Q548fs), and a frame shift mutation at the wild type residue asparagine (N) at amino acid position 756 of SEQ ID NO: 11 (N756fs).

9. A method of treating or alleviating a symptom of a SWI/SNF-associated cancer in a subject in need thereof comprising:
a. determining the expression level of at least one gene selected from the group consisting of neuronal differentiation genes, cell cycle inhibition genes and tumor suppressor genes in a sample obtained from the subject;
b. selecting a subject having a decreased expression level of at least one gene in step a; and
c. administering to the subject selected in step b an effective amount of an EZH2 inhibitor, thereby treating or alleviating a symptom of cancer in the subject.

10. A method of treating or alleviating a symptom of a SWI/SNF-associated cancer in a subject in need thereof comprising:
a. determining the expression level of at least one gene selected from the group consisting of hedgehog pathway genes, myc pathway genes and histone methyltransferase genes in a sample obtained from the subject,
b. selecting a subject having an increased expression level of at least one gene in step a; and
c. administering to the subject selected in step b an effective amount of an EZH2 inhibitor, thereby treating or alleviating a symptom of cancer in the subject.

11. The method of claim 9, wherein the cancer is selected from the group consisting of medulloblastoma, malignant rhabdoid tumor, and atypical teratoid rhabdoid tumor.

12. The method of claim 10, wherein the cancer is selected from the group consisting of medulloblastoma, malignant rhabdoid tumor, and atypical teratoid rhabdoid tumor.

13. The method of claim 9, wherein the neuronal differentiation gene is CD133, DOCK4, or PTPRK.

14. The method of claim 9, wherein the cell cycle inhibition gene is CKDNIA or CDKN2A.

15. The method of claim 9, wherein the tumor suppressor gene is BIN1.

16. The method of claim 10, wherein the hedgehog pathway gene is GLI1 or PTCH1.

17. The method of claim 10, wherein the myc pathway gene is MYC.

18. The method of claim 10, wherein the histone methyltransferase gene is EZH2.

19-32. (canceled)

33. The method of claim 1, wherein the EZH2 inhibitor is: 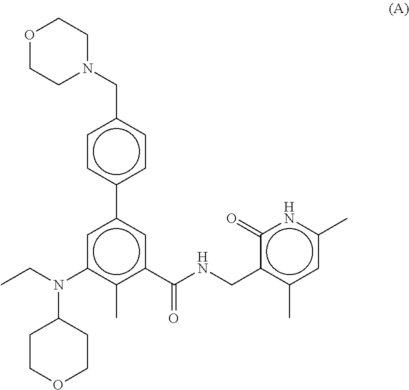
or pharmaceutically acceptable salt thereof.

34. (canceled)

35. The method of claim 9, wherein the EZH2 inhibitor is: 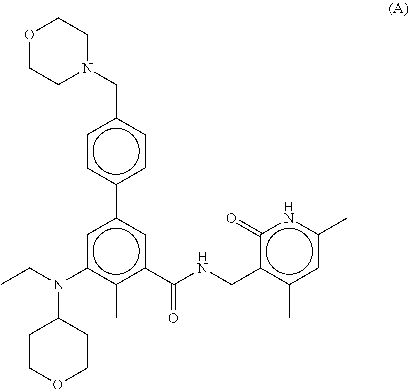
or pharmaceutically acceptable salt thereof.
